SEQUENCE LISTING (1650 sequences)

SEQ ID NO: 1-1507 - skipped (1507 entries)

SEQ ID NO: 1508 - DNA, 250 bp, Homo sapiens
ccggtcccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaag

SEQ ID NO: 1509 - DNA, 250 bp, Homo sapiens
gcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacg

SEQ ID NO: 1510 - DNA, 250 bp, Homo sapiens
tcccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtag

SEQ ID NO: 1511 - DNA, 250 bp, Homo sapiens
cggcgcgctcgccggccgaggtgggatcccgaggcctctccagtccgccgagggcgcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctag

SEQ ID NO: 1512 - DNA, 250 bp, Homo sapiens
gcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagc

SEQ ID NO: 1513 - DNA, 250 bp, Homo sapiens
gtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccg

SEQ ID NO: 1514 - DNA, 250 bp, Homo sapiens
cgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactc

SEQ ID NO: 1515 - DNA, 250 bp, Homo sapiens
ccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccc

SEQ ID NO: 1516 - DNA, 250 bp, Homo sapiens
tagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgcc

SEQ ID NO: 1517 - DNA, 250 bp, Homo sapiens
tgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtccc

SEQ ID NO: 1518 - DNA, 250 bp, Homo sapiens
tagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccct

SEQ ID NO: 1519 - DNA, 250 bp, Homo sapiens
ccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagcc

SEQ ID NO: 1520 - DNA, 250 bp, Homo sapiens
gcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagt

SEQ ID NO: 1521 - DNA, 250 bp, Homo sapiens
ggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggat

SEQ ID NO: 1522 - DNA, 19 bp, Homo sapiens
gtctacggccataccaccc

SEQ ID NO: 1523 - DNA, 38 bp, Homo sapiens
atcgcttctcggccttttggctaagatcaagtgtagta

SEQ ID NO: 1524 - DNA, 250 bp, Homo sapiens
acctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactctgatcgttttttcactgacccggtgaggcgggggg

SEQ ID NO: 1525 - DNA, 250 bp, Homo sapiens
aaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctg

SEQ ID NO: 1526 - DNA, 250 bp, Homo sapiens
ggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgag

SEQ ID NO: 1527 - DNA, 250 bp, Homo sapiens
aactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggc

SEQ ID NO: 1528 - DNA, 102 bp, Homo sapiens
tgaacgcgcccgatctcgtctgatctcggaagctaagcagggtcgggcctggttagtacttggatgggagaccgcctgggaataccgggtgctgtaggcttt

SEQ ID NO: 1529 - DNA, 150 bp, Homo sapiens
tctgttcttatcagtttaatatctgatacgtcctctatccgaggacaatatattaaatggatttttggagcagggagatggaataggagcttgctccgtccactccacgcatcgacctggtattgcagtacctccaggaacggtgcaccc

SEQ ID NO: 1530 - protein, 340 aa, Artificial Sequence
  Description of Artificial Sequence: Synthetic polypeptide
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 1531 - protein, 340 aa, Artificial Sequence
  Description of Artificial Sequence: Synthetic polypeptide
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEASKPKNTIPTVKHGGGSIMLWGCFAAGGTGALHKIDGSMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 1532 - protein, 594 aa, Artificial Sequence
  Description of Artificial Sequence: Synthetic polypeptide
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 1533 - protein, 602 aa, Artificial Sequence
  Description of Artificial Sequence: Synthetic polypeptide
MTMDRVEKNVKKRKYSEDFLQYGFTSIITAGIEKPQCVICCEVLSAESMKPNKLKRHFDSKHPSFAGKDTNYFRSKADGLKKARLDTGGKYHKQNVAAIEASYLVALRIARAMKPHTIAEDLLLPAAKDIVRVMIGDEFVTKLSAISLSNDTVRRRIDDMSADILDQVIQEIKSAPLPIFSIQLDESTDVANCSQLLVYVRYINDGDFKDEFLFCKPLEMTTTARDVFDTVGSFLKEHKISWEKVCGVCTDGAPAMLGCRSGFQRLVLNESPKVIGTHCMIHRQILATKTLPQELQEVMKSVISSVNFVKASTLNSRLFSQLCNELDAPNNALLFHTEVRWLSRGKVLKRVFELRDELKTFFNQKARPQFEALFSDKSELQKIAYLVDIFAILNELNLSLQGPNATCLDLSEKIRSFQMKLQLWQKKLDENKIYMLPTLSAFFEEHDIEPDKRITMIISVKEHLHMLADEISSYFPNLPDTPFALARSPFTVKVEDVPETAQEEFIELINSDAARTDFSTMPVTKFWIKCLQSYPVLSETVLRLLLPFPTTYLCETGFSSLLVIKSKYRSRLVVEDDLRCALAKTAPRISDLVRKKQSQPSH

SEQ ID NO: 1534 - protein, 574 aa, Artificial Sequence
  Description of Artificial Sequence: Synthetic polypeptide
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNYLE

SEQ ID NO: 1535 - protein, 636 aa, Artificial Sequence
  Description of Artificial Sequence: Synthetic polypeptide
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYAATKTKYRNRLDAEDDMRLQLTTIHPDIDNLCNNKQAQKSH

SEQ ID NO: 1536 - protein, 9 aa, Unknown
  Description of Unknown: LAGLIDADG family peptide sequence
LAGLIDADG

SEQ ID NO: 1537 - DNA, 4183 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctaacatgtgcgacgtagcttgggtaggtgagcgattaaccgtccctttaggtaccactagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgataatacgactcagctagcgtttaaacttaagcttgagctcggatccccagtgtggtggaattcgccaccatggtttccgagctgatcaaagaaaacatgcacatgaagctgtacatggaaggcaccgtgaacaaccaccacttcaagtgcaccagcgaaggcgagggcaagccttatgagggcacccagaccatgagaatcaaggccgttgaaggcggccctctgcctttcgcctttgatatcctggccaccagctttatgtacggcagcaagaccttcatcaatcacacccagggcatccccgatttcttcaagcagagcttccccgagggcttcacctgggagagagtgaccacatacgaggatggcggcgtgctgacagccacacaggatacaagtctgcaggacggctgcctgatctacaacgtgaagatccggggcgtgaacttccccagcaatggccccgtgatgcagaagaaaaccctcggctgggaagccagcaccgagacactgtatcctgccgatggtggcctggaaggcagagctgatatggccctgaaactcgttggcggcggacacctgatctgcaatctgaaaaccacctaccggtccaagaagcccgccaagaacctgaagatgcccggcgtgtactacgtggacagacggctggaacggatcaaagaggccgacaaagaaacctacgtggaacagcacgaggtggccgtggccagatactgtgatctgccttctaagctgggccacagatgataatctagagtcgacctgcagaagcttgatatcaccggtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttccggagcggccgcgtttaattgagttgtcatatgttaataacggtatgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctactgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1538 - DNA, 4217 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccattcggtacaattcacgcgtgagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctaacatgtgcgacgtagcttgggtaggtgagcgattaaccgtccctttagggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctggccgccaccatggagatgctccaggggctgctgctgtggctgctgctgagcatgggcggggcacgggcatccagggagccgctgcggccactgtgccgccccatcaatgccaccctggctgccgagaaggaggcctgccccgtgtgcatcaccgtcaacaccaccatctgtgccggctactgccccaccatgatgcgggtgctgcaggcggtcctgccgccagtgccccaggtggtgcgcaactaccgcgaggtgcgcttcgagtccatccggctccctggctgcccgcctggcgtggaccccgtggtctccgttcccgtggctctcagctgtcgttgtgcactctgccgccgcagcacctctgactgtgggggtcccaaggaccaccctttgacctgtgatgacccccacctccaggcctcctcttcctcaaaggaccctccccccagccctccaagtccatccggactcctggagccagcagacaacccgttcctcccgcaataaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtttaattgagttgtcatatgttaataacggtatgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatatctagatccggagagctcctcgaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1539 - DNA, 3752 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccattcggtacaattcacgcgtgagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgatccttgactgtgcctttaaacagcttggaaaattccagaaaatgatgtcatggctttagaagctaacatgtgcgacgtagcttgggtaggtgagcgattaaccgtccctttagggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctggccgccaccatggagatgctccaggggctgctgctgtggctgctgctgagcatgggcggggcacgggcatccagggagccgctgcggccactgtgccgccccatcaatgccaccctggctgccgagaaggaggcctgccccgtgtgcatcaccgtcaacaccaccatctgtgccggctactgccccaccatgatgcgggtgctgcaggcggtcctgccgccagtgccccaggtggtgcgcaactaccgcgaggtgcgcttcgagtccatccggctccctggctgcccgcctggcgtggaccccgtggtctccgttcccgtggctctcagctgtcgttgtgcactctgccgccgcagcacctctgactgtgggggtcccaaggaccaccctttgacctgtgatgacccccacctccaggcctcctcttcctcaaaggaccctccccccagccctccaagtccatccggactcctggagccagcagacaacccgttcctcccgcaataaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtttaattgagttgtcatatgttaataacggtatgtggaaggctactcgaaatgtttgacccaagttaaacaatttaaaggcaatgctaccaaatactaatctagatccggagagctcctcgaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1540 - DNA, 3113 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccattcggtacaattcacgcgtctaagttaattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgcggccgctctagaactagtggatcccccgggctgcaggaattcactagtgatttcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1541 - DNA, 3992 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccattcggtacaattcacgcgtgagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgatccttgactgtgcctttaaacagcttggaaaattccagaaaatgatgtcatggctttagaagctaacatgtgcgacgtagcttgggtaggtgagcgattaaccgtccctttagggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtttaattgagttgtcatatgttaataacggtatgtggaaggctactcgaaatgtttgacccaagttaaacaatttaaaggcaatgctaccaaatactaatctagatccggagagctcctcgaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1542 - protein, 16 aa, Artificial Sequence
  Description of Artificial Sequence: Synthetic peptide
SGSETPGTSESATPES

SEQ ID NO: 1543 - protein, 4 aa, Artificial Sequence
  Description of Artificial Sequence: Synthetic peptide
SGGS

SEQ ID NO: 1544 - protein, 4 aa, Artificial Sequence
  Description of Artificial Sequence: Synthetic peptide
GGGS

SEQ ID NO: 1545 - protein, 5 aa, Artificial Sequence
  Description of Artificial Sequence: Synthetic peptide
GGGGS

SEQ ID NO: 1546 - protein, 5 aa, Artificial Sequence
  Description of Artificial Sequence: Synthetic peptide
EAAAK

SEQ ID NO: 1547 - protein, 1367 aa, Artificial Sequence
  Description of Artificial Sequence: Synthetic polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1548 - protein, 1369 aa, Artificial Sequence
  Description of Artificial Sequence: Synthetic polypeptide
SMDKKYSIGLAIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1549 - DNA, 76 bp, Artificial Sequence
  Description of Artificial Sequence: Synthetic oligonucleotide
gttttagagctagaaatagcaagttaaaataaggctagtccgttatcaacttgaaaaagtgggaccgagtcggtcc

SEQ ID NO: 1550 - protein, 30 aa, Artificial Sequence
  Description of Artificial Sequence: Synthetic polypeptide
MDSLLMNRRKFLYQFKNVRWAKGRRETYLC

SEQ ID NO: 1551 - protein, 28 aa, Artificial Sequence
  Description of Artificial Sequence: Synthetic peptide
PKKRKVEGADKRTADGSEFESPKKKRKV

SEQ ID NO: 1552 - protein, 22 aa, Artificial Sequence
  Description of Artificial Sequence: Synthetic peptide
RKSGKIAAIWKRPRKPKKKRKV

SEQ ID NO: 1553 - protein, 18 aa, Artificial Sequence
  Description of Artificial Sequence: Synthetic peptide
KRTADGSEFESPKKKRKV

SEQ ID NO: 1554 - protein, 17 aa, Artificial Sequence
  Description of Artificial Sequence: Synthetic peptide
KKTELQTTNAENKTKKL

SEQ ID NO: 1555 - protein, 19 aa, Artificial Sequence
  Description of Artificial Sequence: Synthetic peptide
KRGINDRNFWRGENGRKTR

SEQ ID NO: 1556 - protein, 16 aa, Artificial Sequence
  Description of Artificial Sequence: Synthetic peptide
KRPAATKKAGQAKKKK

SEQ ID NO: 1557 - protein, 29 aa, Artificial Sequence
  Description of Artificial Sequence: Synthetic peptide
PKKKRKVEGADKRTADGSEFESPKKKRKV

SEQ ID NO: 1558 - DNA, 306 bp, Artificial Sequence
  Description of Artificial Sequence: Synthetic polynucleotide
tgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaat

SEQ ID NO: 1559 - protein, 102 aa, Artificial Sequence
  Description of Artificial Sequence: Synthetic polypeptide
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 1560 - DNA, 108 bp, Artificial Sequence
  Description of Artificial Sequence: Synthetic polynucleotide
atgatcaagatagctacaaggaagtatcttggcaaacaaaacgtttatgatattggagtcgaaagagatcacaactttgctctgaagaacggattcatagcttctaat

SEQ ID NO: 1561 - protein, 36 aa, Artificial Sequence
  Description of Artificial Sequence: Synthetic polypeptide
MIKIATRKYLGKQNVYDIGVERDHNFALKNGFIASN

SEQ ID NO: 1562 - DNA, 303 bp, Artificial Sequence
  Description of Artificial Sequence: Synthetic polynucleotide
tgcctgtcttatgataccgagatacttaccgttgaatatggcttcttgcctattggaaagattgtcgaagagagaattgaatgcacagtatatactgtagacaagaatggtttcgtttacacacagcccattgctcaatggcacaatcgcggcgaacaagaagtatttgagtactgtctcgaggatggaagcatcatacgagcaactaaagatcataaattcatgaccactgacgggcagatgttgccaatagatgagatattcgagcggggcttggatctcaaacaagtggatggattgcca

SEQ ID NO: 1563 - protein, 101 aa, Artificial Sequence
  Description of Artificial Sequence: Synthetic polypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 1564 - DNA, 159 bp, Artificial Sequence
  Description of Artificial Sequence: Synthetic polynucleotide
atgaagaggactgccgatggatcagagtttgaatctcccaagaagaagaggaaagtaaagataatatctcgaaaaagtcttggtacccaaaatgtctatgatattggagtggagaaagatcacaacttccttctcaagaacggtctcgtagccagcaac

SEQ ID NO: 1565 - protein, 53 aa, Artificial Sequence
  Description of Artificial Sequence: Synthetic polypeptide
MKRTADGSEFESPKKKRKVKIISRKSLGTQNVYDIGVEKDHNFLLKNGLVASN

SEQ ID NO: 1566 - DNA, 62 bp, Unknown
  Description of Unknown: Complement factor 3 (C3) sequence
cactcctccccatcctctccctctgtccctctgtccctctgaccctgcactgtcccagcacc

SEQ ID NO: 1567 - DNA, 122 bp, Unknown
  Description of Unknown: Orosomucoid 1 (ORM1) sequence
caggacacagccttggatcaggacagagacttgggggccatcctgcccctccaacccgacatgtgtacctcagctttttccctcacttgcatcaataaagcttctgtgtttggaacagctaa

SEQ ID NO: 1568 - RNA, 47 nt, Artificial Sequence
  Description of Artificial Sequence: Synthetic oligonucleotide
gggaaauaagagagaaaagaagaguaagaagaaauauaagagccacc

SEQ ID NO: 1569 - RNA, 110 nt, Artificial Sequence
  Description of Artificial Sequence: Synthetic polynucleotide
ugauaauaggcuggagccucgguggccaugcuucuugccccuugggccuccccccagccccuccuccccuuccugcacccguacccccguggucuuugaauaaagucuga

SEQ ID NO: 1570 - DNA, 26 bp, Homo sapiens
ctgacccttctctctctcccccagag

SEQ ID NO: 1571 - DNA, 15 bp, Homo sapiens
tttctctcccacaag

SEQ ID NO: 1572 - RNA, 21 nt, Homo sapiens
cauaaaguagaaagcacuacu

SEQ ID NO: 1573 - RNA, 23 nt, Homo sapiens
uguaguguuuccuacuuuaugga

SEQ ID NO: 1574 - RNA, 24 nt, Homo sapiens
uuuggcaaugguagaacucacacu

SEQ ID NO: 1575 - RNA, 21 nt, Homo sapiens
ugguucuagacuugccaacua

SEQ ID NO: 1576 - RNA, 22 nt, Homo sapiens
uauggcacugguagaauucacu

SEQ ID NO: 1577 - RNA, 22 nt, Homo sapiens
gugaauuaccgaagggccauaa

SEQ ID NO: 1578 - RNA, 22 nt, Homo sapiens
uggagugugacaaugguguuug

SEQ ID NO: 1579 - RNA, 22 nt, Homo sapiens
aacgccauuaucacacuaaaua

SEQ ID NO: 1580 - DNA, 12 bp, Homo sapiens
ctggggctgggg

SEQ ID NO: 1581 - DNA, 12 bp, Unknown
  Description of Unknown: Myoclonic epilepsy of Unverricht and Lundborg repeat sequence
ccccgccccgcg

SEQ ID NO: 1582 - DNA, 16 bp, Adeno-associated virus 2
gcgcgctcgctcgctc

SEQ ID NO: 1583 - DNA, 628 bp, Artificial Sequence
  Description of Artificial Sequence: Synthetic polynucleotide
agatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagct

SEQ ID NO: 1584 - DNA, 1122 bp, Artificial Sequence
  Description of Artificial Sequence: Synthetic polynucleotide
gttaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcctcgacggtatcgataagcttgatatcgaattctagtcgtcgaccactttcacaatctgctagcaacctgaggaggttatcgtacgaaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatcgatctgaccatacacttgagtgacaatgacatccactttgcctttctctccacaggtgtccactcccaggtccaac

SEQ ID NO: 1585 - DNA, 535 bp, Artificial Sequence
  Description of Artificial Sequence: Synthetic polynucleotide
caaatgacttagtttggctaaaatgtaggcttttaaaaatgtgagcactgccaagggtttttccttgttgacccatggatccatcaagtgcaaacattttctaatgcactatatttaagcctgtgcagctagatgtcattcaacatgaaatacattattacaacttgcatctgtctaaaatcttgcatctaaaatgagagacaaaaaatctataaaaatggaaaacatgcatagaaatatgtgagggaggaaaaaattacccccaagaatgttagtgcacgcagtcacacagggagaagactatttttgttttgttttgattgttttgttttgttttggttgttttgttttggtgacctaactggtcaaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcagg

SEQ ID NO: 1586 - DNA, 2428 bp, Artificial Sequence
  Description of Artificial Sequence: Synthetic polynucleotide
ccaccgcggtggcggccgctctagcttccttagcatgacgttccacttttttctaaggtggagcttacttctttgatttgatcttttgtgaaacttttggaaattacccatcttcctaagcttctgcttctctcagttttctgcttgctcattccacttttccagctgaccctgccccctaccaacattgctccacaagcacaaattcatccagagaaaataaattctaagttttatagttgtttggatcgcataggtagctaaagaggtggcaacccacacatccttaggcatgagcttgattttttttgatttagaaccttcccctctctgttcctagactacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcactgtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagaaagggtatagatataaaaattaaaactaatgaaatggcagtcttacacatttttggcagcttatttaaagtcttggtgttaagtacgctggagctgtcacagctaccaatcaggcatgtctgggaatgagtacacggggaccataagttactgacattcgtttcccattccatttgaatacacacttttgtcatggtattgcttgctgaaattgttttgcaaaaaaaaccccttcaaattcatatatattattttaataaatgaattttaatttatctcaatgttataaaaaagtcaattttaataattaggtacttatatacccaataatatctaacaatcatttttaaacatttgtttattgagcttattatggatgaatctatctctatatactctatatactctaaaaaagaagaaagaccatagacaatcatctatttgatatgtgtaaagtttacatgtgagtagacatcagatgctccatttctcactgtaataccatttatagttacttgcaaaactaactggaattctaggacttaaatattttaagttttagctgggtgactggttggaaaattttaggtaagtactgaaaccaagagattataaaacaataaattctaaagttttagaagtgatcataatcaaatattaccctctaatgaaaatattccaaagttgagctacagaaatttcaacataagataattttagctgtaacaatgtaatttgttgtctattttcttttgagatacagttttttctgtctagctttggctgtcctggaccttgctctgtagaccaggttggtcttgaactcagagatctgcttgcctctgccttgcaagtgctaggattaaaagcatgtgccaccactgcctggctacaatctatgttttataagagattataaagctctggctttgtgacattaatctttcagataataagtcttttggattgtgtctggagaacatacagactgtgagcagatgttcagaggtatatttgcttaggggtgaattcaatctgcagcaataattatgagcagaattactgacacttccattttatacattctacttgctgatctatgaaacatagataagcatgcaggcattcatcatagttttctttatctggaaaaacattaaatatgaaagaagcactttattaatacagtttagatgtgttttgccatcttttaatttcttaagaaatactaagctgatgcagagtgaagagtgtgtgaaaagcagtggtgcagcttggcttgaactcgttctccagcttgggatcgacctgcaggcatgcttccatgccaaggcccacactgaaatgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaaccccgggatcccccgggctgcaggaattcgatatcaagcttatcgataccgtcgacctcgagggggggcccggtac

SEQ ID NO: 1587 - DNA, 1437 bp, Artificial Sequence
  Description of Artificial Sequence: Synthetic polynucleotide
ccgggcgtggtggcgcatgtctgtaatcccagctacttgggatgctgaggcaggagaatccttgaacccgggaggtggaggttgcagtgagccgagatcatgccattacgctccagcctgagcaacaagagcaaaactccgtctcaggaaaacaaacaaaaaaacctgcacatatacttctgaatttaaaacaaaagttaaaaaacaaagatttcttggtctctggtcactacctccctcatcagctttgcgcctccactgtcaccctcaggaatgttccacatactcagcgagtatgcttggggggcaaaagggtgaaagatacaaaagcttctgatatctatttaactgatttcacccaaatgctttgaacctgggaatgtacctctccccctcccccacccccaacaggagtgagacaagggccagggctattgcccctgctgactcaatattggctaatcactgcctagaactgataaggtgatcaaatgaccaggtgccttcaacctttaccctggtagaagcctcttattcacctcttttcctgccagagccctccattgggaggggacgggcggaagctgttttctgaatttgttttactgggggtagggtatgttcagtgatcagcatccaggtcattctgggctctcctgttttctccccgtctcattacacattaactcaaaaacggacaagatcatttacacttgccctcttacccgaccctcattcccctaacccccatagccctcaaccctgtccctgatttcaattcctttctcctttcttctgctccccaatatctctctgccaagttgcagtaaagtgggataaggttgagagatgagatctacccataatggaataaagacaccatgagctttccatggtatgatgggttgatggtattccatgggttgatatgtcagagctttccagagaaataacttggaatcctgcttcctgttgcactcaagtccaaggacctcagatctcaaaagaatgaacctcaaatatacctgaagtgtacccccttagcctccactaagagctgtaccccctgcctctcaccccatcaccatgagtcttccatgtgcttgtcctctcctcccccatttctccaacttgtttatcctcacataatccctgccccactgggcccatccatagtccctgtcacctgacagggggtgggtaaacagacaggtatatagccccttcctctccagccagggcaggcacagacaccaaggacagagacgctggctaggtaagataaggaggcaagatgtgtgagcagcatccaaagaggcctgggcttcagttgtggagagggagagagccaggttggaatgggcagcaggtagggagatccctggggaggagctgaagcccatttggcttcagtgtcccccaaacccccaccaccct

SEQ ID NO: 1588 - DNA, 1267 bp, Artificial Sequence
  Description of Artificial Sequence: Synthetic polynucleotide
agctcctggggcctgccctcctcccattagaaaatcctccacttgtcaaaaaggaagccatttgctttgaactccaattccacccccaagaggctgggaccatcttattggagtccttgatgctgtgtgacctgcagtgaccactgccccatcattgctggctgaggtggttggggtccatctggctatctgggcagctgttctcttctctcctttctctcctgtttccagacatgcagtatttccagagagaaggggccactctttggcaaagaacctgtctaacttgctatctatggcaggacctttgaagggttcacaggaagcagcacaaattgatactattccaccaagccatcagctccatctcatccatgccctgtctctcctttaggggtccccttgccaacagaatcacagaggaccagcctgaaagtgcagagacagcagctgaggcacagccaagagctctggctgtattaatgacctaagaagtcaccagaaagtcagaagggatgacatgcagaggcccagcaatctcagctaagtcaactccaccagcctttctagttgcccactgtgtgtacagcaccctggtagggaccagagccatgacagggaataagactagactatgcccttgaggagctcacctctgttcagggaaacaggcgtggaaacacaatggtggtaaagaggaaagaggacaataggattgcatgaaggggatggaaagtgcccaggggaggaaatggttacatctgtgtgaggagtttggtgaggaaagactctaagagaaggctctgtctgtctgggtttggaaggatgtgtaggagtcttctagggggcacaggcacactccaggcataggtaaagatctgtaggtgtggcttgttgggatgaatttcaagtattttggaatgaggacagccatagagacaagggcaggagagaggcgatttaatagattttatgccaatggctccacttgagtttctgataagaacccagaacccttggactccccagtaacattgattgagttgtttatgatacctcatagaatatgaactcaaaggaggtcagtgagtggtgtgtgtgtgattctttgccaacttccaaggtggagaagcctcttccaactgcaggcagagcacaggtggccctgctactggctgcagctccagccctgcctccttctctagcatataaacaatccaacagcctcactgaatcactgctgtgcagggcaggaaagctccatgca

SEQ ID NO: 1589 - DNA, 530 bp, Artificial Sequence
  Description of Artificial Sequence: Synthetic polynucleotide
cggcctctagactcgagccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaaccggt

SEQ ID NO: 1590 - DNA, 661 bp, Artificial Sequence
  Description of Artificial Sequence: Synthetic polynucleotide
gaatgcatggttaactacgtcagaaatgaccagttcaagaggagaatgagattggcttccaaatgttggtcaagagctctacgtagcatgagccaaggatctattgaacttagtaggctcctgtgaccggtgactcttctgtctctagaaatctggggaggtgaccaggtcatacatggcagtcttcccgtgaggaacgttaaactggttggaagttggggttctgaggggaagatgtattcactaggtgacctgtcttctctgcctcggtggcctccatggctgcctgctggccgcacacccccactcagcagaggaatggactttccaatcttgctgagtgtgtttgaccaaaggtggtgctgacttagtggcctaaggtcgtgccctccctcccccactgaatcgataaataatgcgacttatcagaaagagaaagaattgtttacttttaaaccctggatcccataaagggagaggggagaggcctaaagccacagaagctgtggaaggcgccatcctgcctgccacaggaagggccttggactgagaggaccggagctgactgggggtaagtgcggctctcccccggcgcctgccgacccccctgagtgatcaggccgttctttggggtggccgctgaccgagaaatgacgggagg

SEQ ID NO: 1591 - DNA, 1299 bp, Artificial Sequence
  Description of Artificial Sequence: Synthetic polynucleotide
gcagctttgggagtgggcccaggaagtactgaggatagcaggtgagatcccaggaagagatggatgtggggccgagacactggagagagaaacaggactgtcagataaagggcgtctgtgactcctagatctcattatgcctactaccataacctacccccaattcctaatattctcctaccctagagggggggaaattgtcagaaatttggctgcaacactagcaacactactcagtacttgaaatgcatttttgcatttttttcattcaacaaatatttctggaacaactcttatatgccaggcactattttaggagtcagggatatataatggtaaacaagacaggcaaaacaaagcaaagcaacaacaaccatcaccagataagtagacagatgaaagaatttcaagttttagtaagtaaaataaaacaagcaagggtctgaaatggctagataaggtggtcaagaaaggcttcattgagaaggtagcatttaagcaggagtcagctagaaatattgtgaaattccagttacagttctatttgttctgggttggttaaataaagctttttcccccaaggtggaaactaccaagaaagactaattactagtagtggtggtgctctctggaagagagacacctcctgtttctgcctcattactgtcaacccttcacttccaggcactttttgcaaagccctttgccagtcagggaaggcgagaggctgggcatggggcttggacatttgacaacagtgagacattattgtccccagactcactagcccaagggtaaagctgaagaggcttgggcatgccccagaaaggcccctgatgaagcttggaaaaagctgttctctgagtatttctaagtaagtttatctgtgtgtgtggttactaaaagtagtaagtattgctgtctctagctgccttagagcagggcttgacacagtacacagcaatattagttccctccttttctcacctcccccattgtggagataaactcaatcacaaaaggtgatcctcagtctactcacttccctgacttatggatgcctggacccattgccagtgtgagagtcacagctggacgtcagcagtgtagcccagttactgcttgaaaattgctgaagggggttggggggcagctgccgggaaaaaggagtcttggattcagatttctgtccagaccctgaccttatttgcagtgatgtaatcagccaatattggcttagtcctgggagacagcacattcccagtagagttggaggtgggggtggtgctgctgccaact

SEQ ID NO: 1592 - DNA, 252 bp, Artificial Sequence
  Description of Artificial Sequence: Synthetic polynucleotide
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 1593 - DNA, 1594 bp, Artificial Sequence
  Description of Artificial Sequence: Synthetic polynucleotide
cccctgccctcctcctctgccctctcctggcattcctccttcatcatgggaccctcttctaatggatccccaaatgtcagagggtccaagtcctccctccctccaagctcatccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcg

SEQ ID NO: 1594 - DNA, 212 bp, Artificial Sequence
  Description of Artificial Sequence: Synthetic polynucleotide
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 1595 - DNA, 1163 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtga

SEQ ID NO: 1596 - DNA, 510 bp, Artificial Sequence
  Description of Artificial Sequence: Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccca

SEQ ID NO: 1597 - DNA, 39 bp, Artificial Sequence
  Description of Artificial Sequence: Synthetic oligonucleotide
ggtaggcgtgtacggtgggaggcctatataagcagagct

SEQ ID NO: 1598 - DNA, 1212 bp, Artificial Sequence
  Description of Artificial Sequence: Synthetic polynucleotide
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 1599 - DNA, 408 bp, Artificial Sequence
  Description of Artificial Sequence: Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 1600 - RNA, 23 nt, Homo sapiens
aacauucaacgcugucggugagu

SEQ ID NO: 1601 - RNA, 22 nt, Homo sapiens
accacugaccguugacuguacc

SEQ ID NO: 1602 - RNA, 23 nt, Homo sapiens
aacauucauugcugucggugggu

SEQ ID NO: 1603 - RNA, 21 nt, Homo sapiens
cucacugaacaaugaaugcaa

SEQ ID NO: 1604 - RNA, 22 nt, Homo sapiens
aacauucaaccugucggugagu

SEQ ID NO: 1605 - RNA, 22 nt, Homo sapiens
aaccaucgaccguugaguggac

SEQ ID NO: 1606 - RNA, 22 nt, Homo sapiens
accaucgaccguugauuguacc

SEQ ID NO: 1607 - RNA, 20 nt, Homo sapiens
cucacugaucaaugaaugca

SEQ ID NO: 1608 - RNA, 23 nt, Homo sapiens
aacauucauuguugucggugggu

SEQ ID NO: 1609 - RNA, 21 nt, Homo sapiens
ccaccgggggaugaaugucac

SEQ ID NO: 1610 - RNA, 22 nt, Homo sapiens
cguguauuugacaagcugaguu

SEQ ID NO: 1611 - RNA, 22 nt, Homo sapiens
ugucaguuugucaaauacccca

SEQ ID NO: 1612 - RNA, 21 nt, Homo sapiens
cauuauuacuuuugguacgcg

SEQ ID NO: 1613 - RNA, 22 nt, Homo sapiens
ucguaccgugaguaauaaugcg

SEQ ID NO: 1614 - RNA, 48 nt, Artificial Sequence
  Description of Artificial Sequence: Synthetic oligonucleotide
aaggaaauaagagagaaaagaagaguaagaagaaauauaagagccacc

SEQ ID NO: 1615 - RNA, 1020 nt, Artificial Sequence
  Description of Artificial Sequence: Synthetic polynucleotide
augggcaaguccaaggagaucucucaggaccugagaaagaggaucguggaucugcacaagagcggaagcucccugggagcaaucuccaagcgccuggcagugccucggucuagcgugcagaccaucgugcgcaaguacaagcaccacggcaccacacagccuucuuaucggagcggccggagaagggugcugagcccaagggacgagcggacacuggugcgcaaggugcagaucaacccccggaccacagccaaggaucuggugaagaugcuggaggagaccggcacaaagguguccaucucuaccgugaagagagugcuguacaggcacaaccugaagggccacuccgccagaaagaagccucugcugcagaauaggcacaagaaggcaaggcugagguucgcaaccgcacacggcgacaaggaucgcacauuuuggcggaacgugcuguggucugacgagaccaagaucgagcuguucggccacaaugaucacagauacguguggaggaagaagggcgaggccugcaagcccaagaauaccaucccuacagugaagcacggaggaggauccaucaugcuguggggauguuuugcagcaggaggaacaggcgcccugcacaagaucgacggcaucauggaugccgugcaguauguggacauccugaagcagcaccugaagaccucugugagaaagcugaagcugggcaggaaguggguguuccagcacgacaacgauccaaagcacacaagcaaggugguggccaaguggcugaaggacaauaaggugaaggugcuggaguggcccagccaguccccugaucugaacccaaucgagaaucugugggccgagcugaagaagagagugagggcccggagacccaccaaccugacacagcugcaccagcugugccaggaggagugggccaagauccacccaaauuacuguggcaagcugguggagggcuaucccaagaggcugacccaggugaagcaguuuaagggcaacgccacaaaguau

SEQ ID NO: 1616 - RNA, 109 nt, Artificial Sequence
  Description of Artificial Sequence: Synthetic polynucleotide
ugauuaauuaagcugccuucugcggggcuugccuucuggccaugcccuucuucucucccuugcaccuguaccucuuggucuuugaauaaagccugaguaggaagucuag

SEQ ID NO: 1617 - RNA, 76 nt, Artificial Sequence
  Description of Artificial Sequence: Synthetic oligonucleotide
aaaaaaaaaaaaaaaaaaaaaaaaaaaaaaaaaaaaaaaaaaaaaaaaaaaaaaaaaaaaaaaaaaaaaaaaaaaa

SEQ ID NO: 1618 - DNA, 130 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 1619 - DNA, 114 bp, Artificial Sequence
  Description of Artificial Sequence: Synthetic polynucleotide
gcggccgcacgcgtctagttattaatagtaatcaattacggggtcattagttcatagcccatatatggagttccgatccctatacagttgaagtcggaagtttacatacactta

SEQ ID NO: 1620 - DNA, 169 bp, Artificial Sequence
  Description of Artificial Sequence: Synthetic polynucleotide
agttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattc

SEQ ID NO: 1621 - DNA, 150 bp, Artificial Sequence
  Description of Artificial Sequence: Synthetic polynucleotide
cagtgggtcagaagtttacatacactaagttgactgtgcctttaaacagcttggaaaattccagaaaatgatgtcatggctttagaagctaacatgtgcgacgtagcttgggtaggtgagcgattaaccgtccctttaggtaccactagt

SEQ ID NO: 1622 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 1623 - DNA, 63 bp, Artificial Sequence
  Description of Artificial Sequence: Synthetic oligonucleotide
taatacgactcagctagcgtttaaacttaagcttgagctcggatccccagtgtggtggaattc

SEQ ID NO: 1624 - DNA, 696 bp, Artificial Sequence
  Description of Artificial Sequence: Synthetic polynucleotide
atggtttccgagctgatcaaagaaaacatgcacatgaagctgtacatggaaggcaccgtgaacaaccaccacttcaagtgcaccagcgaaggcgagggcaagccttatgagggcacccagaccatgagaatcaaggccgttgaaggcggccctctgcctttcgcctttgatatcctggccaccagctttatgtacggcagcaagaccttcatcaatcacacccagggcatccccgatttcttcaagcagagcttccccgagggcttcacctgggagagagtgaccacatacgaggatggcggcgtgctgacagccacacaggatacaagtctgcaggacggctgcctgatctacaacgtgaagatccggggcgtgaacttccccagcaatggccccgtgatgcagaagaaaaccctcggctgggaagccagcaccgagacactgtatcctgccgatggtggcctggaaggcagagctgatatggccctgaaactcgttggcggcggacacctgatctgcaatctgaaaaccacctaccggtccaagaagcccgccaagaacctgaagatgcccggcgtgtactacgtggacagacggctggaacggatcaaagaggccgacaaagaaacctacgtggaacagcacgaggtggccgtggccagatactgtgatctgccttctaagctgggccacaga

SEQ ID NO: 1625 - DNA, 43 bp, Artificial Sequence
  Description of Artificial Sequence: Synthetic oligonucleotide
taatctagagtcgacctgcagaagcttgatatcaccggtcgat

SEQ ID NO: 1626 - DNA, 589 bp, Artificial Sequence
  Description of Artificial Sequence: Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 1627 - DNA, 23 bp, Artificial Sequence
  Description of Artificial Sequence: Synthetic oligonucleotide
atagcgctgctcgagagatctac

SEQ ID NO: 1628 - DNA, 477 bp, Artificial Sequence
  Description of Artificial Sequence: Synthetic polynucleotide
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 1629 - DNA, 143 bp, Artificial Sequence
  Description of Artificial Sequence: Synthetic polynucleotide
tccggagcggccgcgtttaattgagttgtcatatgttaataacggtatgtggaaggctactcgaaatgtttgacccaagttaaacaatttaaaggcaatgctaccaaatactaattgagtgtatgtaaacttctgacccactg

SEQ ID NO: 1630 - DNA, 169 bp, Artificial Sequence
  Description of Artificial Sequence: Synthetic polynucleotide
ggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggct

SEQ ID NO: 1631 - DNA, 88 bp, Artificial Sequence
  Description of Artificial Sequence: Synthetic oligonucleotide
aaggtgtatgtaaacttccgacttcaactgtatagggatcctctagctactgattttgtaggtaaccacgtgcggaccgagcggccgc

SEQ ID NO: 1632 - DNA, 141 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1633 - DNA, 1615 bp, Artificial Sequence
  Description of Artificial Sequence: Synthetic polynucleotide
gcggccattcggtacaattcacgcgtgagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgatccctatacagttgaagtcggaagtttacatacactta

SEQ ID NO: 1634 - DNA, 138 bp, Artificial Sequence
  Description of Artificial Sequence: Synthetic polynucleotide
cagtgggtcagaagtttacatacactaagttgactgtgcctttaaacagcttggaaaattccagaaaatgatgtcatggctttagaagctaacatgtgcgacgtagcttgggtaggtgagcgattaaccgtcccttta

SEQ ID NO: 1635 - DNA, 307 bp, Artificial Sequence
  Description of Artificial Sequence: Synthetic polynucleotide
gggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 1636 - DNA, 495 bp, Artificial Sequence
  Description of Artificial Sequence: Synthetic polynucleotide
atggagatgctccaggggctgctgctgtggctgctgctgagcatgggcggggcacgggcatccagggagccgctgcggccactgtgccgccccatcaatgccaccctggctgccgagaaggaggcctgccccgtgtgcatcaccgtcaacaccaccatctgtgccggctactgccccaccatgatgcgggtgctgcaggcggtcctgccgccagtgccccaggtggtgcgcaactaccgcgaggtgcgcttcgagtccatccggctccctggctgcccgcctggcgtggaccccgtggtctccgttcccgtggctctcagctgtcgttgtgcactctgccgccgcagcacctctgactgtgggggtcccaaggaccaccctttgacctgtgatgacccccacctccaggcctcctcttcctcaaaggaccctccccccagccctccaagtccatccggactcctggagccagcagacaacccgttcctcccgcaa

SEQ ID NO: 1637 - DNA, 32 bp, Artificial Sequence
  Description of Artificial Sequence: Synthetic oligonucleotide
atcgataccgtcgactcgctgatcagcctcga

SEQ ID NO: 1638 - DNA, 225 bp, Artificial Sequence
  Description of Artificial Sequence: Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 1639 - DNA, 129 bp, Artificial Sequence
  Description of Artificial Sequence: Synthetic polynucleotide
gtttaattgagttgtcatatgttaataacggtatgtggaaggctactcgaaatgtttgacccaagttaaacaatttaaaggcaatgctaccaaatactaattgagtgtatgtaaacttctgacccactg

SEQ ID NO: 1640 - DNA, 66 bp, Artificial Sequence
  Description of Artificial Sequence: Synthetic oligonucleotide
aaggtgtatgtaaacttccgacttcaactgtatatctagatccggagagctcctcgaggcggccgc

SEQ ID NO: 1641 - DNA, 1689 bp, Artificial Sequence
  Description of Artificial Sequence: Synthetic polynucleotide
gcggccattcggtacaattcacgcgtgagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgatccttgactgtgcctttaaacagcttggaaaattccagaaaatgatgtcatggctttagaagctaacatgtgcgacgtagcttgggtaggtgagcgattaaccgtcccttta

SEQ ID NO: 1642 - DNA, 257 bp, Artificial Sequence
  Description of Artificial Sequence: Synthetic polynucleotide
atcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 1643 - DNA, 132 bp, Artificial Sequence
  Description of Artificial Sequence: Synthetic polynucleotide
gtttaattgagttgtcatatgttaataacggtatgtggaaggctactcgaaatgtttgacccaagttaaacaatttaaaggcaatgctaccaaatactaatctagatccggagagctcctcgaggcggccgc

SEQ ID NO: 1644 - DNA, 45 bp, Artificial Sequence
  Description of Artificial Sequence: Synthetic oligonucleotide
gcggccattcggtacaattcacgcgtctaagttaattaactgcag

SEQ ID NO: 1645 - DNA, 1065 bp, Artificial Sequence
  Description of Artificial Sequence: Synthetic polynucleotide
gctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 1646 - DNA, 56 bp, Artificial Sequence
  Description of Artificial Sequence: Synthetic oligonucleotide
gcggccgctctagaactagtggatcccccgggctgcaggaattcactagtgatttc

SEQ ID NO: 1647 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 1648 - DNA, 18 bp, Artificial Sequence
  Description of Artificial Sequence: Synthetic oligonucleotide
gatatcaagcttatcgat

SEQ ID NO: 1649 - DNA, 83 bp, Artificial Sequence
  Description of Artificial Sequence: Synthetic oligonucleotide
cttctgaggcggaaagaaccaattgcattcattttatgtttcaggttcagggggagatgtgggaggttttttaaagcggccgc

SEQ ID NO: 1650 - RNA, 1253 nt, Artificial Sequence
  Description of Artificial Sequence: Synthetic polynucleotide
aaggaaauaagagagaaaagaagaguaagaagaaauauaagagccaccaugggcaaguccaaggagaucucucaggaccugagaaagaggaucguggaucugcacaagagcggaagcucccugggagcaaucuccaagcgccuggcagugccucggucuagcgugcagaccaucgugcgcaaguacaagcaccacggcaccacacagccuucuuaucggagcggccggagaagggugcugagcccaagggacgagcggacacuggugcgcaaggugcagaucaacccccggaccacagccaaggaucuggugaagaugcuggaggagaccggcacaaagguguccaucucuaccgugaagagagugcuguacaggcacaaccugaagggccacuccgccagaaagaagccucugcugcagaauaggcacaagaaggcaaggcugagguucgcaaccgcacacggcgacaaggaucgcacauuuuggcggaacgugcuguggucugacgagaccaagaucgagcuguucggccacaaugaucacagauacguguggaggaagaagggcgaggccugcaagcccaagaauaccaucccuacagugaagcacggaggaggauccaucaugcuguggggauguuuugcagcaggaggaacaggcgcccugcacaagaucgacggcaucauggaugccgugcaguauguggacauccugaagcagcaccugaagaccucugugagaaagcugaagcugggcaggaaguggguguuccagcacgacaacgauccaaagcacacaagcaaggugguggccaaguggcugaaggacaauaaggugaaggugcuggaguggcccagccaguccccugaucugaacccaaucgagaaucugugggccgagcugaagaagagagugagggcccggagacccaccaaccugacacagcugcaccagcugugccaggaggagugggccaagauccacccaaauuacuguggcaagcugguggagggcuaucccaagaggcugacccaggugaagcaguuuaagggcaacgccacaaaguauugauuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0042058 A1
(43) Pub. Date: Feb. 8, 2024

(54) TISSUE-SPECIFIC METHODS AND COMPOSITIONS FOR MODULATING A GENOME

(71) Applicant: Flagship Pioneering Innovations VI, LLC, Cambridge, MA (US)

(72) Inventors: Robert James Citorik, Somerville, MA (US); Jacob Rosenblum Rubens, Cambridge, MA (US); William Edward Salomon, West Roxbury, MA (US); Zi Jun Wang, Arlington, MA (US)

(21) Appl. No.: 18/278,939

(22) PCT Filed: Feb. 25, 2022
(86) PCT No.: PCT/US2022/070834

§ 371 Date: Aug. 25, 2023

Related U.S. Application Data
(60) Provisional application No. 63/244,345, filed on Sep. 15, 2021.
(60) Provisional application No. 63/154,275, filed on Feb. 26, 2021.

(51) Int. Cl. A61K 48/00; C12N 15/10; C12N 15/86; C12N 15/88; C12N 9/12

(52) U.S. Cl. CPC A61K 48/0058; A61K 48/0041; C12N 15/102; C12N 15/86; C12N 15/88; C12N 9/1241

(57) ABSTRACT

The invention provides, inter alia, systems and associated methods, for modifying DNA, such as the genome of a cell. The systems, in certain embodiments, encompass one or more tissue-specific expression-control sequences, such as promoters and microRNA binding sites in addition to a transposase (or a nucleic acid encoding the same) and a template nucleic acid comprising a sequence to be inserted into the genome of a cell, tissue, or subject.

### CROSS-REFERENCE TO RELATED APPLICATIONS

This application claims the benefit of U.S. Provisional Application Nos. 63/154,275, filed Feb. 26, 2021; and 63/244,345, filed Sep. 15, 2021. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 24, 2022, is named V2065-7013WO_SL.txt and is 200,258 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits and are less effective at integrating longer sequences. Other existing approaches, like Cre/loxP, require a first step of inserting a loxP site into the genome and then a second step of inserting a sequence of interest into the loxP site. There is a need in the art for improved proteins for inserting sequences of interest into a genome and preferentially doing so in a tissue-specific manner.

### SUMMARY OF THE INVENTION

This disclosure relates to novel compositions, systems and methods for altering a genome at one or more locations in a host cell, tissue, or subject, in vivo or in vitro. In particular, the invention features compositions, systems and methods for the introduction of exogenous genetic elements into a host genome in a tissue-specific manner.

The invention provides, inter alia, systems and methods for modifying a genome using transposase (or nucleic acids encoding them) Gene Writers™ together with a template nucleic acid (sometimes alternately referred to as template DNA), which includes a heterologous object sequence (DNA to be inserted into the target DNA (genome)), and a sequence specifically bound by the transposase and one or more tissue-specific expression-control sequences, which tissue-specific expression-control sequences are in operative association with at least one of the transposase (if provided as a nucleic acid) and the template nucleic acid. The systems provided by the invention can insert heterologous object sequence(s) into a target DNA strand—e.g., a genome. The heterologous object sequence can be any sequences of interest, including protein coding sequences, non-protein coding sequences, or both protein coding and protein non-coding sequences.

The systems can be provided by any suitable means, including, but not limited to, pharmaceutical formulations, nanoparticles, viral delivery systems, and combinations thereof. Systems provided by the invention, being suitably formulated for delivery, can thus be used in additional aspect of the invention, namely methods of inserting a heterologous object sequence into a target DNA, e.g., a genomic locus, e.g., in a cell, tissue, or organism—e.g., for a therapeutic intervention, e.g., for a disorder or condition.

### BRIEF DESCRIPTION OF THE DRAWINGS

FIG. 1 is a diagram that depicts an embodiment in which the Gene Writing™ polypeptide and DNA template are incorporated on two separate AAVs for co-administration. ITR refers to inverted terminal repeat from AAV genome. IR/DR refers to inverted repeat/direct repeat from transposon.

FIG. 2 is a diagram that depicts certain embodiments of regulatory controls that may be incorporated into the nucleic acid encoding the Gene Writing™ polypeptide and the heterologous object sequence of the DNA template (template nucleic acid). These regulatory elements facilitate upregulation of expression in target cells (tissue-specific promoter/enhancer) and downregulation of expression in non-target cells (miRNA binding sites).

FIG. 3 is a diagram of certain embodiments in which the nucleic acid sequences encoding the Gene Writer™ polypeptide and the DNA template are on a single nucleic acid molecule.

FIG. 4 is a diagram of certain embodiments in which the transposase is provided as an RNA molecule that may include elements for modifying expression of the transposase (e.g., 5′-UTR, 3′-UTR, miRNA binding sites).

FIG. 5 is a diagram of certain embodiments in which the Gene Writer™ polypeptide is provided as a protein that associates with the IR/DR elements of the DNA template and may, in certain embodiments, optionally be pre-associated with the template for administration as a deoxyribonucleoprotein complex.

FIGS. 6A and 6B describes luciferase activity assay for primary cells. LNPs formulated as according to Example 3 were analyzed for delivery of cargo to primary human (A) and mouse (B) hepatocytes, as according to Example 4. The luciferase assay revealed dose-responsive luciferase activity from cell lysates, indicating successful delivery of RNA to the cells and expression of Firefly luciferase from the mRNA cargo.

FIG. 7 shows LNP-mediated delivery of RNA cargo to the murine liver. Firefly luciferase mRNA-containing LNPs were formulated and delivered to mice by i.v., and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours, post-administration.

FIG. 8 shows the expression over time after transfection and/or transduction of the SB100X mRNA LNP and AAVDJ-mKate2 SB transposon. AAVDJ-mKate2 SB transposon alone shows a decrease in mKate2 expression over time as cells divide and episomal AAV expression is diluted with cell divisions. The cells that were co-treated with SB100X mRNA LNP transfection and AAVDJ-mKate2 SB transposon transduction show sustained expression of the fluorescence over time. The sustained expression represents integration into the genome that is not lost with cell division.

FIGS. 9A and 9B. FIG. 9A shows fluorescence images of primary hepatocytes taken either 4 or 7 days after transfection and/or transduction. Brightfield images were taken on day 12. Primary hepatocytes do not divide and there is no expectation of a loss of mKate2 fluorescence expression over time after AAV expression (data not shown). Total fluorescence of episomal expressed mKate2 transposon alone (images at 0 ng SB100X) was weaker when compared to wells that had greater than 1 ng of SB100X mRNA LNP added to them (FIG. 9B). There is no amplification of the AAV in these non-dividing cells thus the integration of mKate2 mediated by SB100X leads to higher expression of mKate2 when compared to the expression only coming from the AAV episome.

FIGS. 10A-10C show the comparison of mKate2 fluorescence over time after administration of SB100X transposase mRNA-LNP and a Sleeping Beauty transposon containing the mKate2 gene. When SB100X was expressed via an mRNA delivered by LNP it mediated expression of mKate2 protein that is approximately 20 times higher than what was expressed with the AAV transposon alone. Expression was sustained over the course of 6 weeks in a dose-dependent fashion where expression of SB100X at 1 mg per kg mediated highest levels of mKate2 expression mediated by the integration activity of the transposase. In FIG. 10A, each set of four bars represents, from left to right, 24 hours, 2 weeks, 4 weeks, and 6 weeks. FIG. 10B shows the increased mKate2 fluorescence in treated mice over 6-weeks post dosing with transposon and SB100X transposase compared to AAV-transposon alone. FIG. 10C shows AAV copy numbers in mouse livers following AAV transduction with mKate2 transposon.

FIG. 11 shows the comparison of mKate2 fluorescence after dosing adult mice (n=3) with different concentrations of SB100X transposase mRNA-LNP and a fixed concentration of Sleeping Beauty transposon containing the mKate2 gene (1×10¹² vg per mouse). When SB100X was expressed via an mRNA delivered by LNP it mediated expression of mKate2 protein that was as high as approximately 85 times higher than what was expressed with the AAV transposon alone. Activity of Sleeping Beauty 100X to integrate mKate2 and mediate 85-fold increase of fluorescence showed a plateau at 2 mpk where concentrations higher (3 mpk) did not show increased levels of fluorescence.

FIGS. 12A-12B are a series of graph showing mKate2 fluorescence and AAV copy numbers, respective, after dosing mice with increasing concentrations of LNP SB100X transposase and a fixed concentration of AAV transposon containing the mKate2 cDNA.

FIG. 13 is a graph showing rhCG serum concentration over two weeks measured by radioimmunoassay.

FIG. 14 is a graph showing qRT PCR analysis of rhCG transcripts in AAV treated mouse livers.

FIG. 15 is a graph showing AAV copy numbers in transduced mouse livers as determined by ddPCR.

FIG. 16 is a graph showing that ApoE-hAAT and SerpTTRmin promoters increased eGFP production with increasing dose of AAV

FIGS. 17A-17B are a series of graph showing that the SerpTTRmin construct delivered a payload reporter gene to tissue throughout the target organ.

FIGS. 18A-18B are a series of graphs showing that dose escalation of the SerpTTRmin construct by 5× increased eGFP signal 3-4 fold, along with AAV copy numbers.

FIG. 19 is a graph showing that animals with either 10 or 20 nAbs titers had reduced eGFP levels by a factor of 2-6 fold compared to animals without nAbs.

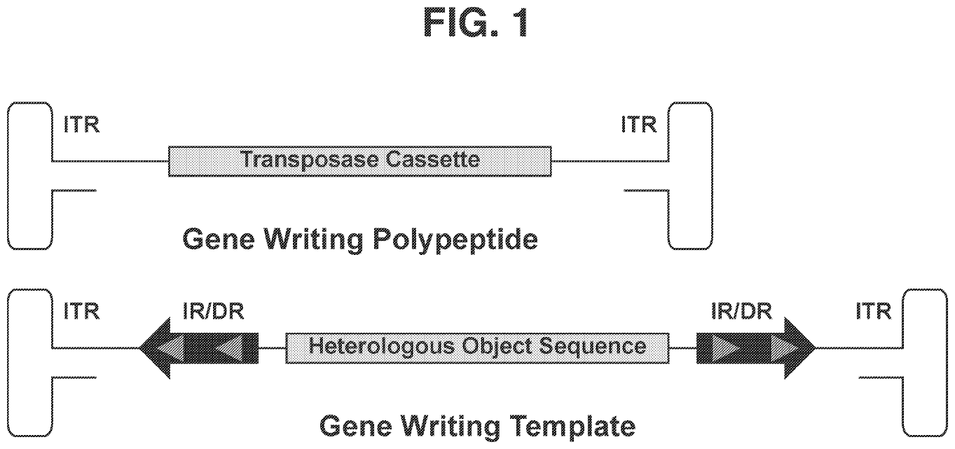

### DETAILED DESCRIPTION

Integration of a nucleic acid of interest (e.g., template nucleic acid, e.g., comprising a heterologous object sequence) into a genome occurs at low frequency, in the absence of a specialized protein to promote the insertion event. Some existing approaches, like CRISPR/Cas9, are more suited for small edits and are less effective at integrating longer sequences. Other existing approaches, like Cre/loxP, require a first step of inserting a loxP site into the genome and then a second step of inserting a sequence of interest into the loxP site. There is a need in the art for improved proteins for inserting sequences of interest into a genome, preferably wherein the integration of the sequence of interest, expression of the sequence of interest, or both insertion and expression of the sequence of interest, are tissue-specific, e.g., inserted, expressed, or inserted and expressed preferentially in a target tissue, such as the lung.

Features of the systems or methods of using them can include one or more of the following enumerated embodiments.
1. A system for modifying DNA in a target tissue comprising:
   a) a transposase protein or a nucleic acid encoding the same;
   b) a template nucleic acid comprising i) a sequence specifically bound by the transposase, and ii) a heterologous object sequence
   c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the transposase.
2. A system of embodiment 1, wherein:
   i) the one or more first tissue-specific expression-control sequences specific to the target tissues comprise a sequence selected from Table 2 or Table 3,
   ii) the heterologous object sequence comprises a sequence selected from Table 4, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB; or
iii) (i) and (ii).
3. The system of any one of the preceding embodiments, wherein the nucleic acid in (b) comprises RNA.
4. The system of any one of the preceding embodiments, wherein the nucleic acid in (b) comprises DNA.
5. The system of any one of the preceding embodiments, wherein the nucleic acid in (b):
   a. is single-stranded or comprises a single-stranded segment, e.g., is single-stranded DNA or comprises a single-stranded segment and one or more double stranded segments;
   b. has inverted terminal repeats; or
   c. both (i) and (ii).
6. The system of any one of the preceding embodiments, wherein the nucleic acid in (b) is double-stranded or comprises a double-stranded segment.
7. The system of any one of the preceding embodiments, wherein (a) comprises a nucleic acid encoding the transposase.
8. The system of embodiment 7, wherein the nucleic acid in (a) comprises RNA.
9. The system of any one of embodiments 7 or 8, wherein the nucleic acid in (a) comprises DNA.
10. The system of any one of embodiments 7-9, wherein the nucleic acid in (a):
   d. is single-stranded or comprises a single-stranded segment, e.g., is single-stranded DNA or comprises a single-stranded segment and one or more double stranded segments;
   e. has inverted terminal repeats; or
   f. both (i) and (ii).
11. The system of any one of embodiments 7-10, wherein the nucleic acid in (a) is double-stranded or comprises a double-stranded segment.
12. The system of any one of the preceding embodiments, wherein the nucleic acid in (a), (b), or (a) and (b) is linear.
13. The system of any one of the preceding embodiments, wherein the nucleic acid in (a), (b), or (a) and (b) is circular, e.g., a plasmid or minicircle.
14. The system of any one of the preceding embodiments, wherein the heterologous object sequence is in operative association with a first promoter.
15. The system of any one of the preceding embodiments, wherein the one or more first tissue-specific expression-control sequences comprises a tissue specific promoter.
16. The system of embodiment 15, wherein the tissue-specific promoter comprises a first promoter in operative association with:
   i. the heterologous object sequence,
   ii. a nucleic acid encoding the transposase, or
   iii. (i) and (ii).
17. The system of any one of the preceding embodiments, wherein the one or more first tissue-specific expression-control sequences comprises a tissue-specific microRNA recognition sequence in operative association with:
   i. the heterologous object sequence,
   ii. a nucleic acid encoding the transposase, or
   iii. (i) and (ii).
18. The system of any one of the preceding embodiments, comprising a tissue-specific promoter, the system further comprising one or more tissue-specific microRNA recognition sequences, wherein:
   i. the tissue specific promoter is in operative association with:
      I. the heterologous object sequence,
      II. a nucleic acid encoding the transposase, or
      III. (I) and (II);
   ii. The one or more tissue-specific microRNA recognition sequences are in operative association with:
      I. the heterologous object sequence,
      II. a nucleic acid encoding the transposase, or
      III. (I) and (II).
19. The system of any one of the preceding embodiments, comprising a nucleic acid encoding the transposase protein, wherein the nucleic acid comprises a promoter in operative association with the nucleic acid encoding the transposase protein.
20. The system of embodiment 19, wherein the nucleic acid encoding the transposase protein comprises one or more second tissue-specific expression-control sequences specific to the target tissue in operative association with the transposase coding sequence.
21. The system of embodiment 20, wherein the one or more second tissue-specific expression-control sequences comprises a tissue specific promoter.
22. The system of embodiment 21, wherein the tissue-specific promoter is the promoter in operative association with the nucleic acid encoding the transposase protein.
23. The system of any one of embodiments 19-22, wherein the one or more second tissue-specific expression-control sequences comprises a tissue-specific microRNA recognition sequence.
24. The system of any one of embodiments 19-23, wherein the promoter in operative association with the nucleic acid encoding the transposase protein is a tissue-specific promoter, the system further comprising one or more tissue-specific microRNA recognition sequences.
25. The system of any one of the preceding embodiments, wherein the one or more first tissue-specific expression-control sequences and, if present, one or more second tissue-specific expression-control sequences comprise a tissue-specific promoter selected from a promoter described in Table 2.
26. The system of any one of the preceding embodiments, wherein the one or more first tissue-specific expression-control sequences and, if present, one or more second tissue-specific expression-control sequences comprises a tissue-specific microRNA recognition sequence described in Table 3.
27. The system of any one of the preceding embodiments, wherein, when provided to an organism, at least: 1, 5, 10, 20, 25, 30, 35, 40, 45, 50, 55, 60, 65, 70, 75, 80, 85, 90, 91, 92, 93, 94, 95, 96, 97, 98, 99%, or more, of incorporation of the heterologous object sequence into the genome of a cell are in cells the target tissue.
28. The system of any one of the preceding embodiments, wherein, when provided to an organism, incorporation of the heterologous object sequence into the genome of a cell in the target tissue is at least: 1, 5, 10, 20, 25, 30, 35, 40, 45, 50, 55, 60, 65, 70, 75, 80, 85, 90, 91, 92, 93, 94, 95, 96, 97, 98, 99%, or more, of all integrations in the organism, e.g., at least: 1, 5, 10, 25, 30, 35, 40, 45, 50, 55, 60, 65, 70, 75, 80, 85, 90, 91, 92, 93, 94, 95, 96, 97, 98, 99%, or more, of the expression of the heterologous object sequence is in a cell in the target tissue.
29. The system of any one of the preceding embodiments, wherein, when provided to an organism, expression of the heterologous object sequence in a cell in the target tissue is at least: 1, 5, 10, 20, 25, 30, 35, 40, 45, 50, 55, 60, 65, 70, 75, 80, 85, 90, 91, 92, 93, 94, 95, 96, 97, 98, 99%, or more, of all expression of the heterologous object sequence in the organism, e.g., at least: 1, 5, 10, 20, 25, 30, 35, 40, 45, 50, 55, 60, 65, 70, 75, 80, 85, 90, 91, 92, 93, 94, 95, 96, 97, 98, 99%, or more, of the expression of the heterologous object sequence is in a cell in the target tissue.
30. The system of any one of embodiments 27-29, wherein the organism is a vertebrate, such as a mammal, such as a human or, in certain embodiments, a non-human mammal, such as a non-human primate, a mouse, a dog, or a pig.
31. The system of any one of the preceding embodiments, further comprising a first recombinant adeno-associated virus (rAAV) capsid protein; wherein at least one of (a) or (b) is associated with the first rAAV capsid protein, wherein the at least one of (a) or (b) is flanked by AAV inverted terminal repeats (ITRs).
32. The system of embodiment 31, wherein (a) and (b) are associated with the first rAAV capsid protein.
33. The system of embodiment 32, wherein (a) and (b) are on a single nucleic acid.
34. The system any one of embodiments 32-33, further comprising a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.
35. The system of any one of embodiments 31-33, wherein the at least one of (a) or (b) is associated with the first or second rAAV capsid protein is dispersed in the interior of the first or second rAAV capsid protein, which first or second rAAV capsid protein is in the form of an AAV capsid particle.
36. The system of any one of embodiments 31-35, wherein the first or second rAAV capsid protein is from an AAV serotype selected from Table 5.
37. The system of any one of embodiments 1-31, further comprising a nanoparticle, wherein the nanoparticle is associated with at least one of (a) or (b).
38. The system of any one of the preceding embodiments, wherein (a) and (b), respectively are associated with:
a) a first rAAV capsid protein and a second rAAV capsid protein
b) a nanoparticle and a first rAAV capsid protein
c) a first rAAV capsid protein
d) a first adenovirus capsid protein
e) a first nanoparticle and a second nanoparticle
f) a first nanoparticle.
39. The system of any one of the preceding embodiments, wherein the target tissue is selected from liver, lung, kidney, skin, stem cell, hematopoietic stem cell, blood cell, immune cell, T cell, NK cell; such as mammalian: liver, lung, kidney, skin, stem cell, hematopoietic stem cell, blood cell, immune cell, T cell, NK cell; such as human: liver, lung, kidney, skin, stem cell, hematopoietic stem cell, blood cell, immune cell, T cell, NK cell.
40. The system of any one of the preceding embodiments, wherein the heterologous object sequence encodes a polypeptide of at least 25, 50, 100, 150, 200, 250, 300, 400, 500, 600, 700, 800, 900, 1000 residues, or more.
41. The system of any one of the preceding embodiments, wherein the heterologous object sequence encodes an enzyme (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, a storage protein, and immune receptor, a synthetic protein (e.g. a chimeric antigen receptor), an antibody, or combinations thereof.
42. The system of any one of the preceding embodiments, wherein the heterologous object sequence comprises a sequence selected from:
   i. a tissue specific promoter or enhancer;
   ii. a non-coding RNA, such as regulatory RNA, a microRNA, an siRNA, an antisense RNA;
   iii. a polyadenylation sequence;
   iv. a splice signal;
   v. a sequence encoding a polypeptide of greater than 250, 300, 400, 500, or 1,000
   amino acids, and optionally up to 7,500 amino acids;
   vi. a sequence encoding a fragment of a mammalian gene but does not encode the
   full mammalian gene, e.g., encodes one or more exons but does not encode a full-length protein;
   vii. a sequence encoding one or more introns;
   viii. a sequence encoding a polypeptide other than a GFP, e.g., is other than a fluorescent protein or is other than a reporter protein;
   ix. is other than a sequence encoding ornithine transcarbamylase, arginosuccinate synthase, ABCB4;
   x. is other than a sequence encoding factor ix;
   xi. is other than CFTR;
   xii. or a combination of any of the foregoing.
43. The system of any one of the preceding embodiments further comprising a pharmaceutically acceptable carrier or diluent.
44. A method of making the system of any one of embodiments 31-36, comprising transforming an AAV packaging cell line with a nucleic acid encoding (a), (b), or (a) and (b) and collecting the first rAAV capsid protein, second rAAV, or first and second rAAV capsid protein and associated nucleic acid(s).
45. One or more AAV packaging cell lines comprising a nucleic acid encoding (a), (b), or (a) and (b) of the system of any one of the preceding embodiments.
46. A method of modifying a target DNA strand in a cell, tissue or subject, comprising administering the system of any preceding embodiment to the cell, tissue or subject, wherein the system inserts the heterologous object sequence into the target DNA strand, thereby modifying the target DNA strand.
47. The method of embodiments 46, wherein the heterologous object sequence is expressed in the cell, tissue, or subject.
48. The method of embodiment 46 or 47, wherein the cell, tissue or subject is a mammalian (e.g., human) cell, tissue or subject.
49. The method of any one of the preceding embodiments, wherein the cell is a hepatocyte.
50. The method of any one of the preceding embodiments, wherein the cell is lung epithelium.
51. The method of any one of the preceding embodiments, wherein the cell is an ionocyte.
52. The method of any one of the preceding embodiments, wherein the cell is a primary cell.
53. The method of any one of the preceding embodiments, where in the cell is not immortalized.
54. A method of treating a mammalian tissue comprising administering the system of any one of embodiments 1-42 to the mammal, thereby treating the tissue, wherein the tissue is deficient in the heterologous object sequence.
55. The method of embodiment 54, wherein:
(i) the mammal has an indication selected from Column 6 of Table 4 or an indication of the lungs (e.g., alpha-1-antitrypsin (AAT) deficiency, cystic fibrosis (CF), primary ciliary dyskinesia (PCD), surfactant protein B (SP-B) deficiency);
(ii) the heterologous object sequence of (b) is selected from Column 1 of Table 4 or, or a fragment derived of any of the foregoing, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB, or (iii) (i) and (ii).
56. The method of any one of the preceding embodiments, wherein (a) and (b) are administered concurrently, wherein optionally (a) and (b) are administered in separate compositions.
57. The method of any one of embodiments 38-54, wherein (a) and (b) are administered in a single composition.
58. The method of any one of embodiments 46-55, wherein (a) and (b) are administered sequentially.
59. The method of any one of the preceding embodiments, wherein less than 1, 2, 3, 4, 5, 10, 15, 20, 25, 30, 35, 40, 45, or 50% of the cells in the target tissue are in G0 phase of the cell cycle (i.e. are post-mitotic).
60. The method of any one of the preceding embodiments, wherein at least 1, 2, 3, 4, 5, 10, 15, 25, 30, 35, 40, 45, or 50% of the cells in the target tissue are in M, G1, S, or G2 phase of the cell cycle (i.e., are mitotic).
61. The method of any one of the preceding embodiments, wherein the transposase is expressed transiently.
62. The method of any one of the preceding embodiments, wherein the transposase is expressed for less than 1, 2, 3, 4, 5, 6, 7, 8, 9, 10, 15, 20, 25, 50, 75, 100, 150, 200, 250, days after administration.
63. The method of any one of the preceding embodiments, wherein the transposase is expressed at a level of less than 1, 2, 3, 4, 5, 6, 7, 8, 9, 10, 15, 20, 25, 50, 75, 99% of the expression level measured at day 1, 2, 3, 4, 5, 6, 7, 8, 9, 10, 15, 20, 25, 50, 75, 100 post administration, when the measurement is taken 2, 3, 4, 5, 6, 7, 8, 9, 10, 15, 20, 25, 50, 75, 100, 150, 200, 250, or more days after administration.
64. The method of any one of the preceding embodiments, wherein the transposase nucleic acid is present transiently.
65. The method of any one of the preceding embodiments, wherein the transposase nucleic acid is no-longer detected 1, 2, 3, 4, 5, 6, 7, 8, 9, 10, 15, 20, 25, 50, 75, 100, 150, 200, or 250 days after administration.
66. The method of any one of the preceding embodiments, wherein the transposase nucleic acid is detected at a level less than 1, 2, 3, 4, 5, 6, 7, 8, 9, 10, 15, 20, 25, 50, 75, 99% of the level measured at day 1, 2, 3, 4, 5, 6, 7, 8, 9, 10, 15, 20, 25, 50, 75, 100 post administration, when the measurement is taken 2, 3, 4, 5, 6, 7, 8, 9, 10, 15, 20, 25, 50, 75, 100, 150, 200, 250, or more days after administration.
67. The method of any one of the embodiments, wherein the heterologous object sequence is expressed permanently.
68. The method of any one of the preceding embodiments, wherein the heterologous object is expressed for at least 1, 2, 3, 4, 5, 6, 7, 8, 9, 10, 15, 20, 25, 50, 75, 100, 150, 200, 250, or more, days after administration.
69. The method of any one of the preceding embodiments, wherein the heterologous object sequence is expressed at a level of at least 1, 2, 3, 4, 5, 6, 7, 8, 9, 10, 15, 20, 25, 50, 75, 99% of the expression level measured at day 1, 2, 3, 4, 5, 6, 7, 8, 9, 10, 15, 20, 25, 50, 75, 100 post administration, when the measurement is taken 2, 3, 4, 5, 6, 7, 8, 9, 10, 15, 20, 25, 50, 75, 100, 150, 200, 250, or more days after administration.
70. The method of any one of the preceding embodiments, wherein the heterologous object sequence is detected permanently.
71. The method of any one of the preceding embodiments, wherein the heterologous object sequence is detected at least 1, 2, 3, 4, 5, 6, 7, 8, 9, 10, 15, 20, 25, 50, 75, 100, 150, 200, 250, or more, days after administration.
72. The method of any one of the preceding embodiments, wherein the heterologous object sequence is detected at a level at least 1, 2, 3, 4, 5, 6, 7, 8, 9, 10, 15, 20, 25, 50, 75, 99% of the level measured at day 1, 2, 3, 4, 5, 6, 7, 8, 9, 10, 15, 20, 25, 50, 75, 100 post administration, when the measurement is taken 2, 3, 4, 5, 6, 7, 8, 9, 10, 15, 20, 25, 50, 75, 100, 150, 200, 250, or more days after administration.
73. The method of any one of the preceding embodiments, wherein the heterologous object is permanently maintained in the genome.
74. The method of any one of the preceding embodiments, wherein the heterologous object is present in the genome for at least 1, 2, 3, 4, 5, 6, 7, 8, 9, 10, 15, 20, 25, 50, 75, 100, 150, 200, 250, or more days after administration.
75. The method of any one of the preceding embodiments, wherein the heterologous object sequence is present in the genome at a level at least 1, 2, 3, 4, 5, 6, 7, 8, 9, 10, 15, 20, 25, 50, 75, 99% of the level measured at day 1, 2, 3, 4, 5, 6, 7, 8, 9, 10, 15, 20, 25, 50, 75, 100 post administration, when the measurement is taken 2, 3, 4, 5, 6, 7, 8, 9, 10, 15, 20, 25, 50, 75, 100, 150, 200, 250, or more days after administration.
76. The method of any of one the preceding embodiments, wherein the heterologous object sequence has an average copy number of about 1, 2, 3, 4, 5, 6, 7, 8, 9, 10 or less in the target tissue.
77. The method of any one of the preceding embodiments, wherein the heterologous object sequence has an average copy number of about 1, 2, 3, 4, 5, 6, 7, 8, 9, 10 in at least 1, 2, 3, 4, 5, 6, 7, 8, 9, 10, 15, 20, 25, 50, 75, 99% of the target tissue.
78. The method of any one of the preceding embodiments, wherein the heterologous object sequence has an average copy number of less than 1, 2, 3, 4, 5, 6, 7, 8, 9, 10 or less in non-target tissue.
79. The method of any of the preceding embodiments wherein the heterologous object sequence has an average copy number of less than 1, 2, 3, 4, 5, 6, 7, 8, 9, 10 in at least 1, 2, 3, 4, 5, 6, 7, 8, 9, 10, 15, 20, 25, 50, 75, 99% in non-target tissue.
80. An isolated nucleic acid comprising a template nucleic acid comprising i) a sequence specifically bound by a transposase ii) a heterologous object sequence, the heterologous object sequence comprising one or more first tissue-specific expression-control sequences specific to a target tissue, optionally wherein the one or more first tissue-specific expression-control sequences specific to the target tissues comprise a sequence selected from Table 2 or Table 3, wherein the one or more first tissue-specific expression-control sequences specific to the target tissue are in operative association with the heterologous object sequence.
81. An isolated nucleic acid comprising a template nucleic acid comprising i) a sequence specifically bound by a transposase ii) a heterologous object sequence, the heterologous object sequence comprising a gene selected from Column 1 of Table 4 or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB, the heterologous object sequence further comprising one or more first tissue-specific expression-control sequences specific to a target tissue, optionally wherein the one or more first tissue-specific expression-control sequences specific to the target tissues comprise a sequence selected from Table 2 or Table 3.
82. The system or method of any of the preceding embodiments, wherein the sequence specifically bound by the transposase comprises one or more inverted repeats, direct repeats, or inverted repeats and direct repeats.
83. A system comprising a first lipid nanoparticle comprising the polypeptide (or DNA or RNA encoding the same) of a Gene Writing™ system (e.g., as described herein); and
   a second lipid nanoparticle comprising a nucleic acid molecule of a Gene Writing™ System (e.g., as described herein).
84. The system or method of any of the preceding embodiments, wherein the system comprises one or more circular RNA molecules (circRNAs).
85. The system or method of any of the preceding embodiments, wherein the circRNA encodes the Gene Writer™ polypeptide.
86. The system or method of any of the preceding embodiments, wherein circRNA is delivered to a host cell.
87. The system or method of any of the preceding embodiments, wherein the circRNA is capable of being linearized, e.g., in a host cell, e.g., in the nucleus of the host cell.
88. The system or method of any of the preceding embodiments, wherein the circRNA 20 comprises a cleavage site.
89. The system or method of any of the preceding embodiments, wherein the circRNA further comprises a second cleavage site.
90. The system or method of any of the preceding embodiments, wherein the cleavage site can be cleaved by a ribozyme, e.g., a ribozyme comprised in the circRNA (e.g., by autocleavage).
91. The system or method of any of the preceding embodiments, wherein the circRNA comprises a ribozyme sequence.
92. The system or method of any of the preceding embodiments, wherein the ribozyme sequence is capable of autocleavage, e.g., in a host cell, e.g., in the nucleus of the host cell.
93. The system or method of any of the preceding embodiments, wherein the ribozyme is an inducible ribozyme.
94. The system or method of any of the preceding embodiments, wherein the ribozyme is a protein-responsive ribozyme, e.g., a ribozyme responsive to a nuclear protein, e.g., a genome-interacting protein, e.g., an epigenetic modifier, e.g., EZH2.
95. The system or method of any of the preceding embodiments, wherein the ribozyme is a nucleic acid-responsive ribozyme.
96. The system or method of any of the preceding embodiments, wherein the catalytic activity (e.g., autocatalytic activity) of the ribozyme is activated in the presence of a target nucleic acid molecule (e.g., an RNA molecule, e.g., an mRNA, miRNA, ncRNA, lncRNA, tRNA, snRNA, or mtRNA).
97. The system or method of any of the preceding embodiments, wherein the ribozyme is responsive to a target protein (e.g., an MS2 coat protein).
98. The system or method of any of the preceding embodiments, wherein the target protein localized to the cytoplasm or localized to the nucleus (e.g., an epigenetic modifier or a transcription factor).
99. The system or method of any of the preceding embodiments, wherein the ribozyme comprises the ribozyme sequence of a B2 or ALU retrotransposon, or a nucleic acid sequence having at least 85%, 90%, 95%, 96%, 97%, 98%, or 99% sequence identity thereto.
100. The system or method of any of the preceding embodiments, wherein the ribozyme comprises the sequence of a tobacco ringspot virus hammerhead ribozyme, or a nucleic acid sequence having at least 85%, 90%, 95%, 96%, 97%, 98%, or 99% sequence identity thereto.
101. The system or method of any of the preceding embodiments, wherein the ribozyme comprises the sequence of a hepatitis delta virus (HDV) ribozyme, or a nucleic acid sequence having at least 85%, 90%, 95%, 96%, 97%, 98%, or 99% sequence identity thereto.
102. The system or method of any of the preceding embodiments, wherein the ribozyme is activated by a moiety expressed in a target cell or target tissue.
103. The system or method of any of the preceding embodiments, wherein the ribozyme is activated by a moiety expressed in a target subcellular compartment (e.g., a nucleus, nucleolus, cytoplasm, or mitochondria).
104. The system or method of any of the preceding embodiments, wherein the ribozyme is comprised in a circular RNA or a linear RNA.
105. The system or method of any of the preceding embodiments, wherein the heterologous ribozyme is capable of cleaving RNA comprising the ribozyme, e.g., 5′ of the ribozyme, 3′ of the ribozyme, or within the ribozyme.
106. The system or method of any of the preceding embodiments, wherein the system, polypeptide, and/or DNA encoding the same, is formulated as a lipid nanoparticle (LNP).
107. The system or method of any of the preceding embodiments, wherein the lipid nanoparticle (or a formulation comprising a plurality of the lipid nanoparticles) lacks reactive impurities (e.g., aldehydes), or comprises less than a preselected level of reactive impurities (e.g., aldehydes).
108. The system or method of any of the preceding embodiments, wherein the lipid nanoparticle (or a formulation comprising a plurality of the lipid nanoparticles) lacks aldehydes, or comprises less than a preselected level of aldehydes.
109. The system or method of any of the preceding embodiments, wherein the lipid nanoparticle is comprised in a formulation comprising a plurality of the lipid nanoparticles.
110. The system or method of any of the preceding embodiments, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.
111. The system or method of any of the preceding embodiments, wherein the lipid nanoparticle formulation is produced using one or more lipid reagents comprising less than 3% total reactive impurity (e.g., aldehyde) content.
112. The system or method of any of the preceding embodiments, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.
113. The system or method of any of the preceding embodiments, wherein the lipid nanoparticle formulation is produced using one or more lipid reagent comprising less than 0.3% of any single reactive impurity (e.g., aldehyde) species.
114. The system or method of any of the preceding embodiments, wherein the lipid nanoparticle formulation is produced using one or more lipid reagents comprising less than 0.1% of any single reactive impurity (e.g., aldehyde) species.
115. The system or method of any of the preceding embodiments, wherein the lipid nanoparticle formulation comprises less than 5%, 4%, 3%, 2%, 1%, 0.9%, 0.8%, 0.7%, 0.6%, 0.4%, 0.3%, 0.2%, or 0.1% total reactive impurity (e.g., aldehyde) content.
116. The system or method any of the preceding embodiments, wherein the lipid nanoparticle formulation comprises less than 3% total reactive impurity (e.g., aldehyde) content.
117. The system or method of any of the preceding embodiments, wherein the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.
118. The system or method of any of the preceding embodiments, wherein the lipid nanoparticle formulation comprises less than 0.3% of any single reactive impurity (e.g., aldehyde) species.
119. The system or method of any of the preceding embodiments, wherein the lipid nanoparticle formulation comprises less than 0.1% of any single reactive impurity (e.g., aldehyde) species.
120. The system or method of any of the preceding embodiments, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.
121. The system or method of any of the preceding embodiments, wherein one or more, or optionally all, of the lipid reagents used for a lipid nanoparticle as described herein or a formulation thereof comprise less than 3% total reactive impurity (e.g., aldehyde) content.
122. The system or method of any of the preceding embodiments, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.3%, 0.2%, or 0.1% of any single reactive impurity (e.g., aldehyde) species.
123. The system or method of any of the preceding embodiments, wherein one or more, or optionally all, of the lipid reagents used for a lipid nanoparticle as described herein or a formulation thereof comprise less than 0.3% of any single reactive impurity (e.g., aldehyde) species.
124. The system or method of any of the preceding embodiments, wherein one or more, or optionally all, of the lipid reagents used for a lipid nanoparticle as described herein or a formulation thereof comprise less than 0.1% of any single reactive impurity (e.g., aldehyde) species.
125. The system or method of any of the preceding embodiments, wherein the total aldehyde content and/or quantity of any single reactive impurity (e.g., aldehyde) species is determined by liquid chromatography (LC), e.g., coupled with tandem mass spectrometry (MS/MS), e.g., according to the method described in Example 5.
126. The system or method of any of the preceding embodiments, wherein the total aldehyde content and/or quantity of reactive impurity (e.g., aldehyde) species is determined by detecting one or more chemical modifications of a nucleic acid molecule (e.g., as described herein) associated with the presence of reactive impurities (e.g., aldehydes), e.g., in the lipid reagents.
127. The system or method of any of the preceding embodiments, wherein the total aldehyde content and/or quantity of aldehyde species is determined by detecting one or more chemical modifications of a nucleotide or nucleoside (e.g., a ribonucleotide or ribonucleoside, e.g., comprised in or isolated from a nucleic acid molecule, e.g., as described herein) associated with the presence of reactive impurities (e.g., aldehydes), e.g., in the lipid reagents, e.g., as described in Example 6.
128. The system or method of any of the preceding embodiments, wherein the chemical modifications of a nucleic acid molecule, nucleotide, or nucleoside are detected by determining the presence of one or more modified nucleotides or nucleosides, e.g., using LC-MS/MS analysis, e.g., as described in Example 6.
129. The system or method of any preceding embodiment, wherein the system, nucleic acid molecule, polypeptide, and/or DNA encoding the same, is formulated as a lipid nanoparticle (LNP).
130. A lipid nanoparticle (LNP) comprising the system, polypeptide (or RNA encoding the same), nucleic acid molecule, or DNA encoding the system or polypeptide, of any preceding embodiment.
131. The LNP of any of the preceding embodiments, comprising a cationic lipid.
132. The LNP of any of the preceding embodiments, wherein the cationic lipid has a structure according to:

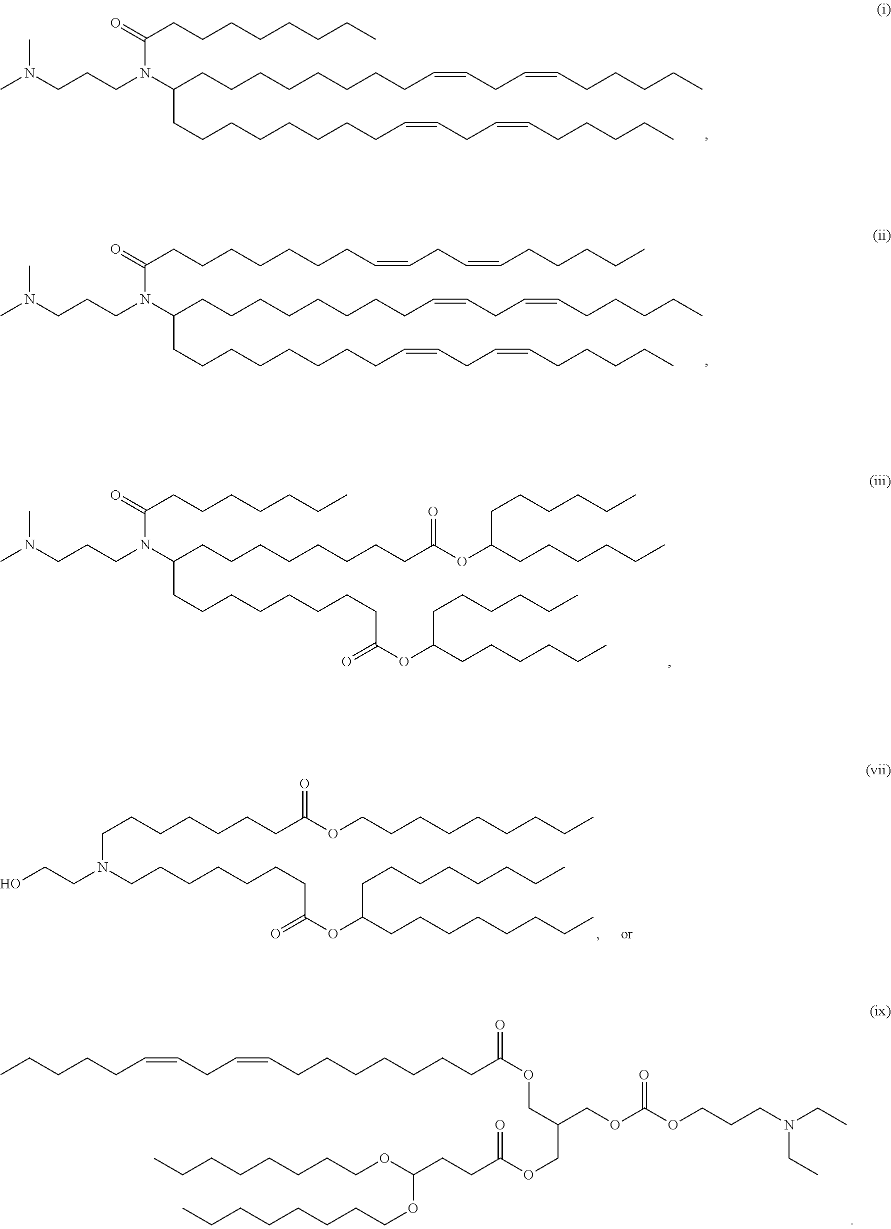

133. The LNP of any of the preceding embodiments, further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.
134. The LNP of any of the preceding embodiments, encapsulating at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the Gene Writer polypeptide
135. The system or method of any of the preceding embodiments, wherein an RNA of the system (e.g., the RNA encoding the polypeptide of (a), or an RNA expressed from a heterologous object sequence integrated into a target DNA) comprises a microRNA binding site, e.g., in a 3′ UTR.
136. The system or method of any of the preceding embodiments, wherein the microRNA binding site is recognized by a miRNA that is present in a non-target cell type, but that is not present (or is present at a reduced level relative to the non-target cell) in a target cell type.
137. The system or method of any of the preceding embodiments, wherein the miRNA is miR-142, and/or wherein the non-target cell is a Kupffer cell or a blood cell, e.g., an immune cell.
138. The system or method of any of the preceding embodiments, wherein the miRNA is miR-182 or miR-183, and/or wherein the non-target cell is a dorsal root ganglion neuron.
139. The system or method of any of the preceding embodiments, wherein the system comprises a first miRNA binding site that is recognized by a first miRNA (e.g., miR-142) and the system further comprises a second miRNA binding site that is recognized by a second miRNA (e.g., miR-182 or miR-183), wherein the first miRNA binding site and the second miRNA binding site are situated on the same RNA or on different RNAs of the system.
140. The system or method of any of the preceding embodiments, wherein the RNA encoding the polypeptide of (a) comprises at least 2, 3, or 4 miRNA binding sites, e.g., wherein the miRNA binding sites are recognized by the same or different miRNAs.
141. The system or method of any of the preceding embodiments, wherein the RNA expressed from a heterologous object sequence integrated into a target DNA comprises at least 2, 3, or 4 miRNA binding sites, e.g., wherein the miRNA binding sites are recognized by the same or different miRNAs.
142. A method of modifying a target DNA strand in a cell, tissue, or subject, the method comprising providing a system comprising:
   a) an mRNA encoding a DNA transposase, wherein the mRNA is formulated as a lipid nanoparticle (LNP); and
   b) a template nucleic acid comprising i) a sequence that specifically binds the transposase, and ii) a heterologous object sequence, wherein the template nucleic acid is associated with a viral capsid protein, e.g., an AAV capsid protein, e.g., a recombinant adeno-associated virus (rAAV) capsid protein; and
   administering the system to the cell, tissue, or subject, wherein the system inserts the heterologous object sequence into the target DNA strand, thereby modifying the target DNA strand.
143. A system comprising:
   a) an mRNA encoding a DNA transposase, wherein the mRNA is formulated as a lipid nanoparticle (LNP); and
   b) a template nucleic acid comprising i) a sequence that specifically binds the transposase, and ii) a heterologous object sequence, wherein the template nucleic acid is associated with a viral capsid protein, e.g., an AAV capsid protein, e.g., a recombinant adeno-associated virus (rAAV) capsid protein
   wherein the system optionally further comprises a pharmaceutically acceptable carrier or diluent.
144. The method or system of embodiment 142 or 143, wherein the template nucleic acid comprises an AAV ITR.
145. The method or system of any of embodiments 142-144, wherein the system further comprises one or more first tissue-specific expression-control sequences (e.g., a tissue-specific expression-control sequence described herein) specific to the target tissue; wherein the one or more first tissue-specific expression-control sequences specific to the target tissue are in operative association with (a), (b), or (a) and (b), wherein optionally the one or more first tissue-specific expression-control sequences comprises a tissue specific promoter (e.g., as described herein) or a tissue-specific microRNA recognition sequence (e.g., as described herein).
146. The method or system of embodiments 145, wherein:
i) the one or more first tissue-specific expression-control sequences specific to the target tissues comprise a sequence selected from Table 2 or Table 3,
ii) the heterologous object sequence comprises a sequence selected from Table 4, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB or paragraph 63; or
iii) (i) and (ii).
147. The method or system of any of embodiments 142-146, wherein the nucleic acid in (b) comprises DNA.
148. The method or system of any of embodiments 142-147, wherein the nucleic acid in (b):
   a. is single-stranded or comprises a single-stranded segment, e.g., is single-stranded DNA or comprises a single-stranded segment and one or more double stranded segments;
   b. has inverted terminal repeats; or
   c. both (i) and (ii).
149. The method or system of any of embodiments 145-148, wherein the one or more first tissue-specific expression-control sequences comprises a tissue specific promoter in operative association with the heterologous object sequence.
150. The method or system of any of embodiments 145-149, wherein the one or more first tissue-specific expression-control sequences comprises a tissue-specific microRNA recognition sequence in operative association with:
   i. the heterologous object sequence,
   ii. a nucleic acid encoding the transposase, or
   iii. (i) and (ii).
151. The method or system of any of embodiments 145-150, wherein the system further comprises one or more second tissue-specific expression-control sequences
152. The method or system of any of embodiments 142-151, wherein, when the system is provided to an organism, incorporation of the heterologous object sequence into the genome of a cell in the target tissue is at least: 1, 5, 10, 20, 25, 30, 35, 40, 45, 50, 55, 60, 65, 70, 75, 80, 85, 90, 91, 92, 93, 94, 95, 96, 97, 98, 99%, or more, of all integrations in the organism, e.g., at least: 1, 5, 10, 20, 25, 30, 35, 40, 45, 50, 55, 60, 65, 70, 75, 80, 85, 90, 91, 92, 93, 94, 95, 96, 97, 98, 99%, or more, of the expression of the heterologous object sequence is in a cell in the target tissue.
153. The method or system of any of embodiments 142-152, wherein, when the system provided to an organism, expression of the heterologous object sequence in a cell in the target tissue is at least: 1, 5, 10, 20, 25, 30, 35, 40, 45, 50, 55, 60, 65, 70, 75, 80, 85, 90, 91, 92, 93, 94, 95, 96, 97, 98, 99%, or more, of all expression of the heterologous object sequence in the organism, e.g., at least: 1, 5, 10, 20, 25, 30, 35, 40, 45, 50, 55, 60, 65, 70, 75, 80, 85, 90, 91, 92, 93, 94, 95, 96, 97, 98, 99%, or more, of the expression of the heterologous object sequence is in a cell in the target tissue.
154. The method or system of any of embodiments 142-153, wherein the rAAV capsid protein is from an AAV serotype selected from Table 5.
155. The method or system of any of embodiments 142-154, wherein the heterologous object sequence encodes a polypeptide of at least 25, 50, 100, 150, 200, 250, 300, 400, 500, 600, 700, 800, 900, 1000 residues, or more.
156. The method or system of any of embodiments 142-155, wherein the heterologous object sequence encodes an enzyme (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, a storage protein, and immune receptor, a synthetic protein (e.g. a chimeric antigen receptor), an antibody, or combinations thereof.

### Polypeptide Component of Gene Writer™ Gene Editor Systems

Gene Writer™ proteins are capable of efficiently writing DNA into a target genome. These proteins can constitute multiple classes of action, but in the context of this application, Gene Writer™ polypeptide will refer to one that is, or is derived from, a DNA transposase. Transposases are sequence-specific DNA binding proteins that also contain a catalytic domain that mediates DNA breakage and joining. These proteins integrate a DNA sequence flanked by recognition sequences into a target DNA sequence (a genomic locus in a target cell). Exemplary transposases, sometimes called Gene Writer™s or Gene Writer™ proteins, herein, comprise an amino acid sequence described in Table 1, or a functional fragment thereof, including variants thereof. A variant of a transposase includes amino acid sequences having at least 70, 75, 80, 85, 86, 87, 88, 89, 90, 91, 92, 93, 94, 95, 96, 97, 98, 99% identity to a reference polypeptide, or a functional fragment thereof, e.g., such as the reference polypeptides in Table 1. A variety of amino acid substitutions for variants of a reference polypeptide are possible, including substitution with non-canonical amino acids. In some embodiments, a variant of a polypeptide comprises conservative substitutions or highly conservative substitutions, relative to the reference sequence. “Conservative substitutions” relative to a reference sequence means a given amino acid substitution has a value of 0 or greater in BLOSUM62. “Highly conservative substitutions” relative to a reference sequence means a given amino acid substitution has a value of 1 or greater (e.g., in some embodiments, 2, or more) in BLOSUM62.

A transposase used in the systems and methods provided by the invention can be part of a fusion protein that includes heterologous domains, such as DNA-binding proteins, DNA bending proteins, and combinations thereof. In certain embodiments a transposase for use consonant with the invention includes Sleeping Beauty (SB), piggyBac (pB), TcBuster, or Space Invaders (SPIN), including variants thereof. Some transposable elements move by breakage and joining mediated only by the transposase, whereas others also involve DNA synthesis and ligation by host proteins to regenerate intact duplex DNA. There are four major classes of DNA-only transposases: DDE transposases, tyrosine-histidine-hydrophobic-histidine (HUH) transposases, tyrosine-transposases, and serine-transposases. DDE transposases break and join DNA by direct transesterification. The other classes of transposases act via covalent-protein DNA intermediates. Eubacteria, archaea, and eukaryotes all contain mobile elements with these four major classes of transposases.

In some embodiments, the transposase-based Gene Writer™ is derived from a DDE-type transposase. In some embodiments, the transposase-based Gene Writer™ is derived from a member of the Tc1/Mariner family. In some embodiments, the transposase-based Gene Writer™ is derived from the Sleeping Beauty transposase. Sleeping Beauty comprises the InterPro domains IPR036388 (Winged helix-like DNA-binding domain superfamily), IPR009057 (Homeobox-like domain superfamily), IPR002492 (Transposase, Tc1-like) and IPR038717 (Tc1-like transposase, DDE domain). In some embodiments, the transposase-based Gene Writer™ is derived from the hyperactive Sleeping Beauty SB100X (WO2019038197 SEQ ID:2, incorporated by reference) or its further derivative hsSB (WO2019038197 SEQ ID:1, incorporated by reference). In other embodiments, the transposase-based Gene Writer™ is derived from a member of the piggyBac family. In some embodiments, the transposase-based Gene Writer™ is derived from the piggyBac transposase. PiggyBac comprises the InterPro domain IPR029526 (PiggyBac transposable element-derived protein). In some embodiments, the transposase-based Gene Writer™ is derived from a hyperactive variant of the piggyBac transposase, e.g., 7pB (Doherty et al. Hum Gene Ther 2012). In some embodiments, the transposase-based Gene Writer™ is derived from the piggyBat transposase. PiggyBat comprises the InterPro domains IPR029526 (PiggyBac transposable element-derived protein) and IPR032718 (PiggyBac transposable element-derived protein 4, C-terminal zinc-ribbon). In other embodiments, the transposase-based Gene Writer™ is derived from a member of the hAT family. In some embodiments, the transposase-based Gene Writer™ is derived from TcBuster or a hyperactive version, e.g., TcBuster V596A (Table 1), e.g., a derivative of WO2018112415, incorporated herein by reference. TcBuster comprises the InterPro domain IPR012337 (Ribonuclease H-like superfamily). In some embodiments, the transposase-based Gene Writer™ is derived from Space Invaders (SPIN) or a hyperactive version, e.g., SPIN_{ON} (Table 1). In some embodiments, the Gene Writer™ system results in the creation of a target site duplication after integration of the template DNA, e.g., a TA dinucleotide duplication or TTAA duplication. In some embodiments, the Gene Writer™ system does not result in a target site duplication after integration of the template DNA.

In certain aspects of the present invention, the transposase of the Gene Writer™ system is based on a wild-type transposase. A wild-type transposase can be used in a Gene Writer™ system or can be modified (e.g., by insertion, deletion, or substitution of one or more residues) to alter the transposase activity for template and/or target DNA sequences. In some embodiments, the transposase is altered from its natural sequence to have altered codon usage, e.g., improved for human cells. In some embodiments, the amino acid sequence of the transposase of a Gene Writer™ system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a transposase whose sequence is referenced in Table 1. A person having ordinary skill in the art is capable of identifying transposases based upon homology to other known transposases using routine tools as Basic Local Alignment Search Tool (BLAST) or with reference to curated conserved domain structures, such as the InterPro domains noted herein, e.g., domains present in Column 3 of Table 1. In some embodiments, transposases are modified, for example, by site-specific mutation. In some embodiments, the transposase is engineered to bind a heterologous template DNA containing recognition sequences other than its native recognition sequences.

| | | SEQ | |
|---|---|---|---|
| | | ID | |
| Name | AA sequence | NO | InterPro Domains |
| SB100X | MGKSKEISQDLRKRIVDLHKSGSSL | 1530 | IPR036388 |
| | GAISKRLAVPRSSVQTIVRKYKHHG | | (Winged helix-like |
| | TTQPSYRSGRRRVLSPRDERTLVRK | | DNA-binding domain |
| | VQINPRTTAKDLVKMLEETGTKVSI | | superfamily); |
| | STVKRVLYRHNLKGHSARKKPLLQN | | IPR009057 |
| | RHKKARLRFATAHGDKDRTFWRNVL | | (Homeobox-like |
| | WSDETKIELFGHNDHRYVWRKKGEA | | domain superfamily); |
| | CKPKNTIPTVKHGGGSIMLWGCFAA | | IPR002492 |
| | GGTGALHKIDGIMDAVQYVDILKQH | | (Transposase, |
| | LKTSVRKLKLGRKWVFQHDNDPKHT | | Tc1-like); |
| | SKVVAKWLKDNKVKVLEWPSQSPDL | | IPR038717 |
| | NPIENLWAELKKRVRARRPTNLTQL | | (Tc1-like |
| | HQLCQEEWAKIHPNYCGKLVEGYPK | | transposase, |
| | RLTQVKQFKGNATKY | | DDE domain) |
| hsSB | MGKSKEISQDLRKRIVDLHKSGSSL | 1531 | IPR036388 |
| | GAISKRLAVPRSSVQTIVRKYKHHG | | (Winged helix-like |
| | TTQPSYRSGRRRVLSPRDERTLVRK | | DNA-binding domain |
| | VQINPRTTAKDLVKMLEETGTKVSI | | superfamily); |
| | STVKRVLYRHNLKGHSARKKPLLQN | | IPR009057 |
| | RHKKARLRFATAHGDKDRTFWRNVL | | (Homeobox-like |
| | WSDETKIELFGHNDHRYVWRKKGEA | | domain superfamily); |
| | SKPKNTIPTVKHGGGSIMLWGCFAA | | IPR002492 |
| | GGTGALHKIDGSMDAVQYVDILKQH | | (Transposase, |
| | LKTSVRKLKLGRKWVFQHDNDPKHT | | Tc1-like); |
| | SKVVAKWLKDNKVKVLEWPSQSPDL | | IPR038717 |
| | NPIENLWAELKKRVRARRPTNLTQL | | (Tc1-like |
| | HQLCQEEWAKIHPNYCGKLVEGYPK | | transposase, |
| | RLTQVKQFKGNATKY | | DDE domain) |
| Hyperactive | MGSSLDDEHILSALLQSDDELVGED | 1532 | IPR029526 |
| piggyBac | SDSEVSDHVSEDDVQSDTEEAFIDE | | (PiggyBac |
| (HyPBase/ | VHEVQPTSSGSEILDEQNVIEQPGS | | transposable |
| 7pB) | SLASNRILTLPQRTIRGKNKHCWST | | element- |
| | SKPTRRSRVSALNIVRSQRGPTRMC | | derived |
| | RNIYDPLLCFKLFFTDEIISEIVKW | | protein) |
| | TNAEISLKRRESMTSATFRDTNEDE | | |
| | IYAFFGILVMTAVRKDNHMSTDDLF | | |
| | DRSLSMVYVSVMSRDRFDFLIRCLR | | |
| | MDDKSIRPTLRENDVFTPVRKIWDL | | |
| | FIHQCIQNYTPGAHLTIDEQLLGFR | | |
| | GRCPFRVYIPNKPSKYGIKILMMCD | | |
| | SGTKYMINGMPYLGRGTQTNGVPLG | | |
| | EYYVKELSKPVHGSCRNITCDNWFT | | |
| | SIPLAKNLLQEPYKLTIVGTVRSNK | | |
| | REIPEVLKNSRSRPVGTSMFCFDGP | | |
| | LTLVSYKPKPAKMVYLLSSCDEDAS | | |
| | INESTGKPQMVMYYNQTKGGVDTLD | | |
| | QMCSVMTCSRKTNRWPMALLYGMIN | | |
| | IACINSFIIYSHNVSSKGEKVQSRK | | |
| | KFMRNLYMGLTSSFMRKRLEAPTLK | | |
| | RYLRDNISNILPKEVPGTSDDSTEE | | |
| | PVMKKRTYCTYCPSKIRRKASASCK | | |
| | KCKKVICREHNIDMCQSCF | | |
| Space | MTMDRVEKNVKKRKYSEDFLQYGFT | 1533 | |
| invaders | SIITAGIEKPQCVICCEVLSAESMK | | |
| (SPIN_{ON}) | PNKLKRHFDSKHPSFAGKDTNYFRS | | |
| | KADGLKKARLDTGGKYHKQNVAAIE | | |
| | ASYLVALRIARAMKPHTIAEDLLLP | | |
| | AAKDIVRVMIGDEFVTKLSAISLSN | | |
| | DTVRRRIDDMSADILDQVIQEIKSA | | |
| | PLPIFSIQLDESTDVANCSQLLVYV | | |
| | RYINDGDFKDEFLFCKPLEMTTTAR | | |
| | DVFDTVGSFLKEHKISWEKVCGVCT | | |
| | DGAPAMLGCRSGFQRLVLNESPKVI | | |
| | GTHCMIHRQILATKTLPQELQEVMK | | |
| | SVISSVNFVKASTLNSRLFSQLCNE | | |
| | LDAPNNALLFHTEVRWLSRGKVLKR | | |
| | VFELRDELKTFFNQKARPQFEALFS | | |
| | DKSELQKIAYLVDIFAILNELNLSL | | |
| | QGPNATCLDLSEKIRSFQMKLQLWQ | | |
| | KKLDENKIYMLPTLSAFFEEHDIEP | | |
| | DKRITMIISVKEHLHMLADEISSYF | | |
| | PNLPDTPFALARSPFTVKVEDVPET | | |
| | AQEEFIELINSDAARTDFSTMPVTK | | |
| | FWIKCLQSYPVLSETVLRLLLPFPT | | |
| | TYLCETGFSSLLVIKSKYRSRLVVE | | |
| | DDLRCALAKTAPRISDLVRKKQSQP | | |
| | SH | | |
| piggyBat | MAQHSDYSDDEFCADKLSNYSCDSD | 1534 | IPR029526 (PiggyBac |
| | LENASTSDEDSSDDEVMVRPRTLRR | | transposable element- |
| | RRISSSSSDSESDIEGGREEWSHVD | | derived protein); |
| | NPPVLEDFLGHQGLNTDAVINNIED | | IPR032718 (PiggyBac |
| | AVKLFIGDDFFEFLVEESNRYYNQN | | transposable element- |
| | RNNFKLSKKSLKWKDITPQEMKKFL | | derived protein 4, |
| | GLIVLMGQVRKDRRDDYWTTEPWTE | | C-terminal |
| | TPYFGKTMTRDRFRQIWKAWHENNN | | zinc-ribbon) |
| | ADIVNESDRLCKVRPVLDYFVPKFI | | |
| | NIYKPHQQLSLDEGIVPWRGRLFFR | | |
| | VYNAGKIVKYGILVRLLCESDTGYI | | |
| | CNMEIYCGEGKRLLETIQTVVSPYT | | |
| | DSWYHIYMDNYYNSVANCEALMKNK | | |
| | FRICGTIRKNRGIPKDFQTISLKKG | | |
| | ETKFIRKNDILLQVWQSKKPVYLIS | | |
| | SIHSAEMEESQNIDRTSKKKIVKPN | | |
| | ALIDYNKHMKGVDRADQYLSYYSIL | | |
| | RRTVKWTKRLAMYMINCALFNSYAV | | |
| | YKSVRQRKMGFKMFLKQTAIHWLTD | | |
| | DIPEDMDIVPDLQPVPSTSGMRAKP | | |
| | PTSDPPCRLSMDMRKHTLQAIVGSG | | |
| | KKKNILRRCRVCSVHKLRSETRYMC | | |
| | KFCNIPLHKGACFEKYHTLKNYLE | | |
| Hyperactive | MMLNWLKSGKLESQSQEQSSCYLEN | 1535 | IPR012337 |
| | SNCLPPTLDSTDIIGEENKAGTTSR | | (Ribonuclease H- |
| | KKRKYDEDYLNFGFTWTGDKDEPNG | | like superfamily) |
| | LCVICEQVVNNSSLNPAKLKRHLDT | | |
| | KHPTLKGKSEYFKRKCNELNQKKHT | | |
| | FERYVRDDNKNLLKASYLVSLRIAK | | |
| | QGEAYTIAEKLIKPCTKDLTTCVFG | | |
| | EKFASKVDLVPLSDTTISRRIEDMS | | |
| | YFCEAVLVNRLKNAKCGFTLQMDES | | |
| | TDVAGLAILLVFVRYIHESSFEEDM | | |
| | LFCKALPTQTTGEEIFNLLNAYFEK | | |
| | HSIPWNLCYHICTDGAKAMVGVIKG | | |
| | VIARIKKLVPDIKASHCCLHRHALA | | |
| | VKRIPNALHEVLNDAVKMINFIKSR | | |
| | PLNARVFALLCDDLGSLHKNLLLHT | | |
| | EVRWLSRGKVLTRFWELRDEIRIFF | | |
| | NEREFAGKLNDTSWLQNLAYIADIF | | |
| TcBuster | SYLNEVNLSLQGPNSTIFKVNSRIN | | |
| (V596A) | SIKSKLKLWEECITKNNTECFANLN | | |
| | DFLETSNTALDPNLKSNILEHLNGL | | |
| | KNTFLEYFPPTCNNISWVENPFNEC | | |
| | GNVDTLPIKEREQLIDIRTDTTLKS | | |
| | SFVPDGIGPFWIKLMDEFPEISKRA | | |
| | VKELMPFVTTYLCEKSFSVYAATKT | | |
| | KYRNRLDAEDDMRLQLTTIHPDIDN | | |
| | LCNNKQAQKSH | | |

While DNA transposon systems may be either random or possess some insertion site preferences, e.g., TA dinucleotide for Sleeping Beauty, TTAA tetranucleotide for piggyBac, it has been shown in the art that transposases can be programmed to have altered preferences for insertion sites. For example, it was shown that using a heterologous DNA binding domain that was fused to (i) the transposase; (ii) another protein that bound to a specific DNA sequence within the transposable element; or (iii) another protein that interacted with the transposase, enabled up to 10⁷-fold enrichment of transgene insertion at the desired target site (Ivics et al. Mol Ther 2007). Additionally, it has been shown that the addition of DNA targeting domains may also serve to limit overexpression inhibition of transposition (Wilson et al. FEBS Lett 2005).

In certain aspects, a DNA-binding domain of a Gene Writer™ polypeptide described herein is selected, designed, or constructed for binding to a desired host DNA target sequence. In certain embodiments, the DNA-binding domain of the transposase is a heterologous DNA-binding protein or domain relative to a native transposon sequence. In some embodiments, the heterologous DNA binding element is a zinc-finger element or a TAL effector element, e.g., a zinc-finger or TAL polypeptide or functional fragment thereof. In some embodiments, the heterologous DNA binding element is a sequence-guided DNA binding element, such as Cas9, Cpf1, or other CRISPR-related protein that has been altered to have no endonuclease activity. In some embodiments, the heterologous DNA binding element retains endonuclease activity. In some embodiments, the heterologous DNA binding element replaces a DNA-binding element of the polypeptide. In specific embodiments, the heterologous DNA-binding domain can be any one or more of Cas9, TAL domain, ZF domain, Myb domain, combinations thereof, or multiples thereof. A person having ordinary skill in the art is capable of identifying DNA binding domains based upon homology to other known DNA binding domains using tools as Basic Local Alignment Search Tool (BLAST). In still other embodiments, DNA-binding domains are modified, for example by site-specific mutation, increasing or decreasing DNA-binding elements (for example, number and/or specificity of zinc fingers), etc., to alter DNA-binding specificity and affinity. In some embodiments, the DNA binding domain is altered from its natural sequence to have altered codon usage, e.g., improved for human cells.

In certain aspects of the present invention, the host site integrated into by the Gene Writer™ system can be in a gene, in an intron, in an exon, an ORF, outside of a coding region of any gene, in a regulatory region of a gene, or outside of a regulatory region of a gene. In other aspects, the Gene Writer™ polypeptide may bind to one or more than one host DNA sequence. In some embodiments, the Gene Writer™ integrates DNA into the genome randomly. In some embodiments the Gene Writer™ integrates the DNA semi-randomly. In some embodiments the Gene Writer™ biases DNA Integration to intergenic or intragenic regions of the genome. In some embodiments the Gene Writer™ biases integrations into the 3′ or 5′ end of genes.

In certain embodiments, the polypeptide of the Gene Writer™ gene editor system, a transposase, further comprises an intracellular localization signal, e.g., a nuclear localization signal (NLS). The nuclear localization signal may be a peptide sequence that promotes the import of the protein into the nucleus. In some embodiments, the nuclear localization signal is at the N-terminus, C-terminus, or in an internal region of the polypeptide. In some embodiments, a plurality of the same or different nuclear localization signals are used. In some embodiments, the nuclear localization signal is less than 5, 10, 25, 50, 75, or 100 amino acids in length. Various polypeptide nuclear localization signals known in the art can be used.

As used in the systems and methods provided here, Gene Writers™ may be provided as either polypeptides, or nucleic acids encoding them.

### Endonuclease Domain:

In order to insert transposon DNA into a target site, some transposases are predicted to nick the target DNA, e.g., HUH transposases, e.g., Helitrons, IS608, IS91, ISCR1 (Thomas and Pritham Microbiol Spectr (2015)). In some embodiments, a Gene Writer comprises a transposase that nicks the target DNA during transposition. In some embodiments, a Gene Writer comprises a transposase that nicks the target DNA during transposition fused to a heterologous DNA-binding domain, e.g., Cas9. In some embodiments, the heterologous DNA-binding domain does not possess endonuclease activity, e.g., dCas9. In some embodiments, the heterologous DNA-binding domain possesses endonuclease activity, e.g., Cas9. In some embodiments, the heterologous DNA-binding domain possesses DNA nickase activity, e.g., Cas9 nickase. In some embodiments, the transposase fused to a nickase, e.g., Cas9 nickase, has been inactivated for endonuclease activity by mutation, such that it can no longer nick the target DNA. In some embodiments, the nicking activity of Cas9 complements the inactivated HUH endonuclease domain to catalyze transposition.

In some embodiments, the Gene Writer polypeptide comprises an endonuclease domain (e.g., a heterologous endonuclease domain). In some embodiments the endonuclease domain or endonuclease/DNA binding domain is altered from its natural sequence to have altered codon usage, e.g. improved for human cells. In some embodiments the endonuclease element is a heterologous endonuclease element, such as Fok1 nuclease, Cas9, or Cas9 nickase. In some embodiments, the heterologous endonuclease domain cleaves both DNA strands and forms double-stranded breaks. In some embodiments, the heterologous endonuclease activity has nickase activity and does not form double stranded breaks. The amino acid sequence of an endonuclease domain of a Gene Writer system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain of a transposon described herein. A person having ordinary skill in the art is capable of identifying endonuclease domains based upon homology to other known endonuclease domains using tools as Basic Local Alignment Search Tool (BLAST). In certain embodiments, the heterologous endonuclease is Cas9 or Cas9 nickase or a functional fragment thereof. In certain embodiments, the heterologous endonuclease is Fok1 or a functional fragment thereof. In certain embodiments, the heterologous endonuclease is a Holliday junction resolvase or homolog thereof, such as the Holliday junction resolving enzyme from Sulfolobus solfataricus—Ssol Hje (Govindaraju et al., Nucleic Acids Research 44:7, 2016). In certain embodiments, the heterologous endonuclease is the endonuclease of the large fragment of a spliceosomal protein, such as Prp8 (Mahbub et al., Mobile DNA 8:16, 2017). In still other embodiments, homologous endonuclease domains are modified, for example by site-specific mutation, to alter DNA endonuclease activity. In still other embodiments, endonuclease domains are modified to remove any latent DNA-sequence specificity.

In some embodiments, the endonuclease domain is capable of nicking a first strand and a second strand. In some embodiments, the first and second strand nicks occur at the same position in the target site but on opposite strands. In some embodiments, the second strand nick occurs in a staggered location, e.g., upstream or downstream, from the first nick. In some embodiments, the endonuclease domain generates a target site deletion if the second strand nick is upstream of the first strand nick. In some embodiments, the endonuclease domain generates a target site duplication if the second strand nick is downstream of the first strand nick. In some embodiments, the endonuclease domain generates no duplication and/or deletion if the first and second strand nicks occur in the same position of the target site (e.g., as described in Gladyshev and Arkhipova Gene 2009, incorporated by reference herein in its entirety). In some embodiments, the endonuclease domain has altered activity depending on protein conformation or RNA-binding status, e.g., which promotes the nicking of the first or second strand (e.g., as described in Christensen et al. PNAS 2006; incorporated by reference herein in its entirety).

In some embodiments, the endonuclease domain comprises a meganuclease, or a functional fragment thereof. In some embodiments, the endonuclease domain comprises a homing endonuclease, or a functional fragment thereof. In some embodiments, the endonuclease domain comprises a meganuclease from the LAGLIDADG (SEQ ID NO: 1536), GIY-YIG, HNH, His-Cys Box, or PD-(D/E) XK families, or a functional fragment or variant thereof, e.g., which possess conserved amino acid motifs, e.g., as indicated in the family names. In some embodiments, the endonuclease domain comprises a meganuclease, or fragment thereof, chosen from, e.g., I-SmaMI (Uniprot F7WD42), I-SceI (Uniprot P03882), I-Anil (Uniprot P03880), I-DmoI (Uniprot P21505), I-CreI (Uniprot P05725), I-TevI (Uniprot P13299), I-OnuI (Uniprot Q4VWW5), or I-BmoI (Uniprot Q9ANR6). In some embodiments, the meganuclease is naturally monomeric, e.g., I-SceI, I-TevI, or dimeric, e.g., I-CreI, in its functional form. For example, the LAGLIDADG meganucleases (“LAGLIDADG” disclosed as SEQ ID NO: 1536) with a single copy of the LAGLIDADG motif (SEQ ID NO: 1536) generally form homodimers, whereas members with two copies of the LAGLIDADG motif (SEQ ID NO: 1536) are generally found as monomers. In some embodiments, a meganuclease that normally forms as a dimer is expressed as a fusion, e.g., the two subunits are expressed as a single ORF and, optionally, connected by a linker, e.g., an I-CreI dimer fusion (Rodriguez-Fomes et al. Gene Therapy 2020; incorporated by reference herein in its entirety). In some embodiments, a meganuclease, or a functional fragment thereof, is altered to favor nickase activity for one strand of a double-stranded DNA molecule, e.g., I-SceI (K1221 and/or K223I) (Niu et al. J Mol Biol 2008), I-Anil (K227M) (McConnell Smith et al. PNAS 2009), I-DmoI (Q42A and/or K120M) (Molina et al. J Biol Chem 2015). In some embodiments, a meganuclease or functional fragment thereof possessing this preference for single-strand cleavage is used as an endonuclease domain, e.g., with nickase activity. In some embodiments, an endonuclease domain comprises a meganuclease, or a functional fragment thereof, which naturally targets or is engineered to target a safe harbor site, e.g., an I-CreI targeting SH6 site (Rodriguez-Fomes et al., supra). In some embodiments, an endonuclease domain comprises a meganuclease, or a functional fragment thereof, with a sequence tolerant catalytic domain, e.g., I-TevI recognizing the minimal motif CNNNG (Kleinstiver et al. PNAS 2012). In some embodiments, a target sequence tolerant catalytic domain is fused to a DNA binding domain, e.g., to direct activity, e.g., by fusing I-TevI to: (i) zinc fingers to create Tev-ZFEs (Kleinstiver et al. PNAS 2012), (ii) other meganucleases to create MegaTevs (Wolfs et al. Nucleic Acids Res 2014), and/or (iii) Cas9 to create TevCas9 (Wolfs et al. PNAS 2016).

In some embodiments, the endonuclease domain comprises a restriction enzyme, e.g., a Type IIS or Type IIP restriction enzyme. In some embodiments, the endonuclease domain comprises a Type IIS restriction enzyme, e.g., FokI, or a fragment or variant thereof. In some embodiments, the endonuclease domain comprises a Type IIP restriction enzyme, e.g., PvuII, or a fragment or variant thereof. In some embodiments, a dimeric restriction enzyme is expressed as a fusion such that it functions as a single chain, e.g., a FokI dimer fusion (Minczuk et al. Nucleic Acids Res 36(12):3926-3938 (2008)).

The use of additional endonuclease domains is described, for example, in Guha and Edgell Int J Mol Sci 18(22):2565 (2017), which is incorporated herein by reference in its entirety.

In some embodiments, an endonuclease domain or DNA binding domain comprises a Streptococcus pyogenes Cas9 (SpCas9) or a functional fragment or variant thereof. In some embodiments, the endonuclease domain or DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T13371, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T13371, T1337V, T1337F, T1337S, T1337N, T1337K, T1337R, T1337H, T1337Q, and T1337M, or corresponding amino acid substitutions thereto.

In some embodiments, the endonuclease domain or DNA binding domain comprises a Cas domain, e.g., a Cas9 domain. In embodiments, the endonuclease domain or DNA binding domain comprises a nuclease-active Cas domain, a Cas nickase (nCas) domain, or a nuclease-inactive Cas (dCas) domain. In embodiments, the endonuclease domain or DNA binding domain comprises a nuclease-active Cas9 domain, a Cas9 nickase (nCas9) domain, or a nuclease-inactive Cas9 (dCas9) domain. In some embodiments, the endonuclease domain or DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises an S. pyogenes or an S. thermophilus Cas9, or a functional fragment thereof. In some embodiments, the endonuclease domain or DNA binding domain comprises a Cas9 sequence, e.g., as described in Chylinski, Rhun, and Charpentier (2013) RNA Biology 10:5, 726-737; incorporated herein by reference. In some embodiments, the endonuclease domain or DNA binding domain comprises the HNH nuclease subdomain and/or the RuvC1 subdomain of a Cas, e.g., Cas9, e.g., as described herein, or a variant thereof. In some embodiments, the endonuclease domain or DNA binding domain comprises Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the endonuclease domain or DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the endonuclease domain or DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the endonuclease domain or DNA binding domain comprises spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the endonuclease domain or DNA-binding domain comprises an amino acid sequence as listed in Table 11 below, or an amino acid sequence having at least 80%, 85%, 90%, 95%, 96%, 97%, 98%, or 99% sequence identity thereto. In some embodiments, the endonuclease domain or DNA-binding domain comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 differences (e.g., mutations) relative to any of the amino acid sequences described herein.

TABLE 11
Each of the Reference Sequences are incorporated by reference in their entirety.

| Name | Amino Acid Sequence or Reference Sequence |
|---|---|
| Streptococcus pyogenes | |
| Cas9 | |
| Exemplary Linker | SGSETPGTSESATPES (SEQ ID NO: 1542) |
| Exemplary Linker Motif | (SGGS)ₙ (SEQ ID NO: 1543) |
| Exemplary Linker Motif | (GGGS)ₙ (SEQ ID NO: 1544) |
| Exemplary Linker Motif | (GGGGS)ₙ (SEQ ID NO: 1545) |
| Exemplary Linker Motif | (G)ₙ |
| Exemplary Linker Motif | (EAAAK)ₙ (SEQ ID NO: 1546) |
| Exemplary Linker Motif | (GGS)ₙ |
| Exemplary Linker Motif | (XP)ₙ |
| Cas9 from Streptococcus | NCBI Reference Sequence: NC_002737.2 and Uniprot Reference |
| pyogenes | Sequence: Q99ZW2 |
| Cas9 from Corynebacterium | NCBI Refs: NC_015683.1, NC_017317.1 |
| ulcerans | |
| Cas9 from Corynebacterium | NCBI Refs: NC_016782.1, NC_016786.1 |
| diphtheria | |
| Cas9 from Spiroplasma | NCBI Ref: NC_021284.1 |
| syrphidicola | |
| Cas9 from Prevotella | NCBI Ref: NC_017861.1 |
| intermedia | |
| Cas9 from Spiroplasma | NCBI Ref: NC_021846.1 |
| taiwanense | |
| Cas9 from Streptococcus | NCBI Ref: NC_021314.1 |
| iniae | |
| Cas9 from Belliella baltica | NCBI Ref: NC_018010.1 |
| Cas9 from Psychroflexus | NCBI Ref: NC_018721.1 |
| torquisl | |
| Cas9 from Streptococcus | NCBI Ref: YP_820832.1 |
| thermophilus | |
| Cas9 from Listeria innocua | NCBI Ref: NP_472073.1 |
| Cas9 from Campylobacter | NCBI Ref: YP_002344900.1 |
| jejuni | |
| Cas9 from Neisseria | NCBI Ref: YP_002342100.1 |
| meningitidis | |
| dCas9 (D10A and H840A) | |
| Catalytically inactive Cas9 | |
| (dCas9) | |
| Cas9 nickase (nCas9) | |
| Catalytically active Cas9 | |
| CasY | ((ncbi.nlm.nih.gov/protein/APG80656.1) |
| | >APG80656.1 CRISPR-associated protein CasY [uncultured |
| | Parcubacteria group bacterium]) |
| CasX | uniprot.org/uniprot/FONN87; uniprot.org/uniprot/FONH53 |
| CasX | >tr\|FONH53\|FONH53_SULIR CRISPR associated protein, |
| | Casx OS = Sulfolobus islandicus (strain REY15A) |
| | GN = SiRe_0771 PE = 4 SV = 1 |
| Deltaproteobacteria CasX | |
| Cas12b/C2c1 | ((uniprot.org/uniprot/TOD7A2#2) sp\|TOD7A2\|C2C1_ALIAG |
| | CRISPR-associated endonuclease C2c1 OS = Alicyclobacillus |
| | acido-terrestris (strain ATCC 49025/DSM 3922/CIP 10613/ |
| | NCIMB 13137/GD3B) GN = c2c1 PE = 1 SV = 1) |
| BhCas12b (Bacillus hisashii) | NCBI Reference Sequence: WP_095142515 |
| BvCas12b (Bacillus sp. V3- | NCBI Reference Sequence: WP_101661451.1 |
| 13) | |
| Wild-type Francisella | |
| novicida Cpf1 | |
| Francisella novicida Cpf1 | |
| D917A | |
| Francisella novicida Cpf1 | |
| E1006A | |
| Francisella novicida Cpf1 | |
| D1255A | |
| Francisella novicida Cpf1 | |
| D917A/E1006A | |
| Francisella novicida Cpf1 | |
| D917A/D1255A | |
| Francisella novicida Cpf1 | |
| E1006A/D1255A | |
| Francisella novicida Cpf1 | |
| D917A/E1006A | |
| SaCas9 | |
| SaCas9n | |
| PAM-binding SpCas9 | |
| PAM-binding SpCas9n | |
| PAM-binding SpEQR Cas9 | |
| PAM-binding SpVQR Cas9 | |
| PAM-binding SpVRER Cas9 | |
| PAM-binding SpVRQR Cas9 | |
| SpyMacCas9 | |

In some embodiments, a Gene Writing polypeptide has an endonuclease domain comprising a Cas9 nickase, e.g., Cas9 H840A. In embodiments, the Cas9 H840A has the following amino acid sequence:

Cas9 nickase (H840A):
(SEQ ID NO: 1547)
DKKYSIGLDIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFL VEEDKKHERHPIFG
NIVDEVAYHEKYPTIYHLRKKLVDSTDKADLRLIYLALAH
MIKFRGHFLIEGDLNPDNSDVDKLFIQLVQTYNQLFEENP
INASGVDAKAILSARLSKSRRLENLIAQLPGEKKNGLFGN
LIALSLGLTPNFKSNFDLAEDAKLQLSKDTYDDDLDNLLA
QIGDQYADLFLAAKNLSDAILLSDILRVNTEITKAPLSAS
MIKRYDEHHQDLTLLKALVRQQLPEKYKEIFFDQSKNGYA
GYIDGGASQEEFYKFIKPILEKMDGTEELLVKLNREDLLR
KQRTFDNGSIPHQIHLGELHAILRRQEDFYPFLKDNREKI
EKILTFRIPYYVGPLARGNSRFAWMTRKSEETITPWNFEE
VVDKGASAQSFIERMTNFDKNLPNEKVLPKHSLLYEYFTV
YNELTKVKYVTEGMRKPAFLSGEQKKAIVDLLFKTNRKVT
VKQLKEDYFKKIECFDSVEISGVEDRFNASLGTYHDLLKI
IKDKDFLDNEENEDILEDIVLTLTLFEDREMIEERLKTYA
HLEDDKVMKQLKRRRYTGWGRLSRKLINGIRDKQSGKTIL
DFLKSDGFANRNFMQLIHDDSLTFKEDIQKAQVSGQGDSL
HEHIANLAGSPAIKKGILQTVKVVDELVKVMGRHKPENIV
IEMARENQTTQKGQKNSRERMKRIEEGIKELGSQILKEHP
VENTQLQNEKLYLYYLQNGRDMYVDQELDINRLSDYDVDA
IVPQSFLKDDSIDNKVLTRSDKNRGKSDNVPSEEVVKKMK
NYWRQLLNAKLITQRKFDNLTKAERGGLSELDKAGFIKRQ
LVETRQITKHVAQILDSRMNTKYDENDKLIREVKVITLKS
KLVSDFRKDFQFYKVREINNYHHAHDAYLNAVVGTALIKK
YPKLESEFVYGDYKVYDVRKMIAKSEQEIGKATAKYFFYS
NIMNFFKTEITLANGEIRKRPLIETNGETGEIVWDKGRDF
ATVRKVLSMPQVNIVKKTEVQTGGFSKESILPKRNSDKLI
ARKKDWDPKKYGGFDSPTVAYSVLVVAKVEKGKSKKLKSV
KELLGITIMERSSFEKNPIDFLEAKGYKEVKKDLIIKLPK
YSLFELENGRKRMLASAGELQKGNELALPSKYVNFLYLAS
HYEKLKGSPEDNEQKQLFVEQHKHYLDEIIEQISEFSKRV
ILADANLDKVLSAYNKHRDKPIREQAENIIHLFTLTNLGA
PAAFKYFDTTIDRKRYTSTKEVLDATLIHQSITGLYETRI
DLSQLGGD

In some embodiments, a Gene Writer polypeptide comprises a dCas9 sequence comprising a D10A and/or H840A mutation (e.g., as a DNA binding domain), e.g., the following sequence:

(SEQ ID NO: 1548)
SMDKKYSIGLAIGTNSVGWAVITDDYKVPSKKFKVLGNTDRHSIKKNLIG
ALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEH
PVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKL
IREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD

In some embodiments, the Cas polypeptide binds a gRNA that directs DNA binding. In some embodiments, the gRNA comprises, e.g., from 5′ to 3′ (1) a gRNA spacer; (2) a gRNA scaffold. In some embodiments:
(1) Is a Cas9 spacer of ˜18-22 nt, e.g., is 20 nt
(2) Is a gRNA scaffold comprising one or more hairpin loops, e.g., 1, 2, of 3 loops for associating the template with a nickase Cas9 domain. In some embodiments, the gRNA scaffold carries the sequence, from 5′ to 3′,

(SEQ ID NO: 1549)
GTTTTAGAGCTAGAAATAGCAAGTTAAAATAAGGCTAGTCCGTTATCAAC
TTGAAAAAGTGGGACCGAGTCGGTCC

A second gRNA associated with the system may help drive complete integration. In some embodiments, the second gRNA may target a location that is 0-200 nt away from the first-strand nick, e.g., 0-50, 50-100, 100-200 nt away from the first-strand nick. In some embodiments, the second gRNA can only bind its target sequence after the edit is made, e.g., the gRNA binds a sequence present in the heterologous object sequence, but not in the initial target sequence.

In some embodiments, a Gene Writing system described herein is used to make an edit in HEK293, K562, U2OS, or HeLa cells. In some embodiment, a Gene Writing system is used to make an edit in primary cells, e.g., primary cortical neurons from E18.5 mice.

In some embodiments, an endonuclease domain (e.g., as described herein) comprises nCAS9, e.g., comprising the H840A mutation.

In some embodiments, the heterologous object sequence (e.g., of a system as described herein) is about 1-50, 50-100, 100-200, 200-300, 300-400, 400-500, 500-600, 600-700, 700-800, 800-900, 900-1000, or more, nucleotides in length.

In some embodiments, a system or method described herein involves a CRISPR DNA targeting enzyme or system described in US Pat. App. Pub. No. 20200063126, 20190002889, or 20190002875 (each of which is incorporated by reference herein in its entirety) or a functional fragment or variant thereof. For instance, in some embodiments, a GeneWriter polypeptide or Cas endonuclease described herein comprises a polypeptide sequence of any of the applications mentioned in this paragraph, and in some embodiments a guide RNA comprises a nucleic acid sequence of any of the applications mentioned in this paragraph.

### DNA Binding Domain:

In certain aspects, the DNA-binding domain of a Gene Writer polypeptide described herein is selected, designed, or constructed for binding to a desired host DNA target sequence. In some embodiments, the heterologous DNA binding element is a zinc-finger element or a TAL effector element, e.g., a zinc-finger or TAL polypeptide or functional fragment thereof. In some embodiments, the heterologous DNA binding element is a sequence-guided DNA binding element, such as Cas9, Cpf1, or other CRISPR-related protein that has been altered to have no endonuclease activity. In some embodiments the heterologous DNA binding element retains endonuclease activity. In some embodiments the heterologous DNA binding element replaces the endonuclease domain of the polypeptide. In specific embodiments, the heterologous DNA-binding domain can be any one or more of Cas9 (e.g., Cas9, Cas9 nickase, dCas9), TAL domain, zinc finger (ZF) domain, Myb domain, combinations thereof, or multiples thereof. In certain embodiments, the heterologous DNA-binding domain is a DNA binding domain described herein. A person having ordinary skill in the art is capable of identifying DNA binding domains based upon homology to other known DNA binding domains using tools as Basic Local Alignment Search Tool (BLAST). In still other embodiments, DNA-binding domains are modified, for example by site-specific mutation, increasing or decreasing DNA-binding elements (for example, number and/or specificity of zinc fingers), etc., to alter DNA-binding specificity and affinity. In some embodiments the DNA binding domain is altered from its natural sequence to have altered codon usage, e.g. improved for human cells.

In some embodiments, the DNA binding domain comprises a meganuclease domain (e.g., as described herein, e.g., in the endonuclease domain section), or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40(2):847-860 (2012), incorporated herein by reference in its entirety. In embodiments, the DNA binding domain comprises one or more modifications relative to a wild-type DNA binding domain, e.g., a modification via directed evolution, e.g., phage-assisted continuous evolution (PACE).

In some embodiments, a polypeptide described herein comprises one or more (e.g., 2, 3, 4, 5) nuclear targeting sequences, for example a nuclear localization sequence (NLS). In some embodiments, the NLS is a bipartite NLS. In some embodiments, an NLS facilitates the import of a protein comprising an NLS into the cell nucleus. In some embodiments, the NLS is fused to the N-terminus of a Gene Writer described herein. In some embodiments, the NLS is fused to the C-terminus of the Gene Writer. In some embodiments, the NLS is fused to the N-terminus or the C-terminus of a Cas domain. In some embodiments, a linker sequence is disposed between the NLS and the neighboring domain of the Gene Writer.

In some embodiments, an NLS comprises the amino acid sequence MDSLLMNRRKFLYQFKNVRWAKGRRETYLC (SEQ ID NO: 1550), PKKRKVEGADKRTADGSEFESPKKKRKV (SEQ ID NO: 1551), RKSGKIAAIWKRPRKPKKKRKV (SEQ ID NO: 1552), KRTADGSEFESPKKKRKV (SEQ ID NO: 1553), KKTELQTTNAENKTKKL (SEQ ID NO: 1554), or KRGINDRNFWRGENGRKTR (SEQ ID NO: 1555), KRPAATKKAGQAKKKK (SEQ ID NO: 1556), or a functional fragment or variant thereof. Exemplary NLS sequences are also described in PCT/EP2000/011690, the contents of which are incorporated herein by reference for their disclosure of exemplary nuclear localization sequences.

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK (SEQ ID NO: 1556), wherein the spacer is bracketed. Another exemplary bipartite NLS has the sequence PKKKRKVEGADKRTADGSEFESPKKKRKV (SEQ ID NO: 1557). Exemplary NLSs are described in International Application WO2020051561, which is herein incorporated by reference in its entirety, including for its disclosures regarding nuclear localization sequences.

### Inteins

In some embodiments, the Gene Writer system comprises an intein. Generally, an intein comprises a polypeptide that has the capacity to join two polypeptides or polypeptide fragments together via a peptide bond. In some embodiments, the intein is a trans-splicing intein that can join two polypeptide fragments, e.g., to form the polypeptide component of a system as described herein. In some embodiments, an intein may be encoded on the same nucleic acid molecule encoding the two polypeptide fragments. In certain embodiments, the intein may be translated as part of a larger polypeptide comprising, e.g., in order, the first polypeptide fragment, the intein, and the second polypeptide fragment. In embodiments, the translated intein may be capable of excising itself from the larger polypeptide, e.g., resulting in separation of the attached polypeptide fragments. In embodiments, the excised intein may be capable of joining the two polypeptide fragments to each other directly via a peptide bond. Exemplary inteins are described herein.

In some embodiments, as described in more detail below, Intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independent chosen from a DNA binding domain, a polymerase domain, and an endonuclease domain.

In some embodiments, a system or method described herein involves an intein that is a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem. 289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C (e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, Intein-N and intein-C may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]˜C. In some embodiments, an intein-C is fused to the N-terminus of the C-terminal portion of the split Cas9, i.e., to form a structure of N-[intein-C]˜[C-terminal portion of the split Cas9]-C. The mechanism of intein-mediated protein splicing for joining the proteins the inteins are fused to (e.g., split Cas9) is described in Shah et al., Chem Sci. 2014; 5(1):446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a Gene Writer (e.g., Cas9-R2Tg) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, an endonuclease domain (e.g., a nickase Cas9 domain) is fused to intein-N and a polypeptide comprising a polymerase domain is fused to an intein-C.

Exemplary nucleotide and amino acid sequences of interns are provided below:

DnaE Intein-N DNA:
(SEQ ID NO: 1558)
TGCCTGTCATACGAAACCGAGATACTGACAGTAGAATATGGCCTTCTGCC
AATCGGGAAGATTGTGGAGAAACGGATAGAATGCACAGTTTACTCTGTCG
ATAACAATGGTAACATTTATACTCAGCCAGTTGCCCAGTGGCACGACCGG
GGAGAGCAGGAAGTATTCGAATACTGTCTGGAGGATGGAAGTCTCATTAG
GGCCACTAAGGACCACAAATTTATGACAGTCGATGGCCAGATGCTGCCTA
TAGACGAAATCTTTGAGCGAGAGTTGGACCTCATGCGAGTTGACAACCTT
CCTAAT
DnaE Intein-N Protein:
(SEQ ID NO: 1559)
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN
DnaE Intein-C DNA:
(SEQ ID NO: 1560)
ATGATCAAGATAGCTACAAGGAAGTATCTTGGCAAACAAAACGTTTATGA
TATTGGAGTCGAAAGAGATCACAACTTTGCTCTGAAGAACGGATTCATAG
CTTCTAAT
Intein-C:
(SEQ ID NO: 1561)
MIKIATRKYLGKQNVYDIGVERDHNFALKNGFIASN
Cfa-N DNA:
(SEQ ID NO: 1562)
TGCCTGTCTTATGATACCGAGATACTTACCGTTGAATATGGCTTCTTGCC
TATTGGAAAGATTGTCGAAGAGAGAATTGAATGCACAGTATATACTGTAG
ACAAGAATGGTTTCGTTTACACACAGCCCATTGCTCAATGGCACAATCGC
GGCGAACAAGAAGTATTTGAGTACTGTCTCGAGGATGGAAGCATCATACG
AGCAACTAAAGATCATAAATTCATGACCACTGACGGGCAGATGTTGCCAA
TAGATGAGATATTCGAGCGGGGCTTGGATCTCAAACAAGTGGATGGATTG
CCA
Cfa-N Protein:
(SEQ ID NO: 1563)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P
Cfa-C DNA:
(SEQ ID NO: 1564)
ATGAAGAGGACTGCCGATGGATCAGAGTTTGAATCTCCCAAGAAGAAGAG
GAAAGTAAAGATAATATCTCGAAAAAGTCTTGGTACCCAAAATGTCTATG
ATATTGGAGTGGAGAAAGATCACAACTTCCTTCTCAAGAACGGTCTCGTA
GCCAGCAAC
Cfa-C Protein:
(SEQ ID NO: 1565)
MKRTADGSEFESPKKKRKVKIISRKSLGTQNVYDIGVEKDHNFLLKNGLV
ASN

### DNA Polymerizing Gene Writers:

Though transposition occurs most frequently in nature via cut- or copy-and-paste some mechanisms, some transposases encode additional domains to permit DNA-dependent DNA polymerization. In some embodiments, a Gene Writer comprises a domain capable of DNA-dependent DNA polymerization. In some embodiments, a Gene Writer comprises a transposase capable of DNA-dependent polymerization, e.g., a Polinton, a Helitron. In some embodiments, a Gene Writer comprises a transposase that replicates through a rolling circle intermediate, e.g., a Helitron. In some embodiments, a Gene Writer comprises an additional helicase domain, e.g., the helicase domain from a transposon, e.g., the helicase domain from a Helitron. In some embodiments, the Gene Writer functions to polymerize DNA at a nick site in a target DNA. In some embodiments, the Gene Writer functions to perform target-primed DNA polymerization, e.g., target-primed DNA-dependent DNA polymerization or target-primed RNA-dependent DNA polymerization (e.g. target-primed reverse transcription).

In some embodiments the transposase comprises a DNA binding domain, an endonuclease domain, and a DNA polymerization domain. In some embodiments the endonuclease and DNA binding domain are heterologous to the DNA polymerization domain. In some embodiments the endonuclease domain and DNA polymerization domain are heterologous to the DNA binding domain. In some embodiments the endonuclease domain is heterologous to the DNA binding domain and the DNA polymerization domain. In some embodiments the DNA binding domain comprises an endonuclease domain. In some embodiments the endonuclease domain nicks DNA. In some embodiments the endonuclease and/or DNA binding domain is an RNA-guided protein, e.g., a Cas protein. In some embodiments the transposase is mutated to have no DNA binding and/or endonuclease activity.

In some embodiments the transposase is localized to a nick by a DNA binding domain. In some embodiments the transposase nicks template DNA. In some embodiments the nick is targeted by a first guide DNA. In some embodiments, the first guide DNA is provided with the template DNA as a separate nucleic acid. In some embodiments, the DNA template and the first guide DNA are part of the same nucleic acid molecule. In some embodiments, the nick is targeted by a first guide RNA. In some embodiments, the first gRNA is provided with the template DNA as a separate nucleic acid. In some embodiments, the template DNA and first gRNA are part of the same nucleic acid molecule, e.g., are a single molecule that is a hybrid of RNA and DNA regions. In some embodiments the transposase nicks target DNA. In some embodiments the transposase anneals a DNA template to nicked target DNA. In some embodiments, the transposase anneals an RNA region of an RNA/DNA hybrid molecule to nicked target DNA. In some embodiments the DNA template is comprises complementary DNA sequence that anneals (e.g., via Watson-crick base-pairing) to the nick. In some embodiments the complementary sequence is at the 3′ or 5′ end of the DNA template. In some embodiments the complementary sequence is complementary to 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more base pairs adjacent to the nicked DNA strand. In some embodiments the DNA template is single stranded. In some embodiments the DNA template is double stranded. In some embodiments the DNA template is linear. In some embodiments the DNA template is circular.

In some embodiments the transposase comprises DNA polymerase activity. In some embodiments the transposase comprises DNA-dependent or RNA-dependent DNA polymerase activity. In some embodiments the transposase is a rolling circle transposase, e.g. a helitron transposase. In some embodiments the DNA polymerase is a rolling circle DNA polymerase, e.g., phi29. In some embodiments the DNA polymerase is described in Wawrzyniak et al., Frontiers of Microbiology, 2017, https://doi.org/10.3389/fmicb.2017.02353. In some embodiments the DNA polymerase is a eukaryotic or prokaryotic DNA polymerase. In some embodiments the DNA polymerase is a thermostable DNA polymerase. In some embodiments the DNA polymerase has been engineered to have increased processivity. In some embodiments the DNA polymerase is engineered to have increased fidelity. In some embodiments the DNA polymerase has at least 1, 2, 3, 4, 5, 6, 7, 8, 9, 10, 20, 30, 40, 50, 60, 70, 80, 90, or 100 amino acid substitutions as compared to a wild-type polymerase.

In some embodiments the annealed template primes DNA polymerization of a new strand of DNA using the DNA template.

In some embodiments the transposase nicks the opposite strand of DNA before DNA polymerization. In some embodiments the transposase nicks the opposite strand of after DNA polymerization. In some embodiments the transposase nicks the opposite strand of before DNA polymerization. In some embodiments the transposase nicks the opposite strand of DNA upstream or downstream (e.g. 5′ or 3′) of the first nick of DNA

In some embodiments the newly polymerized DNA downstream is ligated downstream of the first nick. In some embodiments the transposase ligates the DNA.

In some embodiments the second nick is made by a separate enzyme. In some embodiments the second nick is guided by a second guide DNA.

In some embodiments the transposase catalyzes a transesterification of the template DNA into the target DNA at the site of a first nick. In some embodiments the transposase catalyzes transesterification of the DNA at the site of a second nick. In some embodiments the transposase catalyzes second strand (e.g. complementary strand) DNA synthesis after a first or after a second transesterification reaction.

### Nucleic Acid Features

Elements of systems provided by the invention may be provided as nucleic acids, for example, a template nucleic acid (also referred to herein as, in certain embodiments as template DNA) as described, inter alia, in the claims and enumerated embodiments, as well as, in certain embodiments, a nucleic acid encoding a Gene Writer™ polypeptide—a transposase. In various embodiments, the nucleic acids are in operative association with additional genetic elements, such as tissue-specific expression-control sequence(s) (e.g., tissue-specific promoters and tissue-specific microRNA recognition sequences), as well as additional elements, such as inverted repeats (e.g., inverted terminal repeats, such as elements from or derived from viruses, e.g., AAV ITRs) and tandem repeats, inverted repeats/direct repeats (e.g., transposon inverted repeats, e.g., transposon inverted repeats also containing direct repeats, e.g., inverted repeats also containing direct repeats from the Sleeping Beauty transposon), homology regions (segments with various degrees of homology to a target DNA), UTRs (5′, 3′, or both 5′ and 3′ UTRs), and various combinations of the foregoing. The nucleic acid elements of the systems provided by the invention can be provided in a variety of topologies, including single-stranded, double-stranded, circular, linear, linear with open ends, linear with closed ends, and particular versions of these, such as doggybone DNA (dbDNA), close-ended DNA (ceDNA).

“Operative association”, as used herein to describe a functional relationship between two nucleic acid sequences, such as a 1) promoter and 2) a heterologous object sequence, and means, in such example, the promoter and heterologous object sequence (e.g., a gene of interest) are oriented such that, under suitable conditions, the promoter drives expression of the heterologous object sequence. For instance, the template nucleic acid may be single-stranded, e.g., either the (+) or (−) orientation but an operative association between promoter and heterologous object sequence means whether or not the template nucleic acid will transcribe in a particular state, when it is in the suitable state (e.g., is in the (+) orientation, in the presence of required catalytic factors, and NTPs, etc.), it does accurately transcribe. Operative association applies analogously to other pairs of nucleic acids, including other tissue-specific expression control sequences (such as enhancers, repressors and microRNA recognition sequences), IR/DR, ITRs, UTRs, or homology regions and heterologous object sequences or sequences encoding a transposase.

“Nucleic acid” encompasses RNA, DNA, or combinations thereof, including hetero-polymers containing both oxy and de-oxy nucleotides. The substituent nucleotides can comprise (or consist of) naturally occurring nitrogenous bases A, T, G, C, U, or, in some embodiments can comprise (or consist of) non-canonical or otherwise modified nitrogenous bases. Similarly, the backbone of nucleic acids can be modified in some embodiments. Nucleic acids may be single-stranded, double-stranded, or comprise both single-stranded and double-stranded duplexes, which duplexes may be homo-duplexes (DNA-DNA or RNA-RNA, for example) or hetero-duplexes (DNA-RNA). Additionally, nucleic acids may be linear, while in other embodiments, nucleic acids are circular, e.g., a plasmid or minicircle. In some embodiments, nucleic acids may possess unconnected termini, while in other embodiments, nucleic acids may be covalently closed. In some embodiments, nucleic acids may possess particular topologies, e.g., ceDNA, doggybone DNA, et cetera.

“Tissue-specific expression-control sequence(s)” means nucleic acid elements that preferentially drive or repress transcription, activity, or the half-life of a transcript comprising the heterologous object sequence in the target tissue in a tissue-specific manner: preferentially in an on-target tissue(s), relative to an off-target tissue(s). Exemplary tissue-specific expression-control sequences include tissue-specific promoters, repressors, enhancers, or combinations thereof, as well as tissue-specific microRNA recognition sequences. Tissue specificity refers to on-target (tissue(s) where expression or activity of the template nucleic acid is desired or tolerable) and off-target (tissue(s) where expression or activity of the template nucleic acid is not desired or is not tolerable). For example, a tissue-specific promoter (such as a promoter in a template nucleic acid or controlling expression of a transposase) drives expression preferentially in on-target tissues, relative to off-target tissues. In contrast, a micro-RNA that binds the tissue-specific microRNA recognition sequences (either on a nucleic acid encoding the transposase or on the template nucleic acid, or both) is preferentially expressed in off-target tissues, relative to on-target tissues, thereby reducing expression of a template nucleic acid (or transposase) in off-target tissues. Accordingly, a promoter and a microRNA recognition sequence that are specific for the same tissue, such as the target tissue, have contrasting functions (promote and repress, respectively, with concordant expression levels, i.e., high levels of the microRNA in off-target tissues and low levels in on-target tissues, while promoters drive high expression in on-target tissues and low expression in off-target tissues) with regard to the transcription, activity, or half-life of an associated sequence in that tissue. In certain particular embodiments, tissue-specific expression-control sequence(s) refers to one or more of the sequences in Table 2 or Table 3.

TABLE 2
Exemplary promoters, e.g., hepatocyte-specific promoters

| | | Source of | | |
|---|---|---|---|---|
| | | cis- | | SEQ |
| | Promoter | regulatory | | ID |
| Specificity | Name | elements | Exemplary sequence | NO |
| Hepatocytes | hAAT | α1 | AGATCTTGCTACCAGTGGAACAGCCACTAA | 1583 |
| | (serpin A1 | antitrypsin | GGATTCTGCAGTGAGAGCAGAGGGCCAGCT | |
| | | gene | AAGTGGTACTCTCCCAGAGACTGTCTGACT | |
| | | (Serpina 1 | CACGCCACCCCCTCCACCTTGGACACAGGA | |
| | | gene) | CGCTGTGGTTTCTGAGCCAGGTACAATGAC | |
| | | | TCCTTTCGGTAAGTGCAGTGGAAGCTGTAC | |
| | | | ACTGCCCAGGCAAAGCGTCCGGGCAGCGTA | |
| | | | GGCGGGCGACTCAGATCCCAGCCAGTGGAC | |
| | | | TTAGCCCCTGTTTGCTCCTCCGATAACTGGG | |
| | | | GTGACCTTGGTTAATATTCACCAGCAGCCTC | |
| | | | CCCCGTTGCCCCTCTGGATCCACTGCTTAAA | |
| | | | TACGGACGAGGACAGGGCCCTGTCTCCTCA | |
| | | | GCTTCAGGCACCACCACTGACCTGGGACAG | |
| | | | TGAATGTCCCCCTGATCTGCGGCCGTGACTC | |
| | | | TCTTAAGGTAGCCTTGCAGAAGTTGGTCGT | |
| | | | GAGGCACTGGGCAGGTAAGTATCAAGGTTA | |
| | | | CAAGACAGGTTTAAGGAGACCAATAGAAA | |
| | | | CTGGGCTTGTCGAGACAGAGAAGACTCTTG | |
| | | | CGTTTCTGATAGGCACCTATTGGTCTTACTG | |
| | | | ACATCCACTTTGCCTTTCTCTCCACAGGTGT | |
| | | | CCACTCCCAGTTCAATTACAGCT | |
| Hepatocytes | ApoE.HCR- | Apolipopro- | gttaggctcagaggcacacaggagtttctgggctcaccctgcccccttc | 1584 |
| | hAAT | tein | caacccctcagttcccatcctccagcagctgtttgtgtgctgcctctgaag | |
| | | E/C-I | tccacactgaacaaacttcagcctactcatgtccctaaaatgggcaaaca | |
| | | gene, α1 | ttgcaagcagcaaacagcaaacacacagccctccctgcctgctgacctt | |
| | | antitrypsin | ggagctggggcagaggtcagagacctctctgggcccatgccacctcc | |
| | | gene | aacatccactcgaccccttggaatttcggtggagaggagcagaggttgt | |
| | | | cctggcgtggtttaggtagtgtgagaggggtacccggggatcttgctac | |
| | | | cagtggaacagccactaaggattctgcagtgagagcagagggccagc | |
| | | | taagtggtactctcccagagactgtctgactcacgccaccccctccacct | |
| | | | tggacacaggacgctgtggtttctgagccaggtacaatgactcctttcgg | |
| | | | taagtgcagtggaagctgtacactgcccaggcaaagcgtccgggcag | |
| | | | cgtaggcgggcgactcagatcccagccagtggacttagcccctgtttg | |
| | | | ctcctccgataactggggtgaccttggttaatattcaccagcagcctccc | |
| | | | ccgttgcccctctggatccactgcttaaatacggacgaggacagggcc | |
| | | | ctgtctcctcagcttcaggcaccaccactgacctgggacagtgaatgat | |
| | | | ccccctgatctgcggcctcgacggtatcgataagcttgatatcgaattct | |
| | | | agtcgtcgaccactttcacaatctgctagcaacctgaggaggttatcgta | |
| | | | cgaaattcgctgtctgcgagggccagctgttggggtgagtactccctct | |
| | | | caaaagcgggcatgacttctgcgctaagattgtcagtttccaaaaacga | |
| | | | ggaggatttgatattcacctggcccgcggtgatgcctttgagggtggcc | |
| | | | gcgtccatctggtcagaaaagacaatctttttgttgtcaagcttgaggtgt | |
| | | | ggcaggcttgagatcgatctgaccatacacttgagtgacaatgacatcc | |
| | | | actttgcctttctctccacaggtgtccactcccaggtccaac | |
| Hepatocytes | Enhanced | Trans- | CAAATGACTTAGTTTGGCTAAAATGTAGGC | 1585 |
| | trans- | thyretin | TTTTAAAAATGTGAGCACTGCCAAGGGTTT | |
| | thyretin | gene | TTCCTTGTTGACCCATGGATCCATCAAGTGC | |
| | | | AAACATTTTCTAATGCACTATATTTAAGCCT | |
| | | | GTGCAGCTAGATGTCATTCAACATGAAATA | |
| | | | CATTATTACAACTTGCATCTGTCTAAAATCT | |
| | | | TGCATCTAAAATGAGAGACAAAAAATCTAT | |
| | | | AAAAATGGAAAACATGCATAGAAATATGTG | |
| | | | AGGGAGGAAAAAATTACCCCCAAGAATGTT | |
| | | | AGTGCACGCAGTCACACAGGGAGAAGACT | |
| | | | ATTTTTGTTTTGTTTTGATTGTTTTGTTTTGT | |
| | | | TTTGGTTGTTTTGTTTTGGTGACCTAACTGG | |
| | | | TCAAATGACCTATTAAGAATATTTCATAGA | |
| | | | ACGAATGTTCCGATGCTCTAATCTCTCTAGA | |
| | | | CAAGGTTCATATTTGTATGGGTTACTTATTC | |
| | | | TCTCTTTGTTGACTAAGTCAATAATCAGAAT | |
| | | | CAGCAGGTTTGCAGTCAGATTGGCAGGGAT | |
| | | | AAGCAGCCTAGCTCAGG | |
| Hepatocytes | Alb | Albumin | ccaccgcggtggcggccgctctagcttccttagcatgacgttccacttttt | 1586 |
| | | gene | tctaaggtggagcttacttctttgatttgatcttttgtgaaacttttggaaatta | |
| | | | cccatcttcctaagcttctgcttctctcagttttctgcttgctcattccactttt | |
| | | | ccagctgaccctgccccctaccaacattgctccacaagcacaaattcat | |
| | | | ccagagaaaataaattctaagttttatagttgtttggatcgcataggtagct | |
| | | | aaagaggtggcaacccacacatccttaggcatgagcttgattttttttgatt | |
| | | | tagaaccttcccctctctgttcctagactacactacacattctgcaagcata | |
| | | | gcacagagcaatgttctactttaattactttcattttcttgtatcctcacagcc | |
| | | | tagaaaataacctgcgttacagcatccactcagtatcccttgagcatgag | |
| | | | gtgacactacttaacatagggacgagatggtactttgtgtctcctgctctg | |
| | | | tcagcagggcactgtacttgctgataccagggaatgtttgttcttaaatac | |
| | | | catcattccggacgtgtttgccttggccagttttccatgtacatgcagaaa | |
| | | | gaagtttggactgatcaatacagtcctctgcctttaaagcaataggaaaa | |
| | | | ggccaacttgtctacgtttagtatgtggctgtagaaagggtatagatataa | |
| | | | aaattaaaactaatgaaatggcagtcttacacatttttggcagcttatttaa | |
| | | | agtcttggtgttaagtacgctggagctgtcacagctaccaatcaggcatg | |
| | | | tctgggaatgagtacacggggaccataagttactgacattcgtttcccatt | |
| | | | ccatttgaatacacacttttgtcatggtattgcttgctgaaattgttttgcaaa | |
| | | | aaaaaccccttcaaattcatatatattattttaataaatgaattttaatttatct | |
| | | | caatgttataaaaaagtcaattttaataattaggtacttatatacccaataat | |
| | | | atctaacaatcatttttaaacatttgtttattgagcttattatggatgaatctat | |
| | | | ctctatatactctatatactctaaaaaagaagaaagaccatagacaatcat | |
| | | | ctatttgatatgtgtaaagtttacatgtgagtagacatcagatgctccatttc | |
| | | | tcactgtaataccatttatagttacttgcaaaactaactggaattctaggac | |
| | | | ttaaatattttaagttttagctgggtgactggttggaaaattttaggtaagta | |
| | | | ctgaaaccaagagattataaaacaataaattctaaagttttagaagtgatc | |
| | | | ataatcaaatattaccctctaatgaaaatattccaaagttgagctacagaa | |
| | | | atttcaacataagataattttagctgtaacaatgtaatttgttgtctattttcttt | |
| | | | tgagatacagttttttctgtctagctttggctgtcctggaccttgctctgtag | |
| | | | accaggttggtcttgaactcagagatctgcttgcctctgccttgcaagtgc | |
| | | | taggattaaaagcatgtgccaccactgcctggctacaatctatgttttata | |
| | | | agagattataaagctctggctttgtgacattaatctttcagataataagtctt | |
| | | | ttggattgtgtctggagaacatacagactgtgagcagatgttcagaggta | |
| | | | tatttgcttaggggtgaattcaatctgcagcaataattatgagcagaattac | |
| | | | tgacacttccattttatacattctacttgctgatctatgaaacatagataagc | |
| | | | atgcaggcattcatcatagttttctttatctggaaaaacattaaatatgaaa | |
| | | | gaagcactttattaatacagtttagatgtgttttgccatcttttaatttcttaag | |
| | | | aaatactaagctgatgcagagtgaagagtgtgtgaaaagcagtggtgc | |
| | | | agcttggcttgaactcgttctccagcttgggatcgacctgcaggcatgct | |
| | | | tccatgccaaggcccacactgaaatgctcaaatgggagacaaagagat | |
| | | | taagctcttatgtaaaatttgctgttttacataactttaatgaatggacaaag | |
| | | | tcttgtgcatgggggtgggggtggggttagaggggaacagctccagat | |
| | | | ggcaaacatacgcaagggatttagtcaaacaactttttggcaaagatggt | |
| | | | atgattttgtaatggggtaggaaccaatgaaatgcgaggtaagtatggtt | |
| | | | aatgatctacagttattggttaaagaagtatattagagcgagtctttctgca | |
| | | | cacagatcacctttcctatcaaccccgggatcccccgggctgcaggaat | |
| | | | tcgatatcaagcttatcgataccgtcgacctcgagggggggcccggta | |
| | | | c | |
| Hepatocytes | Apoa2 | Apolipopro- | CCGGGCGTGGTGGCGCATGTCTGTAATCCC | 1587 |
| (e.g., | | tein | AGCTACTTGGGATGCTGAGGCAGGAGAATC | |
| hepatocytes | | A-II gene | CTTGAACCCGGGAGGTGGAGGTTGCAGTGA | |
| from | | | GCCGAGATCATGCCATTACGCTCCAGCCTG | |
| hepatocyte | | | AGCAACAAGAGCAAAACTCCGTCTCAGGAA | |
| progenitors) | | | AACAAACAAAAAAACCTGCACATATACTTC | |
| | | | TGAATTTAAAACAAAAGTTAAAAAACAAAG | |
| | | | ATTTCTTGGTCTCTGGTCACTACCTCCCTCA | |
| | | | TCAGCTTTGCGCCTCCACTGTCACCCTCAGG | |
| | | | AATGTTCCACATACTCAGCGAGTATGCTTG | |
| | | | GGGGGCAAAAGGGTGAAAGATACAAAAGC | |
| | | | TTCTGATATCTATTTAACTGATTTCACCCAA | |
| | | | ATGCTTTGAACCTGGGAATGTACCTCTCCCC | |
| | | | CTCCCCCACCCCCAACAGGAGTGAGACAAG | |
| | | | GGCCAGGGCTATTGCCCCTGCTGACTCAAT | |
| | | | ATTGGCTAATCACTGCCTAGAACTGATAAG | |
| | | | GTGATCAAATGACCAGGTGCCTTCAACCTT | |
| | | | TACCCTGGTAGAAGCCTCTTATTCACCTCTT | |
| | | | TTCCTGCCAGAGCCCTCCATTGGGAGGGGA | |
| | | | CGGGCGGAAGCTGTTTTCTGAATTTGTTTTA | |
| | | | CTGGGGGTAGGGTATGTTCAGTGATCAGCA | |
| | | | TCCAGGTCATTCTGGGCTCTCCTGTTTTCTC | |
| | | | CCCGTCTCATTACACATTAACTCAAAAACG | |
| | | | GACAAGATCATTTACACTTGCCCTCTTACCC | |
| | | | GACCCTCATTCCCCTAACCCCCATAGCCCTC | |
| | | | AACCCTGTCCCTGATTTCAATTCCTTTCTCC | |
| | | | TTTCTTCTGCTCCCCAATATCTCTCTGCCAA | |
| | | | GTTGCAGTAAAGTGGGATAAGGTTGAGAGA | |
| | | | TGAGATCTACCCATAATGGAATAAAGACAC | |
| | | | CATGAGCTTTCCATGGTATGATGGGTTGAT | |
| | | | GGTATTCCATGGGTTGATATGTCAGAGCTTT | |
| | | | CCAGAGAAATAACTTGGAATCCTGCTTCCT | |
| | | | GTTGCACTCAAGTCCAAGGACCTCAGATCT | |
| | | | CAAAAGAATGAACCTCAAATATACCTGAAG | |
| | | | TGTACCCCCTTAGCCTCCACTAAGAGCTGTA | |
| | | | CCCCCTGCCTCTCACCCCATCACCATGAGTC | |
| | | | TTCCATGTGCTTGTCCTCTCCTCCCCCATTTC | |
| | | | TCCAACTTGTTTATCCTCACATAATCCCTGC | |
| | | | CCCACTGGGCCCATCCATAGTCCCTGTCACC | |
| | | | TGACAGGGGGTGGGTAAACAGACAGGTAT | |
| | | | ATAGCCCCTTCCTCTCCAGCCAGGGCAGGC | |
| | | | ACAGACACCAAGGACAGAGACGCTGGCTA | |
| | | | GGTAAGATAAGGAGGCAAGATGTGTGAGC | |
| | | | AGCATCCAAAGAGGCCTGGGCTTCAGTTGT | |
| | | | GGAGAGGGAGAGAGCCAGGTTGGAATGGG | |
| | | | CAGCAGGTAGGGAGATCCCTGGGGAGGAG | |
| | | | CTGAAGCCCATTTGGCTTCAGTGTCCCCCAA | |
| | | | ACCCCCACCACCCT | |
| Hepatocytes | Cyp3a4 | Cyp3a4 gene | AGCTCCTGGGGCCTGCCCTCCTCCCATTAGA | 1588 |
| (e.g., mature | | | AAATCCTCCACTTGTCAAAAAGGAAGCCAT | |
| hepatocytes) | | | TTGCTTTGAACTCCAATTCCACCCCCAAGAG | |
| | | | GCTGGGACCATCTTATTGGAGTCCTTGATGC | |
| | | | TGTGTGACCTGCAGTGACCACTGCCCCATC | |
| | | | ATTGCTGGCTGAGGTGGTTGGGGTCCATCT | |
| | | | GGCTATCTGGGCAGCTGTTCTCTTCTCTCCT | |
| | | | TTCTCTCCTGTTTCCAGACATGCAGTATTTC | |
| | | | CAGAGAGAAGGGGCCACTCTTTGGCAAAGA | |
| | | | ACCTGTCTAACTTGCTATCTATGGCAGGACC | |
| | | | TTTGAAGGGTTCACAGGAAGCAGCACAAAT | |
| | | | TGATACTATTCCACCAAGCCATCAGCTCCAT | |
| | | | CTCATCCATGCCCTGTCTCTCCTTTAGGGGT | |
| | | | CCCCTTGCCAACAGAATCACAGAGGACCAG | |
| | | | CCTGAAAGTGCAGAGACAGCAGCTGAGGC | |
| | | | ACAGCCAAGAGCTCTGGCTGTATTAATGAC | |
| | | | CTAAGAAGTCACCAGAAAGTCAGAAGGGA | |
| | | | TGACATGCAGAGGCCCAGCAATCTCAGCTA | |
| | | | AGTCAACTCCACCAGCCTTTCTAGTTGCCCA | |
| | | | CTGTGTGTACAGCACCCTGGTAGGGACCAG | |
| | | | AGCCATGACAGGGAATAAGACTAGACTATG | |
| | | | CCCTTGAGGAGCTCACCTCTGTTCAGGGAA | |
| | | | ACAGGCGTGGAAACACAATGGTGGTAAAG | |
| | | | AGGAAAGAGGACAATAGGATTGCATGAAG | |
| | | | GGGATGGAAAGTGCCCAGGGGAGGAAATG | |
| | | | GTTACATCTGTGTGAGGAGTTTGGTGAGGA | |
| | | | AAGACTCTAAGAGAAGGCTCTGTCTGTCTG | |
| | | | GGTTTGGAAGGATGTGTAGGAGTCTTCTAG | |
| | | | GGGGCACAGGCACACTCCAGGCATAGGTAA | |
| | | | AGATCTGTAGGTGTGGCTTGTTGGGATGAA | |
| | | | TTTCAAGTATTTTGGAATGAGGACAGCCAT | |
| | | | AGAGACAAGGGCAGGAGAGAGGCGATTTA | |
| | | | ATAGATTTTATGCCAATGGCTCCACTTGAGT | |
| | | | TTCTGATAAGAACCCAGAACCCTTGGACTC | |
| | | | CCCAGTAACATTGATTGAGTTGTTTATGATA | |
| | | | CCTCATAGAATATGAACTCAAAGGAGGTCA | |
| | | | GTGAGTGGTGTGTGTGTGATTCTTTGCCAAC | |
| | | | TTCCAAGGTGGAGAAGCCTCTTCCAACTGC | |
| | | | AGGCAGAGCACAGGTGGCCCTGCTACTGGC | |
| | | | TGCAGCTCCAGCCCTGCCTCCTTCTCTAGCA | |
| | | | TATAAACAATCCAACAGCCTCACTGAATCA | |
| | | | CTGCTGTGCAGGGCAGGAAAGCTCCATGCA | |
| Hepatocytes | LP1B | Apolipopro- | cggcctctagactcgagccctaaaatgggcaaacattgcaagcagcaa | 1589 |
| | | tein | acagcaaacacacagccctccctgcctgctgaccttggagctggggca | |
| | | E/C-I | gaggtcagagacctctctgggcccatgccacctccaacatccactcga | |
| | | gene, α1 | ccccttggaatttcggtggagaggagcagaggttgtcctggcgtggttt | |
| | | antitrypsin | aggtagtgtgagagggtggacacaggacgctgtggtttctgagccagg | |
| | | gene | gggcgactcagatcccagccagtggacttagcccctgtttgctcctccg | |
| | | | ataactggggtgaccttggttaatattcaccagcagcctcccccgttgcc | |
| | | | cctctggatccactgcttaaatacggacgaggacagggccctgtctcct | |
| | | | cagcttcaggcaccaccactgacctgggacagtgaatccggactctaa | |
| | | | ggtaaatataaaatttttaagtgtataatgtgttaaactactgattctaattgtt | |
| | | | tctctcttttagattccaacctttggaactgaaccggt | |
| Hepatocytes | MIR122 | microRNA- | GAATGCATGGTTAACTACGTCAGAAATGAC | 1590 |
| (e.g., | | 122 | CAGTTCAAGAGGAGAATGAGATTGGCTTCC | |
| hepatocytes | | | AAATGTTGGTCAAGAGCTCTACGTAGCATG | |
| from early | | | AGCCAAGGATCTATTGAACTTAGTAGGCTC | |
| stage | | | CTGTGACCGGTGACTCTTCTGTCTCTAGAAA | |
| embryonic | | | TCTGGGGAGGTGACCAGGTCATACATGGCA | |
| liver cells | | | GTCTTCCCGTGAGGAACGTTAAACTGGTTG | |
| and | | | GAAGTTGGGGTTCTGAGGGGAAGATGTATT | |
| endoderm) | | | CACTAGGTGACCTGTCTTCTCTGCCTCGGTG | |
| | | | GCCTCCATGGCTGCCTGCTGGCCGCACACC | |
| | | | CCCACTCAGCAGAGGAATGGACTTTCCAAT | |
| | | | CTTGCTGAGTGTGTTTGACCAAAGGTGGTG | |
| | | | CTGACTTAGTGGCCTAAGGTCGTGCCCTCCC | |
| | | | TCCCCCACTGAATCGATAAATAATGCGACT | |
| | | | TATCAGAAAGAGAAAGAATTGTTTACTTTT | |
| | | | AAACCCTGGATCCCATAAAGGGAGAGGGG | |
| | | | AGAGGCCTAAAGCCACAGAAGCTGTGGAA | |
| | | | GGCGCCATCCTGCCTGCCACAGGAAGGGCC | |
| | | | TTGGACTGAGAGGACCGGAGCTGACTGGGG | |
| | | | GTAAGTGCGGCTCTCCCCCGGCGCCTGCCG | |
| | | | ACCCCCCTGAGTGATCAGGCCGTTCTTTGG | |
| | | | GGTGGCCGCTGACCGAGAAATGACGGGAG | |
| | | | G | |
| | | | See Li et al., 2011, J. Hepatol., 55: 602-611 | |
| Hepatocytes | hemopexin | Hemopexin | GCAGCTTTGGGAGTGGGCCCAGGAAGTACT | 1591 |
| | | gene | GAGGATAGCAGGTGAGATCCCAGGAAGAG | |
| | | | ATGGATGTGGGGCCGAGACACTGGAGAGA | |
| | | | GAAACAGGACTGTCAGATAAAGGGCGTCTG | |
| | | | TGACTCCTAGATCTCATTATGCCTACTACCA | |
| | | | TAACCTACCCCCAATTCCTAATATTCTCCTA | |
| | | | CCCTAGAGGGGGGGAAATTGTCAGAAATTT | |
| | | | GGCTGCAACACTAGCAACACTACTCAGTAC | |
| | | | TTGAAATGCATTTTTGCATTTTTTTCATTCA | |
| | | | ACAAATATTTCTGGAACAACTCTTATATGCC | |
| | | | AGGCACTATTTTAGGAGTCAGGGATATATA | |
| | | | ATGGTAAACAAGACAGGCAAAACAAAGCA | |
| | | | AAGCAACAACAACCATCACCAGATAAGTAG | |
| | | | ACAGATGAAAGAATTTCAAGTTTTAGTAAG | |
| | | | TAAAATAAAACAAGCAAGGGTCTGAAATG | |
| | | | GCTAGATAAGGTGGTCAAGAAAGGCTTCAT | |
| | | | TGAGAAGGTAGCATTTAAGCAGGAGTCAGC | |
| | | | TAGAAATATTGTGAAATTCCAGTTACAGTT | |
| | | | CTATTTGTTCTGGGTTGGTTAAATAAAGCTT | |
| | | | TTTCCCCCAAGGTGGAAACTACCAAGAAAG | |
| | | | ACTAATTACTAGTAGTGGTGGTGCTCTCTGG | |
| | | | AAGAGAGACACCTCCTGTTTCTGCCTCATTA | |
| | | | CTGTCAACCCTTCACTTCCAGGCACTTTTTG | |
| | | | CAAAGCCCTTTGCCAGTCAGGGAAGGCGAG | |
| | | | AGGCTGGGCATGGGGCTTGGACATTTGACA | |
| | | | ACAGTGAGACATTATTGTCCCCAGACTCAC | |
| | | | TAGCCCAAGGGTAAAGCTGAAGAGGCTTGG | |
| | | | GCATGCCCCAGAAAGGCCCCTGATGAAGCT | |
| | | | TGGAAAAAGCTGTTCTCTGAGTATTTCTAA | |
| | | | GTAAGTTTATCTGTGTGTGTGGTTACTAAAA | |
| | | | GTAGTAAGTATTGCTGTCTCTAGCTGCCTTA | |
| | | | GAGCAGGGCTTGACACAGTACACAGCAATA | |
| | | | TTAGTTCCCTCCTTTTCTCACCTCCCCCATTG | |
| | | | TGGAGATAAACTCAATCACAAAAGGTGATC | |
| | | | CTCAGTCTACTCACTTCCCTGACTTATGGAT | |
| | | | GCCTGGACCCATTGCCAGTGTGAGAGTCAC | |
| | | | AGCTGGACGTCAGCAGTGTAGCCCAGTTAC | |
| | | | TGCTTGAAAATTGCTGAAGGGGGTTGGGGG | |
| | | | GCAGCTGCCGGGAAAAAGGAGTCTTGGATT | |
| | | | CAGATTTCTGTCCAGACCCTGACCTTATTTG | |
| | | | CAGTGATGTAATCAGCCAATATTGGCTTAG | |
| | | | TCCTGGGAGACAGCACATTCCCAGTAGAGT | |
| | | | TGGAGGTGGGGGTGGTGCTGCTGCCAACT | |
| Hepatocytes | HLP | Apolipopro- | tgtttgctgcttgcaatgtttgcccattttagggtggacacaggacgctgt | 1592 |
| | | tein, | ggtttctgagccagggggcgactcagatcccagccagtggacttagcc | |
| | | SERINA1 | cctgtttgctcctccgataactggggtgaccttggttaatattcaccagca | |
| | | | gcctcccccgttgcccctctggatccactgcttaaatacggacgaggac | |
| | | | agggccctgtctcctcagcttcaggcaccaccactgacctgggacagt | |
| | | | gaatc | |
| liver | VEC | Vascular | CCCCTGCCCTCCTCCTCTGCCCTCTCCTGGC | 1593 |
| sinusoidal | | endothelial | ATTCCTCCTTCATCATGGGACCCTCTTCTAA | |
| endothelial | | cadherin | TGGATCCCCAAATGTCAGAGGGTCCAAGTC | |
| cells | | gene | CTCCCTCCCTCCAAGCTCATCCATGCCCATG | |
| | | | GCCTCAGATGCCAGCCATAAGCTGTTGGGT | |
| | | | TCCAAACCTCGACTCCAGGCTGGACTCACC | |
| | | | CCTGTCTCCCCCACCAGCCTGACACCTCCAC | |
| | | | CTGGGTATCTAACGAGCATCTCAAACTCAA | |
| | | | CCTGCCTGAGACAGAGGAATCACTATCCCC | |
| | | | TCCTCCTCCAAAAATATCCTTCCATCACACT | |
| | | | CCCCATCTTGTGCTCTGATTTACTAAACGGC | |
| | | | CCTGGGCCCTCTCTTTCTCAGGGTCTCTGCT | |
| | | | TGCCCAGCTATATAATAAAACAAGTTTGGG | |
| | | | ACTTCCCAACCATTCACCCATGGAAAAACA | |
| | | | GAAGCAACTCTTCAAAGGACAGATTCCCAG | |
| | | | GATCTGCCCTGGGAGATTCCAAATCAGTTG | |
| | | | ATCTGGGGTGAGCCCAGTCCTCTGTAGTTTT | |
| | | | TAGAAGCTCCTCCTATGTCTCTCCTGGTCAG | |
| | | | CAGAATCTTGGCCCCTCCCTTCCCCCCAGCC | |
| | | | TCTTGGTTCTTCTGGGCTCTGATCCAGCCTC | |
| | | | AGCGTCACTGTCTTCCACGCCCCTCTTTGAT | |
| | | | TCTCGTTTATGTCAAAAGCCTTGTGAGGATG | |
| | | | AGGCTGTGATTATCCCCATTTTACAGATGA | |
| | | | GGAAACTGTGGCTCCAGGATGACACAACTG | |
| | | | GCCAGAGGTCACATCAGAAGCAGAGCTGG | |
| | | | GTCACTTGACTCCACCCAATATCCCTAAATG | |
| | | | CAAACATCCCCTACAGACCGAGGCTGGCAC | |
| | | | CTTAGAGCTGGAGTCCATGCCCGCTCTGAC | |
| | | | CAGGAGAAGCCAACCTGGTCCTCCAGAGCC | |
| | | | AAGAGCTTCTGTCCCTTTCCCATCTCCTGAA | |
| | | | GCCTCCCTGTCACCTTTAAAGTCCATTCCCA | |
| | | | CAAAGACATCATGGGATCACCACAGAAAAT | |
| | | | CAAGCTCTGGGGCTAGGCTGACCCCAGCTA | |
| | | | GATTTTTGGCTCTTTTATACCCCAGCTGGGT | |
| | | | GGACAAGCACCTTAAACCCGCTGAGCCTCA | |
| | | | GCTTCCCGGGCTATAAAATGGGGGTGATGA | |
| | | | CACCTGCCTGTAGCATTCCAAGGAGGGTTA | |
| | | | AATGTGATGCTGCAGCCAAGGGTCCCCACA | |
| | | | GCCAGGCTCTTTGCAGGTGCTGGGTTCAGA | |
| | | | GTCCCAGAGCTGAGGCCGGGAGTAGGGGTT | |
| | | | CAAGTGGGGTGCCCCAGGCAGGGTCCAGTG | |
| | | | CCAGCCCTCTGTGGAGACAGCCATCCGGGG | |
| | | | CCGAGGCAGCCGCCCACCGCAGGGCCTGCC | |
| | | | TATCTGCAGCCAGCCCAGCCCTCACAAAGG | |
| | | | AACAATAACAGGAAACCATCCCAGGGGGA | |
| | | | AGTGGGCCAGGGCCAGCTGGAAAACCTGA | |
| | | | AGGGGAGGCAGCCAGGCCTCCCTCGCCAGC | |
| | | | GGGGTGTGGCTCCCCTCCAAAGACGGTCGG | |
| | | | CTGACAGGCTCCACAGAGCTCCACTCACGC | |
| | | | TCAGCCCTGGACGGACAGGCAGTCCAACGG | |
| | | | AACAGAAACATCCCTCAGCCCACAGGCACG | |
| | | | GTGAGTGGGGGCTCCCACACTCCCCTCCAC | |
| | | | CCCAAACCCGCCACCCTGCG | |
| ubiquitous | EF1a core | EF1α gene | gggcagagcgcacatcgcccacagtccccgagaagttggggggagg | 1594 |
| | promoter | | ggtcggcaattgaacgggtgcctagagaaggtggcgcggggtaaact | |
| | | | gggaaagtgatgtcgtgtactggctccgcctttttcccgagggtggggg | |
| | | | agaaccgtatataagtgcagtagtcgccgtgaacgttctttttcgcaacg | |
| | | | ggtttgccgccagaacacag | |
| ubiquitous | EF1a | EF1α gene | ggctccggtgcccgtcagtgggcagagcgcacatcgcccacagtccc | 1595 |
| | | | cgagaagttggggggaggggtcggcaattgaaccggtgcctagagaa | |
| | | | ggtggcgcggggtaaactgggaaagtgatgtcgtgtactggctccgcc | |
| | | | tttttcccgaggggggggagaaccgtatataagtgcagtagtcgccgt | |
| | | | gaacgttctttttcgcaacgggtttgccgccagaacacaggtaagtgcc | |
| | | | gtgtgtggttcccgcgggcctggcctctttacgggttatggcccttgcgt | |
| | | | gccttgaattacttccacctggctccagtacgtgattcttgatcccgagct | |
| | | | ggagccaggggcgggccttgcgctttaggagccccttcgcctcgtgct | |
| | | | tgagttgaggcctggcctgggcgctggggccgccgcgtgcgaatctg | |
| | | | gtggcaccttcgcgcctgtctcgctgctttcgataagtctctagccatttaa | |
| | | | aatttttgatgacctgctgcgacgctttttttctggcaagatagtcttgtaaa | |
| | | | tgcgggccaggatctgcacactggtatttcggtttttgggcccgcggcc | |
| | | | ggcgacggggcccgtgcgtcccagcgcacatgttcggcgaggcggg | |
| | | | gcctgcgagcgcggccaccgagaatcggacgggggtagtctcaagct | |
| | | | ggccggcctgctctggtgcctggcctcgcgccgccgtgtatcgccccg | |
| | | | ccctgggcggcaaggctggcccggtcggcaccagttgcgtgagcgg | |
| | | | aaagatggccgcttcccggccctgctccagggggctcaaaatggagg | |
| | | | acgcggcgctcgggagagcggggggtgagtcacccacacaaagg | |
| | | | aaaagggcctttccgtcctcagccgtcgcttcatgtgactccacggagta | |
| | | | ccgggcgccgtccaggcacctcgattagttctggagcttttggagtacg | |
| | | | tcgtctttaggttggggggaggggttttatgcgatggagtttccccacact | |
| | | | gagtgggtggagactgaagttaggccagcttggcacttgatgtaattctc | |
| | | | cttggaatttggcctttttgagtttggatcttggttcattctcaagcctcaga | |
| | | | cagtggttcaaagtttttttcttccatttcaggtgtcgtga | |
| ubiquitous | hPGK | PGK gene | ggggttggggttgcgccttttccaaggcagccctgggtttgcgcaggg | 1596 |
| | | | acgcggctgctctgggcgtggttccgggaaacgcagcggcgccgac | |
| | | | cctgggtctcgcacattcttcacgtccgttcgcagcgtcacccggatctt | |
| | | | cgccgctacccttgtgggccccccggcgacgcttcctgctccgcccct | |
| | | | aagtcgggaaggttccttgcggttcgcggcgtgccggacgtgacaaac | |
| | | | ggaagccgcacgtctcactagtaccctcgcagacggacagcgccagg | |
| | | | gagcaatggcagcgcgccgaccgcgatgggctgtggccaatagcgg | |
| | | | ctgctcagcggggcgcgccgagagcagcggccgggaaggggcggt | |
| | | | gcgggaggcggggtgtggggcggtagtgtgggccctgttcctgcccg | |
| | | | cgcggtgttccgcattctgcaagcctccggagcgcacgtcggcagtcg | |
| | | | gctccctcgttgaccgaatcaccgacctctctcccca | |
| ubiquitous | mCMV | Cytomegalo- | ggtaggcgtgtacggtgggaggcctatataagcagagct | 1597 |
| | | virus | | |
| ubiquitous | Ubc | Ubiquitin C | gtctaacaaaaaagccaaaaacggccagaatttagcggacaatttacta | 1598 |
| | | gene | gtctaacactgaaaattacatattgacccaaatgattacatttcaaaaggt | |
| | | | gcctaaaaaacttcacaaaacacactcgccaaccccgagcgcatagtt | |
| | | | caaaaccggagcttcagctacttaagaagataggtacataaaaccgac | |
| | | | caaagaaactgacgcctcacttatccctcccctcaccagaggtccggc | |
| | | | gcctgtcgattcaggagagcctaccctaggcccgaaccctgcgtcctg | |
| | | | cgacggagaaaagcctaccgcacacctaccggcaggtggccccacc | |
| | | | ctgcattataagccaacagaacgggtgacgtcacgacacgacgaggg | |
| | | | cgcgcgctcccaaaggtacgggtgcactgcccaacggcaccgccata | |
| | | | actgccgcccccgcaacagacgacaaaccgagttctccagtcagtgac | |
| | | | aaacttcacgtcagggtccccagatggtgccccagcccatctcacccg | |
| | | | aataagagctttcccgcattagcgaaggcctcaagaccttgggttcttgc | |
| | | | cgcccaccatgccccccaccttgtttcaacgacctcacagcccgcctca | |
| | | | caagcgtcttccattcaagactcgggaacagccgccattttgctgcgctc | |
| | | | cccccaacccccagttcagggcaaccttgctcgcggacccagactaca | |
| | | | gcccttggcggtctctccacacgcttccgtcccaccgagcggcccggc | |
| | | | ggccacgaaagccccggccagcccagcagcccgctactcaccaagt | |
| | | | gacgatcacagcgatccacaaacaagaaccgcgacccaaatcccggc | |
| | | | tgcgacggaactagctgtgccacacccggcgcgtccttatataatcatc | |
| | | | ggcgttcaccgccccacggagatccctccgcagaatcgccgagaagg | |
| | | | gactacttttcctcgcctgttccgctctctggaaagaaaaccagtgcccta | |
| | | | gagtcacccaagtcccgtcctaaaatgtccttctgctgatactggggttct | |
| | | | aaggccgagtcttatgagcagcgggccgctgtcctgagcgtccgggc | |
| | | | ggaaggatcaggacgctcgctgcgcccttcgtctgacgtggcagcgct | |
| | | | cgccgtgaggaggggggcgcccgcgggaggcgccaaaacccggc | |
| | | | gcggaggcc | |
| ubiquitous | SFFV | Spleen | gtaacgccattttgcaaggcatggaaaaataccaaaccaagaatagag | 1599 |
| | | focus- | aagttcagatcaagggcgggtacatgaaaatagctaacgttgggccaa | |
| | | forming | acaggatatctgcggtgagcagtttcggccccggcccggggccaaga | |
| | | virus | acagatggtcaccgcagtttcggccccggcccgaggccaagaacaga | |
| | | | tggtccccagatatggcccaaccctcagcagtttcttaagacccatcaga | |
| | | | tgtttccaggctcccccaaggacctgaaatgaccctgcgccttatttgaat | |
| | | | taaccaatcagcctgcttctcgcttctgttcgcgcgcttctgcttcccgag | |
| | | | ctctataaaagagctcacaacccctcactcggcgcgccagtcctccgac | |
| | | | agactgagtcgcccggg | |

TABLE 3
Exemplary miRNA sequences

| | miRNA | | | |
|---|---|---|---|---|
| Silenced cell type | name | Mature miRNA | miRNA sequence | SEQ ID NO |
| hematopoietic cells | miR-142 | hsa-miR-142-3p | uguaguguuuccuacuuuaugga | 1573 |
| hematopoietic cells | miR-142 | hsa-miR-142-5p | cauaaaguagaaagcacuacu | 1572 |
| hematopoietic cells | mir-181a-2 | hsa-miR-181a-5p | aacauucaacgcugucggugagu | 1600 |
| hematopoietic cells | mir-181a-2 | hsa-miR-181a-2-3p | accacugaccguugacuguacc | 1601 |
| hematopoietic cells | mir-181b-1 | hsa-miR-181b-5p | aacauucauugcugucggugggu | 1602 |
| hematopoietic cells | mir-181b-1 | hsa-miR-181b-3p | cucacugaacaaugaaugcaa | 1603 |
| hematopoietic cells | mir-181c | hsa-miR-181c-5p | aacauucaaccugucggugagu | 1604 |
| hematopoietic cells | mir-181c | hsa-miR-181c-3p | aaccaucgaccguugaguggac | 1605 |
| hematopoietic cells | mir-181a-1 | hsa-miR-181a | aacauucaacgcugucggugagu | 1600 |
| hematopoietic cells | mir-181a-1 | hsa-miR-181a-3p | accaucgaccguugauuguacc | 1606 |
| hematopoietic cells | mir-181b-2 | hsa-miR-181b-5p | aacauucauugcugucggugggu | 1602 |
| hematopoietic cells | mir-181b-2 | hsa-miR-181b-2-3p | cucacugaucaaugaaugca | 1607 |
| hematopoietic cells | mir-181d | hsa-miR-181d-5p | aacauucauuguugucggugggu | 1608 |
| hematopoietic cells | mir-181d | hsa-miR-181d-3p | ccaccgggggaugaaugucac | 1609 |
| hematopoietic cells | miR-223 | hsa-miR-223-5p | cguguauuugacaagcugaguu | 1610 |
| hematopoietic cells | miR-223 | hsa-miR-223-3p | ugucaguuugucaaauacccca | 1611 |
| pDCs | miR-126 | hsa-miR-126-5p | cauuauuacuuuugguacgcg | 1612 |
| pDCs | miR-126 | hsa-miR-126-3p | ucguaccgugaguaauaaugcg | 1613 |

In some embodiments, a nucleic acid described herein (e.g., template nucleic acid or a template encoding a transposase) comprises a promoter sequence, e.g., a tissue specific promoter. In some embodiments, the tissue-specific promoter is used to increase the target-cell specificity of a Gene Writer™ system. For instance, the promoter can be chosen on the basis that it is active in a target cell type but not active in (or active at a lower level in) a non-target cell type. Thus, even if the nucleic acid encoding the polypeptide was delivered into a non-target cell, it would not drive expression (or only drive low level expression) of the transposase, limiting integration of the DNA template. A system having a tissue-specific promoter sequence in the transposase DNA may also be used in combination with a microRNA binding site, e.g., encoded in the transposase DNA, e.g., as described herein. A system having a tissue-specific promoter sequence in the transposase DNA may also be used in combination with a DNA template containing a heterologous object sequence driven by a tissue-specific promoter, e.g., to achieve higher levels of integration and heterologous object sequence expression in target cells than in non-target cells.

In some embodiments, a nucleic acid described herein (e.g., an RNA encoding a Gene Writer™ polypeptide, or a DNA encoding the RNA, or a template nucleic acid) comprises a microRNA binding site. In some embodiments, the microRNA binding site is used to increase the target-cell specificity of a Gene Writer™ system. For instance, the microRNA binding site can be chosen on the basis that it is recognized by a miRNA that is present in a non-target cell type, but that is not present (or is present at a reduced level relative to the non-target cell) in a target cell type. Thus, when the RNA encoding the Gene Writer™ polypeptide is present in a non-target cell, it would be bound by the miRNA, and when the RNA encoding the Gene Writer™ polypeptide is present in a target cell, it would not be bound by the miRNA (or bound but at reduced levels relative to the non-target cell). While not wishing to be bound by theory, binding of the miRNA to the RNA encoding the Gene Writer™ polypeptide may reduce production of the Gene Writer™ polypeptide, e.g., by degrading the mRNA encoding the polypeptide or by interfering with translation. Accordingly, the heterologous object sequence would be inserted into the genome of target cells more efficiently than into the genome of non-target cells. A system having a microRNA binding site in the RNA encoding the Gene Writer™ polypeptide (or encoded in the DNA encoding the RNA) may also be used in combination with a template DNA whose corresponding RNA is regulated by a second microRNA binding site, e.g., as described herein in the section entitled “Template component of Gene Writer™ gene editor system.”

In some embodiments, a nucleic acid component of a system provided by the invention a sequence (e.g., transposase or a heterologous object sequence) is flanked by untranslated regions (UTRs) that modify protein expression levels. The effects of various 5′ and 3′ UTRs on protein expression are known in the art. For example, in some embodiments, the coding sequence may be preceded by a 5′ UTR that modifies RNA stability or protein translation. In some embodiments, the sequence may be followed by a 3′ UTR that modifies RNA stability or translation. In some embodiments, the sequence may be preceded by a 5′ UTR and followed by a 3′ UTR that modify RNA stability or translation. In some embodiments, the 5′ and/or 3′ UTR may be selected from the 5′ and 3′ UTRs of complement factor 3 (C3) (cactcctccccatcctctccctctgtccctctgtccctctgaccctgcactgtcccagcacc (SEQ ID NO: 1566)) or orosomucoid 1 (ORM1) (caggacacagccttggatcaggacagagacttgggggccatcctgcccctccaacccgacatgtgtacctcagctttttccctcacttgcat caataaagcttctgtgtttggaacagctaa (SEQ ID NO: 1567)) (Asrani et al. RNA Biology 2018). In certain embodiments, the 5′ UTR is the 5′ UTR from C3 and the 3′ UTR is the 3′ UTR from ORM1. In certain embodiments, a 5′ UTR and 3′ UTR for protein expression, e.g., mRNA (or DNA encoding the RNA) for a Gene Writer polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC (SEQ ID NO: 1568) and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 1569), e.g., as described in Richner et al. Cell 168(6): P1114-1125 (2017), the sequences of which are incorporated herein by reference.

In some embodiments, a 5′ and/or 3′ UTR may be selected to enhance protein expression. In some embodiments, a 5′ and/or 3′ UTR may be selected to modify protein expression such that overproduction inhibition is minimized. In some embodiments, UTRs are around a coding sequence, e.g., outside the coding sequence and in other embodiments proximal to the coding sequence. In some embodiments additional regulatory elements (e.g., miRNA binding sites, cis-regulatory sites) are included in the UTRs.

In some embodiments, an open reading frame of a Gene Writer system, e.g., an ORF of an mRNA (or DNA encoding an mRNA) encoding a Gene Writer polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′ (SEQ ID NO: 1568). In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′ (SEQ ID NO: 1569). This combination of 5′ UTR and 3′ UTR has been shown to result in desirable expression of an operably linked ORF by Richner et al. Cell 168(6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g, a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### Viral Vectors and Components Thereof

Viruses are a useful source of delivery vehicles for the systems described herein, in addition to a source of relevant enzymes or domains as described herein, e.g., as sources of polymerases and polymerase functions used herein, e.g., DNA-dependent DNA polymerase. Some enzymes may have multiple activities. In some embodiments, the virus used as a Gene Writer delivery system or a source of components thereof may be selected from a group as described by Baltimore Bacteriol Rev 35(3):235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host.

In some embodiments, virions used to deliver nucleic acid in this invention may also carry enzymes involved in the process of Gene Writing. For example, a virion may contain a polymerase domain that is delivered into a host cell along with the nucleic acid. In some embodiments, a template nucleic acid may be associated with a Gene Writer polypeptide within a virion, such that both are co-delivered to a target cell upon transduction of the nucleic acid from the viral particle. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome. In some embodiments, a viral genome may comprise two or more nucleic acid molecules, e.g., comprise a segmented genome. In some embodiments, a nucleic acid in a virion may be associated with one or proteins. In some embodiments, one or more proteins in a virion may be delivered to a host cell upon transduction. In some embodiments, a natural virus may be adapted for nucleic acid delivery by the addition of virion packaging signals to the target nucleic acid, wherein a host cell is used to package the target nucleic acid containing the packaging signals.

In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

A known challenge with transposition is the process of overproduction inhibition, in which the overexpression of transposase actually reduces the rate of transposition. Accordingly, in some embodiments, the DNA encoding the transposase comprises a promoter that has been optimized for expression levels that limit overproduction inhibition, e.g., a promoter as characterized in Mikkelsen et al. Mol Ther 2003. In some embodiments, overproduction inhibition is limited by the addition of a heterologous DNA binding domain (Wilson et al. FEBS Lett 2005). In some embodiments, the transposase expression cassette is designed such that expression of the ORF encoding the transposase results in a negative feedback loop on expression of the same, e.g., the transposase protein binds and inhibits expression from its promoter. In some embodiments, a cognate recognition sequence of the transposase is used as a binding site for negative feedback regulation, e.g., a left IR/DR or a right IR/DR from the transposon. In some embodiments, a fragment of the recognition sequence that is bound by the transposase is used for negative feedback regulation, e.g., a portion of an IR/DR sequence that is specifically bound by a transposase subunit. In the case of overproduction inhibition being the result of inappropriate assembly of transposase subunits, residues involved in the protein-protein interface can be mutated to destabilize formation of free complexes in the absence of transposon DNA (see, e.g., Gaj et al. J Am Chem Soc 2014).

Circular RNAs (circRNA) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018).

It is contemplated that it may be useful to employ circular and/or linear RNA states during the formulation, delivery, or Gene Writing reaction within the target cell. Thus, in some embodiments of any of the aspects described herein, a Gene Writing system comprises one or more circular RNAs (circRNAs). In some embodiments of any of the aspects described herein, a Gene Writing system comprises one or more linear RNAs. In some embodiments, a nucleic acid as described herein (e.g., a nucleic acid molecule encoding a Gene Writer polypeptide) is a circRNA. In some embodiments, a circular RNA molecule encodes the Gene Writer™ polypeptide. In some embodiments, the circRNA molecule encoding the Gene Writer™ polypeptide is delivered to a host cell. In some embodiments, the circRNA molecule encoding the Gene Writer polypeptide is linearized (e.g., in the host cell) prior to translation.

In some embodiments, nucleic acid (e.g., encoding a Gene Writer polypeptide) is provided as circRNA. In some embodiments, the Gene Writer™ polypeptide is encoded as circRNA. While in certain embodiments the template nucleic acid is a DNA, such as a ssDNA, in some embodiments it can be provided as an RNA, e.g., with a reverse transcriptase.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a Gene Writing system. In some embodiments, nuclear localization of the circRNA results in an increase in autocatalytic activity of the ribozyme and linearization of the circRNA.

In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a Gene Writing system comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a Gene Writing system incorporates one or more ribozymes with inducible specificity to a target tissue or target cell of interest, e.g., a ribozyme that is activated by a ligand or nucleic acid present at higher levels in a target tissue or target cell of interest. In some embodiments, the Gene Writing system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a Gene Writing system is provided as circRNA, e.g., that is activated by linearization. In some embodiments, linearization of a circRNA encoding a Gene Writing polypeptide activates the molecule for translation. In some embodiments, a signal that activates a circRNA component of a Gene Writing system is present at higher levels in on-target cells or tissues, e.g., such that the system is specifically activated in these cells.

In some embodiments, an RNA component of a Gene Writing system is provided as a circRNA that is inactivated by linearization. In some embodiments, a circRNA encoding the Gene Writer polypeptide is inactivated by cleavage and degradation. In some embodiments, a circRNA encoding the Gene Writing polypeptide is inactivated by cleavage that separates a translation signal from the coding sequence of the polypeptide. In some embodiments, a signal that inactivates a circRNA component of a Gene Writing system is present at higher levels in off-target cells or tissues, such that the system is specifically inactivated in these cells.

In some embodiments, nucleic acid (e.g., encoding a transposase, or a template DNA, or both) delivered to cells is covalently closed linear DNA, or so-called “doggybone” DNA. During its lifecycle, the bacteriophage N15 employs protelomerase to convert its genome from circular plasmid DNA to a linear plasmid DNA (Ravin et al. J Mol Biol 2001). This process has been adapted for the production of covalently closed linear DNA in vitro (see, for example, WO2010086626A1). In some embodiments, a protelomerase is contacted with a DNA containing one or more protelomerase recognition sites, wherein protelomerase results in a cut at the one or more sites and subsequent ligation of the complementary strands of DNA, resulting in the covalent linkage between the complementary strands. In some embodiments, nucleic acid (e.g., encoding a transposase, or a template DNA, or both) is first generated as circular plasmid DNA containing a single protelomerase recognition site that is then contacted with protelomerase to yield a covalently closed linear DNA. In some embodiments, nucleic acid (e.g., encoding a transposase, or a template DNA, or both) flanked by protelomerase recognition sites on plasmid or linear DNA is contacted with protelomerase to generate a covalently closed linear DNA containing only the DNA contained between the protelomerase recognition sites. In some embodiments, the approach of flanking the desired nucleic acid sequence by protelomerase recognition sites results in covalently closed circular DNA lacking plasmid elements used for bacterial cloning and maintenance. In some embodiments, the plasmid or linear DNA containing the nucleic acid and one or more protelomerase recognition sites is optionally amplified prior to the protelomerase reaction, e.g., by rolling circle amplification or PCR.

In some embodiments, nucleic acid (e.g., encoding a transposase, or a template DNA, or both) delivered to cells is closed-ended, linear duplex DNA (CELiD DNA or ceDNA). In some embodiments, ceDNA is derived from the replicative form of the AAV genome (Li et al. PLoS One 2013). In some embodiments, the nucleic acid (e.g., encoding a transposase, or a template DNA, or both) is flanked by ITRs, e.g., AAV ITRs, wherein at least one of the ITRs comprises a terminal resolution site and a replication protein binding site (sometimes referred to as a replicative protein binding site). In some embodiments, the ITRs are derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, the ITRs are symmetric. In some embodiments, the ITRs are asymmetric. In some embodiments, at least one Rep protein is provided to enable replication of the construct. In some embodiments, the at least one Rep protein is derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, ceDNA is generated by providing a production cell with (i) DNA flanked by ITRs, e.g., AAV ITRs, and (ii) components required for ITR-dependent replication, e.g., AAV proteins Rep78 and Rep52 (or nucleic acid encoding the proteins). In some embodiments, ceDNA is free of any capsid protein, e.g., is not packaged into an infectious AAV particle. In some embodiments, ceDNA is formulated into LNPs (see, for example, WO2019051289A1).

In some embodiments, the ceDNA vector consists of two self complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE. See, for example, WO2019113310.

In some embodiments, nucleic acid (e.g., encoding a transposase, or a template nucleic acid, or both) delivered to cells is designed as minicircles, where plasmid backbone sequences not pertaining to Gene Writing™ are removed before administration to cells. Minicircles have been shown to result in higher transfection efficiencies and gene expression as compared to plasmids with backbones containing bacterial parts (e.g., bacterial origin of replication, antibiotic selection cassette) and have been used to improve the efficiency of transposition (Sharma et al Mol Ther Nucleic Acids 2013). In some embodiments, the DNA vector encoding the Gene Writer™ polypeptide is delivered as a minicircle. In some embodiments, the DNA vector containing the Gene Writer™ template is delivered as a minicircle. In some embodiments, the bacterial parts are flanked by recombination sites, e.g., attP/attB, loxP, FRT sites. In some embodiments, the addition of a cognate recombinase results in intramolecular recombination and excision of the bacterial parts. In some embodiments, the recombinase sites are recognized by phiC31 recombinase. In some embodiments, the recombinase sites are recognized by Cre recombinase. In some embodiments, the recombinase sites are recognized by FLP recombinase. In addition to plasmid DNA, minicircles can be generated by excising the desired construct, e.g., transposase expression cassettes or therapeutic expression cassette, from a viral backbone. Previously, it has been shown that excision and circularization of the donor sequence from a viral backbone may be important for transposase-mediated integration efficiency (Yant et al Nat Biotechnol 2002). In some embodiments, minicircles are first formulated and then delivered to target cells. In other embodiments, minicircles are formed from a DNA vector (e.g., plasmid DNA, rAAV, scAAV, ceDNA, doggybone DNA) intracellularly by co-delivery of a recombinase, resulting in excision and circularization of the recombinase recognition site-flanked nucleic acid, e.g., a nucleic acid encoding the Gene Writer™ polypeptide, or DNA template, or both.

### Template Component of Gene Writer™ Gene Editor System

The systems and methods provided by the invention include a template nucleic acid, sometimes alternately referred to as template DNA or Gene Writing™ template, which includes a heterologous object sequence (a nucleic acid sequence to be inserted into a DNA segment, such as a genome) and a sequence specifically bound by the transposase (Gene Writer™). The Gene Writing™ template is derived from the observation that though transposase proteins typically move the transposon in which they reside, they are also capable of functioning to mobilize a fragment of DNA that is flanked by the natural ends of the transposon. These ends comprise repeat sequences, which may be inverted repeats or direct repeats (IR/DR), or a combination thereof, and are the natural binding sites of the transposase subunits that are recognized and cleaved during the initial stages of the transposition mechanism to prepare the donor DNA for insertion at an ectopic site.

In some embodiments, the Gene Writing™ template thus comprises a template nucleic acid, e.g., a heterologous object sequence, flanked by the natural IR/DR sequences of the Gene Writing™ transposase. In other embodiments, the Gene Writing™ template comprises a template nucleic acid comprising a heterologous object sequence flanked by mutated IR/DR sequences derived from the natural sequences recognized by the transposase, such that the efficiency of transposition is modulated (e.g., as described in Cui et al. J Mol Biol 2002; Wang et al. Nucleic Acids Res 2017). In some embodiments, modified IR/DR sequences for Sleeping Beauty are used to modulate efficiency of transposition. In some embodiments, various SB transposon designs for IR/DR sequences are used, e.g., pT, pT2, pT4. Improved IR/DR sequences for the SB transposon are incorporated herein by reference, e.g., WO2017158029. In some embodiments, the Gene Writing™ template comprises a heterologous object sequence flanked by synthetic sequences that are designed to be recognized by the transposase, such that the process of excision and transposition into an ectopic site is enabled by the transposase in combination with the synthetic sequences. In some embodiments, the flanking sequences recognized by the transposase are modified such that they facilitate targeting of transposition to a preferred genomic locus.

It has previously been shown that there is a minimal sequence requirement for optimizing function of the transposition of the template DNA (Zayed et al. Mol Ther 2004). Thus, in some embodiments, the transposase binding sites in the IR/DR sequences are located at least 8 bp away from the heterologous object sequence. In some embodiments, the IR/DR sequences are duplicated in a tandem array, as such a “sandwich” approach has been shown to expand efficiency of Sleeping Beauty transposition of larger heterologous object sequence payloads (Zayed et al. Mol Ther 2004).

In some embodiments the template is circularized by the activity of enzymes, such as recombinases to increase transposition activity, as described in Yant el al., Nature Biotechnology 20: 990-1005, 2002.

It is understood that, when a template DNA is described as comprising an open reading frame or the reverse complement thereof, in some embodiments the template DNA is be converted into double stranded DNA (e.g., through second strand synthesis) before it can be transposed.

In certain embodiments, customized DNA template nucleic acid can be identified, designed, engineered and constructed to contain sequences altering or specifying host genome function, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, a customized DNA template nucleic acid can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails.

The template DNA may have some homology to the target DNA. In some embodiments the template DNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the template DNA, the 5′ end of the template DNA, or both the 3′ end of the template DNA and the 5′ end of the template DNA. In some embodiments the template DNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 3′ end of the template DNA, the 5′ end of the template DNA, or both the 3′ end of the template DNA and the 5′ end of the template DNA. In certain embodiments in which IR/DR sequences are present in template DNA, these regions of homology may be dispersed internal to the IR/DR sequences, while in other embodiments in which IR/DR sequences are present in template DNA, these regions of homology may be dispersed outside of the IR/DR sequences.

The template DNA component of a Gene Writer™ genome editing system described herein typically is able to bind the Gene Writer™ genome editing protein of the system. In some embodiments the template DNA has a 3′ region that is capable of binding a Gene Writer™ genome editing protein. In some embodiments the template RNA has a 5′ region that is capable of binding a Gene Writer™ genome editing protein.

In some embodiments, the template DNA may comprise RNA sequence, e.g., be a fusion between RNA and DNA polynucleotides. In some embodiments, the RNA sequence may provide a functional domain to the template molecule. In some embodiments, the RNA sequence may be derived from a gRNA. In some embodiments, the RNA sequence may recruit a protein component of the Gene Writing™ system. In some embodiments, the gRNA sequence may recruit a Cas9 domain of the Gene Writing™ system. In some embodiments, the gRNA sequence may recruit a Cas9 domain fused to the Gene Writing™ transposase, such that the template molecule can confer DNA targeting specificity of transposition activity.

In some embodiments, the object sequence may contain an open reading frame. In some embodiments the template DNA encodes a Kozak sequence. In some embodiments, the template DNA encodes an internal ribosome entry site. In some embodiments, the template DNA encodes a self-cleaving peptide such as a T2A or P2A site. In some embodiments, the template DNA encodes a start codon. In some embodiments, the template DNA encodes a splice acceptor site. In some embodiments, the template DNA encodes a splice donor site. Exemplary splice acceptor and splice donor sites are described in WO2016044416, incorporated herein by reference in its entirety. Exemplary splice acceptor site sequences are known to those of skill in the art and include, by way of example only, CTGACCCTTCTCTCTCTCCCCCAGAG (SEQ ID NO: 1570) (from human HBB gene) and TTTCTCTCCCACAAG (SEQ ID NO: 1571) (from human immunoglobulin-gamma gene). In some embodiments, the template DNA encodes a microRNA binding site downstream of the stop codon. In some embodiments, the template DNA encodes a polyA tail downstream of the stop codon of an open reading frame. In some embodiments, the template DNA encodes one or more exons. In some embodiments, the template DNA encodes one or more introns. In some embodiments, the template DNA encodes a eukaryotic transcriptional terminator. In some embodiments, the template DNA encodes an enhanced translation element or a translation enhancing element. In some embodiments, the template DNA encodes the human T-cell leukemia virus (HTLV-1) R region. In some embodiments, the template DNA encodes a posttranscriptional regulatory element that enhances nuclear export of transcribed RNA, such as that of Hepatitis B Virus (HPRE) or Woodchuck Hepatitis Virus (WPRE). In some embodiments, in the template DNA, the heterologous object sequence encodes a polypeptide and is coded in an antisense direction with respect to the 5′ and 3′ IR/DR. In some embodiments, in the template DNA, the heterologous object sequence encodes a polypeptide and is coded in a sense direction with respect to the 5′ and 3′ IR/DR.

In some embodiments, a nucleic acid described herein (e.g., a template DNA) encodes a microRNA binding site. In some embodiments, the microRNA binding site is used to increase the target-cell-specific expression of a Gene Writer™ system integration. For instance, the microRNA binding site can be chosen on the basis that it is recognized by a miRNA that is present in a non-target cell type, but that is not present (or is present at a reduced level relative to the non-target cell) in a target cell type. Thus, when the template DNA is integrated in a non-target cell, its RNA would be bound by the miRNA, and when the template DNA is integrated in a target cell, its RNA would not be bound by the miRNA (or bound but at reduced levels relative to the non-target cell). While not wishing to be bound by theory, binding of the miRNA to the transcribed RNA may interfere with expression of the heterologous object sequence from the genome. Accordingly, the heterologous object sequence would be expressed from the genome of target cells more efficiently than from the genome of non-target cells. In some embodiments, the miRNA chosen for regulation of the heterologous object sequence is selected from Table 3. A system having a microRNA binding site encoded in the template DNA may also be used in combination with a nucleic acid encoding a Gene Writer™ polypeptide, wherein expression of the Gene Writer™ polypeptide is regulated by a second microRNA binding site, e.g., as described herein, e.g., in the section entitled “Polypeptide component of Gene Writer™ gene editor system”.

In some embodiments, the object sequence may contain a non-coding sequence. For example, the template DNA may comprise a promoter or enhancer sequence. In some embodiments, the template DNA comprises a tissue specific promoter or enhancer, each of which may be unidirectional or bidirectional. In some embodiments, the promoter is an RNA polymerase I promoter, RNA polymerase II promoter, or RNA polymerase III promoter. In some embodiments, the promoter comprises a TATA element. In some embodiments, the promoter comprises a B recognition element. In some embodiments, the promoter has one or more binding sites for transcription factors. In some embodiments, the non-coding sequence is transcribed in an antisense-direction with respect to the 5′ and 3′ IR/DR. In some embodiments, the non-coding sequence is transcribed in a sense direction with respect to the 5′ and 3′ IR/DR.

In some embodiments, a nucleic acid described herein comprises a promoter sequence, e.g., a tissue specific promoter sequence. In some embodiments, the tissue-specific promoter is used to increase the target-cell specificity of a Gene Writer™ system. For instance, the promoter can be chosen on the basis that it is active in a target cell type but not active in (or active at a lower level in) a non-target cell type. Thus, even if the promoter integrated into the genome of a non-target cell, it would not drive expression (or only drive low level expression) of an integrated gene. A system having a tissue-specific promoter sequence in the template DNA may also be used in combination with a microRNA binding site, e.g., encoded in the template DNA or a nucleic acid encoding a Gene Writer™ protein, e.g., as described herein. A system having a tissue-specific promoter sequence in the template DNA may also be used in combination with a DNA encoding a Gene Writer™ polypeptide, driven by a tissue-specific promoter, e.g., to achieve higher levels of Gene Writer™ protein in target cells than in non-target cells.

In some embodiments, the template DNA encodes a microRNA sequence, a siRNA sequence, a guide RNA sequence, a piwi RNA sequence.

In some embodiments, the template DNA comprises a site that coordinates epigenetic modification. In some embodiments, the template DNA comprises an element that inhibits, e.g., prevents, epigenetic silencing. In some embodiments, the template DNA comprises a chromatin insulator. For example, the template DNA comprises a CTCF site or a site targeted for DNA methylation.

In order to promote higher level or more stable gene expression, the template DNA may include features that prevent or inhibit gene silencing. In some embodiments, these features prevent or inhibit DNA methylation. In some embodiments, these features promote DNA demethylation. In some embodiments, these features prevent or inhibit histone deacetylation. In some embodiments, these features prevent or inhibit histone methylation. In some embodiments, these features promote histone acetylation. In some embodiments, these features promote histone demethylation. In some embodiments, multiple features may be incorporated into the template DNA to promote one or more of these modifications. CpG dinucleotides are subject to methylation by host methyl transferases. In some embodiments, the template DNA is depleted of CpG dinucleotides, e.g., does not comprise CpG nucleotides or comprises a reduced number of CpG dinucleotides compared to a corresponding unaltered sequence. In some embodiments, the promoter driving transgene expression from integrated DNA is depleted of CpG dinucleotides.

In some embodiments, the template DNA comprises a gene expression unit composed of at least one regulatory region operably linked to an effector sequence. The effector sequence may be a sequence that is transcribed into RNA (e.g., a coding sequence or a non-coding sequence such as a sequence encoding a miRNA).

In some embodiments, the object sequence of the template DNA is inserted into a target genome in an endogenous intron. In some embodiments, the object sequence of the template DNA is inserted into a target genome and thereby acts as a new exon. In some embodiments, the insertion of the object sequence into the target genome results in replacement of a natural exon or the skipping of a natural exon.

In some embodiments, the heterologous object sequence of the template DNA is inserted into the target genome in a genomic safe harbor site, such as AAVS1, CCR5, or ROSA26. Such targeted insertion can be promoted using methods described herein—such as using regions of homology in the template nucleic acid, a heterologous DNA binding domain, or a combination thereof—and otherwise known to the skilled artisan. In some embodiment, the object sequence of the template DNA is added to the genome in an intergenic or intragenic region. In some embodiments, the object sequence of the template DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template DNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments, the heterologous object sequence is less than 1,000, 1,300, 1500, 2,000, 3,000, 4,000, 5,000, or 7,500 nucleotides in length.

In some embodiments, the genomic safe harbor site is a site in the host genome of a cell described herein, that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconservered element; (vi) has low transcriptional activity (i.e. no mRNA+/−25 kb); (vii) is not in copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C—C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

In some embodiments the genomic safe harbor site is a Natural Harbor™ site. In some embodiments the Natural Harbor™ site is ribosomal DNA (rDNA). In some embodiments the Natural Harbor™ site is 5S rDNA, 18S rDNA, 5.8S rDNA, or 28S rDNA. In some embodiments the Natural Harbor™ site is the Mutsu site in 5S rDNA. In some embodiments the Natural Harbor™ site is the R2 site, the R5 site, the R6 site, the R4 site, the R1 site, the R9 site, or the RT site in 28S rDNA. In some embodiments the Natural Harbor™ site is the R8 site or the R7 site in 18S rDNA. In some embodiments the Natural Harbor™ site is DNA encoding transfer RNA (tRNA). In some embodiments the Natural Harbor™ site is DNA encoding tRNA-Asp or tRNA-Glu. In some embodiments the Natural Harbor™ site is DNA encoding spliceosomal RNA. In some embodiments the Natural Harbor™ site is DNA encoding small nuclear RNA (snRNA) such as U2 snRNA.

Thus, in some aspects, the present disclosure provides a method comprising comprises using a GeneWriter system described herein to inserting a heterologous object sequence into a Natural Harbor™ site. In some embodiments, the Natural Harbor™ site is a site described in Table 4A below. In some embodiments, the heterologous object sequence is inserted within 20, 100, 150, 200, 250, 500, or 1000 base pairs of the Natural Harbor™ site. In some embodiments, the heterologous object sequence is inserted within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of the Natural Harbor™ site. In some embodiments, the heterologous object sequence is inserted into a site having at least 70%, 75%, 80%, 85%, 90%, 95%, 96%, 97%, 98%, or 99% identity to a sequence shown in Table 4A. In some embodiments, the heterologous object sequence is inserted within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of a site having at least 70%, 75%, 80%, 85%, 90%, 95%, 96%, 97%, 98%, or 99% identity to a sequence shown in Table 4A. In some embodiments, the heterologous object sequence is inserted within a gene indicated in Column 5 of Table 4A, or within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of the gene.

TABLE 4A
Natural Harbor™ sites. Column 1 indicates a retrotransposon that inserts into the
Natural Harbor™ site. Column 2 indicates the gene at the Natural Harbor™ site. Columns 3
and 4 show exemplary human genome sequence 5′ and 3′ of the insertion site (for example, 250
bp). Columns 5 and 6 list the example gene symbol and corresponding Gene ID.

| | | | | Example | |
|---|---|---|---|---|---|
| Target | Target | | | Gene | Example |
| Site | Gene | 5′ flanking sequence | 3′ flanking sequence | Symbol | Gene ID |
| R2 | 28S rDNA | CCGGTCCCCCCCGCCGGGT | GTAGCCAAATGCCTCGTCA | RNA28SN1 | 106632264 |
| | | CCGCCCCCGGGGCCGCGG | TCTAATTAGTGACGCGCAT | | |
| | | TTCCGCGCGGCGCCTCGCC | GAATGGATGAACGAGATT | | |
| | | TCGGCCGGCGCCTAGCAG | CCCACTGTCCCTACCTACT | | |
| | | CCGACTTAGAACTGGTGC | ATCCAGCGAAACCACAGC | | |
| | | GGACCAGGGGAATCCGAC | CAAGGGAACGGGCTTGGC | | |
| | | TGTTTAATTAAAACAAAGC | GGAATCAGCGGGGAAAGA | | |
| | | ATCGCGAAGGCCCGCGGC | AGACCCTGTTGAGCTTGAC | | |
| | | GGGTGTTGACGCGATGTG | TCTAGTCTGGCACGGTGAA | | |
| | | ATTTCTGCCCAGTGCTCTG | GAGACATGAGAGGTGTAG | | |
| | | AATGTCAAAGTGAAGAAA | AATAAGTGGGAGGCCCCC | | |
| | | TTCAATGAAGCGCGGGTA | GGCGCCCCCCCGGTGTCCC | | |
| | | AACGGCGGGAGTAACTAT | CGCGAGGGGCCCGGGGC | | |
| | | GACTCTCTTAAG (SEQ ID | GGGGTCCGCCG (SEQ ID | | |
| | | NO: 1508) | NO: 1513) | | |
| R4 | 28S rDNA | GCGGTTCCGCGCGGCGCC | CGCATGAATGGATGAACG | RNA28SN1 | 106632264 |
| | | TCGCCTCGGCCGGCGCCTA | AGATTCCCACTGTCCCTAC | | |
| | | GCAGCCGACTTAGAACTG | CTACTATCCAGCGAAACCA | | |
| | | GTGCGGACCAGGGGAATC | CAGCCAAGGGAACGGGCT | | |
| | | CGACTGTTTAATTAAAACA | TGGCGGAATCAGCGGGGA | | |
| | | AAGCATCGCGAAGGCCCG | AAGAAGACCCTGTTGAGC | | |
| | | CGGCGGGTGTTGACGCGA | TTGACTCTAGTCTGGCACG | | |
| | | TGTGATTTCTGCCCAGTGC | GTGAAGAGACATGAGAGG | | |
| | | TCTGAATGTCAAAGTGAA | TGTAGAATAAGTGGGAGG | | |
| | | GAAATTCAATGAAGCGCG | CCCCCGGCGCCCCCCCGGT | | |
| | | GGTAAACGGCGGGAGTAA | GTCCCCGCGAGGGGCCCG | | |
| | | CTATGACTCTCTTAAGGTA | GGGCGGGGTCCGCCGGCC | | |
| | | GCCAAATGCCTCGTCATCT | CTGCGGGCCGCCGGTGAA | | |
| | | AATTAGTGACG (SEQ ID | ATACCACTACTC (SEQ ID | | |
| | | NO: 1509) | NO: 1514) | | |
| R5 | 28S rDNA | TCCCCCCCGCCGGGTCCGC | CCAAATGCCTCGTCATCTA | RNA28SN1 | 106632264 |
| | | CCCCGGGGCCGCGGTTCC | ATTAGTGACGCGCATGAAT | | |
| | | GCGCGGCGCCTCGCCTCG | GGATGAACGAGATTCCCA | | |
| | | GCCGGCGCCTAGCAGCCG | CTGTCCCTACCTACTATCCA | | |
| | | ACTTAGAACTGGTGCGGA | GCGAAACCACAGCCAAGG | | |
| | | CCAGGGGAATCCGACTGT | GAACGGGCTTGGCGGAAT | | |
| | | TTAATTAAAACAAAGCATC | CAGCGGGGAAAGAAGACC | | |
| | | GCGAAGGCCCGCGGCGGG | CTGTTGAGCTTGACTCTAG | | |
| | | TGTTGACGCGATGTGATTT | TCTGGCACGGTGAAGAGA | | |
| | | CTGCCCAGTGCTCTGAATG | CATGAGAGGTGTAGAATA | | |
| | | TCAAAGTGAAGAAATTCA | AGTGGGAGGCCCCCGGCG | | |
| | | ATGAAGCGCGGGTAAACG | CCCCCCCGGTGTCCCCGCG | | |
| | | GCGGGAGTAACTATGACT | AGGGGCCCGGGGCGGGG | | |
| | | CTCTTAAGGTAG (SEQ ID | TCCGCCGGCCC (SEQ ID | | |
| | | NO: 1510) | NO: 1515) | | |
| R9 | 28S rDNA | CGGCGCGCTCGCCGGCCG | TAGCTGGTTCCCTCCGAAG | RNA28SN1 | 106632264 |
| | | AGGTGGGATCCCGAGGCC | TTTCCCTCAGGATAGCTGG | | |
| | | TCTCCAGTCCGCCGAGGGC | CGCTCTCGCAGACCCGACG | | |
| | | GCACCACCGGCCCGTCTCG | CACCCCCGCCACGCAGTTT | | |
| | | CCCGCCGCGCCGGGGAGG | TATCCGGTAAAGCGAATG | | |
| | | TGGAGCACGAGCGCACGT | ATTAGAGGTCTTGGGGCC | | |
| | | GTTAGGACCCGAAAGATG | GAAACGATCTCAACCTATT | | |
| | | GTGAACTATGCCTGGGCA | CTCAAACTTTAAATGGGTA | | |
| | | GGGCGAAGCCAGAGGAA | AGAAGCCCGGCTCGCTGG | | |
| | | ACTCTGGTGGAGGTCCGT | CGTGGAGCCGGGCGTGGA | | |
| | | AGCGGTCCTGACGTGCAA | ATGCGAGTGCCTAGTGGG | | |
| | | ATCGGTCGTCCGACCTGG | CCACTTTTGGTAAGCAGAA | | |
| | | GTATAGGGGCGAAAGACT | CTGGCGCTGCGGGATGAA | | |
| | | AATCGAACCATCTAG (SEQ | CCGAACGCC (SEQ ID NO: | | |
| | | ID NO: 1511) | 1516) | | |
| R8 | 18S rDNA | GCATTCGTATTGCGCCGCT | TGAAACTTAAAGGAATTG | RNA18SN1 | 106631781 |
| | | AGAGGTGAAATTCTTGGA | ACGGAAGGGCACCACCAG | | |
| | | CCGGCGCAAGACGGACCA | GAGTGGAGCCTGCGGCTT | | |
| | | GAGCGAAAGCATTTGCCA | AATTTGACTCAACACGGGA | | |
| | | AGAATGTTTTCATTAATCA | AACCTCACCCGGCCCGGAC | | |
| | | AGAACGAAAGTCGGAGGT | ACGGACAGGATTGACAGA | | |
| | | TCGAAGACGATCAGATAC | TTGATAGCTCTTTCTCGATT | | |
| | | CGTCGTAGTTCCGACCATA | CCGTGGGTGGTGGTGCAT | | |
| | | AACGATGCCGACCGGCGA | GGCCGTTCTTAGTTGGTGG | | |
| | | TGCGGCGGCGTTATTCCCA | AGCGATTTGTCTGGTTAAT | | |
| | | TGACCCGCCGGGCAGCTTC | TCCGATAACGAACGAGAC | | |
| | | CGGGAAACCAAAGTCTTT | TCTGGCATGCTAACTAGTT | | |
| | | GGGTTCCGGGGGGAGTAT | ACGCGACCCCCGAGCGGT | | |
| | | GGTTGCAAAGC (SEQ ID | CGGCGTCCC (SEQ ID NO: | | |
| | | NO: 1512) | 1517) | | |
| R4- | tRNA-Asp | | | TRD-GTC1- | 100189207 |
| 2_SRa | | | | 1 | |
| LIN25_ | tRNA-Glu | | | TRE-CTC1-1 | 100189384 |
| SM | | | | | |
| R1 | 28S rDNA | TAGCAGCCGACTTAGAACT | ACCTACTATCCAGCGAAAC | RNA28SN1 | 106632264 |
| | | GGTGCGGACCAGGGGAAT | CACAGCCAAGGGAACGGG | | |
| | | CCGACTGTTTAATTAAAAC | CTTGGCGGAATCAGCGGG | | |
| | | AAAGCATCGCGAAGGCCC | GAAAGAAGACCCTGTTGA | | |
| | | GCGGCGGGTGTTGACGCG | GCTTGACTCTAGTCTGGCA | | |
| | | ATGTGATTTCTGCCCAGTG | CGGTGAAGAGACATGAGA | | |
| | | CTCTGAATGTCAAAGTGAA | GGTGTAGAATAAGTGGGA | | |
| | | GAAATTCAATGAAGCGCG | GGCCCCCGGCGCCCCCCC | | |
| | | GGTAAACGGCGGGAGTAA | GGTGTCCCCGCGAGGGGC | | |
| | | CTATGACTCTCTTAAGGTA | CCGGGGCGGGGTCCGCCG | | |
| | | GCCAAATGCCTCGTCATCT | GCCCTGCGGGCCGCCGGT | | |
| | | AATTAGTGACGCGCATGA | GAAATACCACTACTCTGAT | | |
| | | ATGGATGAACGAGATTCC | CGTTTTTTCACTGACCCGG | | |
| | | CACTGTCCCT (SEQ ID NO: | TGAGGCGGGGGG (SEQ ID | | |
| | | 1518) | NO: 1524) | | |
| R6 | 28S rDNA | CCCCCCGCCGGGTCCGCCC | AAATGCCTCGTCATCTAAT | RNA28SN1 | 106632264 |
| | | CCGGGGCCGCGGTTCCGC | TAGTGACGCGCATGAATG | | |
| | | GCGGCGCCTCGCCTCGGC | GATGAACGAGATTCCCACT | | |
| | | CGGCGCCTAGCAGCCGAC | GTCCCTACCTACTATCCAG | | |
| | | TTAGAACTGGTGCGGACC | CGAAACCACAGCCAAGGG | | |
| | | AGGGGAATCCGACTGTTT | AACGGGCTTGGCGGAATC | | |
| | | AATTAAAACAAAGCATCGC | AGCGGGGAAAGAAGACCC | | |
| | | GAAGGCCCGCGGCGGGTG | TGTTGAGCTTGACTCTAGT | | |
| | | TTGACGCGATGTGATTTCT | CTGGCACGGTGAAGAGAC | | |
| | | GCCCAGTGCTCTGAATGTC | ATGAGAGGTGTAGAATAA | | |
| | | AAAGTGAAGAAATTCAAT | GTGGGAGGCCCCCGGCGC | | |
| | | GAAGCGCGGGTAAACGGC | CCCCCCGGTGTCCCCGCGA | | |
| | | GGGAGTAACTATGACTCTC | GGGGCCCGGGGGGGGGT | | |
| | | TTAAGGTAGCC (SEQ ID | CCGCCGGCCCTG (SEQ ID | | |
| | | NO: 1519) | NO: 1525) | | |
| R7 | 18S rDNA | GCGCAAGACGGACCAGAG | GGAGCCTGCGGCTTAATTT | RNA18SN1 | 106631781 |
| | | CGAAAGCATTTGCCAAGA | GACTCAACACGGGAAACC | | |
| | | ATGTTTTCATTAATCAAGA | TCACCCGGCCCGGACACG | | |
| | | ACGAAAGTCGGAGGTTCG | GACAGGATTGACAGATTG | | |
| | | AAGACGATCAGATACCGT | ATAGCTCTTTCTCGATTCC | | |
| | | CGTAGTTCCGACCATAAAC | GTGGGTGGTGGTGCATGG | | |
| | | GATGCCGACCGGCGATGC | CCGTTCTTAGTTGGTGGAG | | |
| | | GGCGGCGTTATTCCCATGA | CGATTTGTCTGGTTAATTC | | |
| | | CCCGCCGGGCAGCTTCCG | CGATAACGAACGAGACTC | | |
| | | GGAAACCAAAGTCTTTGG | TGGCATGCTAACTAGTTAC | | |
| | | GTTCCGGGGGGAGTATGG | GCGACCCCCGAGCGGTCG | | |
| | | TTGCAAAGCTGAAACTTAA | GCGTCCCCCAACTTCTTAG | | |
| | | AGGAATTGACGGAAGGGC | AGGGACAAGTGGCGTTCA | | |
| | | ACCACCAGGAGT (SEQ ID | GCCACCCGAG (SEQ ID | | |
| | | NO: 1520) | NO: 1526) | | |
| RT | 28S rDNA | GGCCGGGCGCGACCCGCT | AACTGGCTTGTGGCGGCC | RNA28SN1 | 106632264 |
| | | CCGGGGACAGTGCCAGGT | AAGCGTTCATAGCGACGTC | | |
| | | GGGGAGTTTGACTGGGGC | GCTTTTTGATCCTTCGATG | | |
| | | GGTACACCTGTCAAACGGT | TCGGCTCTTCCTATCATTGT | | |
| | | AACGCAGGTGTCCTAAGG | GAAGCAGAATTCACCAAG | | |
| | | CGAGCTCAGGGAGGACAG | CGTTGGATTGTTCACCCAC | | |
| | | AAACCTCCCGTGGAGCAG | TAATAGGGAACGTGAGCT | | |
| | | AAGGGCAAAAGCTCGCTT | GGGTTTAGACCGTCGTGA | | |
| | | GATCTTGATTTTCAGTACG | GACAGGTTAGTTTTACCCT | | |
| | | AATACAGACCGTGAAAGC | ACTGATGATGTGTTGTTGC | | |
| | | GGGGCCTCACGATCCTTCT | CATGGTAATCCTGCTCAGT | | |
| | | GACCTTTTGGGTTTTAAGC | ACGAGAGGAACCGCAGGT | | |
| | | AGGAGGTGTCAGAAAAGT | TCAGACATTTGGTGTATGT | | |
| | | TACCACAGGGAT (SEQ ID | GCTTGGC (SEQ ID NO: | | |
| | | NO: 1521) | 1527) | | |
| Mutsu | 5S rDNA | GTCTACGGCCATACCACCC | TGAACGCGCCCGATCTCGT | RNA5S1 | 100169751 |
| | | (SEQ ID NO: 1522) | CTGATCTCGGAAGCTAAGC | | |
| | | | AGGGTCGGGCCTGGTTAG | | |
| | | | TACTTGGATGGGAGACCG | | |
| | | | CCTGGGAATACCGGGTGC | | |
| | | | TGTAGGCTTT (SEQ ID NO: | | |
| | | | 1528) | | |
| Utopia/ | U2 | ATCGCTTCTCGGCCTTTTG | TCTGTTCTTATCAGTTTAAT | RNU2-1 | 6066 |
| Keno | snRNA | GCTAAGATCAAGTGTAGT | ATCTGATACGTCCTCTATC | | |
| | | A (SEQ ID NO: 1523) | CGAGGACAATATATTAAAT | | |
| | | | GGATTTTTGGAGCAGGGA | | |
| | | | GATGGAATAGGAGCTTGC | | |
| | | | TCCGTCCACTCCACGCATC | | |
| | | | GACCTGGTATTGCAGTACC | | |
| | | | TCCAGGAACGGTGCACCC | | |
| | | | (SEQ ID NO: 1529) | | |

### Additional Functional Characteristics for Gene Writers™

A Gene Writer as described herein may, in some instances, be characterized by one or more functional measurements or characteristics. In some embodiments, the DNA binding domain (e.g., target binding domain) has one or more of the functional characteristics described below. In some embodiments, the template binding domain has one or more of the functional characteristics described below. In some embodiments, an endonuclease domain has one or more of the functional characteristics described below. In some embodiments, a polymerase domain has one or more of the functional characteristics described below. In some embodiments, the template (e.g., template DNA) has one or more of the functional characteristics described below. In some embodiments, the target site altered by the Gene Writer has one or more of the functional characteristics described below following alteration by the Gene Writer.

Gene Writer Polypeptide

DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from the Tc1-like element Sleeping Beauty. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 pM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

Template Binding Domain

In some embodiments, the template binding domain is capable of binding to a template DNA with greater affinity than a reference DNA binding domain. In some embodiments, the reference DNA binding domain is a DNA binding domain from the Tc1-like element Sleeping Beauty. In some embodiments, the template binding domain is capable of binding to a template DNA with an affinity between 100 pM-10 nM (e.g., between 100 pM-1 nM or 1 nM-10 nM). In some embodiments, the affinity of a DNA binding domain for its template DNA is measured in vitro, e.g., by thermophoresis, e.g., as described in Asmari et al. Methods 146:107-119 (2018) (incorporated by reference herein in its entirety). In some embodiments, the affinity of a DNA binding domain for its template DNA is measured in cells (e.g., by FRET or ChIP-Seq).

In some embodiments, the DNA binding domain is associated with the template DNA in vitro with at least 50% template DNA bound in the presence of 10 nM competitor DNA, e.g., as described in Yant et al. Mol Cell Biol 24(20):9239-9247 (2004) (incorporated by reference herein in its entirety). In some embodiments, the DNA binding domain is associated with the template DNA in cells (e.g., in HEK293T cells) at a frequency at least about 5-fold or 10-fold higher than with a scrambled DNA. In some embodiments, the frequency of association between the DNA binding domain and the template DNA or scrambled DNA is measured by ChIP-seq, e.g., as described in He and Pu (2010), supra.

Endonuclease Domain

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro or in a cell (e.g., a HEK293T cell) at a frequency at least about 5-fold or 10-fold higher than with a scrambled dsDNA. In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23(19):3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from the Helitron transposase Helraiser.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

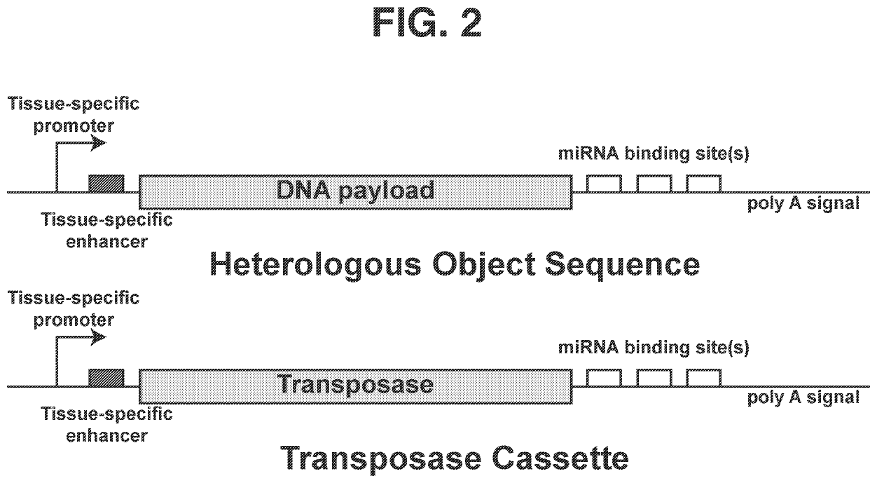

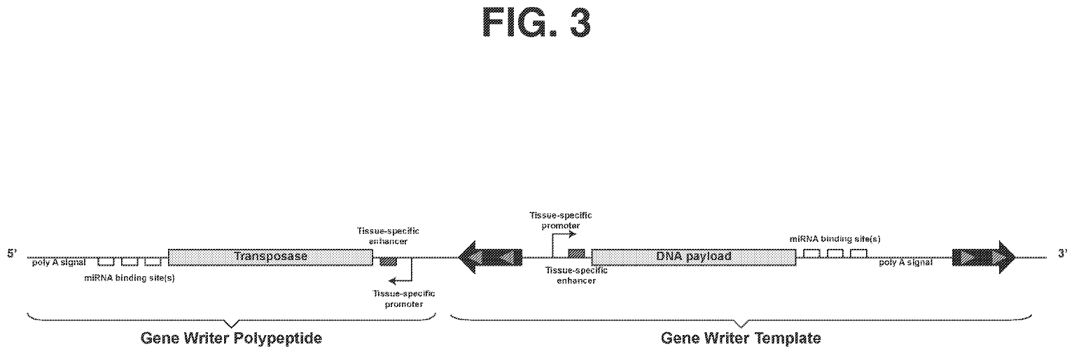

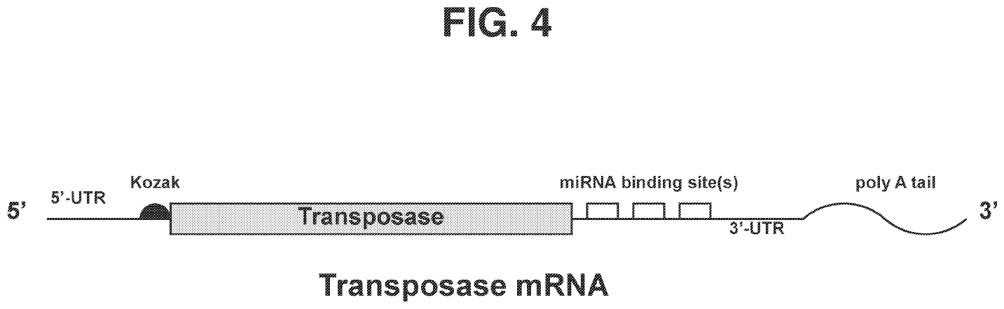

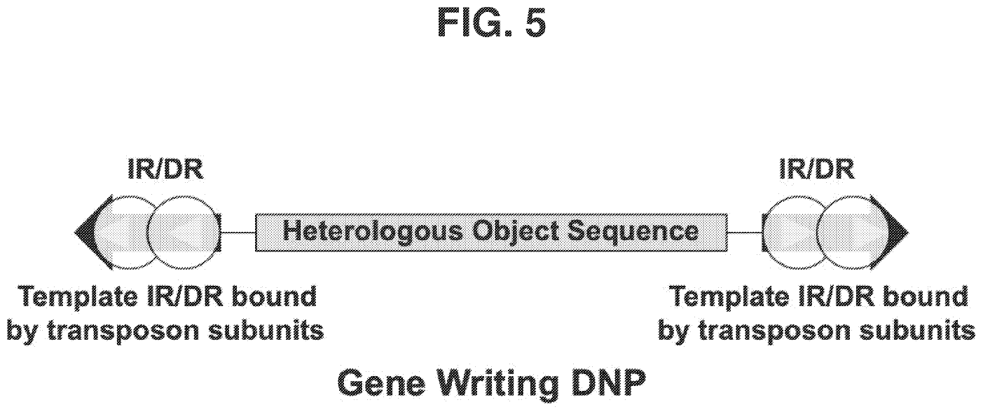

In some embodiments, the endonuclease domain releases the target after cleavage. In some embodiments, the endonuclease domain releases the target after cleavage. In some embodiments, release of the target is indicated indirectly by assessing for multiple turnovers by the enzyme, e.g., as described in Yourik at al. RNA 25(1):35-44 (2019) (incorporated herein by reference in its entirety) and shown in FIG. 2. In some embodiments, the kₑₓₚ of an endonuclease domain is 1×10⁻³-1×10⁻⁵ min⁻¹ as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360(6387):436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

Writing Domain

In some embodiments, a polymerase domain has a higher processivity in vitro relative to a reference polymerase domain. In some embodiments, the reference polymerase domain is a polymerase domain from the Helitron transposase Helraiser.

In some embodiments, the polymerase domain has a high processivity in vitro, e.g., produces an average primer extension length of greater than about 10 nt, e.g., greater than about 10-50, 50-100 nt. In some embodiments, the polymerase domain has a higher processivity in vitro than a reference polymerase domain, e.g., produces an average primer extension length of greater than about 10 nt, e.g., greater than about 10-50, 50-100 nt compared to the reference domain. In embodiments, the in vitro premature termination rate is determined as described in Wang et al. Nucl Acids Res 32(3):1197-1207 (2004) (incorporated by reference herein its entirety).

In some embodiments, the writing domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein in its entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template DNA (e.g., a template DNA having a length of at least 0.05, 0.1, 0.5, 0.6, 0.7, 0.8, 0.9, 1, 1.5, 2, 3, 4, or 5 kb, e.g., a length between 0.5-0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the polymerase domain is capable of polymerizing dNTPs in vitro. In embodiments, the polymerase domain is capable of polymerizing dNTPs in vitro at a rate between 0.1-50 nt/sec (e.g., between 0.1-1, 1-10, or 10-50 nt/sec). In embodiments, polymerization of dNTPs by the polymerase domain is measured by a single-molecule assay, e.g., as described in Schwartz and Quake (2009) PNAS 106(48):20294-20299 (incorporated by reference in its entirety).

In some embodiments, the polymerase domain has an in vitro error rate (e.g., misincorporation of nucleotides) of between 1×10⁻³-1×10⁻⁴ or 1×10⁻⁴-1×10⁻⁵ substitutions/nt, e.g., as described in Lee et al. Nucl Acids Res 44(13):e118 (2016) (incorporated herein by reference in its entirety). In some embodiments, the polymerase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the polymerase domain specifically binds a specific DNA template with higher frequency (e.g., about 5 or 10-fold higher frequency) than any scrambled DNA template, e.g., when expressed in cells (e.g., HEK293T cells). In embodiments, frequency of specific binding between the polymerase domain and the template DNA are measured by ChIP-seq, e.g., as described in He and Pu (2010), supra.

Target Site

In some embodiments, after Gene Writing, the target site surrounding the integrated sequence contains a limited number of insertions or deletions, for example, in less than about 50% or 10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, where a Gene Writer is intended to make a specific target site duplication or target site deletion, the target site sequence contains a limited number of insertions or deletions outside of the intended insertion or deletion, e.g., as determined by long-read amplicon sequencing of the target site, e.g., as described in Karst et al. (2020), supra. In some embodiments, the target site does not show multiple insertion events, e.g., head-to-tail or head-to-head duplications, e.g., as determined by long-read amplicon sequencing of the target site, e.g., as described in Karst et al. (2020), supra. In some embodiments, the target site contains an integrated sequence corresponding to the template DNA. In some embodiments, the target site does not contain insertions resulting from non-template DNA, e.g., endogenous or vector DNA, e.g., AAV ITRs, in more than about 1% or 10% of events, e.g., as determined by long-read amplicon sequencing of the target site, e.g., as described in Karst et al. (2020), supra. In some embodiments, the target site contains the integrated sequence corresponding to the template DNA.

In some embodiments, a Gene Writer described herein is capable of site-specific editing of target DNA, e.g., insertion of template DNA into a target DNA. In some embodiments, a site-specific Gene Writer is capable of generating an edit, e.g., an insertion, that is present at the target site with a higher frequency than any other site in the genome. In some embodiments, a site-specific Gene Writer is capable of generating an edit, e.g., an insertion in a target site at a frequency of at least 2, 3, 4, 5, 10, 50, 100, or 1000-fold that of the frequency at all other sites in the human genome. In some embodiments, the location of integration sites is determined by unidirectional sequencing, e.g., unidirectional sequencing as described in Example 1. The incorporation of unique molecular identifiers (UMI) in the adapters or primers used in library preparation allows the quantification of discrete insertion events, which can be compared between on-target insertions and all other insertions to determine the preference for the defined target site.

In some embodiments, a Gene Writing system is used to edit a target DNA sequence that is present at a single location in the human genome. In some embodiments, a Gene Writing system is used to edit a target DNA sequence that is present at a single location in the human genome on a single homologous chromosome, e.g., is haplotype-specific. In some embodiments, a Gene Writing system is used to edit a target DNA sequence that is present at a single location in the human genome on two homologous chromosomes. In some embodiments, a Gene Writing system is used to edit a target DNA sequence that is present in multiple locations in the genome, e.g., at least 2, 3, 4, 5, 10, 20, 50, 100, 200, 500, 1000, 5000, 10000, 100000, 200000, 500000, 1000000 (e.g., Alu elements) locations in the genome.

In some embodiments, a Gene Writer system is able to edit a genome without introducing undesirable mutations. In some embodiments, a Gene Writer system is able to edit a genome by inserting a template, e.g., template DNA, into the genome. In some embodiments, the resulting modification in the genome contains minimal mutations relative to the template DNA sequence. In some embodiments, the average error rate of genomic insertions relative to the template DNA is less than 10⁻⁴, 10⁻⁵, or 10⁻⁶ mutations per nucleotide. In some embodiments, the number of mutations relative to a template DNA that is introduced into a target cell averages less than 1, 2, 3, 4, 5, 10, 20, 30, 40, 50, 60, 70, 80, 90, or 100 nucleotides per genome. In some embodiments, the error rate of insertions in a target genome is determined by long-read amplicon sequencing across known target sites, e.g., as described in Karst et al. (2020), supra, and comparing to the template DNA sequence. In some embodiments, errors enumerated by this method include nucleotide substitutions relative to the template sequence. In some embodiments, errors enumerated by this method include nucleotide deletions relative to the template sequence. In some embodiments, errors enumerated by this method include nucleotide insertions relative to the template sequence. In some embodiments, errors enumerated by this method include a combination of one or more of nucleotide substitutions, deletions, or insertions relative to the template sequence.

Efficiency of integration events can be used as a measure of editing of target sites or target cells by a Gene Writer system. In some embodiments, a Gene Writer system described herein is capable of integrating a heterologous object sequence in a fraction of target sites or target cells. In some embodiments, a Gene Writer system is capable of editing at least 1%, 2%, 5%, 10%, 20%, 30%, 40%, 50%, 60%, 70%, 80%, 90%, 95%, 96%, 97%, 98%, 99%, 99.5%, 99.9% or 100% of target loci as measured by the detection of the edit when amplifying across the target and analyzing with long-read amplicon sequencing, e.g., as described in Karst et al. (2020). In some embodiments, a Gene Writer system is capable of editing cells at an average copy number of at least 0.1, e.g., at least 0.1, 0.5, 1, 2, 3, 4, 5, 10, or 100 copies per genome as normalized to a reference gene, e.g., RPP30, across a population of cells, e.g., as determined by ddPCR with transgene-specific primer-probe sets, e.g., as according to the methods in Lin et al. Hum Gene Ther Methods 27(5):197-208 (2016).

In some embodiments, the copy number per cell is analyzed by single-cell ddPCR (sc-ddPCR), e.g., as according to the methods of Igarashi et al. Mol Ther Methods Clin Dev 6:8-16 (2017), incorporated herein by reference in its entirety. In some embodiments, at least 1%, e.g., at least 1%, 2%, 5%, 10%, 20%, 30%, 40%, 50%, 60%, 70%, 80%, 90%, 95%, 96%, 97%, 98%, 99%, 99.5%, 99.9% or 100%, of target cells are positive for integration as assessed by sc-ddPCR using transgene-specific primer-probe sets. In some embodiments, the average copy number is at least 0.1, e.g., at least 0.1, 0.5, 1, 2, 3, 4, 5, 10, or 100 copies per cell as measured by sc-ddPCR using transgene-specific primer-probe sets.

Additional Gene Writer Characteristics

In some embodiments, the system may result in complete writing without requiring endogenous host factors. In some embodiments, the system may result in complete writing without the need for DNA repair. In some embodiments, the system may result in complete writing without eliciting a DNA damage response.

In some embodiments, the system does not require DNA repair by the NHEJ pathway, homologous recombination repair pathway, base excision repair pathway, or any combination thereof. Participation by a DNA repair pathway can be assayed, for example, via the application of DNA repair pathway inhibitors or DNA repair pathway deficient cell lines. For example, when applying DNA repair pathway inhibitors, PrestoBlue cell viability assay can be performed first to determine the toxicity of the inhibitors and whether any normalization should be applied. SCR7 is an inhibitor for NHEJ, which can be applied at a series of dilutions during Gene Writer™ delivery. PARP protein is a nuclear enzyme that binds as homodimers to both single- and double-strand breaks. Thus, its inhibitors can be used in the test of relevant DNA repair pathways, including homologous recombination repair pathway and base excision repair pathway. The experiment procedure is the same with that of SCR7. Cell lines with deficient core proteins of nucleotide excision repair (NER) pathway can be used to test the effect of NER on Gene Writing™. After the delivery of the Gene Writer™ system into the cell, ddPCR can used to evaluate the insertion of a heterologous object sequence in the context of inhibition of DNA repair pathways. Sequencing analysis can also be performed to evaluate whether certain DNA repair pathways play a role. In some embodiments, Gene Writing™ into the genome is not decreased by the knockdown of a DNA repair pathway described herein. In some embodiments, Gene Writing™ into the genome is not decreased by more than 50% by the knockdown of the DNA repair pathway.

### Evolved Variants of Gene Writers

In some embodiments, the invention provides evolved variants of Gene Writers. Evolved variants can, in some embodiments, be produced by mutagenizing a reference Gene Writer, or one of the fragments or domains comprised therein. In some embodiments, one or more of the domains (e.g., the polymerase, DNA binding (including, for example, sequence-guided DNA binding elements), or endonuclease domain) is evolved. One or more of such evolved variant domains can, in some embodiments, be evolved alone or together with other domains. An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing, a reference Gene Writer, or fragment or domain thereof, comprises mutagenizing the reference Gene Writer or fragment or domain thereof. In embodiments, the mutagenesis comprises a continuous evolution method (e.g., PACE) or non-continuous evolution method (e.g., PANCE), e.g., as described herein. in some embodiments, the evolved Gene Writer, or a fragment or domain thereof, comprises one or more amino acid variations introduced into its amino acid sequence relative to the amino acid sequence of the reference Gene Writer, or fragment or domain thereof. In embodiments, amino acid sequence variations may include one or more mutated residues (e.g., conservative substitutions, non-conservative substitutions, or a combination thereof) within the amino acid sequence of a reference Gene Writer, e.g., as a result of a change in the nucleotide sequence encoding the gene writer that results in, e.g., a change in the codon at any particular position in the coding sequence, the deletion of one or more amino acids (e.g., a truncated protein), the insertion of one or more amino acids, or any combination of the foregoing. The evolved variant Gene Writer may include variants in one or more components or domains of the Gene Writer (e.g., variants introduced into a polymerase domain, endonuclease domain, DNA binding domain, or combinations thereof).

In some aspects, the invention provides Gene Writers, systems, kits, and methods using or comprising an evolved variant of a. Gene Writer, e.g., employs an evolved variant of a Gene Writer or a Gene Writer produced or producible by PACE or PANCE. In embodiments the unevolved reference Gene Writer is a Gene Writer as disclosed herein.

The term “phage-assisted continuous evolution (PACE),” as used herein, generally refers to continuous evolution that employs phage as viral vectors. Examples of PACE technology have been described, for example, in International PCT Application No. PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and international PCT Application, PCT/US20161027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat (Them Biol. 13(12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to Gene Writers may be readily appreciated by the skilled artisan by reference to, inter alto, the foregoing references. Additional exemplary methods for directing continuous evolution of genome-modifying proteins or systems, e.g, in a population of host cells, e.g., using phage particles, can be applied to generate evolved variants of Gene Writers, or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594 issued May 5, 2015; U.S. Pat. No. 9,771,574 issued Sep. 26, 2017; U.S. Pat. No. 9,394,537, issued Jul. 19, 2016; international PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed. Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and international Patent Publication No. PCT/US2019/47996, filed Aug. 23, 2019, each of which is incorporated herein by reference in its entirety.

In some non-limiting illustrative embodiments, a method of evolution of a evolved variant Gene Writer, of a fragment or domain thereof, comprises: (a) contacting a population of host cells with a population of viral vectors comprising the gene of interest (the starting Gene Writer or fragment or domain thereof), wherein: (1) the host cell is amenable to infection by the viral vector; (2) the host. cell expresses viral genes required Par the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (h) contacting the host cells with a mutagen, using host cells with mutations that elevate mutation rate (e.g, either by carrying a mutation plasmid or some genome modification—e.g., proofing-impaired DNA polymerase, SOS genes, such as UmuC, and/or UmuD′, and/or RecA, which mutations, if plasmid-hound, may be under control of an inducible promoter), or a combination thereof. In some embodiments, the method comprises (c) incubating the population of host. cells under conditions allowing for viral replication and the production of viral particles, wherein host cells are removed from the host cell population, and fresh, uninfected. host. cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant Gene Writer, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gill, but otherwise comprise gI, gII, gIV, gV, gVI, gVII, gVIII, gIX, and a gX. In some embodiments, the generation of infectious VSV particles involves the envelope, protein VSV-G. Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust. the time a host cell remains in a. population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml, about 5-10⁷ cells/ml, about 10⁸ cells/ml, about 5-10⁸ cells/ml, about 10⁹ cells/ml, about 5-10⁹ cells/ml, about 10¹⁰ cells/ml, or about 5-10¹⁰ cells/ml.

### Promoters

In some embodiments, one or more promoter or enhancer elements are operably linked to a nucleic acid encoding a Gene Writer protein or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbamylase promoter and enhancer may be used to control expression of the ornithine transcarbamylase gene in a system or method provided by the invention for correcting ornithine transcarbamylase deficiencies. In some embodiments, the promoter is a promoter of Table 27 or a functional fragment or variant thereof.

Exemplary tissue specific promoters that are commercially available can be found, for example, at a uniform resource locator (e.g., https://www.invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR. In some embodiments, the 5′ UTR comprises an intron. In other embodiments, these include composite promoters, which combine promoter elements of different origins or were generated by assembling a distal enhancer with a minimal promoter of the same origin.

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (//epd.epfl.ch//index.php).

TABLE 27
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 promoter | pancreatic acinar cells |
| Endoglin promoter | endothelial cells |
| fibronectin promoter | differentiating cells, healing tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb promoter | Liver |
| SV40/bAlb promoter | Liver |
| SV40/Cd3 promoter | Leukocytes and platelets |
| SV40/CD45 promoter | hematopoeitic cells |
| NSE/RU5′ promoter | Mature Neurons |

TABLE 12
Additional exemplary cell or tissue-specific promoters

| Promoter | Gene Description | Gene Specificity |
|---|---|---|
| APOA2 | Apolipoprotein A-II | Hepatocytes (from hepatocyte |
| | | progenitors) |
| SERPINA1 | Serpin peptidase inhibitor, clade A | Hepatocytes |
| (hAAT) | (alpha-1 antiproteinase, antitrypsin), | (from definitive endoderm |
| | member 1 (also named alpha 1 anti-tryps in) | stage) |
| CYP3A | Cytochrome P450, family 3, | Mature Hepatocytes |
| | subfamily A, polypeptide | |
| MIR122 | MicroRNA 122 | Hepatocytes |
| | | (from early stage embryonic |
| | | liver cells) |
| | | and endoderm |

Pancreatic specific promoters

| | | |
|---|---|---|
| INS | Insulin | Pancreatic beta cells |
| | | (from definitive endoderm stage) |
| IRS2 | Insulin receptor substrate 2 | Pancreatic beta cells |
| Pdx1 | Pancreatic and duodenal | Pancreas |
| | homeobox 1 | (from definitive endoderm stage) |
| Alx3 | Aristaless-like homeobox 3 | Pancreatic beta cells |
| | | (from definitive endoderm stage) |
| Ppy | Pancreatic polypeptide | PP pancreatic cells |
| | | (gamma cells) |

Cardiac specific promoters

| | | |
|---|---|---|
| Myh6 | Myosin, heavy chain 6, cardiac | Late differentiation marker of cardiac |
| (aMHC) | muscle, alpha | muscle cells (atrial specificity) |
| MYL2 | Myosin, light chain 2, regulatory, | Late differentiation marker of cardiac |
| (MLC-2v) | cardiac, slow | muscle cells (ventricular specificity) |
| ITNNl3 | Troponin I type 3 (cardiac) | Cardiomyocytes |
| (cTnl) | | (from immature state) |
| ITNNl3 | Troponin I type 3 (cardiac) | Cardiomyocytes |
| (cTnl) | | (from immature state) |
| NPPA | Natriuretic peptide precursor A (also | Atrial specificity in adult cells |
| (ANT) | named Atrial Natriuretic Factor) | |
| Slc8a1 | Solute carrier family 8 | Cardiomyocytes from early |
| (Next) | (sodium/calcium exchanger), member | developmental stages |
| | 1 | |

CNS specific promoters

| | | |
|---|---|---|
| SYN1 | Synapsin I | Neurons |
| (hSyn) | | |
| GFAP | Glial fibrillary acidic protein | Astrocytes |
| INA | lntemexin neuronal intermediate | Neuroprogenitors |
| | filament protein, alpha (a-internexin) | |
| NES | Nestin | Neuroprogenitors and ectoderm |
| MOBP | Myelin-associated oligodendrocyte | Oligodendrocytes |
| | basic protein | |
| MBP | Myelin basic protein | Oligodendrocytes |
| TH | Tyrosine hydroxylase | Dopaminergic neurons |
| FOXA2 | Forkhead box A2 | Dopaminergic neurons (also used as a |
| (HNF3 | | marker of endoderm) |
| beta) | | |

Skin specific promoters

| | | |
|---|---|---|
| FLG | Filaggrin | Keratinocytes from granular layer |
| K14 | Keratin 14 | Keratinocytes from granular |
| | | and basal layers |
| TGM3 | Transglutaminase 3 | Keratinocytes from granular layer |

Immune cell specific promoters

| | | |
|---|---|---|
| ITGAM | lntegrin, alpha M (complement | Monocytes, macrophages, granulocytes, |
| (CD11B) | component 3 receptor 3 subunit) | natural killer cells |

Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Ferl14 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Oct4 | POU class 5 homeobox 1 | Pluripotent cells |
| (POU5F1) | | (germ cells, ES cells, iPS cells) |
| NANOG | Nanog homeobox | Pluripotent cells |
| | | (ES cells, iPS cells) |
| Synthetic | Synthetic promoter based on a Oct-4 | Pluripotent cells (ES cells, iPS cells) |
| Oct4 | core enhancer element | |
| T | Brachyury | Mesoderm |
| brachyury | | |
| NES | Nestin | Neuroprogenitors and Ectoderm |
| SOX17 | SRY (sex determining region Y)-box | Endoderm |
| | 17 | |
| FOXA2 | Forkhead box A2 | Endoderm (also used as a marker of |
| (HNFJ | | dopaminergic neurons) |
| beta) | | |
| MIR122 | MicroRNA 122 | Endoderm and hepatocytes |
| | | (from early stage embryonic liver cells~ |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544; incorporated herein by reference in its entirety).

In some embodiments, a nucleic acid encoding a Gene Writer or template nucleic acid is operably linked to a control element, e.g., a transcriptional control element, such as a promoter. The transcriptional control element may, in some embodiment, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific, enolase (NSE) promoter (see, e.g., EMBL HSENO2, X51956): an aromatic amino acid decarboxylase (AADC) promoter, a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol, Brain Res. 16:274; Boundy et al, (1998) J. Neurosci. 18:9989; and Kaneda. et al. (1991) Neuron 6:583-594. a GnRH promoter (see, e.g., Radovick et al. (1991) Proc. Natl. Acad. Sci. USA 88:3402-3406); an L7 promoter (see, e.g., Oberdick et al. (1.990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIIα) promoter (see, e.g., Mayford et al, (1996) Proc. Natl. Acad. Sci. USA 93:13250; and Casanova et al. (2001) Genesis 31:37); CMV enhancer/platelet-derived growth factor-β promoter (see, e.g., Liu et al. (2004) Gene Therapy 11:52-60); and the like.

Adipocyte-specific spatially restricted promoters include, but, are not limited to, the aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem, 277:15703); a stearoyl-CoA desaturase-1 (SCD1) promoter (Tabor et al. (1999) J. Biol. Chem, 274:20603); a leptin promoter (see, e.g., Mason et al. (1998) Endocrinol. 139:1013; and Chen et al. (1999) Biochem. Biophys. Res. Comm. 262:1873; an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al, (1989) Proc. Natl. Acad, Sci. USA 86:7490); a resistin promoter (see, e.g., Seo et al. (2003) Molec. Endocrinol. 17:1522); and the like.

Cardiomyocyte-specific spatially restricted promoters include, but are not limited to, control sequences derived from the following genes: myosin light chain-2, α-myosin heavy chain, AE3, cardiac troponin C. cardiac actin, and the like. Franz et al, (1997) Cardiovasc. Res, Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to, an SM22a promoter (see, e.g., Akyürek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM22a promoter, within which lie two CArG dements, has been shown to mediate vascular smooth muscle cell-specific expression (see, e.g., Kim, et al. (1997) Mol. Cell Biol, 17, 2266-2278; Li, et al, (1.996) J. Cell Biol. 132, 849-859; and Moessler, et al. (1996) Development 122, 2415-2425).

Photoreceptor-specific spatially restricted promoters include, but are not limited to, a. rhodopsin promoter; a rhodopsin kinase promoter (Young et al. (2003) Ophthalmol. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama, et al. (1.992) Exp Eye Res. 55:225); and the like.

Nonlimiting Exemplary Cells-Specific Promoters

Cell-specific promoters known in the art may be used to direct expression of a Gene Writer protein, e.g., as described herein. Nonlimiting exemplary mammalian cell-specific promoters have been characterized and used in mice expressing Cre recombinase in a. cell-specific manner. Certain nonlimiting exemplary mammalian cell-specific promoters are listed in Table 1 of U.S. Pat. No. 9,845,481, incorporated herein by reference.

In some embodiments, the cell-specific promoter is a promoter that is active in plants. Many exemplary cell-specific plant promoters are known in the art. See, U.S. Pat. Nos. 5,783,393; 5,880,330; 5,981,727; 7,557,264; 6,291,666; 7,132,526; and 7,323,622; and U.S. Publication Nos. 2010/0269226; 2007/0180580; 2005/0034192; and 2005/0086712, which are incorporated by reference herein in their entireties for any purpose.

In some embodiments, a vector as described herein comprises an expression cassette. The term “expression cassette”, as used herein, refers to a. nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term “operatively linked” refers to the association of two or more nucleic acid fragments on a single nucleic acid fragment so that the function of one is affected h the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. in certain embodiments, the promoter is a. heterologous promoter. The term “heterologous promoter”, as used herein, refers to a promoter that is not found to be operatively linked to a given encoding sequence in nature. In certain embodiments, an expression cassette may comprise additional elements, for example, an intron, an enhancer, a polyadenylation site, a woodchuck response element (WRE), and/or other elements known to affect expression levels of the encoding sequence. A promoter typically controls the expression of a coding sequence or functional RNA. In certain embodiments, a promoter sequence comprises proximal and more distal upstream elements and can further comprise an enhancer element. An enhancer can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoter include, but are not limited to, the phosphoglycerate kinase (PKG) promoter, CAG (composite. of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein, Such promoter sequences are commercially available from, e.g., Stratagene (San Diego, CA). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof is used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities. in some cases, the tissue-specific regulatory sequences bind tissue-specific, transcription factors that induce transcription in a tissue specific manner. Various tissue-specific regulatory sequences (e.g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (NICK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (APP) promoter, Arbuthnot et al., Hum. Gene Ther., 7:1503-14 (1996)), bone osteocalcin promoter (Stein et al., Mol. Biol. Rep. 24:185-96 (1997)); bone sialoprotein promoter (Chen et al., Bone Miner. Res., 11:654-64 (1996)), CD2 promoter (Hansal et al., J. 161:1063-8 (1998); immunoglobulin heavy chain promoter; T cell receptor α-chain promoter, neuronal such as neuron-specific enolase (NSE) promoter (Andersen et Cell. Mol. 13:503-15 (1993)), neurofilament light-chain gene promoter (Piccioli et Proc. Natl. Acad. Sci. USA. 88:5611-5 (1991)), and the neuron-specific vgf gene promoter (Piccioli et al., Neuron, 15:373-84 (1995)), and others. Additional exemplary promoter sequences are described, for example, in U.S. Pat. No. 10,300,146 (incorporated herein by reference in its entirety). In some embodiments, a tissue-specific regulatory element, e.g. a tissue-specific promoter, is selected from one known to be operably linked to a gene that is highly expressed in a given tissue, e.g., as measured by RNA-seq protein expression data, or a combination thereof, Methods for analyzing tissue specificity by expression are taught in Fagerberg et al, Mol Cell Proteomics 13(2):397-406 (2014), which is incorporated herein by reference in its entirety.

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a gene writer and gene writer template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

Ira some embodiments, the sequence encodes an RNA with a hairpin. In some embodiments, the hairpin RNA is a guide RNA, a template RNA, shRNA, or a microRNA. In some embodiments, the first promoter is an RNA polymerase I promoter. In some embodiments, the first promoter is an RNA polymerase II promoter. In some embodiments, the second promoter is an RNA polymerase III promoter. In some embodiments, the second promoter is a U6 or H1 promoter. In some embodiments, the nucleic acid construct comprises the structure of AAV construct B1 or B2.

Without wishing to be hound by theory, multicistronic expression constructs may not achieve optimal expression levels as compared to expression systems containing only one cistron, One of the suggested causes of lower expression levels achieved with multicistronic expression constructs comprising two or more promoter elements is the phenomenon of promoter interference (see, e.g., Curtin J A. Dane A P, Swanson A, Alexander I E, Ginn S L. Bidirectional promoter interference between two widely used internal heterologous promoters in a late-generation lentiviral construct. Gene Ther, 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). in some embodiments, the problem of promoter interference may be overcome, e.g., by producing multicistronic expression constructs comprising only one promoter driving transcription of multiple encoding nucleic acid sequences separated by internal ribosomal entry sites, or by separating cistrons comprising their own promoter with transcriptional insulator elements. in some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

MicroRNAs

miRNAs and other small interfering nucleic acids generally regulate gene expression via target RNA transcript cleavage/degradation or translational repression of the target messenger RNA (MRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarily to the mature miRNA. Useful transgene products may include, for example, miRNAs or miRNA. binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Pat. No. 10,300,146, 22:25-25:48, incorporated by reference, in some embodiments, one or more binding sites for one or more of the foregoing miRNAs are incorporated in a transgene, e.g., a transgene delivered by a. rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety). For liver-specific Gene Writing, however, overexpression of miR-122 may be utilized instead of using binding sites to effect miR-122-specific degradation. This miRNA is positively associated with hepatic differentiation and maturation, as well as enhanced expression of liver specific genes. Thus, in some embodiments, the coding sequence for miR-122 may be added to a component of a Gene Writing system to enhance a liver-directed therapy.

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods, Epub Aug. 12, 2007; incorporated by reference herein in its entirety). In some embodiments, microRNA sponges, or other miR inhibitors, are used with the AAVs. microRNA. sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a Gene Writing system (e.g., mRNA encoding a Gene Writer polypeptide or a heterologous object sequence expressed from the genome after successful Gene Writing) in a portion of cells. In some embodiments, it is advantageous to restrict expression of a component of a Gene Writing system to select cell types within a tissue of interest.

For example, it is known that in a given tissue, e.g., liver, macrophages and immune cells, e.g., Kupffer cells in the liver, may engage in uptake of a delivery vehicle for one or more components of a Gene Writing system. In some embodiments, at least one binding site for at least one miRNA highly expressed in macrophages and immune cells, e.g., Kupffer cells, is included in at least one component of a Gene Writing system, e.g., nucleic acid encoding a Gene Writing polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing Gene Writer levels and/or Gene Writer activity in cells in which Gene Writer expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12(569):eaba9188 (2020), incorporated herein by reference in its entirety). In some embodiments, at least one miRNA binding site may be incorporated into a nucleic acid component of a Gene Writing system to reduce expression of a system component in a neuron, e.g., a dorsal root ganglion neuron. In some embodiments, the at least one miRNA binding site incorporated into a nucleic acid component of a Gene Writing system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a Gene Writing system to reduce expression of a system component in a neuron is a binding site of miR-183, e.g., mature miRNA hsa-miR-183- or hsa-miR-183-3p. In some embodiments, combinations of miRNA binding sites may be used to enhance the restriction of expression of one or more components of a Gene Writing system to a tissue or cell type of interest.

The table below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE 10
Exemplary miRNA from off-target cells and tissues

| | miRNA | | | |
|---|---|---|---|---|
| Silenced cell type | name | Mature miRNA | miRNA sequence | SEQ ID NO |
| Kupffer cells | miR-142 | hsa-miR-142-5p | cauaaaguagaaagcacuacu | 1572 |
| Kupffer cells | miR-142 | hsa-miR-142-3p | uguaguguuuccuacuuuaugga | 1573 |
| Dorsal root ganglion | miR-182 | hsa-miR-182-5p | uuuggcaaugguagaacucacacu | 1574 |
| neurons | | | | |
| Dorsal root ganglion | miR-182 | hsa-miR-182-3p | ugguucuagacuugccaacua | 1575 |
| neurons | | | | |
| Dorsal root ganglion | miR-183 | hsa-miR-183-5p | uauggcacugguagaauucacu | 1576 |
| neurons | | | | |
| Dorsal root ganglion | miR-183 | hsa-miR-183-3p | gugaauuaccgaagggccauaa | 1577 |
| neurons | | | | |
| Hepatocytes | miR-122 | hsa-miR-122-5p | uggagugugacaaugguguuug | 1578 |
| Hepatocytes | miR-122 | hsa-miR-122-3p | aacgccauuaucacacuaaaua | 1579 |

Anticrispr Systems for Regulating GeneWriter Activity

Various approaches for modulating Cas molecule activity may be used in conjunction with the systems and methods described herein. For instance, in some embodiments, a polypeptide described herein (e.g., a Cas molecule or a GeneWriter comprising a Cas domain) can be regulated using an anticrispr agent (e.g., an anticrispr protein or anticrispr small molecule). In some embodiments, the Cas molecule or Cas domain comprises a responsive intein such as, for example, a 4-hydroxytamoxifen (4-HT)-responsive intein, an iCas molecule (e.g., iCas9); a 4-HT-responsive Cas (e.g., allosterically regulated Cas9 (arC9) or dead Cas9 (dC9)). The systems and methods described herein can also utilize a chemically-induced dimerization system of split protein fragments (e.g., rapamycin-mediated dimerization of FK506 binding protein 12 (FKBP) and FKBP rapamycin binding domain (FRB), an abscisic acid-inducible ABI-PYL1 and gibberellin-inducible GID1-GAI heterodimerization domains); a dimer of BCL-xL peptide and BH3 peptides, a A385358 (A3) small molecule, a degron system (e.g., a FKBP-Cas9 destabilized system, an auxin-inducible degron (AID) or an E. coli DHFR degron system), an aptamer or aptazyme fused with gRNA (e.g., tetracycline- and theophylline-responsive bioswitches), AcrIIA2 and AcrIIA4 proteins, and BRD0539.

In some embodiments, a small molecule-responsive intein (e.g., 4-hydroxytamoxifen (4-HT)-responsive intein) is inserted at specific sites within a Cas molecule (e.g., Cas9). In some embodiments, the insertion of a 4HT-responsive intein disrupts Cas9 enzymatic activity. In some embodiments, a Cas molecule (e.g., iCas9) is fused to the hormone binding domain of the estrogen receptor (ERT2). In some embodiments, the ligand binding domain of the human estrogen receptor-α can be inserted into a Cas molecule (e.g., Cas9 or dead Cas9 (dC9)), e.g., at position 231, yielding a 4HT-responsive anticrispr Cas9 (e.g., arC9 or dC9). In some embodiments, dCas9 can provide 4-HT dose-dependent repression of Cas9 function. In some embodiments, arC9 can provide 4-HT dose-dependent control of Cas9 function. In some embodiments, a Cas molecule (e.g., Cas9) is fused to split protein fragments. In some embodiments, chemically-induced dimerization of split protein fragments (e.g., rapamycin-mediated dimerization of FK506 binding protein 12 (FKBP) and FKBP rapamycin binding domain (FRB)) can induce low levels of Cas9 molecule activity. In some embodiments, a chemically-induced dimerization system (e.g., abscisic acid-inducible ABI-PYL1 and gibberellin-inducible GID1-GAI heterodimerization domains) can induce a dose-dependent and reversible transcriptional activation/repression of Cas9. In some embodiments, a Cas9 inducible system (ciCas9) comprises the replacement of a Cas molecule (e.g., Cas9) REC2 domain with a BCL-xl peptide and attachment of a BH3 peptide to the N- and C-termini of the modified Cas9.BCL. In some embodiments, the interaction between BCL-xL and BH3 peptides can keep Cas9 in an inactive state. In some embodiments, a small molecule (e.g., A-385358 (A3)) can disrupt the interaction between BLC-xl and BH3 peptides to activate Cas9. In some embodiments, a Cas9 inducible system can exhibit dose-dependent control of nuclease activity. In some embodiments, a degron system can induce degradation of a Cas molecule (e.g., Cas9) upon activation or deactivation by an external factor (e.g., small-molecule ligand, light, temperature, or a protein). In some embodiments, a small molecule BRD0539 inhibits a Cas molecule (e.g., Cas9) reversibly. Additional information on anticrispr proteins or anticrispr small molecules can be found, for example, in Gangopadhyay, S. A. et al. Precision control of CRISPR-Cas9 using small molecules and light, Biochemistry, 2019, Maji, B. et al. A high-throughput platform to identify small molecule inhibitors of CRISPR-Cas9, and Pawluk Anti-CRISPR: discovery, mechanism and function Nature Reviews Microbiology volume 16, pages 12-17(2018), each of which is incorporated by reference in its entirety.

Self-Inactivating Modules for Regulating GeneWriter Activity

In some embodiments the Gene Writer systems described herein includes a self-inactivating module. The self-inactivating module leads to a decrease of expression of the Gene Writer polypeptide, the Gene Writer template, or both. Without wishing to be bound by the theory, the self-inactivating module provides for a temporary period of Gene Writer expression prior to inactivation. Without wishing to be bound by theory, the activity of the Gene Writer polypeptide at a target site introduces a mutation (e.g. a substitution, insertion, or deletion) into the DNA encoding the Gene Writer polypeptide or Gene Writer template which results in a decrease of Gene Writer polypeptide or template expression. In some embodiments of the self-inactivating module, a target site for the Gene Writer polypeptide is included in the DNA encoding the Gene Writer polypeptide or Gene Writer template. In some embodiments, one, two, three, four, five, or more copies of the target site are included in the DNA encoding the Gene Writer polypeptide or Gene Writer template. In some embodiments, the target site in the DNA encoding the Gene Writer polypeptide or Gene Writer template is the same target site as the target site on the genome. In some embodiments, the target site is a different target site than the target site on the genome. In some embodiments the target side is nicked. The target site may be incorporated into an enhancer, a promoter, an untranslated region, an exon, an intron, an open reading frame, or a stuffer sequence.

In some embodiments, upon inactivation, the decrease of expression is 25%, 50%, 60%, 70%, 80%, 90%, 95%, 99%, 99.9%, or more lower than a Gene Writing system that does not contain the self-inactivating module. In some embodiments, a Gene Writer system that contains the self-inactivating module has a 5%, 10%, 15%, 20%, 25%, 30%, 40%, 50%, 60%, 70%, 80%, 90%, 95% 99%, or higher rate of integrations in target sites than off-target sites compared to a Gene Writing system that does not contain the self-inactivation module. a Gene Writer system that contains the self-inactivating module has a 10%, 15%, 20%, 25%, 30%, 40%, 50%, 60%, 70%, 80%, 90%, 95% 99%, or higher efficiency of target site modification compared to a Gene Writing system that does not contain the self-inactivation module. In some embodiments, the self-inactivating module is included when the Gene Writer polypeptide is delivered as DNA, e.g. via a viral vector.

Self-inactivating modules have been described for nucleases. See, e.g. in Li et al A Self-Deleting AAV-CRISPR System for In Vivo Genome Editing, Mol Ther Methods Clin Dev. 2019 Mar. 15; 12: 111-122, P. Singhal, Self-Inactivating Cas9: a method for reducing exposure while maintaining efficacy in virally delivered Cas9 applications (available at www.editasmedicine.com/wp-content/uploads/2019/10/aef_asgct_poster_2017_final_-_present_5-11-17_515pm1_1494537387_1494558495_1497467403.pdf), and Epstein and Schaffer Engineering a Self-Inactivating CRISPR System for AAV Vectors Targeted Genome Editing Il Volume 24, SUPPLEMENT 1, S50, May 1, 2016, and WO2018106693A1.

Small Molecules

In some embodiments a polypeptide described herein (e.g., a Gene Writer polypeptide) is controllable via a small molecule. In some embodiments the polypeptide is dimerized via a small molecule.

In some embodiment, the polypeptide is controllable via. Chemical Induction of Dimerization (CID) with small molecules. CID is generally used to generate switches of protein function to alter cell physiology. An exemplary high specificity, efficient dimerizer is rimiducid (AP1903), which has two identical, protein-binding surfaces arranged tail-to-tail, each with high affinity and specificity for a mutant of FKBP12: FKBP12(F36V) (FKBP12v36, F_{V36} or Fᵥ). Attachment of one or more Fv domains onto one or more cell signaling molecules that normally rely on homodimerization can convert that protein to rimiducid control. Homodimerization with rimiducid is used in the context of an inducible caspase safety switch. This molecular switch that is controlled by a distinct dimerizer ligand, based on the heterodimerizing small molecule, rapamycin, or rapamycin analogs (“rapalogs”), Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of MTOR. Provided in some embodiments of the present application are molecular switches that greatly augment the use of rapamycin, rapalogs and rimiducid as agents for therapeutic applications.

In some embodiments of the dual switch technology, a, hon such as AP1903 (rimiducid), directly induces dimerization or multimerization of polypeptides comprising an FKBP12 multimerizing region. In other embodiments, a polypeptide comprising an FKBP12 multimerization is multimerized, or aggregated by binding to a heterodimerizer, such as rapamycin or a rapalog, which also binds to an FRB or FRB variant multimerizing region on a chimeric polypeptide, also expressed in the modified cell, such as, for example, a chimeric antigen receptor. Rapamycin is a natural product macrolide that binds with high affinity (<1 nM) to FKBP12 and together initiates the high-affinity, inhibitory interaction with the FKBP-Rapamycin-Binding (FRB) domain of mTOR. FRB is small (89 amino acids) and can thereby be used as a protein “tag” or “handle” when appended to many proteins. Coexpression. of a FRB-fused protein with a FKBP12-fused protein renders their approximation rapamycin-inducible (12-16). This can serve as the basis for a cell safety switch regulated by the orally available ligand, rapamycin, or derivatives of rapamycin (rapalogs) that do not inhibit mTOR at a low, therapeutic dose but instead bind with selected, Caspase-9-fused mutant FRB domains, (see Sabatini D M, et al., Cell. 1994; 78(1):35-43; Brown F J, et al., Nature. 1994; 369(6483):756-8; Chen J, et al., Proc Natl. Acad. Sci USA, 1995; 92(10:4947-51; and Choi J. Science. 1996; 273(5272):23942).

In some embodiments, two levels of control are provided in the therapeutic cells. In embodiments, the first level of control may be tunable, i.e., the level of removal of the therapeutic cells may be controlled so that it results in partial removal of the therapeutic cells. In some embodiments, the chimeric antigen polypeptide comprises a binding site for rapamycin, or a rapamycin analog. In embodiments. also present in the therapeutic cell is a suicide gene, such as, for example, one encoding a caspase polypeptide. Using this controllable first level, the need for continued therapy may, in some embodiments, be balanced with the need to eliminate or reduce the level of negative side effects. In some embodiments, a rapamycin analog, a rapalog is administered to the patient, which then hinds to both the caspase polypeptide and the chimeric antigen receptor, thus recruiting the caspase polypeptide to the location, and aggregating the caspase polypeptide. Upon aggregation, the caspase polypeptide induces apoptosis. The amount of rapamycin or rapamycin analog administered to the patient may vary; if the removal of a lower level of cells by apoptosis is desired, a lower level of rapamycin or rapamycin may be administered to the patient. In some embodiments, the second level of control may be designed to achieve the maximum level of cell elimination. This second level may be based, for example, on the use of rimiducid, or AP1903, if there is a need to rapidly eliminate up to 100% of the therapeutic cells, the AP1903 may be administered to the patient. The multimeric AP1903 binds to the caspase polypeptide, leading to multimerization of the caspase, polypeptide and apoptosis. In certain examples, second level may also be tunable, or controlled, by the level of AP1903 administered to the subject.

In certain embodiments, small molecules can be used to control genes, as described in for example, U.S. Ser. No. 10/584,351 at 47:53-56:47 (incorporated by reference herein in its entirety), together suitable ligands for the control features, e.g., in U.S. Ser. No. 10/584,351 at 56:48, et seq. as well as U10046049 at 43:27-52:20, incorporated by reference as well as the description of ligands for such control systems at 52:21, et seq.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

The disclosure provides, in part, a nucleic acid, e.g., vector, encoding a Gene Writer polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a Gene Writer polypeptide is integrated into a target cell genome (e.g., upon administration to a target cell, tissue, organ, or subject). In some embodiments, a vector encoding a Gene Writer polypeptide is not integrated into a target cell genome (e.g., upon administration to a target cell, tissue, organ, or subject). In some embodiments, a vector comprising a template nucleic acid (e.g., template DNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a Gene Writer polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

RNAs (e.g., a gRNA or an mRNA, e.g., an mRNA encoding a GeneWriter) may also be produced as described herein. In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gRNA is produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered, e.g., be less than 37° C. (e.g., between 0-10 C, 10-20 C, or 20-30 C), to result in a higher proportion of full-length transcripts (see Krieg Nucleic Acids Res 18:6463 (1990), which is herein incorporated by reference in its entirety). In some embodiments, a protocol for improved synthesis of long transcripts is employed to synthesize a long RNA, e.g., an RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a Gene Writer polypeptide) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC (SEQ ID NO: 1568) and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 1569), or functional fragments or variants thereof, and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

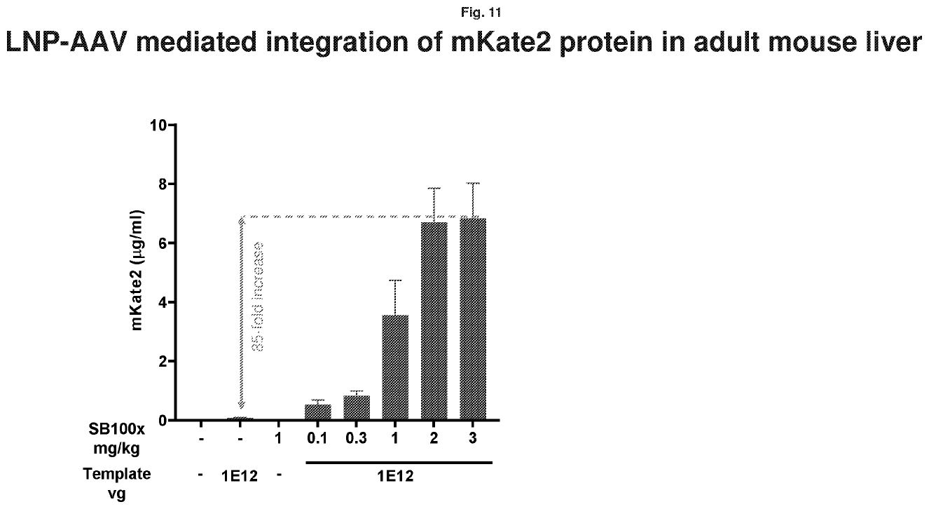

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

In some embodiments, RNA segments may be connected using an Azide-Alkyne Huisgen Cycloaddition. reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitation, the use of halogens (F—, Br—, I—)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

By way of illustration, a tracrRNA is typically around 80 nucleotides in length. Such RNA molecules may be produced, for example, by processes such as in vitro transcription or chemical synthesis. In some embodiments, when chemical synthesis is used to produce such RNA molecules, they may be produced as a single synthesis product or by linking two or more synthesized RNA segments to each other. In embodiments, when three or more RNA segments are connected to each other, different methods may be used to link the individual segments together. Also, the RNA segments may be connected to each other in one pot (e.g., a container, vessel, well, tube, plate, or other receptacle), all at the same time, or in one pot at different times or in different pots at different times. In a non-limiting example, to assemble RNA Segments 1, 2 and 3 in numerical order, RNA Segments 1 and 2 may first be connected, 5′ to 3′, to each other. The reaction product may then be purified for reaction mixture components (e.g., by chromatography), then placed in a second pot, for connection of the 3′ terminus with the 5′ terminus of RNA Segment 3. The final reaction product may then be connected to the 5′ terminus of RNA Segment 3.

In another non-limiting example, RNA Segment 1 (about 30 nucleotides) is the target locus recognition sequence of a crRNA and a portion of Hairpin Region 1. RNA Segment 2 (about 35 nucleotides) contains the remainder of Hairpin Region 1 and some of the linear tracrRNA between Hairpin Region 1 and Hairpin Region 2. RNA Segment 3 (about 35 nucleotides) contains the remainder of the linear tracrRNA between Hairpin Region 1 and Hairpin Region 2 and all of Hairpin Region 2. In this example, RNA Segments 2 and 3 are linked, 5′ to 3′, using click chemistry. Further, the 5′ and 3′ end termini of the reaction product are both phosphorylated. The reaction product is then contacted with RNA Segment 1, having a 3′ terminal hydroxyl group, and T4 RNA ligase to produce a guide RNA molecule.

A number of additional linking chemistries may be used to connect RNA segments according to method of the invention. Some of these chemistries are set out in Table 6 of US20160102322A1, which is incorporated herein by reference in its entirety.

### Kits, Articles of Manufacture, and Pharmaceutical Compositions

In an aspect the disclosure provides a kit comprising a Gene Writer or a Gene Writing system, e.g., as described herein. In some embodiments, the kit comprises a Gene Writer polypeptide (or a nucleic acid encoding the polypeptide) and a template DNA. In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), Gene Writers, and/or Gene Writer systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a Gene Writer or a Gene Writing system, e.g., as described herein. In some embodiments, the pharmaceutical composition further comprises a pharmaceutically acceptable carrier or excipient. In some embodiments, the pharmaceutical composition comprises a template DNA.

### Chemistry, Manufacturing, and Controls (CMC)

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a Gene Writer™ system, polypeptide, and/or template nucleic acid (e.g., template DNA) conforms to certain quality standards. In some embodiments, a Gene Writer™ system, polypeptide, and/or template nucleic acid (e.g., template DNA) produced by a method described herein conforms to certain quality standards. Accordingly, the disclosure is directed, in some aspects, to methods of manufacturing a Gene Writer™ system, polypeptide, and/or template nucleic acid that conforms to certain quality standards, e.g., in which said quality standards are assayed. The disclosure is also directed, in some aspects, to methods of assaying said quality standards in a Gene Writer™ system, polypeptide, and/or template nucleic acid. In some embodiments, quality standards include, but are not limited to, one or more (e.g., 1, 2, 3, 4, 5, 6, 7, 8, 9, 10, 11, or 12) of the following:
(i) the length of the template DNA or the mRNA encoding the GeneWriter polypeptide, e.g., whether the DNA or mRNA has a length that is above a reference length or within a reference length range, e.g., whether at least 80, 85, 90, 95, 96, 97, 98, or 99% of the DNA or mRNA present is greater than 100, 125, 150, 175, or 200 nucleotides long;
(ii) the presence, absence, and/or length of a polyA tail on the mRNA, e.g., whether at least 80, 85, 90, 95, 96, 97, 98, or 99% of the mRNA present contains a polyA tail (e.g., a polyA tail that is at least 5, 10, 20, 30, 50, 70, 100 nucleotides in length);
(iii) the presence, absence, and/or type of a 5′ cap on the mRNA, e.g., whether at least 80, 85, 90, 95, 96, 97, 98, or 99% of the mRNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-′P), 5-methoxyuridine (5-MO-U), 5-methylcytidine (5mC), or a locked nucleotide) in the mRNA, e.g., whether at least 80, 85, 90, 95, 96, 97, 98, or 99% of the mRNA present contains one or more modified nucleotides;
(v) the stability of the template DNA or the mRNA (e.g., over time and/or under a pre-selected condition), e.g., whether at least 80, 85, 90, 95, 96, 97, 98, or 99% of the DNA or mRNA remains intact (e.g., greater than 100, 125, 150, 175, or 200 nucleotides long) after a stability test;
(vi) the potency of the template DNA or the mRNA in a system for modifying DNA, e.g., whether at least 1% of target sites are modified after a system comprising the DNA or mRNA is assayed for potency;
(vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof;
(ix) the presence, absence, and/or type of one or more artificial, synthetic, or non-canonical amino acids (e.g., selected from ornithine, β-alanine, GABA, 6-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
(x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:
(a) less than 1% (e.g., less than 0.5%, 0.4%, 0.3%, 0.2%, or 0.1%) DNA template relative to the RNA encoding the polypeptide, e.g., on a molar basis;
(b) less than 1% (e.g., less than 0.5%, 0.4%, 0.3%, 0.2%, or 0.1%) uncapped RNA relative to the RNA encoding the polypeptide, e.g., on a molar basis;
(c) less than 1% (e.g., less than 0.5%, 0.4%, 0.3%, 0.2%, or 0.1%) partial length RNAs relative to the RNA encoding the polypeptide, e.g., on a molar basis;
(d) substantially lacks unreacted cap dinucleotides.

### Applications

Using the systems described herein, optionally using any of delivery modalities described herein (including viral delivery modalities, such as AAVs), the invention also provides applications (methods) for modifying DNA molecule, such as nuclear DNA, i.e., in the genome of a cell, whether in vitro, ex vivo, in situ, or in vivo, e.g., in a tissue in an organism, such as a subject including mammalian subjects, such as a human. By integrating coding genes into a template, the Gene Writer™ system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the template nucleic acid encodes a promotor region specific to the therapeutic needs of the host cell, for example a tissue specific promotor or enhancer. In still other embodiments, a promotor can be operably linked to a coding sequence.

In certain aspects, the invention this provides methods of modifying a target DNA strand in a cell, tissue or subject, comprising administering a system as described herein (optionally by a modality described herein) to the cell, tissue or subject, where the system inserts the heterologous object sequence into the target DNA strand, thereby modifying the target DNA strand. In certain embodiments, the heterologous object sequence is thus expressed in the cell, tissue, or subject. In some embodiments, the cell, tissue or subject is a mammalian (e.g., human) cell, tissue or subject. Exemplary cells thus modified include a hepatocyte, lung epithelium, an ionocyte. Such a cell may be a primary cell or otherwise not immortalized. In related aspects, the invention also provides methods of treating a mammalian tissue comprising administering the a system as described herein to the mammal, thereby treating the tissue, wherein the tissue is deficient in the heterologous object sequence. In certain embodiments of any of the foregoing aspects and embodiments, the transposase is provided as a nucleic acid, which is present transiently.

In some embodiments, the Gene Writer™ gene editor system can provide therapeutic transgenes expressing, e.g., replacement blood factors or replacement enzymes, e.g., lysosomal enzymes. For example, the compositions, systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease. For example, the compositions, systems and methods described herein are useful to express, in a target human genome factor I, II, V, VII, X, XI, XII or XIII for blood factor deficiencies.

In some embodiments, the heterologous object sequence encodes an intracellular protein (e.g., a cytoplasmic protein, a nuclear protein, an organellar protein such as a mitochondrial protein or lysosomal protein, or a membrane protein). In some embodiments, the heterologous object sequence encodes a membrane protein, e.g. and/or an endogenous human membrane protein. In some embodiments, the heterologous object sequence encodes an extracellular protein. In some embodiments, the heterologous object sequence encodes an enzyme, a structural protein, a signaling protein, a regulatory protein, a transport protein, a sensory protein, a motor protein, a defense protein, or a storage protein. Other proteins include a immune receptor protein, e.g. a synthetic immune receptor protein such as a chimeric antigen receptor protein (CAR), a T cell receptor, a B cell receptor, or an antibody.

A Gene Writing™ system may be used to treat indications of the liver. In exemplary embodiments, the liver diseases preferred for therapeutic application of Gene Writing™ include, e.g., ornithine transcarbamylase (OTC) deficiency, carbamoyl phosphate synthetase I deficiency, citrullinemia type I, Crigler-Najjar syndrome, glycogen storage disorder IV, homozygous familial hypercholesterolemia, maple syrup urine disease, methylmalonic acidemia, progressive familial intrahepatic cholestasis 1, progressive familial intrahepatic cholestasis 2, propionic acidemia. In some embodiments, OTC deficiency is addressed by delivering all or a fragment of an OTC gene. In some embodiments, OTC deficiency is addressed by delivering a complete OTC gene expression cassette to a genome that complements the function of the mutated gene. In some embodiments, a fragment of the OTC gene is used that replaces the pathogenic mutation at its endogenous locus. In other embodiments, a Gene Writing™ system is used to address a condition selected from Column 6 of Table 4 or an indication of the lungs (e.g., alpha-1-antitrypsin (AAT) deficiency, cystic fibrosis (CF), primary ciliary dyskinesia (PCD), surfactant protein B (SP-B) deficiency) by delivering all or a fragment of a gene expression cassette encoding the corresponding gene indicated in Column 1 of Table 4, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB. In some embodiments, all or a fragment of said gene expression cassette is delivered to the endogenous locus of the pathogenic mutation. In some embodiments, all or a fragment of said gene expression cassette is integrated at a separate locus in the genome and complements the function of the mutated gene.

In certain embodiments a Gene Writer™ system provides a heterologous object sequence comprising a gene in Table 4, or all or a fragment of any of the following genes:

TABLE 4
SERPINA1,
CFTR, DNAI1, DNAH5, ARMC4, CCDC39, CCDC40, CCDC65, CCDC103, CCDC114,
CFAP298, DNAAF1, DNAAF2, DNAAF3, DNAAF4, DNAFF5, DNAH8, DNAH11, DNAI2,
DNAL1, DRC1, HYDIN, LRRC6, NME8, OFD1, RPGR, RSPH1, RSPH4A, RSPH9, SPAG1,
ZMYND10, or SFTPB.

Table 5 of WO2020014209, incorporated herein by reference.

A Gene Writing™ system may be used to treat indications of the lungs. In exemplary embodiments, the lung diseases preferred for therapeutic application of Gene Writing™ include, e.g., alpha-1-antitrypsin (AAT) deficiency, cystic fibrosis (CF), primary ciliary dyskinesia (PCD), surfactant protein B (SP-B) deficiency. In some embodiments, AAT deficiency is addressed by delivering all or a fragment of a SERPINA1 gene (UniProt E9KL23). In some embodiments, AAT deficiency is addressed by delivering a complete SERPINA1 gene expression cassette to a genome that complements the function of the mutated gene. In some embodiments, a fragment of the SERPINA1 gene is used that replaces the SERPINA1 PiZ mutation at its endogenous locus. In some embodiments, a fragment of the SERPINA1 gene is used that replaces the SERPINA1 PiS mutation at its endogenous locus. In some embodiments, a fragment of the SERPINA1 gene is used that replaces a mutation other than PiZ or PiS at its endogenous locus. In other embodiments, CF is addressed by delivering all or a fragment of a CFTR gene. In some embodiments, CF is addressed by delivering a complete CFTR (UniProt P13569) or CFTRΔR gene expression cassette (i.e., including a coding sequence and required regulatory features) to a genome that complements the function of the mutated gene. In some embodiments, a fragment of the CFTR gene is used that replaces the ΔF508 mutation at its endogenous locus. In some embodiments, a fragment of the CFTR gene is used that replaces a mutation other than ΔF508 at its endogenous locus. In other embodiments, PCD is addressed by delivering all or a fragment of a gene responsible for PCD. In some embodiments, PCD is addressed by delivering all or a fragment of a DNAI1 gene. In some embodiments, PCD is addressed by delivering all or a fragment of a DNAH5 gene. In some embodiments, PCD is addressed by delivering all or a fragment of a gene responsible for PCD other than DNAI1 or DNAH5, e.g., ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10. In still other embodiments, SP-B deficiency is addressed by delivering all or a fragment of a SFTPB gene. In some embodiments, SP-B deficiency is addressed by delivering a complete SFTPB gene expression cassette to a genome that complements the function of the mutated gene. In some embodiments, a fragment of the SFTPB gene is used that replaces a mutation in SFTPB at its endogenous locus.

In some embodiments, a Gene Writer™ system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a Gene Writer™ system described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a Gene Writer™ system described herein described herein is administered by enteral administration (e.g., oral, rectal, gastrointestinal, sublingual, sublabial, or buccal administration). In some embodiments, a Gene Writer™ system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a Gene Writer™ system described herein is administered by topical administration (e.g., transdermal administration).

In some embodiments, a Gene Writer™ system as described herein can be used to modify an animal cell, plant cell, or fungal cell. In some embodiments, a Gene Writer™ system as described herein can be used to modify a mammalian cell (e.g., a human cell). In some embodiments, a Gene Writer™ system as described herein can be used to modify a cell from a livestock animal (e.g., a cow, horse, sheep, goat, pig, llama, alpaca, camel, yak, chicken, duck, goose, or ostrich). In some embodiments, a Gene Writer™ system as described herein can be used as a laboratory tool or a research tool, or used in a laboratory method or research method, e.g., to modify an animal cell, e.g., a mammalian cell (e.g., a human cell), a plant cell, or a fungal cell.

In some embodiments, a Gene Writer™ system as described herein can be used to express a protein, template, or heterologous object sequence (e.g., in an animal cell, e.g., a mammalian cell (e.g., a human cell), a plant cell, or a fungal cell). In some embodiments, a Gene Writer™ system as described herein can be used to express a protein, template, or heterologous object sequence under the control of an inducible promoter (e.g., a small molecule inducible promoter). In some embodiments, a Gene Writing system or payload thereof is designed for tunable control, e.g., by the use of an inducible promoter. For example, a promoter, e.g., Tet, driving a gene of interest may be silent at integration, but may, in some instances, activated upon exposure to a small molecule inducer, e.g., doxycycline. In some embodiments, the tunable expression allows post-treatment control of a gene (e.g., a therapeutic gene), e.g., permitting a small molecule-dependent dosing effect. In embodiments, the small molecule-dependent dosing effect comprises altering levels of the gene product temporally and/or spatially, e.g., by local administration. In some embodiments, a promoter used in a system described herein may be inducible, e.g., responsive to an endogenous molecule of the host and/or an exogenous small molecule administered thereto.

### Additional Suitable Indications

Exemplary suitable diseases and disorders that can be treated by the systems or methods provided herein, for example, those comprising Gene Writers, include, without limitation: Baraitser-Winter syndromes 1 and 2; Diabetes mellitus and insipidus with optic atrophy and deafness; Alpha-1-antitrypsin deficiency; Heparin cofactor II deficiency; Adrenoleukodystrophy; Keppen-Lubinsky syndrome; Treacher collins syndrome 1; Mitochondrial complex I, II, III, III (nuclear type 2, 4, or 8) deficiency; Hypermanganeseinia with dystonia, polycythemia and cirrhosis; Carcinoid tumor of intestine; Rhabdoid tumor predisposition syndrome 2; Wilson disease; Hyperphenylalaninemia, bh4-deficient, a, due to partial pts deficiency, BH4-deficient, D, and non-pku; Hyperinsulinemic hypoglycemia familial 3, 4, and 5; Keratosis follicularis; Oral-facial-digital syndrome; SeSAME syndrome; Deafness, nonsyndromic sensorineural, mitochondrial; Proteinuria; Insulin-dependent diabetes mellitus secretory diarrhea syndrome; Moyamoya disease 5; Diamond-Blackfan anemia 1, 5, 8, and 10; Pseudoachondroplastic spondyloepiphyseal dysplasia syndrome; Brittle cornea syndrome 2; Methylmalonic acidemia with homocystinuria; Adams-Oliver syndrome 5 and 6; autosomal recessive Agammaglohulinemia 2; Cortical malformations, occipital; Febrile seizures, familial, 11; Mucopolysaccharidosis type VI, type VI (severe), and type VII; Marden Walker like syndrome; Pseudoneonatal adrenoleukodystrophy; Spheroid body myopathy; Cleidocranial dysostosis; Multiple Cutaneous and Mucosal Venous Malformations; Liver failure acute infantile; Neonatal intrahepatic cholestasis caused by citrin deficiency; Ventricular septal defect 1; Ochlodentodigital dysplasia; Wilms tumor 1; Weill-Marchesani-like syndrome: Renal adysplasia; Cataract 4, autosomal dominant, autosomal dominant, multiple types, with microcornea, coppock-like, juvenile, with microcomea and glucosuria, and nuclear diffuse nonprogressive; Odontohypophosphatasia; Cerebro-oculo-facio-skeletal syndrome; Schizophrenia 15; Cerebral amyloid angiopathy, APP-related; Hemophagocytic lymphohistiocytosis, familial, 3; Porphobilinogen synthase deficiency; Episodic ataxia type 2; Trichorhinophalangeal syndrome type 3; Progressive familial heart block type IB; Glioma susceptibility 1; Lichtenstein-Knorr Syndrome; Hypohidrotic X-linked ectodermal dysplasia; Bartter syndrome types 3, 3 with hypercalciuria, and 4; Carbonic anhydrase VA deficiency, hyperammonemia clue to; Cardiomyopathy; Poikiloderma, hereditary fibrosing, with tendon contractures, myopathy, and pulmonary fibrosis; Combined d-2- and 1-2-hydroxyglutaric aciduria; Arginase deficiency; Cone-rod dystrophy 2 and 6; Smith-Lemli-Opitz syndrome; Mucolipidosis III Gamma; Blau syndrome; Werner syndrome; Meningioma; Iodotyrosyl coupling defect; Dubin-Johnson syndrome; 3-Oxo-5 alpha-steroid delta 4-dehydrogenase deficiency; Boucher Neuhauser syndrome; Iron accumulation in brain; Mental Retardation, X-Linked 102 and syndromic 13; familial, Pituitary adenoma predisposition; Hypoplasia. of the corpus callosum; Hyperalphalipoproteinemia 2; Deficiency of ferroxidase; Growth hormone insensitivity with immunodeficiency; Marinesco-Sj\xc3\xb6gren syndrome; Martsolf syndrome; Gaze palsy, familial horizontal, with progressive scoliosis; Mitchell-Riley syndrome; Hypocalciuric hypercalcemia, familial, types 1 and 3; Rubinstein-Taybi syndrome; Epstein syndrome; Juvenile retinoschisis; Becker muscular dystrophy; Loeys-Dietz syndrome 1, 2, 3; Congenital muscular hypertrophy-cerebral syndrome; Familial juvenile gout; Spermatogenic failure 11, 3, and 8; Orofacial cleft 11 and 7, Cleft lip/palate-ectodermal dysplasia syndrome; Mental retardation, X-linked, nonspecific, syndromic, Hedera type, and syndromic, wu type; Combined oxidative phosphorylation deficiencies 1, 3, 4, 12, 15, and 25; Frontotemporal dementia; Kniest dysplasia; Familial cardiomyopathy; Benign familial hematuria; Pheochromocytoma; Aminoglycoside-induced deafness; Gamma-aminobutyric acid transaminase deficiency; Oculocutaneous albinism type IB, type 3, and type 4; Renal coloboma syndrome; CNS hypomyelination; Hennekam lymphangiectasia-lymphedema syndrome 2; Migraine, familial basilar; Distal spinal muscular atrophy, X-linked 3; X-linked periventricular heterotopia; Microcephaly; Mucopolysaccharidosis, MPS-I-H/S, MPS-II, MPS-III-A, MPS-III-B, MPS-III-C, MPS-IV-A, MPS-IV-B; infantile Parkinsonism-dystonia; Frontotemporal dementia with TDP43 inclusions, TARDBP-related; Hereditary diffuse gastric cancer; Sialidosis type I and II; Microcephaly-capillary malformation syndrome; Hereditary breast and ovarian cancer syndrome; Brain small vessel disease with hemorrhage; Non-ketotic hyperglycinemia; Navajo neurohepatopathy; Auriculocondylar syndrome 2; Spastic paraplegia 15, 2, 3, 35, 39, 4, autosomal dominant, 55, autosomal recessive, and 5A; Autosomal recessive cutis laxa type IA and IB; Hemolytic anemia, nonspherocytic, due to glucose phosphate isomerase deficiency; Hutchinson-Gilford syndrome; Familial amyloid nephropathy with urticaria and deafness; Supravalvar aortic stenosis; Diffuse palmoplantar keratoderma, Bothnian type; Holt-Oram syndrome; Coffin Siris/Intellectual Disability; Left-right axis malformations; Rapadilino syndrome; Nanophthalmos 2; Craniosynostosis and dental anomalies; Paragangliomas 1; Snyder Robinson syndrome; Ventricular fibrillation; Activated PI3K-delta syndrome; Howel-Evans syndrome; Larsen syndrome, dominant type; Van Maldergem syndrome 2; MYH-associated polyposis; 6-pyinvoyl-tetrahydropterin synthase deficiency; Alagille syndromes 1 and 2; Lymphangiomyomatosis; Muscle eye brain disease; WFS1-Related Disorders; Primary hypertrophic osteoarthropathy, autosomal recessive 2; Infertility; Nestor-Guillermo progeria. syndrome; Mitochondrial trifunctional protein deficiency; Hypoplastic, left heart syndrome 2; Primary dilated cardiomyopathy; Retinitis pigmentosa; Hirschsprung disease 3; Upshaw-Schulman syndrome; Desbuquois dysplasia 2; Diarrhea 3 (secretory sodium, congenital, syndromic) and 5 (with tufting enteropathy, congenital); Pachyonychia congenita 4 and type 2; Cerebral autosomal dominant and recessive arteriopathy with subcortical infarcts and leukoencephalopathy; Vi tel li form dystrophy; type II, type IV, IV (combined hepatic and myopathic), type V, and type VI; Atypical Rett syndrome; Atrioventricular septal defect 4; Papillon-Lef\xc3\xa8vre syndrome; Leber amaurosis; X-linked hereditary motor and sensory, neuropathy; Progressive sclerosing poliodystrophy; Goldmann-Favre syndrome; Renal-hepatic-pancreatic dysplasia; Pallister-Hall syndrome; Amyloidogenic transthyretin amyloidosis; Melnick-Needles syndrome; Hyperimmunoglobulin E syndrome; Posterior column ataxia with retinitis pigmentosa; Chondrodysplasia punctata 1, X-linked recessive and 2 X-linked dominant; Ectopia lentis, isolated autosomal recessive and dominant; Familial cold urticarial; adenomatous polyposis 1 and 3; Porokeratosis 8, disseminated superficial actinic type; PIK3CA Related Overgrowth Spectrum; Cerebral cavernous malformations 2; Exudative vitreoretinopathy 6; Megalencephaly cutis marmorata telangiectatica congenital; TARP syndrome; Diabetes mellitus, permanent neonatal, with neurologic features; Short-rib thoracic dysplasia 11 or 3 with or without polydactyly; Hypertrichotic osteochondrodysplasia; beta Thalassemia; Niemann-Pick disease type C1, C2, type A, and type C1, adult form; Charcot-Marie-Tooth disease types IB, 2B2, 2C, 2F, 21, 2U (axonal), 1C (demyelinating), dominant intermediate C, recessive intermediate A, 2A2, 4C, 4D, 4H, IF, IVF, and X; Tyrosinemia type I; Paroxysmal atrial fibrillation; UV-sensitive syndrome; Tooth agenesis, selective, 3 and 4; Merosin deficient congenital muscular dystrophy; Long-chain 3-hydroxyacyl-CoA dehydrogenase deficiency; Congenital aniridia; Left ventricular noncompaction 5; Deficiency of aromatic-L-amino-acid decarboxylase; Coronary heart disease; Leukonychia totalis; Distal arthrogryposis type 2B; Retinitis pigmentosa 10, 11, 12, 14, 15, 17, and 19; Robinow Sorauf syndrome; Tenorio Syndrome; Prolactinoma; Neurofibromatosis, type land type 2; Congenital muscular dystrophy-dystroglycanopathy with brain and eye anomalies, types A2, A7, A8, A11, and A14; Heterotaxy, visceral, 2, 4, and 6, autosomal; Jankovic Rivera syndrome; Lipodystrophy, familial partial, type 2 and 3; Hemoglobin H disease, nondeletional; Multicentric osteolysis, nodulosis and arthropathy; Thyroid agenesis; deficiency of Acyl-CoA dehydrogenase family, member 9; Alexander disease; Phytanic acid storage disease; Breast-ovarian cancer, familial 1, 2, and 4; Prolific dehydrogenase deficiency; Childhood hypophosphatasia; Pancreatic agenesis and congenital heart disease; Vitamin D-dependent rickets, types land 2; Iridogoniodysgenesis dominant type and type 1; Autosomal recessive hypohidrotic ectodermal dysplasia syndrome; Mental retardation, X-linked, 3, 21, 30, and 72; Hereditary hemorrhagic telangiectasia type 2; Blepharophimosis, ptosis, and epicanthus inversus; Adenine phosphoribosyltransferase deficiency; Seizures, benign familial infantile, 2; Acrodysostosis 2, with or without hormone resistance; Tetralogy of Fallot; Retinitis pigmentosa 2, 20, 25, 35, 36, 38, 39, 4, 40, 43, 45, 48, 66, 7, 70, 72; Lysosomal acid lipase deficiency; Eichsfeld type congenital muscular dystrophy; Walker-Warburg congenital muscular dystrophy; TNF receptor-associated periodic fever syndrome (TRAPS); Progressive myoclonus epilepsy with ataxia; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Long QT syndrome; Dicarboxylic aminoaciduria; Brachydactyly types A1 and A2; Pseudoxanthoma elasticum-like disorder with multiple coagulation factor deficiency; Multisystemic smooth muscle dysfunction syndrome; Syndactyly Cenani Lenz, type; Joubert syndrome 1, 6, 7, 9/15 (digenic), 14, 16, and 17, and Orofaciodigital syndrome xiv; Digitorenocerebral syndrome; Retinoblastoma; Dyskinesia, familial, with facial myokymia; Hereditary sensory and autonomic neuropathy type IIB and IIA; hyperinsulinism; Megalencephalic leukoencephalopathy with subcortical cysts land 2a; Aase syndrome; Wiedemann-Steiner syndrome; Ichthyosis exfoliativa; Myotonia congenital; Granulomatous disease, chronic, X-linked, variant; Deficiency 2-methylbutyryl-CoA dehydrogenase; Sarcoidosis, early-onset; Glaucoma, congenital and Glaucoma, congenital, Coloboma; Breast cancer, susceptibility to; Ceroid lipofuscinosis neuronal 2, 6, 7, and 10; Congenital generalized lipodystrophy type 2; Fructose-bisphosphatase deficiency; Congenital contractual arachnodactyly; Lynch syndrome 1 and 11; Phosphoglycerate dehydrogenase deficiency; Burn-Mckeown syndrome; Myocardial infarction 1; Achromatopsia 2 and 7; Retinitis Pigmentosa 73; Protan defect; Polymicrogyria, asymmetric, bilateral frontoparietal; Spinal muscular atrophy, distal, autosomal recessive, 5; Methylmalonic aciduria due to methylmalonyl-CoA mutase deficiency; Familial porencephaly; Hurler syndrome; Oto-palato-digital syndrome, types I and II; Sotos syndrome 1 or 2; Cardioencephalomyopathy, fatal infantile, due to cytochrome c oxidase deficiency; Parastremmatic dwarfism; Thyrotropin releasing hormone resistance, generalized; Diabetes mellitus, type 2, and insulin-dependent, 20; Thoracic aortic aneurysms and aortic dissections; Estrogen resistance; Maple syrup urine disease type 1A and type 3; Hypospadias 1 and 2, X-linked; Metachromatic leukodystrophy juvenile, late infantile, and adult types; Early T cell progenitor acute lymphoblastic leukemia; Neuropathy, Hereditary Sensory, Type IC; Mental retardation, autosomal dominant 31; Retinitis pigmentosa. 39; Breast cancer, early-onset; May-Hegglin anomaly; Gaucher disease type 1 and Subacute neuronopathic; Temtamy syndrome; Spinal muscular atrophy, lower extremity predominant 2, autosomal dominant; Fanconi anemia, complementation group E, I, N, and O; Alkaptonuria; Hirschsprung disease; Combined malonic and methylmalonic aciduria; Arrhythmogenic right ventricular cardiomyopathy types 5, 8, and 10; Congenital lipomatous overgrowth, vascular malformations, and epidermal nevi; Timothy syndrome; Deficiency of guanidinoacetate methyltransferase; Myoclonic dystonia; Kanzaki disease; Neutral 1 amino acid transport defect; Neurohypophyseal diabetes insipidus; Thyroid hormone metabolism, abnormal; Benign scapuloperoneal muscular dystrophy with cardiomyopathy; Hypoglycemia with deficiency of glycogen synthetase in the liver; Hypertrophic cardiomyopathy; Myasthenic Syndrome, Congenital. 11, associated with acetylcholine receptor deficiency; Mental retardation. X-linked syndromic 5; Stormorken syndrome; Aplastic anemia; Intellectual disability; Normokalemic periodic paralysis, potassium-sensitive; Danon disease; Nephronophthisis 13, 15 and 4; Thyrotoxic periodic paralysis and Thyrotoxic periodic paralysis 2; Infertility associated with multi-tailed spermatozoa and excessive DNA; Glaucoma, primary open angle, juvenile-onset; Afibrinogenemia and congenital Afibrinogenemia; Polycystic kidney disease 2, adult type, and infantile type; porphyria cutanea tarda; Cerebello-oculo-renal syndrome (nephronophthisis, oculomotor apraxia and cerebellar abnormalities); Frontotemporal Dementia Chromosome 3-Linked and Frontotemporal dementia ubiquitin-positive; Metatrophic dysplasia; Immunodeficiency-centromeric instability-facial anomalies syndrome 2; Anemia, nonspherocytic hemolytic, due to G6PD deficiency; Bronchiectasis with or without elevated sweat chloride 3; Congenital myopathy with fiber type disproportion; Carney complex, type 1; Cryptorchidism, unilateral or bilateral; Ichthyosis bullosa of Siemens; Isolated lutropin deficiency; DFNA 2 Nonsyndromic Hearing Loss; Klein-Waardenberg syndrome; Gray platelet syndrome; Bile acid synthesis defect, congenital, 2; 46, XY sex reversal, type 1, 3, and 5; Acute intermittent porphyria; Cornelia de Fange syndromes 1 and 5; Hyperglycinuria; Cone-rod dystrophy 3; Dysfibrinogenemia; Karak syndrome; Congenital muscular dystrophy-dystroglycanopathy without mental retardation, type B5; Infantile nystagmus, X-linked; Dyskeratosis congenita, autosomal recessive, 1, 3, 4, and 5; Microcephaly with or without chorioretinopathy, lymphedema, or mental retardation; Hyperlysinemia; Bardet-Biedl syndromes 1, 11, 16, and 19; Autosomal recessive centronuclear myopathy; Frasier syndrome; Caudal regression syndrome; Fibrosis of extraocular muscles, congenital, 1, 2, 3a (with or without extraocular involvement), 3b; Prader-Willi-like syndrome; Malignant melanoma; Bloom syndrome; Darier disease, segmental; Multicentric osteolysis nephropathy; Hemochromato s type 1, 2B, and 3; Cerebellar ataxia infantile with progressive external ophthalmoplegia and Cerebellar ataxia, mental retardation, and dysequilibrium syndrome 2; Hypoplastic left heart syndrome; Epilepsy, Hearing Loss, And Mental Retardation Syndrome; Transferrin serum level quantitative trait locus 2; Ocular albinism, type Marian syndrome; Congenital muscular dystrophy-dystroglycanopathy with brain and eye anomalies, type A14 and E14; Hyperammonemia, type III; Cryptophthalmos syndrome; Alopecia universalis congenital; Adult hypophosphatasia; Mannose-binding protein deficiency; Bull eye macular dystrophy; Autosomal dominant torsion dystonia 4; Nephrotic syndrome, type 3, type 5, with or without ocular abnormalities, type 7, and type 9; Seizures, Early infantile epileptic encephalopathy 7; Persistent hyperinsulinemic hypoglycemia of infancy; Thrombocytopenia, X-linked; Neonatal hypotonia; Orstavik Lindemann Solberg syndrome; Pulmonary hypertension, primary, 1, with hereditary hemorrhagic telangiectasia; Pituitary dependent hypercortisolism; Epidermodysplasia verruciformis; Epidermolysis bullosa, junctional, localisata variant; Cytochrome c oxidase deficiency; Kindler syndrome; Myosclerosis, autosomal recessive; Truncus arteriosus; Duane syndrome type 2; ADULT syndrome; Zellweger syndrome spectrum; Leukoencephalopathy with ataxia, with Brainstem and Spinal Cord Involvement and Lactate Elevation, with vanishing White matter, and progressive, with ovarian failure; Antithrombin III deficiency; Holoprosencephaly 7; Roberts-SC phocomelia syndrome; Mitochondrial DNA-depletion syndrome 3 and 7, hepatocerebral types, and 13 (encephalomyopathic type); Porencephaly 2; Microcephaly, normal intelligence and immunodeficiency; Giant axonal neuropathy; Sturge-Weber syndrome, Capillary malformations, congenital, 1; Fabry disease and Fabry disease, cardiac variant; Glutamate formiminotransferase deficiency; Fanconi-Bickel syndrome; Acromicric dysplasia; Epilepsy, idiopathic generalized, susceptibility to, 12; Basal ganglia calcification, idiopathic, 4; Polyglucosan body myopathy 1 with or without immunodeficiency; Malignant tumor of prostate; Congenital ectodermal dysplasia of face; Congenital heart disease; Age-related macular degeneration 3, 6, 11, and 12; Congenital myotonia, autosomal dominant and recessive forms; Hypomagnesemia 1, intestinal; Sulfite oxidase deficiency, isolated; Pick disease; Plasminogen deficiency, type 1; Syndactyly type 3; Cone-rod dystrophy amelogenesis imperfecta; Pseudoprimary hyperaldosteronism; Terminal osseous dysplasia; Bartter syndrome antenatal type 2; Congenital muscular dystrophy-dystroglycanopathy with mental retardation, types B2, B3, B5, and B15; Familial infantile myasthenia; Lymphoproliferative syndrome 1, 1 (X-linked), and 2; Hypercholesterolaemia and Hypercholesterolemia, autosomal recessive; Neoplasm of ovary; Infantile GM1 gangliosidosis; Syndromic X-linked mental retardation 16; Deficiency of ribose-5-phosphate isomerase; Alzheimer disease, types, 1, 3, and 4; Andersen Tawil syndrome; Multiple synostoses syndrome 3; Chilbain lupus 1; Hemophagocytic lymphohistiocytosis, familial, 2; Axenfeld-Rieger syndrome type 3; Myopathy, congenital with cores; Osteoarthritis with mild chondrodysplasia; Peroxisome biogenesis disorders; Severe congenital neutropenia; Hereditary neuralgic amyotrophy; Palmoplantar keratoderma, nonepidermolytic, focal or diffuse; Dysplasminogenemia; Familial colorectal cancer; Spastic ataxia 5, autosomal recessive, Charlevoix-Saguenay type, 1, 10, or 11, autosomal recessive; Frontometaphyseal dysplasia land 3; Hereditary factors II, IX, VIII deficiency disease; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Ichthyosis prematurity syndrome; Stickler syndrome type 1; Focal segmental glomerulosclerosis 5; 5-Oxoprolinase deficiency; Microphthalmia syndromic 5, 7, and 9; Juvenile polyposis/hereditary hemorrhagic telangiectasia. syndrome; Deficiency of butyryl-CoA dehydrogenase; Maturity-onset diabetes of the young, type 2; Mental retardation, syndromic, Claes-Jensen type, X-linked; Deafness, cochlear, with myopia and intellectual impairment, without vestibular involvement, autosomal dominant, K-linked 2; Spondylocarpotarsal synostosis syndrome; Sting-associated vasculopathy. infantile-onset; Neutral lipid storage disease with myopathy; Immune dysfunction with T-cell inactivation due to calcium entry defect 2; Cardiofaciocutaneous syndrome; Corticosterone methyloxidase type 2 deficiency; Hereditary myopathy with early respiratory failure; Interstitial nephritis, karyomegalic; Trimethylaminuria; Hyperimmunoglobulin D with periodic fever; Malignant hyperthermia susceptibility type 1; Trichomegaly with mental retardation, dwarfism and pigmentary degeneration of retina; Breast adenocarcinoma; Complement factor B deficiency; Ulrich congenital muscular dystrophy; Left ventricular noncompaction cardiomyopathy; Fish-eye disease; Finnish congenital nephrotic syndrome; Limb-girdle muscular dystrophy, type IB, 2A, 2B, 2D, C1, C5, C9, C14; Idiopathic fibrosing alveolitis, chronic form; Primary familial hypertrophic cardiomyopathy; Angiotensin converting enzyme, benign serum increase; Cd8 deficiency, familial; Proteus syndrome; Glucose-6-phosphate transport defect; Borjeson-Forssman-Lehmann syndrome; Zellweger syndrome; Spinal muscular atrophy, type II; Prostate cancer, hereditary, 2; Thrombocytopenia, platelet dysfunction, hemolysis, and imbalanced globin synthesis; Congenital disorder of glycosylation types IB, ID, 1G, 1H, 1J, IK, IN, IP, 2C, 2J, 2K, that; Junctional epidermolysis bullosa gravis of Herlitz; Generalized epilepsy with febrile seizures plus 3, type 1, type 2; Schizophrenia 4; Coronary artery disease, autosomal dominant 2; Dyskeratosis congenita, autosomal dominant, 2 and 5; Subcortical laminar heterotopia, X-linked; Adenylate kinase deficiency; X-linked severe combined immunodeficiency; Coproporphyria; Amyloid Cardiomyopathy, Transthyretin-related; Hypocalcemia, autosomal dominant 1; Brugada syndrome; Congenital myasthenic syndrome, acetazolamide-responsive; Primary hypomagnesemia; Sclerosteosis; Frontotemporal dementia and/or amyotrophic lateral sclerosis 3 and 4; Mevalonic aciduria; Schwannomatosis 2; Hereditary motor and sensory neuropathy with optic atrophy; Porphyria cutanea tarda; Osteochondritis dissecans; Seizures, benign familial neonatal, 1, and/or myokymia; Long QT syndrome, LQT1 subtype; Mental retardation, anterior maxillary protrusion, and strabismus; Idiopathic hypercalcemia of infancy; Hypogonadotropic hypogonadism 11 with or without anosmia; Polycystic lipomembranous osteodysplasia with sclerosing leukoencephalopathy; Primary autosomal recessive microcephaly 10, 2, 3, and 5; Interrupted aortic arch; Congenital megakaryocytic thrombocytopenia; Hermansky-Pudlak syndrome 1, 3, 4, and 6; Long QT syndrome 1, 2, 2/9, 2/5, (digenic), 3, 5 and 5, acquired, susceptibility to; Andermann syndrome; Retinal cone dystrophy 3B; Erythropoietic protoporphyria; Sepiapterin reductase deficiency; Very long chain acyl-CoA dehydrogenase deficiency; Hyperferritinemia cataract syndrome; Silver spastic paraplegia syndrome; Charcot-Marie-Tooth disease; Atrial septal defect 2; Carnevale syndrome; Hereditary insensitivity to pain with anhidrosis; Catecholaminergic polymorphic ventricular tachycardia; Hypokalemic periodic paralysis 1 and 2; Sudden infant death syndrome; Hypochromic microcytic anemia with iron overload; GLUT1 deficiency syndrome 2; Leukodystrophy, Hypomyelinating, 11 and 6; Cone monochromatism; Osteopetrosis autosomal dominant type 1 and 2, recessive 4, recessive 1, recessive 6; Severe congenital neutropenia 3, autosomal recessive or dominant; Methionine adenosyltransferase deficiency, autosomal dominant; Paroxysmal familial ventricular fibrillation; Pyruvate kinase deficiency of red cells; Schneckenbecken dysplasia; Torsades de pointer; Distal myopathy Markesbery-Griggs type; Deficiency of UDPglucose-hexose-1-phosphate uridylyltransferase; Sudden cardiac death; Neu-Laxova syndrome 1; Atransferrinemia; Hyperparathyroidism 1 and 2; Cutaneous malignant melanoma 1; Symphalangism, proximal, lb; Progressive pseudorheumatoid dysplasia; Werdnig-Hoffmann disease; Achondrogenesis type 2; Holoprosencephaly 2, 3, 7, and 9; Schindler disease, type 1; Cerebroretinal microangiopathy with calcifications and cysts; Heterotaxy, visceral,)(linked; Tuberous sclerosis syndrome; Kartagener syndrome; Thyroid hormone resistance, generalized, autosomal dominant; Bestrophinopadiy, autosomal recessive; Nail disorder, nonsyndromic congenital, 8; Mohr-Tranehjaerg syndrome; Cone-rod dystrophy 12; Hearing impairment; Ovarioleukodystrophy; Renal tubular acidosis, proximal, with ocular abnormalities and mental retardation; Dihydropteridine reductase deficiency; Focal epilepsy with speech disorder with or without mental retardation; Ataxia-telangiectasia syndrome; Brown-Vialotto- Van laere, syndrome and Brown-Vialetto-Van Laere syndrome 2; Cardiomyopathy; Peripheral demyelinating neuropathy, central dysmyelination; Corneal dystrophy, Fuchs endothelial, 4; Cowden syndrome 3; Dystonia 2 (torsion, autosomal recessive), 3 (torsion,)(linked), 5 (Dopa-responsive type), 10, 12, 16, 25, 26 (Myoclonic); Epiphyseal dysplasia, multiple, with myopia and conductive deafness; Cardiac conduction defect, nonspecific; Branchiootic syndromes 2 and 3; Peroxisome biogenesis disorder 14B, 2A, 4A, 5B, 6A, 7A, and 7B; Familial renal glucosuria; Candidiasis, familial, 2, 5, 6, and 8; Autoimmune disease, multisystem, infantile-onset; Early infantile epileptic encephalopathy 2, 4, 7, 9, 10, 11, 13, and 14; Segawa syndrome, autosomal recessive; Deafness, autosomal dominant 3a, 4, 12, 13, 15, autosomal dominant nonsyndromic sensorineural 17, 20, and 65; Congenital dyserythropoietic anemia, type I and II; Enhanced s-cone syndrome; Adult neuronal ceroid lipofuscinosis; Atrial fibrillation, familial, 11, 12, 13, and 16; Norma disease; Osteosarcoma; Partial albinism; Biotinidase deficiency; Combined cellular and humoral immune defects with granulomas; Alpers encephalopathy; Holocarboxylase synthetase deficiency; Maturity-onset diabetes of the young, type 1, type 2, type 11, type 3, and type 9; Variegate porphyria; infantile cortical hyperostosis; Testosterone 17-beta-dehydrogenase deficiency; L-2-hydroxyglutaric aciduria; Tyrosinase-negative oculocutaneous albinism; Primary ciliary dyskinesia 24; Pontocerebellar hypoplasia type 4; Ciliary dyskinesia, primary, 7, 11, 15, 20 and 22; Idiopathic basal ganglia calcification 5; Brain atrophy; Craniosynostosis 1 and 4; Keratoconus 1; Rasopathy; Congenital adrenal hyperplasia and Congenital adrenal hypoplasia, X-linked; Mitochondrial DNA depletion syndrome 11, 12 (cardiomyopathic type), 2, 4B (MNGIE type), 8B (MNGIE type); Brachydactyly with hypertension; Cornea plana 2; Aarskog syndrome; Multiple epiphyseal dysplasia 5 or Dominant; Corneal endothelial dystrophy type 2; Aminoacylase 1 deficiency; Delayed speech and language development; Nicolaides-Baraitser syndrome; Enterokinase deficiency; Ectrodactyly, ectodermal dysplasia, and cleft lip/palate syndrome 3; Arthrogryposis multiplex congenita, distal, X-linked; Perrault syndrome 4; Jervell and Lange-Nielsen syndrome 2; Hereditary Nonpolyposis Colorectal Neoplasms; Robinow syndrome, autosomal recessive, autosomal recessive, with brachy-syn-polydactyly; Neurofibrosarcoma; Cytochrome-c oxidase deficiency; Vesicoureteral reflux 8; Dopamine beta. hydroxylase deficiency; Carbohydrate-deficient glycoprotein syndrome type I and II; Progressive intrahepatic cholestasis 3; Benign familial neonatal-infantile seizures; Pancreatitis, chronic, susceptibility to; Rhizomelic chondrodysplasia punctata type 2 and type 3; Disordered steroidogenesis due to cytochrome p450 oxidoreductase deficiency; Deafness with labyrinthine aplasia microtia and microdontia (AMM); Rothmund-Thomson syndrome; Cortical dysplasia, complex, with other brain malformations 5 and 6; Myasthenia, familial infantile, 1; Trichorhinophalangeal dysplasia type I; Worth disease; Splenic hypoplasia; Molybdenum cofactor deficiency, complementation group A; Sebastian syndrome; Progressive familial intrahepatic cholestasis 2 and 3; Weill-Marchesani syndrome 1 and 3; Microcephalic osteodysplastic primordial dwarfism type 2; Surfactant metabolism dysfunction, pulmonary, 2 and 3; Severe X-linked myotubular myopathy; Pancreatic cancer 3; Platelet-type bleeding disorder 15 and 8; Tyrosinase-positive oculocutaneous albinism; Borrone Di Rocco Crovato syndrome; ATR-X syndrome; Sucrase-isomaltase deficiency; Complement component 4. partial deficiency of, due to dysfunctional c1 inhibitor; Congenital central hypoventilation; Infantile hypophosphatasia; Plasminogen activator inhibitor type 1 deficiency; Malignant lymphoma, non-Hodgkin; Hyperornithinemia-hyperammonemia-homocitrullinuria syndrome; Schwartz Jampel syndrome type 1; Fetal hemoglobin quantitative trait locus 1; Myopathy, distal, with anterior tibial onset; Noonan syndrome 1 and 4, LEOPARD syndrome 1; Glaucoma 1, open angle, e, F, and C; Kenny-Caffey syndrome type 2; pTEN hamartoma tumor syndrome; Duchenne muscular dystrophy; Insulin-resistant diabetes mellitus and acanthosis nigricans; Microphthalmia, isolated 3, 5, 6, 8, and with coloboma 6; Raine syndrome; Premature ovarian failure 4, 5, 7, and 9; Allan-Hemdon-Dudley syndrome; Citrullinemia type I; Alzheimer disease, familial, 3, with spastic paraparesis and apraxia; Familial hemiplegic migraine types 1 and 2; Ventriculomegaly with cystic kidney disease; Pseudoxanthoma elasticum; Homocysteinemia due to MTHFR deficiency, CBS deficiency, and Homocystinuria, pyridoxine responsive; Dilated cardiomyopathy 1A, 1AA, 1C, 1G, IBB, 1DD, IFF, 1HH, II, IKK, IN, IS, 1Y, and 3B; Muscle AMP guanine oxidase deficiency; Familial cancer of breast; Hereditary sideroblastic anemia; Myoglobinuria, acute recurrent, autosomal recessive; Neuroferritinopathy; Cardiac arrhythmia; Glucose transporter type 1 deficiency syndrome; Holoprosencephaly sequence; Angiopathy, hereditary, with nephropathy, aneurysms, and muscle cramps; Isovaleryl-CoA dehydrogenase deficiency; Kallmann syndrome 1, 2, and 6; Permanent neonatal diabetes mellitus; Acrocallosal syndrome, Schinzel type; Gordon syndrome; MYH9 related disorders; Donnai Barrow syndrome; Severe congenital neutropenia and 6, autosomal recessive; Charcot-Marie-Tooth disease, types ID and IVF; Coffin-Lowry syndrome; mitochondrial 3-hydroxy-3-methylglutaryl-CoA synthase deficiency; Hypomagnesemia, seizures, and mental retardation; Ischiopatellar dysplasia; Multiple congenital anomalies—hypotonia—seizures syndrome 3; Spastic paraplegia 50, autosomal recessive; Short stature with nonspecific skeletal abnormalities; Severe myoclonic epilepsy in infancy; Propionic academia; Adolescent nephronophthisis; Macrocephaly, macrosomia, facial dysmorphism syndrome; Stargardt disease 4; Ehlers-Danlos syndrome type 7 (autosomal recessive), classic type, type 2 (progeroid), hydroxylysine-deficient, type 4, type 4 variant, and due to tenascin-X deficiency; Myopia 6; Coxa pima; Familial cold autoinflammatory syndrome 2; Malformation of the heart and great vessels; von Willebrand disease type 2M and type 3; Deficiency of galactokinase; Brugada syndrome 1; X-linked ichthyosis with steryl-sulfatase deficiency; Congenital ocular coloboma; Histiocytosis-lymphadenopathy plus syndrome; Aniridia, cerebellar ataxia, and mental retardation; Left ventricular noncompaction 3; Amyotrophic lateral sclerosis types 1, 6, 15 (with or without frontotemporal dementia), 22 (with or without frontotemporal dementia), and 10; Osteogenesis imperfecta type 12, type 5, type 7, type 8, type 1, type III, with normal sclerae, dominant form, recessive perinatal lethal; Hematologic neoplasm; Favism, susceptibility to; Pulmonary Fibrosis And/Or Bone Marrow Failure, Telomere-Related, 1 and 3; Dominant hereditary optic atrophy; Dominant dystrophic epidermolysis bullosa. with absence of skin; Muscular dystrophy, congenital, megaconial type; Multiple gastrointestinal atresias; McCune-Albright syndrome; Mail patella syndrome; McLeod neuroacanthocytosis syndrome; Common variable immunodeficiency 9; Partial hypoxanthine-guanine phosphoribosyltransferase deficiency; Pseudohypoaldosteronism type 1 autosomal dominant and recessive and type 2; Urocanate hydratase deficiency, Heterotopia; Meckel syndrome type 7; Ch\xc3\xa9diak-Higashi syndrome, Chediak-Higashi syndrome, adult type; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Insulin resistance; Deficiency of steroid 11-beta-monooxygenase; Popliteal pterygium syndrome; Pulmonary arterial hypertension related to hereditary hemorrhagic telangiectasia; Deafness, autosomal recessive 1A, 2, 3, 6, 8, 9, 12, 15, 16, 18b, 22, 28, 31, 44, 49, 63, 77, 86, and 89; Primary hyperoxaluria, type I, type, and type III; Paramyotonia congenita of von Eulenburg; Desbuquois syndrome; Carnitine palmitoyltransferase I, II, II (late onset), and II (infantile) deficiency; Secondary hypothyroidism; Mandibulofacial dysostosis, Treacher Collins type, autosomal recessive; Cowden syndrome 1; Li-Fraumeni syndrome 1; Asparagine synthetase deficiency; Malattia leventinese; Optic atrophy 9; Infantile convulsions and paroxysmal choreoathetosis, familial; Ataxia with vitamin E deficiency; Islet cell hyperplasia; Miyoshi muscular dystrophy 1; Thrombophilia, hereditary, due to protein C deficiency, autosomal dominant and recessive; Fechtner syndrome; Properdin deficiency, X-linked; Mental retardation, stereotypic movements, epilepsy, and/or cerebral malformations; Creatine deficiency, X-linked; Pilomatrixoma; Cyanosis, transient neonatal and atypical nephropathic; Adult onset ataxia with oculomotor apraxia; Hemangioma, capillary infantile; PC-K6a; Generalized dominant dystrophic epidermolysis bullosa; Pelizaeus-Merzbacher disease; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Benign familial neonatal seizures 1 and 2; Primary pulmonary hypertension; Lymphedema, primary, with myelodysplasia; Congenital long QT syndrome; Familial exudative vitreoretinopathy, X-linked; Autosomal dominant hypohidrotic ectodermal dysplasia; Primordial dwarfism; Familial pulmonary capillary hemangiomatosis; Carnitine acylcarnitine translocase deficiency; Visceral myopathy; Familial Mediterranean fever and mediterranean fever, autosomal dominant; Combined partial and complete 17-alpha-hydroxylase/17, 20-lyase deficiency; Oto-palato-digital syndrome, type I; Nephrolithiasis/osteoporosis, hypophosphatemic, 2; Familial type 1 and 3 hyperlipoproteinemia; Phenotypes; CHARGE association; Fuhrmann syndrome; Hypotrichosis-lymphedema-telangiectasia syndrome; Chondrodysplasia Blomstrand type; Acroerythrokeratoderma; Slowed nerve conduction velocity, autosomal dominant; Hereditary cancer-predisposing syndrome; Craniodiaphyseal dysplasia, autosomal dominant; Spinocerebellar ataxia autosomal recessive 1 and 16; Proprotein convertase 1/3 deficiency; D-2-hydroxyglutaric aciduria 2; Hyperekplexia 2 and Hyperekplexia hereditary; Central core disease; Opitz G/BBB syndrome; Cystic fibrosis; Thiel-Behnke conical dystrophy; Deficiency of bisphosphoglycerate mutase; Mitochondrial short-chain Enoyl-CoA Hydratase 1 deficiency; Ectodermal dysplasia skin fragility syndrome; Wolfram-like syndrome, autosomal dominant; Microcytic anemia; Pyruvate carboxylase deficiency; Leukocyte adhesion deficiency type I and III; Multiple endocrine neoplasia, types land 4; Transient billions dermolysis of the newborn; Primrose syndrome; Non-small cell lung cancer; Congenital muscular dystrophy; Lipase deficiency combined; COLE-CARPENTER SYNDROME 2; Atrioventricular septal defect and common atrioventricular junction; Deficiency of xanthine oxidase; Waardenburg syndrome type 1, 4C, and 2E (with neurologic involvement); Stickier syndrome, types 1 (nonsyndromic ocular) and 4; Corneal fragility keratoglobus, blue sclerae and joint hypermobility; Microspherophakia; Chudley-McCullough syndrome; Epidermolysa bullosa simplex and limb girdle muscular dystrophy, simplex with mottled pigmentation, simplex with pyloric atresia, simplex, autosomal recessive, and with pyloric atresia; Rett disorder; Abnormality of neuronal migration; Growth hormone deficiency with pituitary anomalies: Leigh disease; Keratosis pahnoplantatis striata 1; Weissenbacher-Zweymuller syndrome; Medium-chain acyl-coenzyme A dehydrogenase deficiency; UDPglucose-4-epimerase deficiency; susceptibility to Autism, X-linked 3; Rhegmatogenous retinal detachment, autosomal dominant; Familial febrile seizures 8; Ulna and fibula absence of with severe limb deficiency; Left ventricular noncompaction 6; Centromeric instability of chromosomes 1, 9 and 16 and immunodeficiency; Hereditary diffuse leukoencephalopathy with spheroids; Cushing syndrome; Dopamine receptor d2, reduced brain density of; C-like syndrome; Renal dysplasia, retinal pigmentary dystrophy, cerebellar ataxia and skeletal dysplasia; Ovarian dysgenesis 1; Pierson syndrome; Polyneuropathy, hearing loss, ataxia, retinitis pigmentosa, and cataract; Progressive intrahepatic cholestasis; autosomal dominant, autosomal recessive, and X-linked recessive Alport syndromes; Angelman syndrome; Amish infantile epilepsy syndrome; Autoimmune lymphoproliferative syndrome, type 1a; Hydrocephalus; Marfanoid habitus; Bare lymphocyte syndrome type 2, complementation group E; Recessive dystrophic epidermolysis bullosa; Factor H, VII X, v and factor viii, combined deficiency of 2, xiii, a subunit, deficiency; Zonular pulverulent cataract 3; Warts, hypogammaglobulinemia, infections, and myelokathexis; Benign hereditary chorea; Deficiency of hyaluronoglucosaminidase; Microcephaly, hiatal hernia and nephrotic syndrome; Growth and mental retardation, mandibulofacial dysostosis, microcephaly, and cleft palate; Lymphedema, hereditary, id; Delayed puberty; Apparent mineralocorticoid excess; Generalized arterial calcification of infancy 2; METHYLMALONIC ACIDURIA, mut(0) TYPE; Congenital heart disease, multiple types, 2; Familial hypoplastic, glomerulocystic kidney; Cerebrooculofacioskeletal syndrome 2; Stargardt disease 1; Mental retardation, autosomal recessive 15, 44, 46, and 5; Prolidase deficiency; Methylmalonic aciduria chiB type; Oguchi disease; Endocrine-cerebroosteodysplasia; Lissencephaly 1, 2 (X-linked), 3, 6 (with microcephaly), X-linked; Somatotroph adenoma; Gamstorp-Wolfart, syndrome; Lipid proteinosis; Inclusion body myopathy 2 and 3; Enlarged vestibular aqueduct syndrome; Osteoporosis with pseudoglioma; Acquired long QT syndrome; Phenylketorturia; CHOPS syndrome; Global developmental delay; Bietti crystalline corneoretinal dystrophy; Noonan syndrome-like disorder with or without juvenile myelomonocytic leukemia; Congenital erythropoietic porphyria; Atrophia bulborum hereditaria; Paragangliomas 3; Van der Woude syndrome; Aromatase deficiency; Birk Barrel mental retardation dysmorphism syndrome; Amyotrophic lateral sclerosis type 5; Methemoglobinemia types 11 and 2; Congenital stationary night blindness, type 1A, IB, 1C, IE, IF, and 2A; Seizures; Thyroid cancer, follicular; Lethal congenital contracture syndrome 6; Distal hereditary motor neuronopathy type 2B; Sex cord-stromal tumor; Epileptic encephalopathy, childhood-onset, early infantile, 1, 19, 23, 25, 30, and 32; Myofibrillar myopathy 1 and ZASP-related; Cerebellar ataxia infantile with progressive external ophthalmoplegia; Purine-nucleoside phosphorylase deficiency; Forebrain defects; Epileptic encephalopathy Lennox-Gastaut type Obesity; 4, Left ventricular noncompaction 10; Vetheij syndrome; Mowat-Wilson syndrome; Odontotrichomelic syndrome; Patterned dystrophy of retinal pigment epithelium; Lig4 syndrome; Barakat syndrome; IRAK4 deficiency; Somatotroph adenoma; Branched-chain ketoacid dehydrogenase kinase deficiency; Cystinuria; Familial aplasia of the vermis; Succinyl-CoA acetoacetate transferase deficiency; Scapuloperoneal spinal muscular atrophy; Pigmentary retinal dystrophy; Glanzmann thrombasthenia; Primary open angle glaucoma juvenile onset 1; Aicardi Goutieres syndromes 1, 4, and 5; Renal dysplasia; Intrauterine growth retardation, metaphyseal dysplasia, adrenal hypoplusia congenita, and genital anomalies; Beaded hair; Short stature, onychodysplasia, facial dysmorphism, and hypotrichosis; Metachromatic leukodystrophy; Cholestanol storage disease; Three M syndrome 2; Leber congenital amaurosis 11, 12, 13, 16, 4, 7, and 9; Mandibuloacral dysplasia with type A or B lipodystrophy, atypical; Meier-Gorlin syndromes land 4; Hypotrichosis 8 and 12; Short QT syndrome 3; Ectodermal dysplasia 1 ib; Anonychia; Pseudohypoparathyroidism type 1A, Pseudopseudohypoparathyroidism; Leher optic atrophy; Bainbridge-Ropers syndrome; Weaver syndrome; Short stature, auditory canal atresia, mandibular hypoplasia, skeletal abnormalities; Deficiency of alpha-mannosicktse; Macular dystrophy, vitelliform, adult-onset; Glutaric aciduria, type 1; Gangliosidosis GM1 type1 (with cardiac involvement) 3; Mandibuloacral dysostosis; Hereditary lymphedema type I; Atrial standstill 2; Kabuki make-up syndrome; Bethlem myopathy and Bethlem myopathy 2; Myeloperoxidase deficiency; Fleck corneal dystrophy; Hereditary acrodermatitis enteropathica; Hypobetalipoproteinemia, familial, associated with apob32; Cockayne syndrome type A, Hyperparathyroidism, neonatal severe; Ataxia-telangiectasia-like disorder; Pendred syndrome; I blood group system; Familial benign pemphigus; Visceral heterotaxy 5, autosomal; Nephrogenic diabetes insipidus, Nephrogenic diabetes insipidus, X-linked; Minicore myopathy with external ophthalmoplegia; Perry syndrome; hypohidrotic/hair/tooth type, autosomal recessive; Hereditary pancreatitis; Mental retardation and microcephaly with pontine and cerebellar hypoplasia; Glycogen storage disease 0 (muscle), II (adult form), IXa2, IXc, type 1A; Osteopathia striata with cranial sclerosis; Gluthathione synthetase deficiency; Brugada syndrome and Brugada syndrome 4; Endometrial carcinoma; Hypohidrotic ectodermal dysplasia with immune deficiency; Cholestasis, intrahepatic, of pregnancy 3; Bernard-Soulier syndrome, types A1 and A2 (autosomal dominant); Salla disease; Ornithine aminotransferase deficiency; PTEN hamartoma tumor syndrome; Distichiasis-lymphedema syndrome; Corticosteroid-binding globulin deficiency; Adult neuronal ceroid lipofuscinosis; Dejerine-Sottas disease; Tetraamelia, autosomal recessive; Senior-Loken syndrome 4 and 5, Glutaric acidemia IIA and IIB; Aortic aneurysm, familial thoracic 4, 6, and 9; Hyperphosphatasia with mental retardation syndrome 2, 3, and 4; Dyskeratosis congenita X-linked; Arthrogryposis, renal dysfunction, and cholestasis 2; Bannayan-Riley-Ruvalcaba syndrome; 3-Methylglutaconic aciduria; Isolated 17,20-lyase deficiency; Gorlin syndrome; Hand foot uterus syndrome; Tay-Sachs disease, B1 variant, Gm2-gangliosidosis (adult), Gm2-gangliosidosis (adult-onset); Dowling-degas disease 4; Parkinson disease 14, 15, 19 (juvenile-onset), 2, 20 (early-onset), 6, (autosomal recessive early-onset, and 9; Ataxia, sensory, autosomal dominant; Congenital microvillous atrophy; Myoclonic-Atonic Epilepsy; Tangier disease; 2-methyl-3-hydroxybutyric aciduria; renal hyperuricemia; Schizencephaly; Mitochondrial DNA depletion syndrome 4B, MNGIE type; Feingold syndrome 1; Renal carnitine transport defect; Familial hypercholesterolemia; Townes-Brocks-branchiootorenal-like syndrome; Griscelli syndrome type 3; Meckel-Gruber syndrome; Bullous ichthyosiform erythroderma; Neutrophil immunodeficiency syndrome; Myasthenic Syndrome, Congenital, 17, 2A (slow-channel), 4B (fast-channel), and without tubular aggregates; Microvascular complications of diabetes 7; McKusick Kaufman syndrome; Chronic granulomatous disease, autosomal recessive cytochrome b-positive, types 1 and 2; Arginino succinate lyase deficiency; Mitochondrial phosphate carrier and pyruvate carrier deficiency; Lattice corneal dystrophy type III; Ectodermal dysplasia-syndactyly syndrome 1; Hypomyelinating leukodystrophy 7; Mental retardation, autosomal dominant 12, 13, 15, 24, 3, 30, 4, 5, 6, and 9; Generalized epilepsy with febrile seizures plus, types 1 and 2; Psoriasis susceptibility 2; Frank Ter Haar syndrome; Thoracic aortic aneurysms and aortic dissections; Crouzon syndrome; Granulosa cell tumor of the ovary; Epidermolytic palmoplantar keratoderma; Leri Weill dyschondrosteosis; 3 beta-Hydroxysteroid dehydrogenase deficiency; Familial restrictive cardiomyopathy 1; Autosomal dominant progressive external ophthalmoplegia with mitochondrial DNA deletions 1 and 3; Antley-Bixler syndrome with genital anomalies and disordered steroidogenesis; Hajdu-Cheney syndrome; Pigmented nodular adrenocortical disease, primary, 1; Episodic pain syndrome, familial, 3; Dejerine-Sottas syndrome, autosomal dominant; FG syndrome and FG syndrome 4; Dendritic cell, monocyte. B lymphocyte, and natural killer lymphocyte deficiency; Hypothyroidism, congenital, nongoitrous, 1; Miller syndrome; Nemaline myopathy 3 and 9; Oligodontia-colorectal cancer syndrome; Cold-induced sweating syndrome 1; Van Buchem disease type 2; Glaucoma 3, primary congenital, d; Citrullinemia, type I and II; Nonaka myopathy; Congenital muscular dystrophy due to partial LAMA2 deficiency; Myoneural gastrointestinal encephalopathy syndrome; Leigh syndrome due to mitochondrial complex I deficiency; Medulloblastoma; Pyruvate dehydrogenase El-alpha deficiency; Carcinoma of colon; Nance-Horan syndrome; Sandhoff disease, adult and infantil types; Arthrogryposis renal dysfunction cholestasis syndrome; Autosomal recessive hypophosphatemic bone disease; Doyne honeycomb retinal dystrophy; Spinocerebellar ataxia 14, 21, 35, 40, and 6; Lewy body dementia; RRM2B-related mitochondrial disease; Brody myopathy; Megalencephaly-polymicrogyria-polydactyly-hydrocephalus syndrome 2; Usher syndrome, types 1, IB, ID, 1G, 2A, 2C, and 2D; hypocalcification type and hypomaturation type, IIA1 Amelogenesis imperfecta; Pituitary, hormone deficiency, combined 1, 2, 3, and 4; Cushing symphalangism; Renal tubular acidosis, distal, autosomal recessive, with late-onset sensorineural hearing loss, or with hemolytic anemia; Infantile nephronophthisis; Juvenile polyposis syndrome; Sensory ataxic neuropathy, dysarthria, and ophthalmoparesis; Deficiency of 3-hydroxyacyl-CoA dehydrogenase; Parathyroid carcinoma; X-linked agammaglobulinemia; Megaloblastic anemia, thiamine-responsive with diabetes mellitus and sensorineural deafness; Multiple sulfatase deficiency; Neurodegeneration with brain iron accumulation 4 and 6; Cholesterol monooxygenase (side-chain cleaving) deficiency; hemolytic anemia. due to Adenylosuccinate lyase deficiency; Myoclonus with epilepsy with ragged red fibers; Pitt-Hopkins syndrome; Multiple pterygium syndrome Escobar type; Homocystinuria-Megaloblastic anemia due to defect in cobalamin metabolism, cblE complementation. type; Cholecystitis; Spherocytosis types 4 and 5; Multiple congenital anomalies; Xeroderma pigmentosum, complementation group b. group D, group E, and group C; Leiner disease; Groenouw conical dystrophy type 1; Coenzyme Q10 deficiency, primary 1, 4, and 7; Distal spinal muscular atrophy, congenital nonprogressive; Warburg micro syndrome 2 and 4; Bile acid synthesis defect, congenital, 3; Acth-independent macronodular adrenal hyperplasia 2; Acrocapitofemoral dysplasia; Paget disease of bone, familial; Severe neonatal-onset encephalopathy with microcephaly; Zimmermann-Laband syndrome and Zimmermann-Laband syndrome 2; Reifenstein syndrome; Familial hypokalemia-hypomagnesemia; Photosensitive trichothiodystrophy; Adult junctional epidermolysis bullosa; Lung cancer; Freeman-Sheldon syndrome; Hyperinsulinism-hyperammonemia syndrome; Posterior polar cataract type 2; Sclerocornea, autosomal recessive; juvenile GM>1<gangliosidosis; Cohen syndrome; Hereditary Paraganglioma-Pheochromocytoma Syndromes; Neonatal insulin-dependent diabetes mellitus; Hypochondrogenesis: Floating-Harbor syndrome; Cutis laxa with osteodystrophy and with severe pulmonary, gastrointestinal, and urinary abnormalities; Congenital contractures of the limbs and face, hypotonia, and developmental delay; Dyskeratosis congenita autosomal dominant and autosomal dominant, 3; Histiocytic medullary reticulosis; Costello syndrome; Immunodeficiency 15, 16, 19, 30, 31C, 38, 40, 8, due to defect in cd3-zeta, with hyper IgM type 1 and 2, and X-Linked with magnesium defect, Epstein-Ban vims infection, and neoplasia; Atrial septal defects 2, 4, and 7 (with or without atrioventricular conduction defects); GTP cyclohydrolase I deficiency; Talipes equinovarus; Phosphoglycerate kinase 1 deficiency; Tuberous sclerosis 1 and 2; Autosomal recessive congenital ichthyosis 1, 2, 3, 4A, and 4B; and Familial hypertrophic cardiomyopathy 1, 2, 3, 4, 7, 10, 23 and 24.

### Indications by Tissue

Additional suitable diseases and disorders that can be treated by the systems and methods provided herein include, without limitation, diseases of the central nervous system (CNS) (see exemplary diseases and affected genes in Table 13), diseases of the eye (see exemplary diseases and affected genes in Table 14), diseases of the heart (see exemplary diseases and affected genes in Table 15), diseases of the hematopoietic stem cells (HSC) (see exemplary diseases and affected genes in Table 16), diseases of the kidney (see exemplary diseases and affected genes in Table 17), diseases of the liver (see exemplary diseases and affected genes in Table 18), diseases of the lung (see exemplary diseases and affected genes in Table 19), diseases of the skeletal muscle (see exemplary diseases and affected genes in Table 20), and diseases of the skin (see exemplary diseases and affected genes in Table 21). Table 22 provides exemplary protective mutations that reduce risks of the indicated diseases. In some embodiments, a Gene Writer system described herein is used to treat an indication of any of Tables 13-21. In some embodiments, a Gene Writer system described herein is used to supply a functional (e.g., wild type) gene of any of Tables 13-21.

TABLE 13
CNS diseases and genes affected.

| | Gene |
|---|---|
| Disease | Affected |
| Alpha-mannosidosis | MAN2B1 |
| Ataxia-telangiectasia | ATM |
| CADASIL | NOTCH3 |
| Canavan disease | ASPA |
| Carbamoyl-phosphate synthetase 1 deficiency | CPS1 |
| CLN1 disease | PPT1 |
| CLN2 Disease | TPP1 |
| CLN3 Disease (Juvenile neuronal ceroid | CLN3 |
| lipofuscinosis, Batten Disease) | |
| Coffin-Lowry syndrome | RPS6KA3 |
| Congenital myasthenic syndrome 5 | COLQ |
| Cornelia de Lange syndrome (NIPBL) | NIPBL |
| Cornelia de Lange syndrome (SMC1A) | SMC1A |
| Dravet syndrome (SCN1A) | SCN1A |
| Glycine encephalopathy (GLDC) | GLDC |
| GM1 gangliosidosis | GLB1 |
| Huntington's Disease | HTT |
| Hydrocephalus with stenosis of the aqueduct of | L1CAM |
| Sylvius | |
| Leigh Syndrome | SURF1 |
| Metachromatic leukodystrophy (ARSA) | ARSA |
| MPS type 2 | IDS |
| MPS type 3 | Type 3a: SGSH |
| | Type 3b: NAGLU |
| Mucolipidosis IV | MCOLN1 |
| Neurofibromatosis Type 1 | NF1 |
| Neurofibromatosis type 2 | NF2 |
| Pantothenate kinase-associated neurodegeneration | PANK2 |
| Pyridoxine-dependent epilepsy | ALDH7A1 |
| Rett syndrome (MECP2) | MECP2 |
| Sandhoff disease | HEXB |
| Semantic dementia (Frontotemporal dementia) | MAPT |
| Spinocerebellar ataxia with axonal neuropathy (Ataxia | SETX |
| with Oculomotor Apraxia) | |
| Tay-Sachs disease | HEXA |
| X-linked Adrenoleukodystrophy | ABCD1 |

TABLE 14
Eye diseases and genes affected.

| Disease | Gene Affected |
|---|---|
| Achromatopsia | CNGB3 |
| Amaurosis Congenita (LCA1) | GUCY2D |
| Amaurosis Congenita (LCA10) | CEP290 |
| Amaurosis Congenita (LCA2) | RPE65 |
| Amaurosis Congenita (LCA8) | CRB1 |
| Choroideremia | CHM |
| Cone Rod Dystrophy (ABCA4) | ABCA4 |
| Cone Rod Dystrophy (CRX) | CRX |
| Cone Rod Dystrophy (GUCY2D) | GUCY2D |
| Cystinosis, Ocular Nonnephropathic | CTNS |
| Lattice corneal dystrophy type I | TGFBI |
| Macular Corneal Dystrophy (MCD) | CHST6 |
| Optic Atrophy | OPA1 |
| Retinitis Pigmentosa (AR) | USH2A |
| Retinitis Rigmentosa (AD) | RHO |
| Stargardt Disease | ABCA4 |
| Vitelliform Macular Dystrophy | BEST1; PRPH2 |

TABLE 15
Heart diseases and genes affected.

| | Gene |
|---|---|
| Disease | Affected |
| Arrhythmogenic right ventricular cardiomyopathy (ARVC) | PKP2 |
| Barth syndrome | TAZ |
| Becker muscular dystrophy | DMD |
| Brugada syndrome | SCN5A |
| Catecholaminergic polymorphic ventricular tachycardia | RYR2 |
| (RYR2) | |
| Dilated cardiomyopathy (LMNA) | LMNA |
| Dilated cardiomyopathy (TTN) | TTN |
| Duchenne muscular dystrophy | DMD |
| Emery-Dreifuss Muscular Dystrophy Type I | EMD |
| Familial hypertrophic cardiomyopathy | MYH7 |
| Familial hypertrophic cardiomyopathy | MYBPC3 |
| Jervell Lange-Nielsen syndrome | KCNQ1 |
| LCHAD deficiency | HADHA |
| Limb-girdle muscular dystrophy type 1B (Emery-Dreifuss | LMNA |
| EDMD2) | |
| Limb-girdle muscular dystrophy, type 2D | SGCA |
| Long QT syndrome 1 (Romano Ward) | KCNQ1 |

TABLE 16
HSC diseases and genes affected.

| | Gene |
|---|---|
| Disease | Affected |
| ADA-SCID | ADA |
| Adrenoleukodystrophy (CALD) | ABCD1 |
| Alpha-mannosidosis | MAN2B1 |
| Chronic granulomatous disease | CYBB; CYBA; |
| | NCF1; NCF2; |
| | NCF4 |
| Common variable immunodeficiency | TNFRSF13B |
| Fanconi anemia | FANCA; FANCC; |
| | FANCG |
| Gaucher disease | GBA |
| Globoid cell leukodystrophy (Krabbe disease) | GALC |
| Hemophagocytic lymphohistiocytosis | PRF1; STX11; |
| | STXBP2; UNC13D |
| IL-7R SCID | IL7R |
| JAK-3 SCID | JAK3 |
| Malignant infantile osteopetrosis- autosomal | TCIRG1; |
| recessive osteopetrosis | Many genes |
| | implicated |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Paroxysmal Nocturnal Hemoglobinuria | PIGA |
| Pompe disease | GAA |
| Pyruvate kinase deficiency (PKD) | PKLR |
| RAG 1/2 Deficiency (SCID with granulomas) | RAG1/RAG2 |
| Severe Congenital Neutropenia | ELANE; HAX1 |
| Sickle cell disease (SCD) | HBB |
| Tay Sachs | HEXA |
| Thalassemia | HBB |
| Wiskott-Aldrich Syndrome | WAS |
| X-linked agammaglobulinemia | BTK |
| X-linked SCID | IL2RG |

TABLE 17
Kidney diseases and genes affected.

| | Gene |
|---|---|
| Disease | Affected |
| Alport syndrome | COL4A5 |
| Autosomal dominant polycystic kidney disease (PKD1) | PKD1 |
| Autosomal dominant polycystic kidney disease (PKD2) | PDK2 |
| Autosomal dominant tubulointerstitial kidney disease | MUC1 |
| (MUC1) | |
| Autosomal dominant tubulointerstitial kidney disease | UMOD |
| (UMOD) | |
| Autosomal recessive polycystic kidney disease | PKHD1 |
| Congenital nephrotic syndrome | NPHS2 |
| Cystinosis | CTNS |

TABLE 18
Liver diseases and genes affected.

| | Gene |
|---|---|
| Disease | Affected |
| Acute intermittent porphyria | HMBS |
| Alagille syndrome | JAG1 |
| Alpha-1-antitrypsin deficiency | SERPINA1 |
| Carbamoyl phosphate synthetase I deficiency | CPS1 |
| Citrullinemia I | ASS1 |
| Crigler-Najjar | UGT1A1 |
| Fabry | LPL |
| Familial chylomicronemia syndrome | GLA |
| Gaucher | GBE1 |
| GSD IV | GBA |
| Heme A | F8 |
| Heme B | F9 |
| Hereditary amyloidosis (hTTR) | TTR |
| Hereditary angioedema | SERPING1 |
| | (KLKB1 |
| | for CRISPR) |
| HoFH | LDLRAP1 |
| Hypercholesterolemia | PCSK9 |
| Methylmalonic acidemia | MMUT |
| MPS II | IDS |
| MPS III | Type IIIa: SGSH |
| | Type IIIb: NAGLU |
| | Type IIIc: HGSNAT |
| | Type IIId: GNS |
| MPS IV | Type IVA: GALNS |
| | Type IVB: GLB1 |
| MPS VI | ARSB |
| MSUD | Type Ia: BCKDHA |
| | Type Ib: BCKDHB |
| | Type II: DBT |
| OTC Deficiency | OTC |
| Polycystic Liver Disease | PRKCSH |
| Pompe | GAA |
| Primary Hyperoxaluria 1 | AGXT (HAO1 or |
| | LDHA for CRISPR) |
| Progressive familial intrahepatic cholestasis type 1 | ATP8B1 |
| Progressive familial intrahepatic cholestasis type 2 | ABCB11 |
| Progressive familial intrahepatic cholestasis type 3 | ABCB4 |
| Propionic acidemia | PCCB; PCCA |
| Wilson's Disease | ATP7B |
| Glycogen storage disease, Type 1a | G6PC |
| Glycogen storage disease, Type IIIb | AGL |
| Isovaleric acidemia | IVD |
| Wolman disease | LIPA |

TABLE 19
Lung diseases and genes affected.

| | Gene |
|---|---|
| Disease | Affected |
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency (pulmonary | SFTPB |
| surfactant metabolism dysfunction 1) | |

TABLE 20
Skeletal muscle diseases and genes affected.

| | Gene |
|---|---|
| Disease | Affected |
| Becker muscular dystrophy | DMD |
| Becker myotonia | CLCN1 |
| Bethlem myopathy | COL6A2 |
| Centronuclear myopathy, X-linked (myotubular) | MTM1 |
| Congenital myasthenic syndrome | CHRNE |
| Duchenne muscular dystrophy | DMD |
| Emery-Dreifuss muscular dystrophy, AD | LMNA |
| Facioscapulohumeral Muscular Dystrophy | DUX4 - D4Z4 |
| | chromosomal |
| | region |
| Hyperkalemic periodic paralysis | SCN4A |
| Hypokalemic periodic paralysis | CACNA1S |
| Limb-girdle muscular dystrophy 2A | CAPN3 |
| Limb-girdle muscular dystrophy 2B | DYSF |
| Limb-girdle muscular dystrophy, type 2D | SGCA |
| Miyoshi muscular dystrophy 1 | DYSF |
| Paramyotonia congenita | SCN4A |
| Thomsen myotonia | CLCN1 |
| VCP myopathy (IBMPFD) 1 | VCP |

TABLE 21
Skin diseases and genes affected.

| | Gene |
|---|---|
| Disease | Affected |
| Epidermolysis Bullosa Dystrophica Dominant | COL7A1 |
| Epidermolysis Bullosa Dystrophica Recessive | COL7A1 |
| (Hallopeau-Siemens) | |
| Epidermolysis Bullosa Junctional | LAMB3 |
| Epidermolysis Bullosa Simplex | KRT5; KRT14 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

TABLE 22
Exemplary protective mutations that reduce disease risk.

| Disease | Gene | Exemplary Protective Mutation |
|---|---|---|
| Alzheimer's | APP | A673T |
| Parkinson's | SGK1 | |
| Diabetes (Type II) | SLC30A8 | p.Arg138X; p.Lys34SerfsX50 |
| Cardiovascular | PCSK9 | R46L |
| Disease | | |
| Cardiovascular | ASGR1 | NM_001671.4, c.284-36_283 + 33delCTGGGGCTGGGG |
| Disease | | (“CTGGGGCTGGGG” disclosed as SEQ ID NO: 1580); |
| | | NP_001662.1, p.W158X |
| Cardiovascular | NPC1L1 | p.Arg406X |
| Disease | | |
| Cardiovascular | APOC3 | R19X; IVS2 + 1G→A; A43T |
| Disease | | |
| Cardiovascular | LPA | |
| Disease | | |
| Cardiovascular | ANGPTL4 | E40K |
| Disease | | |
| Cardiovascular | ANGPTL3 | p.Ser17Ter; p.Asn121fs; p.Asn147fs; c.495 + 6T→C |
| Disease | | |
| HIV infection | CCR5 | CCR5-delta32 |

### Pathogenic Mutations

In some embodiments, the systems or methods provided herein can be used to ameliorate the effects of a pathogenic mutation. The pathogenic mutation can be a genetic mutation that increases an individual's susceptibility or predisposition to a certain disease or disorder. In some embodiments, the pathogenic mutation is a disease-causing mutation in a gene associated with a disease or disorder. In some embodiments, the systems or methods provided herein can be used to supply a wild-type sequence corresponding to the pathogenic mutation.

Table 23 provides exemplary indications (column 1), underlying genes (column 2), and pathogenic mutations that can be addressed using the systems or methods described herein (column 3).

TABLE 23
Indications, genes, and causitive pathogenic mutations.

| Disease | Gene | Pathogenic Mutation^{#} |
|---|---|---|
| Achromatopsia | CNGB3 | 1148delC |
| Alpha-1 Antitrypsin Deficiency | SERPINA1 | E342K |
| Alpha-1 Antitrypsin Deficiency | SERPINA1 | E342K |
| Alpha-1 Antitrypsin Deficiency | SERPINA1 | R48C (R79C) |
| Amaurosis Congenita (LCA10) | CEP290 | 2991 + 1655A > G |
| Andersen- Tawil syndrome | KCNJ2 | R218W |
| Arrhythmogenic right ventricular cardiomyopathy | PKP2 | c.235C > T |
| (ARVC) | | |
| associated with congenital factor XI deficiency | F11 | E117* |
| associated with congenital factor XI deficiency | F11 | F283L |
| ATTR amyloidosis | TTR | V50M/N30M |
| autosomal dominant deafness | COCH | G88E |
| autosomal dominant deafness | TECTA | Y1870C |
| autosomal dominant Parkinson's disease | SNCA | A53T |
| autosomal dominant Parkinson's disease | SNCA | A30P |
| Autosomal dominant rickets | FGF23 | R176Q |
| autosomal recessive deafness | CX30 | T5M |
| autosomal recessive deafness | DFNB59 | R183W |
| autosomal recessive deafness | TMC1 | Y182C |
| autosomal recessive hypercholesterolemia | ARH | Q136* |
| Blackfan-Diamond anemia | RPS19 | R62Q |
| blue-cone monochromatism | OPN1LW | C203R |
| Brugada syndrome | SCN5A | E1784K |
| CADASIL syndrome | NOTCH3 gene | R90C |
| CADASIL syndrome | NOTCH3 gene | R141C |
| Canavan disease | ASPA | E285A |
| Canavan disease | ASPA | Y231X |
| Canavan disease | ASPA | A305E |
| carnitine palmitoyltransferase II deficiency | CPT2 | S113L |
| choroideremia | CHM | R293* |
| choroideremia | CHM | R270* |
| choroideremia | CHM | A117A |
| Citrullinemia Type I | ASS | G390R |
| classic galactosemia | GALT | Q188R |
| classic horoocystoinuria | CBS | T191M |
| classic homocystemuria | CBS | G307S |
| CLN2 Disease | TPP1 | c.509 − 1 G > C |
| CLN2 Disease | TPP1 | c.622 C < T |
| CLN2 Disease | TPP1 | c.851 G > T |
| cone-rod dystrophy | GUCY2D | R838C |
| congenital factor V deficiency | F5 | R506Q |
| congenital factor V deficiency | F5 | R534Q |
| congenital factor VII deficiency | F7 | A294V |
| congenital factor VII deficiency | F7 | C310F |
| congenital factor VII deficiency | F7 | R304Q |
| congenital factor VII deficiency | F7 | QI00R |
| Creutzfeldt- Jakob disease (CJD) | PRNP | E200K |
| Creutzfeldt- Jakob disease (CJD) | PRNP | M129V |
| Creutzfeldt- Jakob disease (CJD) | PRNP | P102L |
| Creutzfeldt- Jakob disease (CJD) | PRNP | D178N |
| cystic fibrosis | CFTR | G551D |
| cystic fibrosis | CFTR | W1282* |
| cystic fibrosis | CFTR | R553* |
| cystic fibrosis | CFTR | R117H |
| cystic fibrosis | CFTR | delta F508 |
| eystinosis | CTNS | W138* |
| Darier disease | ATP2A2 | N767S |
| Darier disease | ATP2A2 | N767S |
| Darier disease | ATP2A2 | N767S |
| Epidermolysis Bullosa Junctional | LAMB3 | R42X |
| Epidermolysis Bullosa Junctional | LAMB3 | R635X |
| familial amyotrophic lateral sclerosis (ALS) | SOD1 | A4V |
| familial amyotrophic lateral sclerosis (ALS) | SOD1 | H46R |
| familial amyotrophic lateral sclerosis (ALS) | SOD1 | G37R |
| Gaucher disease | GBA | N370S |
| Gaucher disease | GBA | N370S |
| Gaucher disease | GBA | L444P |
| Gaucher disease | GBA | L444P |
| Gaucher disease | GBA | L483P |
| glutarvl-CoA dehydrogenase deficiency | GCDH | R138G |
| glutaryl-CoA dehydrogenase deficiency | GCDH | M263V |
| glutaryl-CoA dehydrogenase deficiency | GCDH | R402W |
| glycine encephalopathy | GLDC | A389V |
| glycine encephalopathy | GLDC | G771R |
| glycine encephalopathy | GLDC | T269M |
| hemophilia A | F8 | R2178C |
| hemophilia A | F8 | R550C |
| hemophilia A | F8 | R2169H |
| hemophilia A | F8 | R1985Q |
| hemophilia B | F9 | T342M |
| hemophilia B | F9 | R294Q |
| hemophilia B | F9 | R43Q |
| hemophilia B | F9 | R191H |
| hemophilia B | F9 | G106S |
| hemophilia B | F9 | A279T |
| hemophilia B | F9 | R75* |
| hemophilia B | F9 | R294* |
| hemophilia B | F9 | R379Q |
| Hereditary antithrombin deficiency type I | SERPINCI | R48C (R79C) |
| hereditary chronic pancreatitis | PRSS1 | R122H |
| Hunter syndrome | IDS | R88C |
| Hunter syndrome | IDS | G374G |
| Hurler syndrome (MPS1) | IDUA | Q70* |
| Hurler syndrome (MPS1) | IDUA | W402* |
| Hyperkalemic periodic paralysis | SCN4A | T704M |
| Hyperkalemic periodic paralysis | SCN4A | M1592V |
| Hyperkalemic periodic paralysis | CACNA1S | p.Arg528X |
| Hyperkalemic periodic paralysis | CACNA1S | p.Arg1239 |
| intermittent porphyria | HMBS | RI73W |
| isolated agammaglobulinemia | E47 | E555K |
| Lattice corneal dystrophy type I | TGFBI | Arg124Cys |
| LCHAD deficiency | HADHA | Glu474Gln |
| Leber congenital amaurosis 2 | RPE65 | R44* |
| Leber congenital amaurosis 2 | RPE65 | IVS1 |
| Leber congenital amaurosis 2 | RPE65 | G-A, +5 |
| Lesch-Nyhan syndrome | HPRTI | R51* |
| Lesch-Nyhan syndrome | HPRTI | R170* |
| Limb-girdle muscular dystrophy, type 2D | SGCA | Arg77Cys |
| Marteauz- Lamy Syndrome (MSPVI) | ARSB | Y210C |
| Mediterranean G6PD deficiency | G6PD | S188D |
| medium-chain acyl-CoA dehydrogenase deficiency | ACADM | K329E |
| medium-chain acyl-CoA dehydrogenase deficiency | ACADM | K329E |
| medium-chain acyl-CoA dehydrogenase deficiency | ACADM | K329E |
| Meesmann epithelial corneal dystrophy | KRT12 | L132P |
| metachfoniatic leukodystrophy | ARSA | P426L |
| metachromatic leukodystrophy | ARSA | c.459 + 1G > A |
| Morquio Syndrome (MPSIVA) | GALNS | R386C |
| Mucolipidosis IV | MCOLN1 | 406-2A > G |
| Mucolipidosis IV | MCOLN1 | 511_6943del |
| Neimann-Pick disease type A | SMPDI | L302P |
| Neuronal ceroid lipofuscinosis (NCL) | CLN2 | R208* |
| neuronal ceroid lipofuscinosis 1 | PPT1 | R151* |
| Parkinson's disease | LRRK2 | G2019S |
| Pendred syndrome | PDS | T461P |
| Pendred syndrome | PDS | L236P |
| Pendred syndrome | PDS | c.1001 + 1G > A |
| Pendred syndrome | PDS | IVS8, +1 G > A, |
| phenylketonuria | PAH | R408W |
| phenylketonuria | PAH | I65T |
| phenylketonuria | PAH | R261Q |
| phenylketonuria | PAH | IVS10-11G > A |
| phenylketonuria | PCDH15 | R245* |
| phenylketonuria | PCDH15 | R245* |
| Pompe disease | GAA | c.−32 − 13T > G |
| Primary ciliary dyskinesia | DNAI1 | IVS1 + 2_3insT |
| Primary ciliary dyskinesia | DNAH5 | 10815delT |
| primary hypoxalimia | AGXT | G170R |
| Progressive familial intrahepatic cholestasis type 2 | ABCB11 | D482G (c.1445A > G) |
| Progressive familial intrahepatic cholestasis type 2 | ABCB11 | E297G |
| Propionic acidemia | PCCB; PCCA | c.1218_1231del14ins12 |
| pseudoxanthoma elasticum | ABCC6 | R1141* |
| Pyruvate kinase deficiency (PKD) | PKLR | c.1456c −> T |
| retinitis pigmentos | USH2a | C759F |
| retinitis pigmentosa | IMPDHI | D226N |
| retinitis pigmentosa | PDE6A | V685M |
| retinitis pigmentosa | PDE6A | D670G |
| retinitis pigmentosa | PRPF3 | T494M |
| retinitis pigmentosa | PRPF8 | H2309R |
| retinitis pigmentosa | RHO | P23H |
| retinitis pigmentosa | RHO | P347L |
| retinitis pigmentosa | RHO | P347L |
| retinitis pigmentosa | RHO | D190N |
| retinitis pigmentosa | RPI | R667* |
| retinitis pigmentosa/Usher syndrome type 1C | USH1C | V72V |
| Rett syndrome | MECP2 | R106W |
| Rett syndrome | MECP2 | R133C |
| Rett syndrome | MECP2 | R306C |
| Rett syndrome | MECP2 | R168* |
| Rett syndrome | MECP2 | R255* |
| Sanfilippo syndrome A (MPSIIIA) | SGSH | R74C |
| Sanfilippo syndrome A (MPSIIIA) | SGSH | R245H |
| Sanfilippo syndrome B (MPSIIIB) | NAGLU | R297* |
| Sanfilippo syndrome B (MPSIIIB) | NAGLU | Y140C |
| severe combined immunodeficiency | ADA | G216R |
| severe combined immunodeficiency | ADA | G216R |
| severe combined immunodeficiency | ADA | Q3* |
| sickle cell disease | HBB | E6V |
| sickle cell disease | HBB | E6V |
| sickle cell disease | HBB | E6V |
| sickle cell disease | HBB | E26K |
| sickle cell disease | HBB | E26K |
| sickle cell disease | HBB | E7K |
| sickle cell disease | HBB | c.−138C > T |
| sickle cell disease | HBB | IVS2 |
| sickle cell disease | HBB | 654 C > T |
| Sly Syndrome (MPSVII) | GUSB | L175F |
| Stargardt disease | ABCA4 | A1038V |
| Stargardt disease | ABCA4 | A1038V |
| Stargardt disease | ABCA4 | L541P |
| Stargardt disease | ABCA4 | G1961E |
| Stargardt disease | ABCA4 | G1961E |
| Stargardt disease | ABCA4 | G1961E |
| Stargardt disease | ABCA4 | G1961E |
| Stargardt disease | ABCA4 | c.2588G > C |
| Stargardt disease | ABCA4 | c.5461 − 10 T > C |
| Stargardt disease | ABCA4 | c.5714 + 5G > A |
| Tay Sachs | HEXA | InsTATC1278 |
| tyrosinemia type 1 | FAH | P261L |
| Usher syndrome type 1F | PCDH15 | R245* |
| variegate porphyria | PPOX | R59W |
| VCP myopathy (IBMPFD) 1 | VCP | R1555X |
| von Gierke disease | G6PC | Q347* |
| von Gierke disease | G6PC | Q347* |
| von Gierke disease | G6PC | Q347* |
| von Gierke disease | G6PC | R83C |
| Wilson's Disease | ATP7B | E297G |
| X-linked myotubular myopathy | MTMI | c.1261 − 10A > G |
| X-linked retinoschisis | RS1 | R102W |
| X-linked retinoschisis | RS1 | R141C |

^{#}See J T den Dunnen and S E Antonarakis, Hum Mutat. 2000; 15(1): 7-12, herein incorporated by reference in its entirety, for details of the nomenclatures of gene mutations.
*means a stop codon.

### Compensatory Edits

In some embodiments, the systems or methods provided herein can be used to introduce a compensatory edit. In some embodiments, the compensatory edit is at a position of a gene associated with a disease or disorder, which is different from the position of a disease-causing mutation. In some embodiments, the compensatory mutation is not in the gene containing the causitive mutation. In some embodiments, the compensatory edit can negate or compensate for a disease-causing mutation. In some embodiments, the compensatory edit can be introduced by the systems or methods provided herein to suppress or reverse the mutant effect of a disease-causing mutation.

Table 24 provides exemplary indications (column 1), genes (column 2), and compensatory edits that can be introduced using the systems or methods described herein (column 3). In some embodiments, the compensatory edits provided in Table 24 can be introduced to suppress or reverse the mutant effect of a disease-causing mutation.

TABLE 24
Indications, genes, compensatory edits,
and exemplary design features.

| Disease | Gene | Nucleotide Change^{#} |
|---|---|---|
| Alpha-1 Antitrypsin Deficiency | SERPINAI | F51L |
| Alpha-1 Antitrypsin Deficiency | SERPINAI | M374I |
| Alpha-1 Antitrypsin Deficiency | SERPINAI | A348V/A347V |
| Alpha-1 Antitrypsin Deficiency | SERPINAI | K387R |
| Alpha-1 Antitrypsin Deficiency | SERPINAI | T59A |
| Alpha-1 Antitiypsin Deficiency | SERPINAI | T68A |
| ATTR amyloidosis | TTR | A108V |
| ATTR amyloidosis | TTR | R104H |
| ATTR amyloidosis | TTR | T119M |
| Cystic fibroses | CFTR | R555K |
| Cystic fibrosis | CFTR | F409L |
| Cystic fibrosis | CFTR | F433L |
| Cystic fibrosis | CFTR | H667R |
| Cystic fibrosis | CFTR | R1070W |
| Cystic fibrosis | CFTR | R29K |
| Cystic fibrosis | CFTR | R553Q |
| Cystic fibrosis | CFTR | 1539T |
| Cystic fibrosis | CFTR | G550E |
| Cystic fibroses | CFTR | F429S |
| Cystic fibrosis | CFTR | Q637R |
| Sickle cell disease | HBB | A70T |
| Sickle cell disease | HBB | A70V |
| Sickle cell disease | HBB | L88P |
| Sickle cell disease | HBB | F85L and/or F85P |
| Sickle cell disease | HBB | E22G |
| Sickle cell disease | HBB | G16D and/or G16N |

^{#}See J T den Dunnen and S E Antonarakis, Hum Mutat. 2000; 15(1): 7-12, herein incorporated by reference in its entirety, for details of the nomenclatures of gene mutations.

### Regulatory Edits

In some embodiments, the systems or methods provided herein can be used to introduce a regulatory edit. In some embodiments, the regulatory edit is introduced to a regulatory sequence of a gene, for example, a gene promoter, gene enhancer, gene repressor, or a sequence that regulates gene splicing. In some embodiments, the regulatory edit increases or decreases the expression level of a target gene. In some embodiments, the target gene is the same as the gene containing a disease-causing mutation. In some embodiment, the target gene is different from the gene containing a disease-causing mutation. For example, the systems or methods provided herein can be used to upregulate the expression of fetal hemoglobin by introducing a regulatory edit at the promoter of bcl11a, thereby treating sickle cell disease.

Table 25 provides exemplary indications (column 1), genes (column 2), and regulatory edits that can be introduced using the systems or methods described herein (column 3).

TABLE 25
Indications, genes, and compensatory regulatory edits.

| Disease | Gene | Nucleotide Change^{#} |
|---|---|---|
| homozygous familial hypercholesterolaemia | LDLR | c.81C > T |
| Porphyrias | ALAS1 | c.3G > A |
| Porphyrias | ALAS1 | c.2T > C |
| Porphyrias | ALAS1 | c.46C > T |
| Porphyrias | ALAS1 | c.91C > T |
| Porphyrias | ALAS1 | c.91C > T |
| Porphyrias | ALAS1 | c.226C > T |
| Porphyrias | ALAS1 | c.226C > T |
| Porphyrias | ALAS1 | c.226C > T |
| Porphyrias | ALAS1 | c.229C > T |
| Porphyrias | ALAS1 | c.247C > T |
| Porphyrias | ALAS1 | c.247C > T |
| Porphyrias | ALAS1 | c.250C > T |
| Porphyrias | ALAS1 | c.250C > T |
| Porphyrias | ALAS1 | c.340C > T |
| Porphyrias | ALAS1 | c.340C > T |
| Porphyrias | ALAS1 | c.349C > T |
| Porphyrias | ALAS1 | c.391C > T |
| Porphyrias | ALAS1 | c.391C > T |
| Porphyrias | ALAS1 | c.403C > T |
| Porphyrias | ALAS1 | c.403C > T |
| Porphyrias | ALAS1 | c.199 + 1G > A |
| Porphyrias | ALAS1 | c.199 + 1G > A |
| Porphyrias | ALAS1 | c.199 + 1G > A |
| Porphyrias | ALAS1 | c.199 + 1G > A |
| Porphyrias | ALAS1 | c.199 + 2T > C |
| Porphyrias | ALAS1 | c.199 + 2T > C |
| Porphyrias | ALAS1 | c.199 + 2T > C |
| Porphyrias | ALAS1 | c.199 + 2T > C |
| Porphyrias | ALAS1 | c.200 − 2A > G |
| Porphyrias | ALAS1 | c.427 + 1G > A |
| Porphyrias | ALAS1 | c.427 + 2T > C |
| Porphyrias | ALAS1 | c.1165 + 1G > A |
| Porphyrias | ALAS1 | c.1165 + 2T > C |
| Porphyrias | ALAS1 | c.1166 − 1A > G |
| Porphyrias | ALAS1 | c.1331 − 2A > G |
| sickle cell disease | BCL11A | c.386-24278G > A |
| sickle cell disease | BCL11A | c.386-24983T > C |
| sickle cell disease | HBG1 | c.−167C > T |
| sickle cell disease | HBG1 | c.−170G > A |
| sickle cell disease | HBG1 | c.−249C > T |
| sickle cell disease | HBG2 | c.−211C > T |
| sickle cell disease | HBG2 | c.−228T > C |
| sickle cell disease | HBG1/2 | C.−198 T > C |
| sickle cell disease | HBG1/2 | C.−198 T > C |
| sickle cell disease | HBG1/2 | C.−198 T > C |
| sickle cell disease | HBG1/2 | C.−198 T > C |
| sickle cell disease | HBG1/2 | C.−198 T > C |
| sickle cell disease | HBG1/2 | C.−198 T > C |
| sickle cell disease | HBG1/2 | C.−198 T > C |
| sickle cell disease | HBG1/2 | C.−175 T > C |
| sickle cell disease | HBG1/2 | C.−175 T > C |
| sickle cell disease | HBG1/2 | C.−175 T > C |
| sickle cell disease | HBG1/2 | C.−175 T > C |
| sickle cell disease | HBG1/2 | C.−175 T > C |
| sickle cell disease | HBG1/2 | C.−114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | c. −90 BCL11A Binding |
| sickle cell disease | HBG1/2 | c. −90 BCL11A Binding |
| sickle cell disease | HBG1/2 | C. −202 C > T, −201 C > |
| | | T, −198 T > C, −197 C > |
| | | T, −196 C > T, −195 C > G |
| sickle cell disease | HBG1/2 | C. −197 C > T, −196 C > |
| | | T, −195 C > G |

^{#}See J T den Dunnen and S E Antonarakis, Hum Mutat. 2000; 15(1): 7-12, herein incorporated by reference in its entirety, for details of the nomenclatures of gene mutations.

### Repeat Expansion Diseases

In some embodiments, the systems or methods provided herein can be used to treat a repeat expansion disease, for example, a repeat expansion disease provided in Table 26. Table 26 provides the indication (column 1), the gene (column 2), minimal repeat sequence of the repeat that is expanded in the condition (column 3), and the location of the repeat relative to the listed gene for each indication (column 4). In some embodiments, the systems or methods provided herein, for example, those comprising Gene Writers, can be used to treat repeat expansion diseases by resetting the number of repeats at the locus according to a customized DNA template.

TABLE 26
Exemplary repeat expansion diseases, genes, causal repeats, and repeat locations.

| Disease | Gene | Causal repeat | Repeat location |
|---|---|---|---|
| myotonic dystrophy 1 | DMPK/DM1 | CTG | 3′ UTR |
| myotonic dystrophy 2 | ZNF9/CNBP | CCTG | Intron 1 |
| dentatorubral-pallidoluysian atrophy | ATN1 | CAG | Coding |
| fragile X mental retardation | FMR1 | CGG | 5′ UTR |
| syndrome | | | |
| fragile X E mental retardation | FMR2 | GCC | 5′ UTR |
| Friedreich's ataxia | FXN | GAA | Intron |
| fragile X tremor ataxia syndrome | FMR1 | CGG | 5′ UTR |
| Huntington's disease | HTT | CAG | Coding |
| Huntington's disease-like 2 | JPH3 | CTG | 3′ UTR, coding |
| myoclonic epilepsy of Unverricht and | CSTB | CCCCGCCCCGCG | Promoter |
| Lundborg | | (SEQ ID NO: 1581) | |
| oculopharyngeal muscular dystrophy | PABPN1 | GCG | Coding |
| spinal and bulbar muscular atrophy | AR | CAG | Coding |
| spinocerebellar ataxia 1 | ATXN1 | CAG | Coding |
| spinocerebellar ataxia 2 | ATXN2 | CAG | Coding |
| spinocerebellar ataxia 3 | ATXN3 | CAG | Coding |
| spinocerebellar ataxia 6 | CACNA1A | CAG | Coding |
| spinocerebellar ataxia 7 | ATXN7 | CAG | Coding |
| spinocerebellar ataxia 8 | ATXN8 | CTG/CAG | CTG/CAG |
| | | | (ATXN8) |
| spinocerebellar ataxia 10 | ATXN10 | ATTCT | Intron |
| spinocerebellar ataxia 12 | PPP2R2B | CAG | Promoter, 5′ |
| | | | UTR? |
| spinocerebellar ataxia 17 | TBP | CAG | Coding |
| Syndromic/non-syndromic X-linked | ARX | GCG | Coding |
| mental retardation | | | |

### Exemplary Heterologous Object Sequences

In some embodiments, the systems or methods provided herein comprise a heterologous object sequence, wherein the heterologous object sequence or a reverse complementary sequence thereof, encodes a protein (e.g., an antibody) or peptide. In some embodiments, the therapy is one approved by a regulatory agency such as FDA.

In some embodiments, the protein or peptide is a protein or peptide from the THPdb database (Usmani et al. PLoS One 12(7):e0181748 (2017), herein incorporated by reference in its entirety. In some embodiments, the protein or peptide is a protein or peptide disclosed in Table 28. In some embodiments, the systems or methods disclosed herein, for example, those comprising Gene Writers, may be used to integrate an expression cassette for a protein or peptide from Table 28 into a host cell to enable the expression of the protein or peptide in the host. In some embodiments, the sequences of the protein or peptide in the first column of Table 28 can be found in the patents or applications provided in the third column of Table 28, incorporated by reference in their entireties.

In some embodiments, the protein or peptide is an antibody disclosed in Table 1 of Lu et al. J Biomed Sci 27(1):1 (2020), herein incorporated by reference in its entirety. In some embodiments, the protein or peptide is an antibody disclosed in Table 29. In some embodiments, the systems or methods disclosed herein, for example, those comprising Gene Writers, may be used to integrate an expression cassette for an antibody from Table 29 into a host cell to enable the expression of the antibody in the host. In some embodiments, a system or method described herein is used to express an agent that binds a target of column 2 of Table 29 (e.g., a monoclonal antibody of column 1 of Table 29) in a subject having an indication of column 3 of Table 29.

TABLE 28
Exemplary protein and peptide therapeutics.

| Therapeutic peptide | Category | Patent Number |
|---|---|---|
| Lepirudin | Antithrombins and Fibrinolytic | CA1339104 |
| | Agents | |
| Cetuximab | Antineoplastic Agents | CA1340417 |
| Dor se alpha | Enzymes | CA2184581 |
| Denileukin diftitox | Antineoplastic Agents | |
| Etanercept | Immunosuppressive Agents | CA2476934 |
| Bivalirudin | Antithrombins | U.S. Pat. No. 7,582,727 |
| Leuprolide | Antineoplastic Agents | |
| Peginterferon alpha-2a | Immunosuppressive Agents | CA2203480 |
| Alteplase | Thrombolytic Agents | |
| Interferon alpha-n1 | Antiviral Agents | |
| Darbepoetin alpha | Anti-anemic Agents | CA2165694 |
| Reteplase | Fibrinolytic Agents | CA2107476 |
| Epoetin alpha | Hematinics | CA1339047 |
| Salmon Calcitonin | Bone Density Conservation | U.S. Pat. No. 6,440,392 |
| | Agents | |
| Interferon alpha-n3 | Immunosuppressive Agents | |
| Pegfilgrastim | Immunosuppressive Agents | CA1341537 |
| Sargramostim | Immunosuppressive Agents | CA1341150 |
| Secretin | Diagnostic Agents | |
| Peginterferon alpha-2b | Immunosuppressive Agents | CA1341567 |
| Asparagi se | Antineoplastic Agents | |
| Thyrotropin alpha | Diagnostic Agents | U.S. Pat. No. 5,840,566 |
| Antihemophilic Factor | Coagulants and Thrombotic agents | CA2124690 |
| A kinra | Antirheumatic Agents | CA2141953 |
| Gramicidin D | Anti-Bacterial Agents | |
| Intravenous | Immunologic Factors | |
| Immunoglobulin | | |
| Anistreplase | Fibrinolytic Agents | |
| Insulin Regular | Antidiabetic Agents | |
| Tenecteplase | Fibrinolytic Agents | CA2129660 |
| Menotropins | Fertility Agents | |
| Interferon gamma-1b | Immunosuppressive Agents | U.S. Pat. No. 6,936,695 |
| Interferon alpha-2a, | | CA2172664 |
| Recombi nt | | |
| Coagulation factor VIIa | Coagulants | |
| Oprelvekin | Antineoplastic Agents | |
| Palifermin | Anti-Mucositis Agents | |
| Glucagon recombi nt | Hypoglycemic Agents | |
| Aldesleukin | Antineoplastic Agents | |
| Botulinum Toxin Type B | Antidystonic Agents | |
| Omalizumab | Anti-Allergic Agents | CA2113813 |
| Lutropin alpha | Fertility Agents | U.S. Pat. No. 5,767,251 |
| Insulin Lispro | Hypoglycemic Agents | U.S. Pat. No. 5,474,978 |
| Insulin Glargine | Hypoglycemic Agents | U.S. Pat. No. 7,476,652 |
| Collage se | | |
| Rasburicase | Gout Suppressants | CA2175971 |
| Adalimumab | Antirheumatic Agents | CA2243459 |
| Imiglucerase | Enzyme Replacement Agents | U.S. Pat. No. 5,549,892 |
| Abciximab | Anticoagulants | CA1341357 |
| Alpha-1-protei se inhibitor | Serine Protei se Inhibitors | |
| Pegaspargase | Antineoplastic Agents | |
| Interferon beta-1a | Antineoplastic Agents | CA1341604 |
| Pegademase bovine | Enzyme Replacement Agents | |
| Human Serum Albumin | Serum substitutes | U.S. Pat. No. 6,723,303 |
| Eptifibatide | Platelet Aggregation Inhibitors | U.S. Pat. No. 6,706,681 |
| Serum albumin iodo ted | Diagnostic Agents | |
| Infliximab | Antirheumatic Agents, Anti- | CA2106299 |
| | Inflammatory Agents, Non- | |
| | Steroidal, Dermatologic Agents, | |
| | Gastrointesti 1 Agents and | |
| | Immunosuppressive Agents | |
| Follitropin beta | Fertility Agents | U.S. Pat. No. 7,741,268 |
| Vasopressin | Antidiuretic Agents | |
| Interferon beta-1b | Adjuvants, Immunologic and | CA1340861 |
| | Immunosuppressive Agents | |
| Interferon alphacon-1 | Antiviral Agents and | CA1341567 |
| | Immunosuppressive Agents | |
| Hyaluronidase | Adjuvants, Anesthesia and | |
| | Permeabilizing Agents | |
| Insulin, porcine | Hypoglycemic Agents | |
| Trastuzumab | Antineoplastic Agents | CA2103059 |
| Rituximab | Antineoplastic Agents, | CA2149329 |
| | Immunologic Factors and | |
| | Antirheumatic Agents | |
| Basiliximab | Immunosuppressive Agents | CA2038279 |
| Muromo b | Immunologic Factors and | |
| | Immunosuppressive Agents | |
| Digoxin Immune Fab | Antidotes | |
| (Ovine) | | |
| Ibritumomab | | CA2149329 |
| Daptomycin | | U.S. Pat. No. 6,468,967 |
| Tositumomab | | |
| Pegvisomant | Hormone Replacement Agents | U.S. Pat. No. 5,849,535 |
| Botulinum Toxin Type A | Neuromuscular Blocking Agents, | CA2280565 |
| | Anti-Wrinkle Agents and | |
| | Antidystonic Agents | |
| Pancrelipase | Gastrointesti 1 Agents and Enzyme | |
| | Replacement Agents | |
| Streptoki se | Fibrinolytic Agents and | |
| | Thrombolytic Agents | |
| Alemtuzumab | | CA1339198 |
| Alglucerase | Enzyme Replacement Agents | |
| Capromab | Indicators, Reagents and | |
| | Diagnostic Agents | |
| Laronidase | Enzyme Replacement Agents | |
| Urofollitropin | Fertility Agents | U.S. Pat. No. 5,767,067 |
| Efalizumab | Immunosuppressive Agents | |
| Serum albumin | Serum substitutes | U.S. Pat. No. 6,723,303 |
| Choriogo dotropin alpha | Fertility Agents and Go dotropins | U.S. Pat. No. 6,706,681 |
| Antithymocyte globulin | Immunologic Factors and | |
| | Immunosuppressive Agents | |
| Filgrastim | Immunosuppressive Agents, | CA1341537 |
| | Antineutropenic Agents and | |
| | Hematopoietic Agents | |
| Coagulation factor ix | Coagulants and Thrombotic | |
| | Agents | |
| Becaplermin | Angiogenesis Inducing Agents | CA1340846 |
| Agalsidase beta | Enzyme Replacement Agents | CA2265464 |
| Interferon alpha-2b | Immunosuppressive Agents | CA1341567 |
| Oxytocin | Oxytocics, Anti-tocolytic Agents | |
| | and Labor Induction Agents | |
| Enfuvirtide | HIV Fusion Inhibitors | U.S. Pat. No. 6,475,491 |
| Palivizumab | Antiviral Agents | CA2197684 |
| Daclizumab | Immunosuppressive Agents | |
| Bevacizumab | Angiogenesis Inhibitors | CA2286330 |
| Arcitumomab | Diagnostic Agents | U.S. Pat. No. 8,420,081 |
| Arcitumomab | Diagnostic Agents | U.S. Pat. No. 7,790,142 |
| Eculizumab | | CA2189015 |
| Panitumumab | | |
| Ranibizumab | Ophthalmics | CA2286330 |
| Idursulfase | Enzyme Replacement Agents | |
| Alglucosidase alpha | Enzyme Replacement Agents | CA2416492 |
| Exe tide | Hypoglycemic Agents | U.S. Pat. No. 6,872,700 |
| Mecasermin | | U.S. Pat. No. 5,681,814 |
| Pramlintide | | U.S. Pat. No. 5,686,411 |
| Galsulfase | Enzyme Replacement Agents | |
| Abatacept | Antirheumatic Agents and | CA2110518 |
| | Immunosuppressive Agents | |
| Cosyntropin | Hormones and Diagnostic Agents | |
| Corticotropin | | |
| Insulin aspart | Hypoglycemic Agents and | U.S. Pat. No. 5,866,538 |
| | Antidiabetic Agents | |
| Insulin detemir | Antidiabetic Agents | U.S. Pat. No. 5,750,497 |
| Insulin glulisine | Antidiabetic Agents | U.S. Pat. No. 6,960,561 |
| Pegaptanib | Intended for the prevention of | |
| | respiratory distress syndrome | |
| | (RDS) in premature infants at high | |
| | risk for RDS. | |
| Nesiritide | | |
| Thymalphasin | | |
| Defibrotide | Antithrombins | |
| tural alpha interferon OR | | |
| multiferon | | |
| Glatiramer acetate | | |
| Preotact | | |
| Teicoplanin | Anti-Bacterial Agents | |
| Ca kinumab | Anti-Inflammatory Agents and | |
| | Monoclo 1 antibodies | |
| Ipilimumab | Antineoplastic Agents and | CA2381770 |
| | Monoclo 1 antibodies | |
| Sulodexide | Antithrombins and Fibrinolytic | |
| | Agents and Hypoglycemic Agents | |
| | and Anticoagulants and | |
| | Hypolipidemic Agents | |
| Tocilizumab | | CA2201781 |
| Teriparatide | Bone Density Conservation | U.S. Pat. No. 6,977,077 |
| | Agents | |
| Pertuzumab | Monoclo 1 antibodies | CA2376596 |
| Rilo cept | Immunosuppressive Agents | U.S. Pat. No. 5,844,099 |
| Denosumab | Bone Density Conservation | CA2257247 |
| | Agents and Monoclo 1 antibodies | |
| Liraglutide | | U.S. Pat. No. 6,268,343 |
| Golimumab | Antipsoriatic Agents and Monoclo | |
| | 1 antibodies and TNF inhibitor | |
| Belatacept | Antirheumatic Agents and | |
| | Immunosuppressive Agents | |
| Buserelin | | |
| Velaglucerase alpha | Enzymes | U.S. Pat. No. 7,138,262 |
| Tesamorelin | | U.S. Pat. No. 5,861,379 |
| Brentuximab vedotin | | |
| Taliglucerase alpha | Enzymes | |
| Belimumab | Monoclo 1 antibodies | |
| Aflibercept | Antineoplastic Agents and | U.S. Pat. No. 7,306,799 |
| | Ophthalmics | |
| Asparagi se erwinia | Enzymes | |
| chrysanthemi | | |
| Ocriplasmin | Ophthalmics | |
| Glucarpidase | Enzymes | |
| Teduglutide | | U.S. Pat. No. 5,789,379 |
| Raxibacumab | Anti-Infective Agents and | |
| | Monoclo 1 antibodies | |
| Certolizumab pegol | TNF inhibitor | CA2380298 |
| Insulin, isophane | Hypoglycemic Agents and | |
| | Antidiabetic Agents | |
| Epoetin zeta | | |
| Obinutuzumab | Antineoplastic Agents | |
| Fibrinolysin aka plasmin | | U.S. Pat. No. 3,234,106 |
| Follitropin alpha | | |
| Romiplostim | Colony-Stimulating Factors and | |
| | Thrombopoietic Agents | |
| Luci ctant | Pulmo ry surfactants | U.S. Pat. No. 5,407,914 |
| talizumab | Immunosuppressive agents | |
| Aliskiren | Renin inhibitor | |
| Ragweed Pollen Extract | | |
| Secukinumab | Inhibitor | US20130202610 |
| Somatotropin Recombi nt | Hormone Replacement Agents | CA1326439 |
| Drotrecogin alpha | Antisepsis | CA2036894 |
| Alefacept | Dermatologic and | |
| | Immunosupressive agents | |
| OspA lipoprotein | Vaccines | |
| Uroki se | | U.S. Pat. No. 4,258,030 |
| Abarelix | Anti-Testosterone Agents | U.S. Pat. No. 5,968,895 |
| Sermorelin | Hormone Replacement Agents | |
| Aprotinin | | U.S. Pat. No. 5,198,534 |
| Gemtuzumab ozogamicin | Antineoplastic agents and | U.S. Pat. No. 5,585,089 |
| | Immunotoxins | |
| Satumomab Pendetide | Diagnostic Agents | |
| Albiglutide | Drugs used in diabetes; alimentary | |
| | tract and metabolism; blood | |
| | glucose lowering drugs, excl. | |
| | insulins. | |
| Alirocumab | | |
| Ancestim | | |
| Antithrombin alpha | | |
| Antithrombin III human | | |
| Asfotase alpha | Enzymes Alimentary Tract and | |
| | Metabolism | |
| Atezolizumab | | |
| Autologous cultured | | |
| chondrocytes | | |
| Beractant | | |
| Bli tumomab | Antineoplastic Agents | US20120328618 |
| | Immunosuppressive Agents | |
| | Monoclo 1 antibodies | |
| | Antineoplastic and | |
| | Immunomodulating Agents | |
| C1 Esterase Inhibitor | | |
| (Human) | | |
| Coagulation Factor XIII A- | | |
| Subunit (Recombi nt) | | |
| Conestat alpha | | |
| Daratumumab | Antineoplastic Agents | |
| Desirudin | | |
| Dulaglutide | Hypoglycemic Agents; Drugs | |
| | Used in Diabetes; Alimentary | |
| | Tract and Metabolism; Blood | |
| | Glucose Lowering Drugs, Excl. | |
| | Insulins | |
| Elosulfase alpha | Enzymes; Alimentary Tract and | |
| | Metabolism | |
| Elotuzumab | | US2014055370 |
| Evolocumab | Lipid Modifying Agents, Plain; | |
| | Cardiovascular System | |
| Fibrinogen Concentrate | | |
| (Human) | | |
| Filgrastim-sndz | | |
| Gastric intrinsic factor | | |
| Hepatitis B immune | | |
| globulin | | |
| Human calcitonin | | |
| Human Clostridium tetani | | |
| toxoid immune globulin | | |
| Human rabies virus | | |
| immune globulin | | |
| Human Rho(D) immune | | |
| globulin | | |
| Hyaluronidase (Human | | U.S. Pat. No. 7,767,429 |
| Recombi nt) | | |
| Idarucizumab | Anticoagulant | |
| Immune Globulin Human | Immunologic Factors; | |
| | Immunosuppressive Agents; Anti- | |
| | Infective Agents | |
| Vedolizumab | Immunosupressive agent, | US2012151248 |
| | Antineoplastic agent | |
| Ustekinumab | Deramtologic agent, | |
| | Immunosuppressive agent, | |
| | antineoplastic agent | |
| Turoctocog alpha | | |
| Tuberculin Purified Protein | | |
| Derivative | | |
| Simoctocog alpha | Antihaemorrhagics: blood | |
| | coagulation factor VIII | |
| Siltuximab | Antineoplastic and | U.S. Pat. No. 7,612,182 |
| | Immunomodulating Agents, | |
| | Immunosuppressive Agents | |
| Sebelipase alpha | Enzymes | |
| Sacrosidase | Enzymes | |
| Ramucirumab | Antineoplastic and | US2013067098 |
| | Immunomodulating Agents | |
| Prothrombin complex | | |
| concentrate | | |
| Poractant alpha | Pulmo ry Surfactants | |
| Pembrolizumab | Antineoplastic and | US2012135408 |
| | Immunomodulating Agents | |
| Peginterferon beta-1a | | |
| Ofatumumab | Antineoplastic and | U.S. Pat. No. 8,337,847 |
| | Immunomodulating Agents | |
| Obiltoxaximab | | |
| Nivolumab | Antineoplastic and | US2013173223 |
| | Immunomodulating Agents | |
| Necitumumab | | |
| Metreleptin | | US20070099836 |
| Methoxy polyethylene | | |
| glycol-epoetin beta | | |
| Mepolizumab | Antineoplastic and | US2008134721 |
| | Immunomodulating Agents, | |
| | Immunosuppressive Agents, | |
| | Interleukin Inhibitors | |
| Ixekizumab | | |
| Insulin Pork | Hypoglycemic Agents, | |
| | Antidiabetic Agents | |
| Insulin Degludec | | |
| Insulin Beef | | |
| Thyroglobulin | Hormone therapy | U.S. Pat. No. 5,099,001 |
| Anthrax immune globulin | Plasma derivative | |
| human | | |
| Anti-inhibitor coagulant | Blood Coagulation Factors, | |
| complex | Antihemophilic Agent | |
| Anti-thymocyte Globulin | Antibody | |
| (Equine) | | |
| Anti-thymocyte Globulin | Antibody | |
| (Rabbit) | | |
| Brodalumab | Antineoplastic and | |
| | Immunomodulating Agents | |
| C1 Esterase Inhibitor | Blood and Blood Forming Organs | |
| (Recombi nt) | | |
| Ca kinumab | Antineoplastic and | |
| | Immunomodulating Agents | |
| Chorionic Go dotropin | Hormones | U.S. Pat. No. 6,706,681 |
| (Human) | | |
| Chorionic Go dotropin | Hormones | U.S. Pat. No. 5,767,251 |
| (Recombi nt) | | |
| Coagulation factor X | Blood Coagulation Factors | |
| human | | |
| Dinutuximab | Antibody, Immunosuppresive | US20140170155 |
| | agent, Antineoplastic agent | |
| Efmoroctocog alpha | Antihemophilic Factor | |
| Factor IX Complex | Antihemophilic agent | |
| (Human) | | |
| Hepatitis A Vaccine | Vaccine | |
| Human Varicella-Zoster | Antibody | |
| Immune Globulin | | |
| Ibritumomab tiuxetan | Antibody, Immunosuppressive | CA2149329 |
| | Agents | |
| Lenograstim | Antineoplastic and | |
| | Immunomodulating Agents | |
| Pegloticase | Enzymes | |
| Protamine sulfate | Heparin Antagonists, Hematologic | |
| | Agents | |
| Protein S human | Anticoagulant plasma protein | |
| Sipuleucel-T | Antineoplastic and | U.S. Pat. No. 8,153,120 |
| | Immunomodulating Agents | |
| Somatropin recombi nt | Hormones, Hormone Substitutes, | CA1326439, CA2252535, |
| | and Hormone Antagonists | U.S. Pat. No. 5,288,703, |
| | | U.S. Pat. No. 5,849,700, |
| | | U.S. Pat. No. 5,849,704, |
| | | U.S. Pat. No. 5,898,030, |
| | | U.S. Pat. No. 6,004,297, |
| | | U.S. Pat. No. 6,152,897, |
| | | U.S. Pat. No. 6,235,004, |
| | | U.S. Pat. No. 6,899,699 |
| Susoctocog alpha | Blood coagulation factors, | |
| | Antihaemorrhagics | |
| Thrombomodulin alpha | Anticoagulant agent, Antiplatelet | |
| | agent | |

TABLE 29
Exemplary monoclonal antibody therapies.

| mAb | Target | Indication |
|---|---|---|
| Muromonab-CD3 | CD3 | Kidney transplant rejection |
| Abciximab | GPIIb/IIIa | Prevention of blood dots in angioplasty |
| Rituximab | CD20 | Non-Hodgkin lymphoma |
| Palivizumab | RSV | Prevention of respiratory syncytial virus |
| | | infection |
| Infliximab | TNFα | Crohn's disease |
| Trastuzumab | HER2 | Breast cancer |
| Alemtuzumab | CD52 | Chronic myeloid leukemia |
| Adalimumab | TNFα | Rheumatoid arthritis |
| Ibritumomab | CD20 | Non-Hodgkin lymphoma |
| tiuxetan | | |
| Omalizumab | IgE | Asthma |
| Cetuximab | EGER | Colorectal cancer |
| Bevacizumab | VEGF-A | Colorectal cancer |
| Natalizumab | ITGA4 | Multiple sclerosis |
| Panitumumab | EGFR | Colorectal cancer |
| Ranibizumab | VEGF-A | Macular degeneration |
| Eculizumab | C5 | Paroxysmal nocturnal hemoglobinuria |
| Certolizumab | TNFα | Crohn's disease |
| pegol | | |
| Ustekinumab | IL-12/23 | Psoriasis |
| Canakinumab | IL-1β | Muckle-Wells syndrome |
| Golimumab | TNFα | Rheumatoid and psoriatic arthritis, ankylosing |
| | | spondylitis |
| Ofatumumab | CD20 | Chronic lymphocytic leukemia |
| Tocilizumab | IL-6R | Rheumatoid arthritis |
| Denosumab | RANKL | Bone loss |
| Belimumab | BLyS | Systemic lupus erythematosus |
| Ipilimumab | CTLA-4 | Metastatic melanoma |
| Brentuximab | CD30 | Hodgkin lymphoma, systemic anaplastic large |
| vedotin | | cell lymphoma |
| Pertuzumab | HER2 | Breast Cancer |
| Trastuzumab | HER2 | Breast cancer |
| emtansine | | |
| Raxibacumab | B. anthrasis PA | Anthrax infection |
| Obinutuzumab | CD20 | Chronic lymphocytic leukemia |
| Siltuximab | IL-6 | Castleman disease |
| Ramucirumab | VEGFR2 | Gastric cancer |
| Vedolizumab | α4β7 integrin | Ulcerative colitis, Crohn disease |
| Blinatumomab | CD19, CD3 | Acute lymphoblastic leukemia |
| Nivolumab | PD-1 | Melanoma, non-small cell lung cancer |
| Pembrolizumab | PD-1 | Melanoma |
| Idarucizumab | Dabigatran | Reversal of dabigatran-induced anticoagulation |
| Necitumumab | EGFR | Non-small cell lung cancer |
| Dinutuximab | GD2 | Neuroblastoma |
| Secukinumab | IL-17α | Psoriasis |
| Mepolizumab | IL-5 | Severe eosinophilic asthma |
| Alirocurnab | PCSK9 | High cholesterol |
| Evoloeumab | PCSK9 | High cholesterol |
| Daratumumab | CD38 | Multiple myeloma |
| Elotuzumab | SLAMF7 | Muitiple myeloma |
| Ixekizumab | IL-17α | Psoriasis |
| Reslizumab | IL-5 | Asthma |
| Olaratumab | PDGFRα | Soft tissue sarcoma |
| Bezlotoxumab | Clostridium | Prevention of Clostridium difficile infection |
| | difficile enterotoxin B | recurrence |
| Atezolizumab | PD-L1 | Bladder cancer |
| Obiltoxaximab | B. anthrasis PA | Prevention of inhalational anthrax |
| Inotuzumab | CD22 | Acute lymphoblastic leukemia |
| ozogamicin | | |
| Brodalumab | IL-17R | Plaque psoriasis |
| Guselkumab | IL-23 p19 | Plaque psoriasis |
| Dupilumab | IL-4Rα | Atopic dermatitis |
| Sarilumab | IL-6R | Rheumatoid arthritis |
| Avelumab | PD-L1 | Merkel cell carcinoma |
| Ocrelizumab | CD20 | Multiple sclerosis |
| Emicizumab | Factor IXa, X | Hemophilia A |
| Benralizumab | IL-5Rα | Asthma |
| Gemtuzumab | CD33 | Acute myeloid leukemia |
| ozogamicin | | |
| Durvalumab | PD-L1 | Bladder cancer |
| Burosumab | FGF23 | X-linked hypophosphatemia |
| Lanadelumab | Plasma kallikrein | Hereditary angioedema attacks |
| Mogamulizumab | CCR4 | Mycosis fungoides or Sézary syndrome |
| Erenumab | CGRPR | Migraine prevention |
| Galcanezumab | CGRP | Migraine prevention |
| Tildrakizumab | IL-23 p19 | Plaque psoriasis |
| Cemiplimab | PD-1 | Cutaneous squamous cell carcinoma |
| Emapalumab | IFNγ | Primary hemophagocytic lymphohistiocytosis |
| Fremanezumab | CGRP | Migraine prevention |
| Ibalizumab | CD4 | HIV infection |
| Moxetumomab | CD22 | Hairy cell leukemia |
| pasudodox | | |
| Ravulizumab | C5 | Paroxysmal nocturnal hemoglobinuria |
| Caplacizumab | von Willebrand factor | Acquired thrombotic thrombocytopenic purpura |
| Romosozumab | Sclerostin | Osteoporosis in postmenopausal women at |
| | | increased risk of fracture |
| Risankizumab | IL-23 p19 | Plaque psoriasis |
| Polatuzumab | CD79β | Diffuse large B-cell lymphoma |
| vedotin | | |
| Brolucizumab | VEGF-A | Macular degeneration |
| Crizanlizumab | P-selectin | Sickle cell disease |

### Plant-Modification Methods

Gene Writer systems described herein may be used to modify a plant or a plant part (e.g., leaves, roots, flowers, fruits, or seeds), e.g., to increase the fitness of a plant.

A. Delivery to a Plant

Provided herein are methods of delivering a Gene Writer system described herein to a plant. Included are methods for delivering a Gene Writer system to a plant by contacting the plant, or part thereof, with a Gene Writer system. The methods are useful for modifying the plant to, e.g., increase the fitness of a plant.

More specifically, in some embodiments, a nucleic acid described herein (e.g., a nucleic acid encoding a GeneWriter) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime-editing systems for precise genome editing, 2020, Plant Communications.

In one aspect, provided herein is a method of increasing the fitness of a plant, the method including delivering to the plant the Gene Writer system described herein (e.g., in an effective amount and duration) to increase the fitness of the plant relative to an untreated plant (e.g., a plant that has not been delivered the Gene Writer system).

An increase in the fitness of the plant as a consequence of delivery of a Gene Writer system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2×-fold, 5×-fold, 10×-fold, 25×-fold, 50×-fold, 75×-fold, 100×-fold, or more than 100×-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a Gene Writer system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents.

Accordingly, provided herein is a method of modifying a plant, the method including delivering to the plant an effective amount of any of the Gene Writer systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some instances, the increase in plant fitness is an increase (e.g., by about 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the Gene Writer systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the Gene Writer systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the Gene Writer system described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

B. Application Methods

A plant described herein can be exposed to any of the Gene Writer system compositions described herein in any suitable manner that permits delivering or administering the composition to the plant. The Gene Writer system may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the plant-modifying composition. Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the Gene Writer system is delivered to a plant, the plant receiving the Gene Writer system may be at any stage of plant growth. For example, formulated plant-modifying compositions can be applied as a seed-coating or root treatment in early stages of plant growth or as a total plant treatment at later stages of the crop cycle. In some instances, the plant-modifying composition may be applied as a topical agent to a plant.

Further, the Gene Writer system may be applied (e.g., in the soil in which a plant grows, or in the water that is used to water the plant) as a systemic agent that is absorbed and distributed through the tissues of a plant. In some instances, plants or food organisms may be genetically transformed to express the Gene Writer system.

Delayed or continuous release can also be accomplished by coating the Gene Writer system or a composition with the plant-modifying composition(s) with a dissolvable or bioerodable coating layer, such as gelatin, which coating dissolves or erodes in the environment of use, to then make the plant-modifying com Gene Writer system position available, or by dispersing the agent in a dissolvable or erodable matrix. Such continuous release and/or dispensing means devices may be advantageously employed to consistently maintain an effective concentration of one or more of the plant-modifying compositions described herein.

In some instances, the Gene Writer system is delivered to a part of the plant, e.g., a leaf, seed, pollen, root, fruit, shoot, or flower, or a tissue, cell, or protoplast thereof. In some instances, the Gene Writer system is delivered to a cell of the plant. In some instances, the Gene Writer system is delivered to a protoplast of the plant. In some instances, the Gene Writer system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the Gene Writer system is delivered to a plant embryo.

C. Plants

A variety of plants can be delivered to or treated with a Gene Writer system described herein. Plants that can be delivered a Gene Writer system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the Gene Writer system is delivered to a plant part, the plant part may be modified by the plant-modifying agent. Alternatively, the Gene Writer system may be distributed to other parts of the plant (e.g., by the plant's circulatory system) that are subsequently modified by the plant-modifying agent.

### Delivery Modalities

Nucleic acid elements of systems provided by the invention, used in the methods provided by the invention, can be delivered by a variety of modalities. In embodiments where the system comprises two separate nucleic acid molecules (e.g., the transposase and template nucleic acids are separate molecules), the two molecules may be delivered by the same modality, while in other embodiments, the two molecules are delivered by different modalities. The composition and systems described herein may be used in vitro or in vivo. In some embodiments the system or components of the system are delivered to cells (e.g., mammalian cells, e.g., human cells), e.g., in vitro, ex vivo, or in vivo. In some embodiments, the cells are eukaryotic cells, e.g., cells of a multicellular organism, e.g., an animal, e.g., a mammal (e.g., human, swine, bovine) a bird (e.g., poultry, such as chicken, turkey, or duck), or a fish. In some embodiments, the cells are non-human animal cells (e.g., a laboratory animal, a livestock animal, or a companion animal). In some embodiments, the cell is a stem cell (e.g., a hematopoietic stem cell), a fibroblast, or a T cell. In some embodiments, the cell is a non-dividing cell, e.g., a non-dividing fibroblast or non-dividing T cell. The skilled artisan will understand that the components of the Gene Writer™ system may be delivered in the form of polypeptide, nucleic acid (e.g., DNA, RNA), and combinations thereof.

For instance, delivery can use any of the following combinations for delivering the transposase (e.g., as DNA encoding the transposase protein, as RNA encoding the transposase protein, or as the protein itself) and the template nucleic acid (e.g., as DNA):
Transposase DNA+template DNA
Transposase RNA+template DNA
Transposase protein+template DNA
Transposase virus+template virus
Transposase virus+template DNA
Transposase DNA+template virus
Transposase RNA+template virus
Transposase protein+template virus

As indicated above, in some embodiments, the DNA or RNA that encodes the transposase protein is delivered using a virus (e.g. an AAV), and in some embodiments, the template DNA is delivered using a virus (e.g., an AAV). In some embodiments, the template DNA is delivered using a virus (e.g., an AAV), and the transposase is delivered via an mRNA encoding the transposase, formulated as an LNP. In some embodiments, a template DNA suitable for delivery using AAV comprises a sequence that promotes packaging by the AAV capsid (e.g., ITRs), and a sequence that promotes association with the transposase (e.g., IRs).

In some embodiments the system and/or components of the system are delivered as nucleic acid. For example, the Gene Writer™ polypeptide may be delivered in the form of a DNA or RNA encoding the polypeptide, and the template DNA may be delivered in the form of DNA. In some embodiments, the system or components of the system are delivered on 1, 2, 3, 4, or more distinct nucleic acid molecules. In some embodiments the system or components of the system are delivered as a combination of DNA and RNA. In some embodiments, the system or components of the system are delivered as a combination of DNA and protein. In some embodiments, the Gene Writer™ genome editor polypeptide is delivered as a protein.

In some embodiments, the system or components of the system are delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

A variety of nanoparticles can be used for delivery, such as a liposome, a lipid nanoparticle, a cationic lipid nanoparticle, an ionizable lipid nanoparticle, a polymeric nanoparticle, a gold nanoparticle, a dendrimer, a cyclodextrin nanoparticle, a micelle, or a combination of the foregoing.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

A variety of nanoparticles can be used for delivery, such as a liposome, a cationic lipid nanoparticle, an ionizable lipid nanoparticle, a polymeric nanoparticle, a gold nanoparticle, a dendrimer, a cyclodextrin nanoparticle, a micelle, or a combination of the foregoing.

Exemplary nanoparticles include lipid nanoparticles (LNPs), which are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see for example Patent Application WO2020014209, the teachings of which relating to fusosome design, preparation, and usage are incorporated herein by reference).

Host factors known to involved in transposition are known in the literature, e.g., a DNA-bending protein, such as the DNA-bending protein HMGB1 (Zayed et al. Nucleic Acids Res 2003). In some embodiments, the Gene Writer™ system also comprises a composition for transiently expressing a DNA-bending factor in the recipient cell. In some embodiments, the Gene Writer™ system also comprises a composition for transiently increasing the amount of HMGB1 in the recipient cell. In some embodiments, HMGB1 protein, (or DNA or RNA encoding the HMGB1 protein), may be provided to the cell. In some embodiments, the nucleic acid encoding HMGB1 may be on the same molecule as the nucleic acid encoding the transposase. In some embodiments, the nucleic acid encoding HMGB1 may be on a separate nucleic acid. It is understood that, similarly to the other components of the system, the nucleic acid encoding HMGB1 may be provided in a delivery system in conjunction with or separately from the other components of the Gene Writing™ system, e.g., virus, vesicle, LNP, exosome, fusosome.

In some embodiments, the protein component(s) of the Gene Writing™ system may be pre-associated with the DNA template. For example, in some embodiments, the Gene Writer™ polypeptide may be first combined with the DNA template to form a deoxyribonucleoprotein (DNP) complex. In some embodiments, the DNP may be delivered to cells via, e.g., transfection, nucleofection, virus, vesicle, LNP, exosome, fusosome. In some embodiments, the template DNA may be first associated with a DNA-bending factor, e.g., HMGB1, in order to facilitate excision and transposition when subsequently contacted with the transposase component. Additional description of DNP delivery is found, for example, in Guha and Calos J Mol Biol (2020), which is herein incorporated by reference in its entirety.

A Gene Writer™ system can be introduced into cells, tissues and multicellular organisms. In some embodiments the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

### Lipid Nanoparticles

The methods and systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the Gene Writer or template nucleic acid) can be varied as desired. For example, the total lipid to nucleic acid (mass or weight) ratio can be from about 10:1 to about 30:1.

In some embodiments, an ionizable lipid may be a cationic lipid, a ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a GeneWriter), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., a mRNA encoding the Gene Writer polypeptide.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13,16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety). In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01) e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(24(2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is; Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

Some non-limiting example of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a GeneWriter) includes,

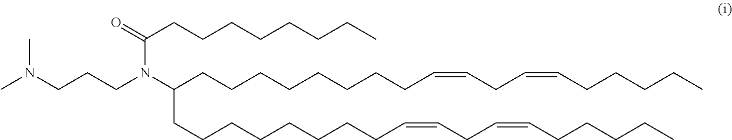 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a GeneWriter composition described herein to the liver and/or hepatocyte cells.

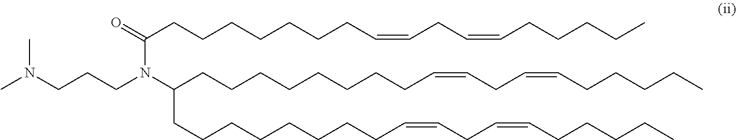 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a GeneWriter composition described herein to the liver and/or hepatocyte cells.

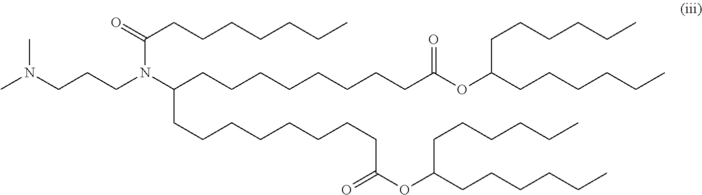 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a GeneWriter composition described herein to the liver and/or hepatocyte cells.

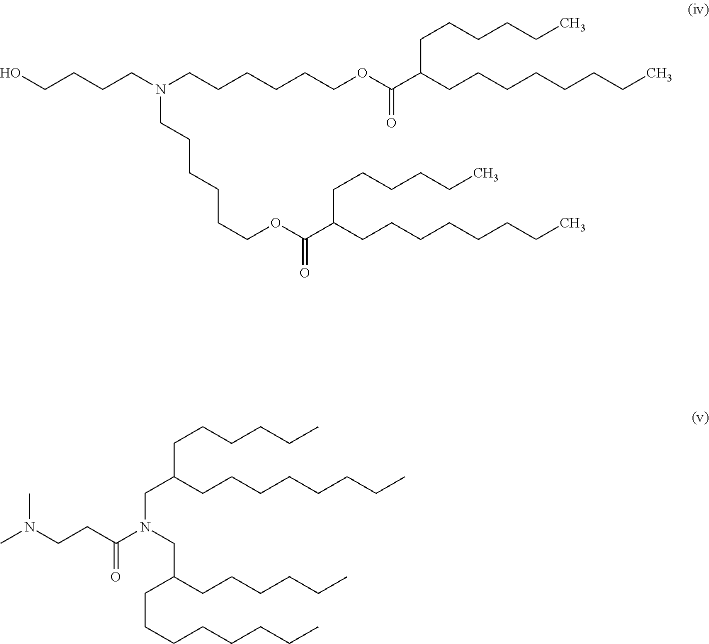

In some embodiments an LNP comprising Formula (v) is used to deliver a GeneWriter composition described herein to the liver and/or hepatocyte cells.

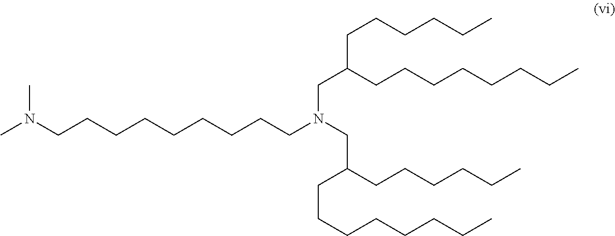 [SMILES: CCCCCCCCC(CCCCCC)CN(CCCCCCCCCN(C)C)CC(CCCCCC)CCCCCCCC]

In some embodiments an LNP comprising Formula (vi) is used to deliver a GeneWriter composition described herein to the liver and/or hepatocyte cells.

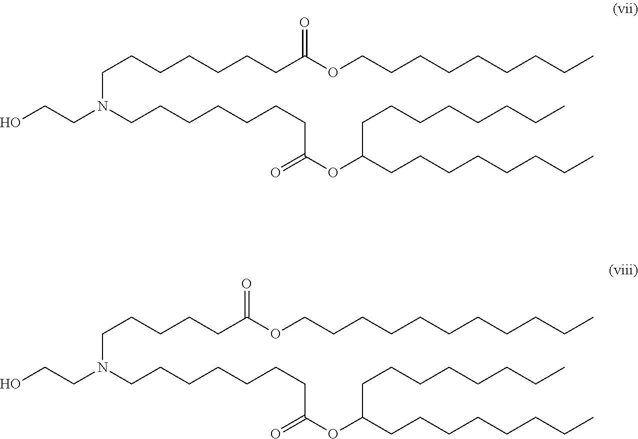 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a GeneWriter composition described herein to the liver and/or hepatocyte cells.

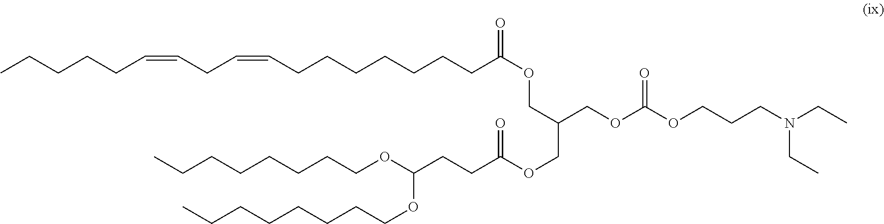 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a GeneWriter composition described herein to the liver and/or hepatocyte cells.

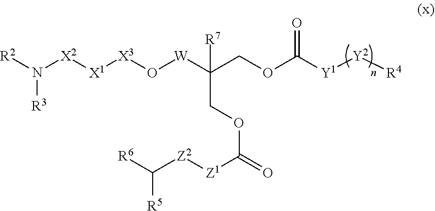

wherein
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, Of R² taken together with R³ and the nitrogen atom to which they are attached form a 5-, 6-, or 7-membered ring, Y¹ is C2-12 alkylene, Y² is selected from

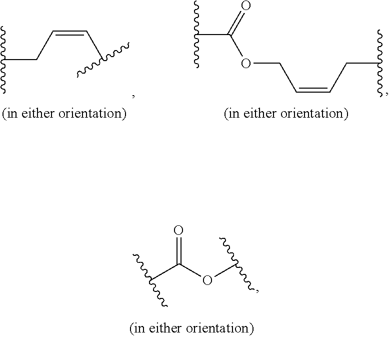 [SMILES: */C=C\C* ; *C/C=C\COC(*)=O ; *OC(*)=O]

n is to 3, R⁴ is Ci-15 alkyl, Z¹ is Ci-6 alkylene or a direct bond,
Z² is

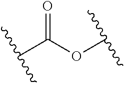 [SMILES: *OC(*)=O]

(in either orientation) or absent, provided that if Z¹ is a direct bond, Z is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

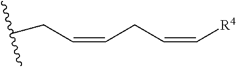

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a GeneWriter composition described herein to the liver and/or hepatocyte cells.

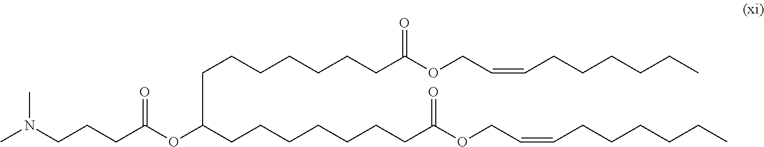 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a GeneWriter composition described herein to the liver and/or hepatocyte cells.

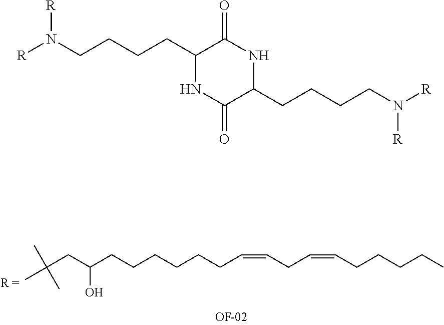

where R=

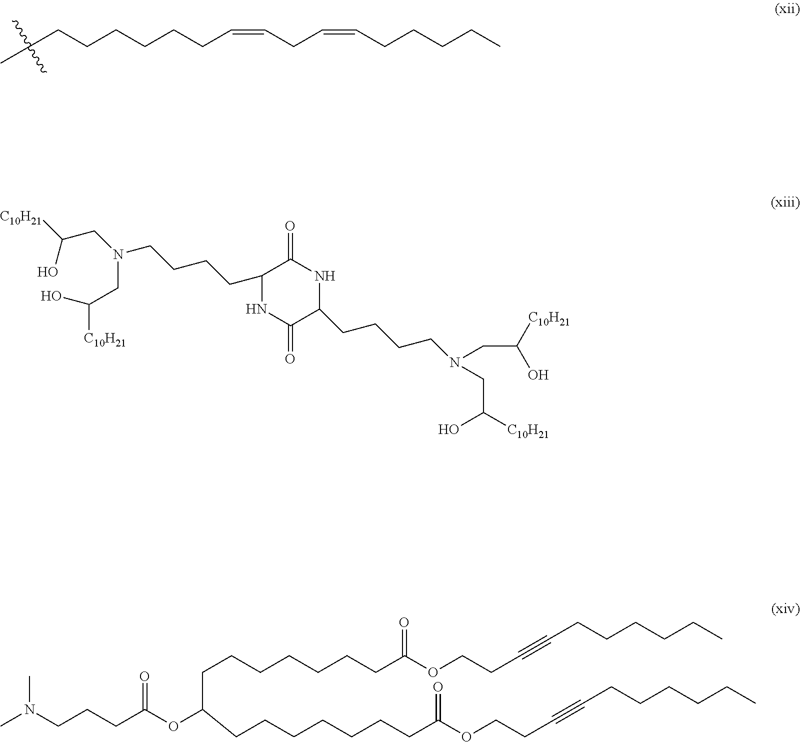

In some embodiments an LNP comprises a compound of Formula (xiii) and a compound of Formula (xiv).

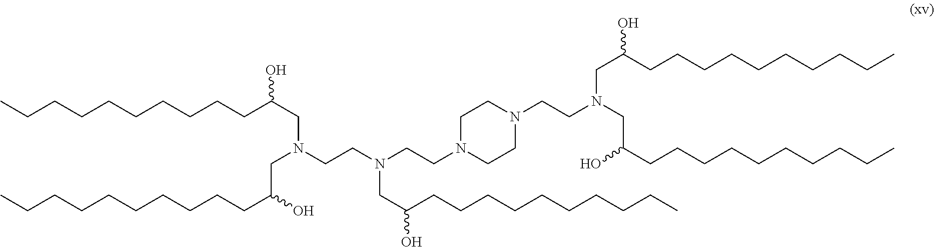 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a GeneWriter composition described herein to the liver and/or hepatocyte cells.

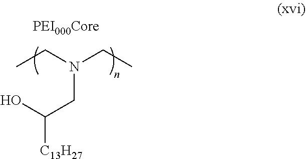

In some embodiments an LNP comprising a formulation of Formula (xvi) is used to deliver a GeneWriter composition described herein to the lung endothelial cells.

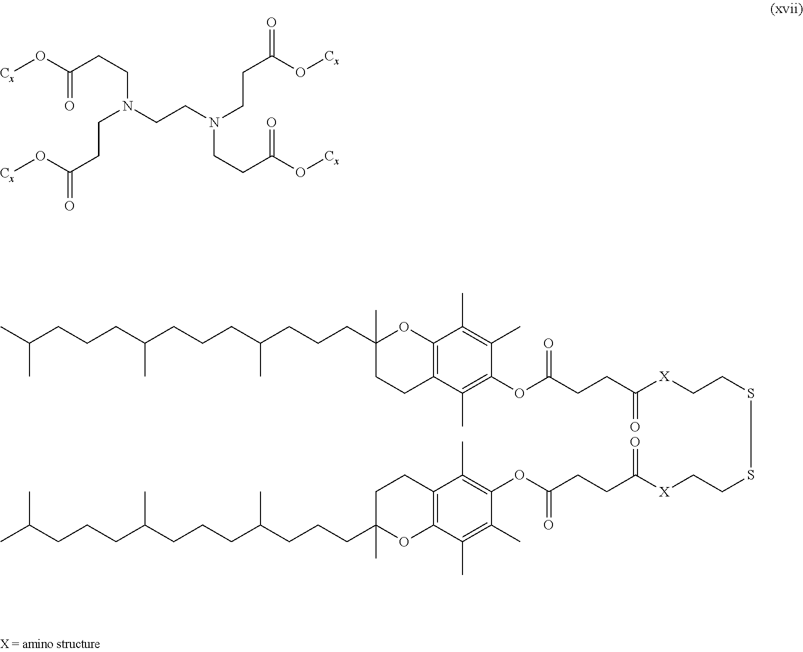

where X=

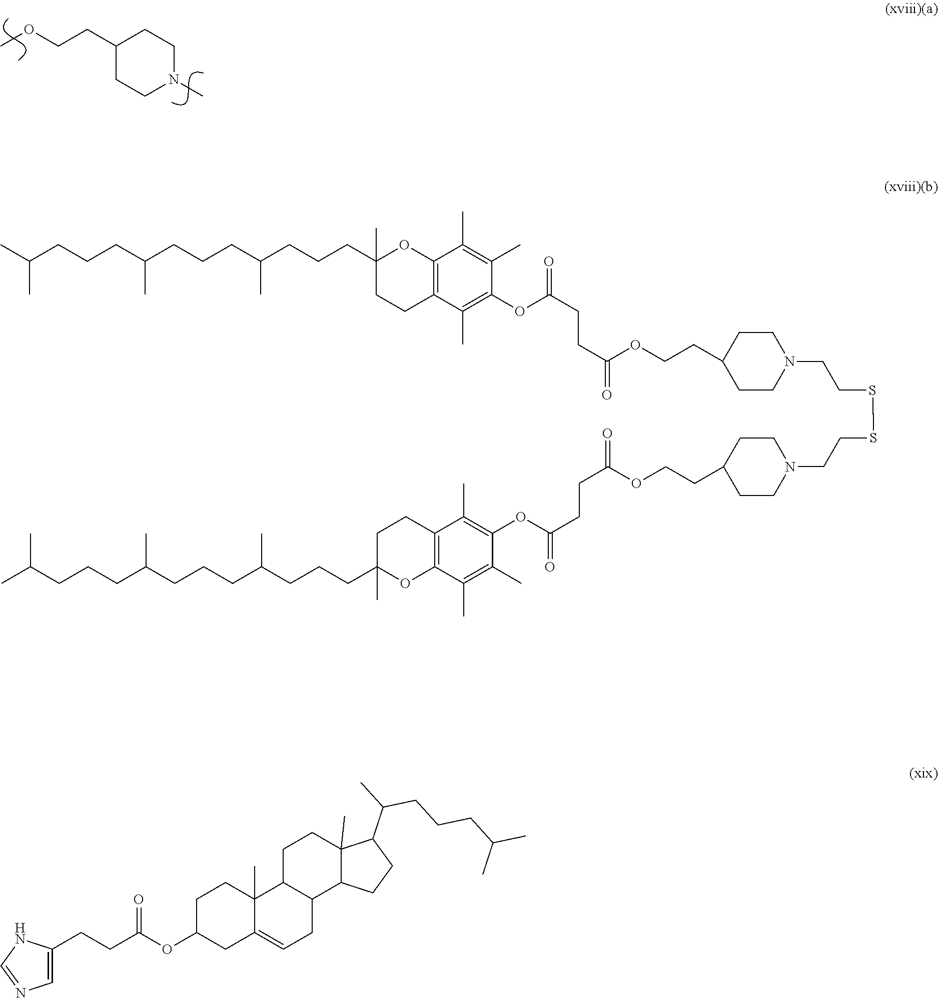

In some embodiments, a lipid compound used to form lipid nanoparticles for the delivery of compositions described herein, e.g., nucleic acid (e.g., RNA) described herein (e.g., a template nucleic acid or a nucleic acid encoding a GeneWriter) is made by one of the following reactions:

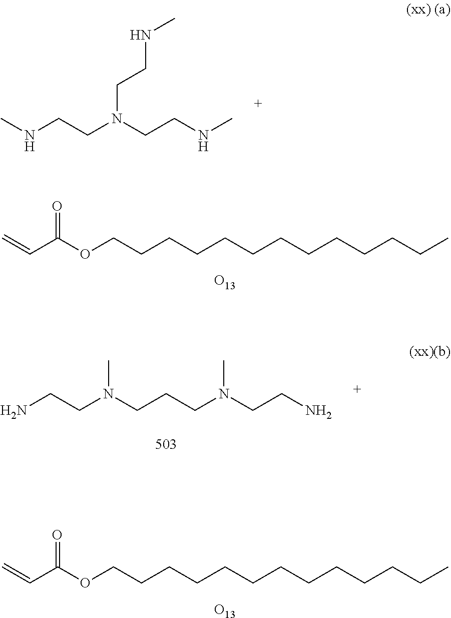

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), 1,2-dioleoyl-sn-glycero-3-phosphoethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS). In some embodiments, the non-cationic lipid may have the following structure

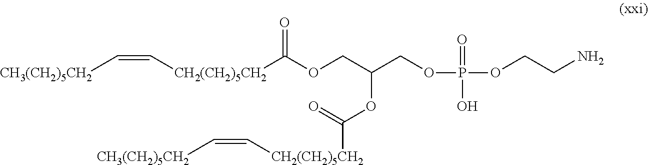

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, 1,2-dimyristoyl-sn-glycerol, methoxypoly ethylene glycol (DMG-PEG-2K), or a mixture thereof. Additional exemplary PEG-lipid conjugates are described, for example, in U.S. Pat. No. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

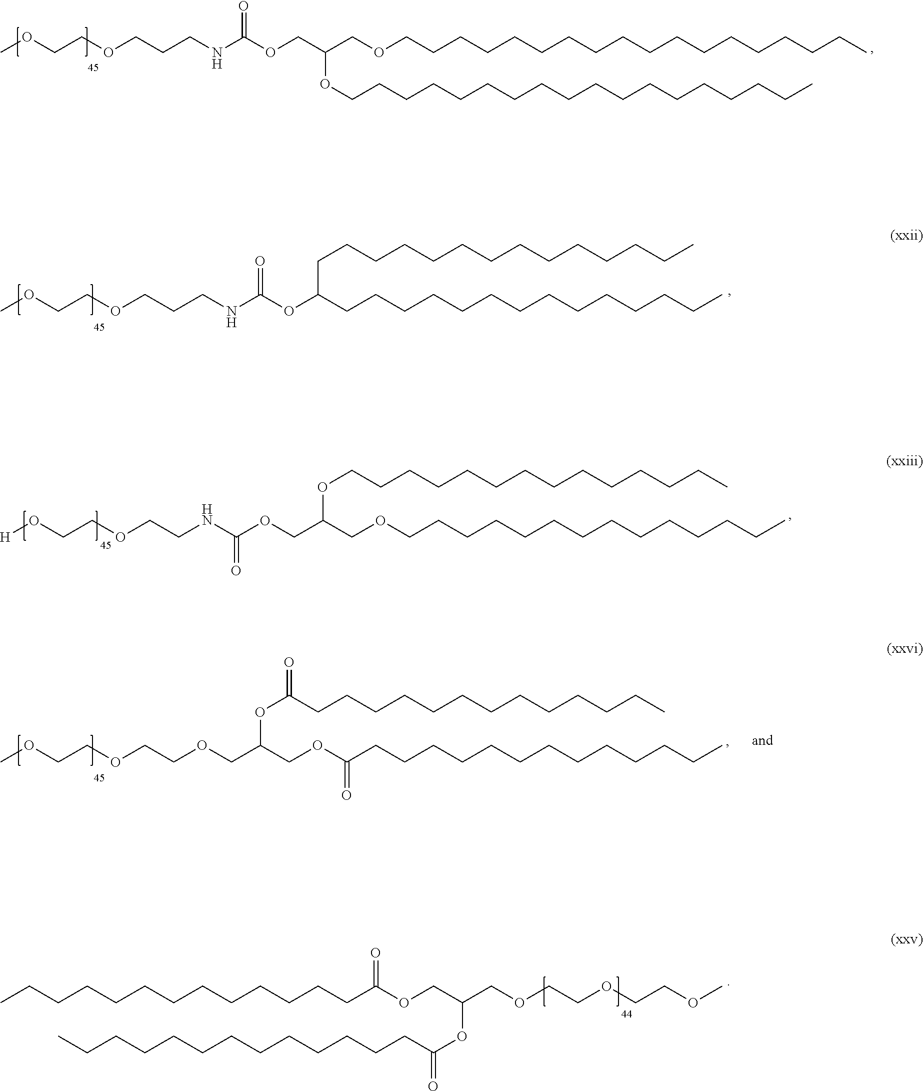

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments an LNP comprises a compound of Formula (xix), a compound of Formula (xxi) and a compound of Formula (xxv). In some embodiments a LNP comprising a formulation of Formula (xix), Formula (xxi) and Formula (xxv) is used to deliver a GeneWriter composition described herein to the lung or pulmonary cells.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5: 1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts).

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., according to the method described in Example 6. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., as described in Example 7. In embodiments, chemical modifications of a nucleic acid molecule, nucleotide, or nucleoside are detected by determining the presence of one or more modified nucleotides or nucleosides, e.g., using LC-MS/MS analysis, e.g., as described in Example 7.

In some embodiments, a nucleic acid (e.g., RNA) described herein (e.g., a template nucleic acid or a nucleic acid encoding a GeneWriter) does not comprise an aldehyde modification or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotide.

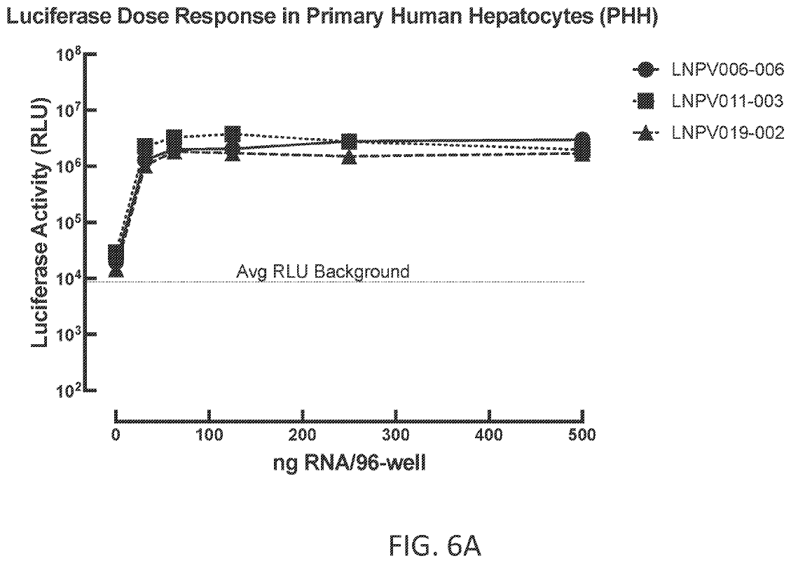

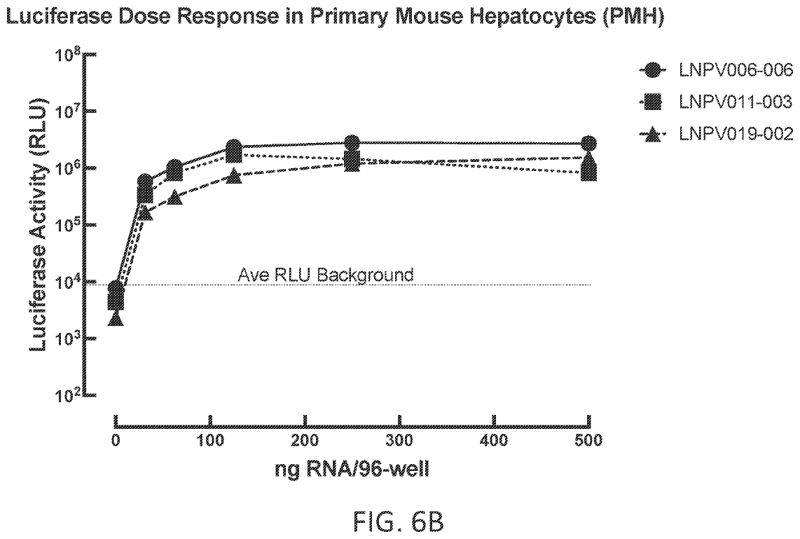

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a Gene Writer system may be prepared as a single LNP formulation, e.g., an LNP formulation comprises mRNA encoding for the Gene Writer polypeptide and an RNA template. Ratios of nucleic acid components may be varied in order to maximize the properties of a therapeutic. In some embodiments, the ratio of RNA template to mRNA encoding a Gene Writer polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a Gene Writer polypeptide. In some embodiments, the system may comprise more than two nucleic acid components formulated into LNPs. In some embodiments, the system may comprise a protein, e.g., a Gene Writer polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20. In some embodiments, the polydispersity index of a LNP is about 0.01-0.1, e.g., about 0.02-0.06, e.g., about 0.04.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., Gene Writer polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of Gene Writer LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 10¹⁴ vg/kg.

Viral Vectors Incorporated into Gene Writing™ Systems

One particular embodiment useful for delivering all or part of a system provided by the invention, e.g., for use in methods provided by the invention, include viral vectors. Viral packaging of nucleic acids is an approach well-known in the art for facilitating delivery of nucleic acids into target cells. Systems derived from different viruses have been employed for the delivery of transposons, e.g., integrase-deficient lentivirus, adenovirus, adeno-associated virus (AAV), herpes simplex virus, and baculovirus (reviewed in Hodge et al. Hum Gene Ther 2017; Narayanavari et al. Crit Rev Biochem Mol Biol 2017; Boehme et al. Curr Gene Ther 2015).

Adenoviruses are common viruses that have long been used as gene delivery vehicles given well-defined biology, genetic stability, high transduction efficiency, and ease of large-scale production (see, for example, review by Lee et al. Genes & Diseases 2017). They possess linear dsDNA genomes and come in a variety of serotypes that differ in tissue and cell tropisms. In order to prevent replication of infectious virus in recipient cells, adenovirus genomes used for packaging are deleted of some or all endogenous viral proteins, which are provided in trans in viral production cells. This renders the genomes helper-dependent, meaning they can only be replicated and packaged into viral particles in the presence of the missing components provided by so-called helper functions. A helper-dependent adenovirus system with all viral ORFs removed may be compatible with packaging foreign DNA of up to ˜37 kb (Parks et al. J Virol 1997). In some embodiments, an adenoviral vector is used to deliver DNA corresponding to the transposase or DNA template component of the Gene Writing™ system, or both are contained on separate or the same adenoviral vector. In some embodiments, the adenovirus is a helper-dependent adenovirus (HD-AdV) that is incapable of self-packaging. In some embodiments, the adenovirus is a high-capacity adenovirus (HC-AdV) that has had all or a substantial portion of endogenous viral ORFs deleted, while retaining the necessary sequence components for packaging into adenoviral particles. For this type of vector, the only adenoviral sequences required for genome packaging are noncoding sequences: the inverted terminal repeats (ITRs) at both ends and the packaging signal at the 5′-end (Jager et al. Nat Protoc 2009). In some embodiments, the adenoviral genome also comprises stuffer DNA to meet a minimal genome size for optimal production and stability (see, for example, Hausl et al. Mol Ther 2010). Adenoviruses have been used in the art for the delivery of transposons to various tissues. In some embodiments, an adenovirus is used to deliver a Gene Writing™ system to the liver. In some embodiments, a HC-AdV construct based on Ad5 is used to deliver a Gene Writing™ system to the liver (see, for example, HC-AdV as described in Jager et al. Nat Protoc 2009). For example, a high-capacity adenoviral vector (HC-AdV) was used to deliver a Sleeping Beauty system to integrate cFIX to complement hemophilia B in canines (Hausl et al. Mol Ther 2010). In some embodiments, an adenovirus is used to deliver a Gene Writing™ system to lung tissue. In some embodiments, the adenovirus delivering a Gene Writing™ system to lung tissue is a serotype previously shown to reach this tissue, e.g., Ad5 (Cooney et al. Mol Ther 2015).

In some embodiments, an adenovirus is used to deliver a Gene Writing™ system to HSCs, e.g., HDAd5/35⁺⁺. HDAd5/35⁺⁺ is an adenovirus with modified serotype 35 fibers that de-target the vector from the liver (Wang et al. Blood Adv 2019). In some embodiments, the adenovirus that delivers a Gene Writing™ system to HSCs utilizes a receptor found abundantly expressed specifically on primitive HSCs, e.g., CD46.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129). In some embodiments, one or more Gene Writing™ nucleic acid components is flanked by ITRs derived from AAV for viral packaging. See, e.g., WO2019113310.

In some embodiments, one or more components of the Gene Writing™ system are carried via at least one AAV vector. In some embodiments, the at least one AAV vector is selected for tropism to a particular cell, tissue, organism. In some embodiments, the AAV vector is pseudotyped, e.g., AAV2/8, wherein AAV2 describes the design of the construct but the capsid protein is replaced by that from AAV8. It is understood that any of the described vectors could be pseudotype derivatives, wherein the capsid protein used to package the AAV genome is derived from that of a different AAV serotype. Without wishing to be limited in vector choice, a list of exemplary AAV serotypes can be found in Table 5. In some embodiments, an AAV to be employed for Gene Writing™ may be evolved for novel cell or tissue tropism as has been demonstrated in the literature (e.g., Davidsson et al. Proc Natl Acad Sci USA 2019).

In some embodiments, the AAV delivery vector is a vector which has two AAV inverted terminal repeats (ITRs) and a nucleotide sequence of interest (for example, a sequence coding for a Gene Writer™ polypeptide or a DNA template, or both), each of said ITRs having an interrupted (or noncontiguous) palindromic sequence, i.e., a sequence composed of three segments: a first segment and a last segment that are identical when read 5′→3′ but hybridize when placed against each other, and a segment that is different that separates the identical segments. Such sequences, notably the ITRs, form hairpin structures. See, for example, WO2012123430.

The term “inverted terminal repeats” or “ITRs” as used herein refers to AAV viral cis-elements named so because of their symmetry. These elements are essential for efficient multiplication of an AAV genome. It is hypothesized that the minimal defining elements indispensable for ITR function are a Rep-binding site (RBS; 5′-GCGCGCTCGCTCGCTC-3′ (SEQ ID NO: 1582) for AAV2) and a terminal resolution site (TRS; 5′-AGTTGG-3′ for AAV2) plus a variable palindromic sequence allowing for hairpin formation. According to the present invention, an ITR comprises at least these three elements (RBS, TRS and sequences allowing the formation of an hairpin). In addition, in the present invention, the term “ITR” refers to ITRs of known natural AAV serotypes (e.g. ITR of a serotype 1, 2, 3, 4, 5, 6, 7, 8, 9, 10 or 11 AAV, or any ITRs of serotypes present in Table 5), to chimeric ITRs formed by the fusion of ITR elements derived from different serotypes, and to functional variant thereof. By functional variant of an ITR, it is referred to a sequence presenting a sequence identity of at least 80%, 85%, 90%, preferably of at least 95% with a known ITR, allowing multiplication of the sequence that includes said ITR in the presence of Rep proteins.

Conventionally, AAV virions with capsids are produced by introducing a plasmid or plasmids encoding the rAAV or scAAV genome, Rep proteins, and Cap proteins (Grimm et al, 1998). Upon introduction of these helper plasmids in trans, the AAV genome is “rescued” (i.e., released and subsequently recovered) from the host genome, and is further encapsidated to produce infectious AAV. In some embodiments, one or more Gene Writing™ nucleic acids are packaged into AAV particles by introducing the ITR-flanked nucleic acids into a packaging cell in conjunction with the helper functions.

In some embodiments, the AAV genome is a so called self-complementary genome (referred to as scAAV), such that the sequence located between the ITRs contains both the desired nucleic acid sequence (e.g., DNA encoding the Gene Writer™ polypeptide or template DNA, or both) in addition to the reverse complement of the desired nucleic acid sequence, such that these two components can fold over and self-hybridize. In some embodiments, the self-complementary modules are separated by an intervening sequence that permits the DNA to fold back on itself, e.g., forms a stem-loop. An scAAV has the advantage of being poised for transcription upon entering the nucleus, rather than being first dependent on ITR priming and second-strand synthesis to form dsDNA. In some embodiments, one or more Gene Writing™ components is designed as an scAAV, wherein the sequence between the AAV ITRs contains two reverse complementing modules that can self-hybridize to create dsDNA.

TABLE 5
Viral delivery modalities

| Target | | |
|---|---|---|
| Tissue | Vehicle | Reference |
| Liver | AAV (AAV8¹, AAVrh.8¹, | 1. Wang et al., Mol. Ther. 18, |
| | AAVhu.37¹, AAV2/8, | 118-25 (2010) |
| | AAV2/rh10², AAV9, AAV2, | 2. Ginn et al., JHEP Reports, |
| | NP40³, NP59^{2, 3}, AAV3B⁵, | 100065 (2019) |
| | AAV-DJ⁴, AAV-LK01⁴, | 3. Paulk et al., Mol. Ther. 26, |
| | AAV-LK02⁴, AAV-LK03⁴, | 289-303 (2018). |
| | AAV-LK19⁴ | 4. L. Lisowski et al., Nature. |
| | Adenovirus (Ad5, HC-AdV⁶) | 506, 382-6 (2014). |
| | | 5. L. Wang et al., Mol. Ther. |
| | | 23, 1877-87 (2015). |
| | | 6. Hausl Mol Ther (2010) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV6¹, AAV-LK19² | 1. Petek et al., Mol. Ther. |
| | | (2010) |
| | | 2. L. Lisowski et al., Nature. |
| | | 506, 382-6 (2014). |
| HSCs | HDAd5/35⁺⁺ | Wang et al. Blood Adv (2019) |

In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a protein described herein and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a Gene Writer described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific Gene Writing, the expression of the Gene Writer and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a Gene Writer, promoter, and transcription terminator can fit into a single viral vector. SpCas9 (4.1 kb) may, in some instances, be difficult to package into AAV. Therefore, in some embodiments, a Gene Writer is used that is shorter in length than other Gene Writers or base editors. In some embodiments, the Gene Writers are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAVS or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol. 82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV 12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 5 herein.

In some embodiments, a pharmaceutical composition (e.g., comprising an AAV as described herein) has less than 10% empty capsids, less than 8% empty capsids, less than 7% empty capsids, less than 5% empty capsids, less than 3% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ pg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ pg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10⁵ pg by 1.0×10¹³ vg, less than 8.0×10⁵ pg by 1.0×10¹³ vg or less than 6.8×10⁵ pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP <85> (incorporated by reference in its entirety) is less than 1.0 EU/mL, less than 0.8 EU/mL or less than 0.75 EU/mL. In embodiments, the osmolarity of a pharmaceutical composition according to USP, for example, USP <785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 μm per container, less than 1000 particles that are greater than 25 μm per container, less than 500 particles that are greater than 25 μm per container or any intermediate value. In embodiments, the pharmaceutical composition contains less than 10,000 particles that are greater than 10 μm per container, less than 8000 particles that are greater than 10 μm per container or less than 600 particles that are greater than 10 pm per container.

In one embodiment, the pharmaceutical composition has a genomic titer of 0.5 to 5.0×10¹³ vg/mL, 1.0 to 4.0×10¹³ vg/mL, 1.5 to 3.0×10¹³ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10⁵ pg of residual DNA plasmid per 1.0×10¹³ vg, less than about 1.1×10⁵ pg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 μm in size per container, less than about 6000 particles that are >10 μm in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 μg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between ±15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### EXEMPLIFICATION

### Example 1: Application of a Gene Writer™ System for Delivering Therapeutic Gene to Liver in a Human Chimeric Liver Mouse Model

This example describes a Gene Writer™ genome editing system delivered to the liver in vivo for integration and stable expression of a genetic payload. The promoter and miRNA recognition sequence for expression control and the therapeutic gene are intended to exemplify the approach and are selected from Tables 2, 3, and 4, respectively.

In this example, human hepatocytes derived from patients with OTC deficiency are engrafted into a mouse model (Ginn et al JHEP Reports 2019) and a Gene Writer™ system is used to deliver an OTC expression cassette for integration into liver cells. The Gene Writer™ polypeptide component comprises an expression cassette for the Sleeping Beauty transposase derivative SB100X (Table 1) and the template component comprises an expression cassette for the human OTC gene (Table 4) flanked by the IR/DR sequences required for binding and mobilization by SB100X. In this example, both the transposase and template expression cassettes additionally comprise the hAAT promoter (Table 2) for hepatocyte-specific expression and a miRNA recognition sequence complementary to the seed sequence of miR-142 (Table 3) for downregulating expression in hematopoetic cells.
Gene Writer™ polypeptide component: rAAV2/NP59.hAAT.SB100X
Mutated Gene Writer™ polypeptide: rAAV2/NP59.hAAT.dSB100X
Gene Writer™ template component: rAAV2/NP59.hAAT.OTC
Reporter Gene Writer™ template component: rAAV2/NP59.hAAT.GFP

Eight to 12-week-old female Fah^{-/-}Rag2^{-/-}Il2rg^{-/-} (FRG) mice are engrafted with human hepatocytes, isolated from pediatric donors or purchased from Lonza (Basel, Switzerland), as described previously (Azuma et al Nat Biotechnol 2007). Engrafted mice are cycled on and off 2-(2-nitro-4-trifluoro-methylbenzoyl)-1,3-cyclohexanedione (NTBC) in drinking water to promote liver repopulation. Blood is collected every two weeks and at the end of the experiment to measure the levels of human albumin, used as a marker to estimate the level of engraftment, in serum by enzyme-linked immunosorbent assay (ELISA; B ethyl Laboratories, Inc., Montgomery, TX). Eleven weeks after engraftment, mice are treated with the Gene Writer™s packaged in NP59, a highly human hepatotropic AAV capsid. The following vectors are administered by i.p. injection:
Active Gene Writing™ of therapeutic: (1) and (3)
Active Gene Writing™ of reporter: (1) and (4)
No integration machinery therapeutic control: (2) and (3)
No integration machinery reporter control: (2) and (4)

After vector injection, mice are cycled on NTBC for another 5 weeks before being euthanized. DNA and RNA are subsequently extracted from liver lysates by standard methods. OTC expression is subsequently assayed by performing RT-qPCR on isolated RNA samples using sequence-specific primers. To confirm integration of construct and analyze genomic locations, unidirectional sequencing is performed on genomic DNA samples by using specific primers annealing to the inserted gene to read outward into the surrounding genomic sequence on a MiSeq.

### Example 2: Application of a Gene Writer™ System for Delivering Therapeutic Gene to Liver in an Infant or Adult Mouse Model of a Disease

This example describes a Gene Writer™ genome editing system delivered to the liver in vivo for integration and stable expression of a genetic payload. The promoter and miRNA recognition sequence for expression control and the therapeutic gene are intended to exemplify the approach and are selected from Tables 2, 3, and 4, respectively.

In this example, an OTC deficient mouse model is used to assess a Gene Writer™ system designed to deliver an OTC expression cassette for integration into liver cells. The Gene Writer™ polypeptide component comprises an expression cassette for the Sleeping Beauty transposase derivative SB100X (Table 1) and the template component comprises an expression cassette for the human OTC gene (Table 4) flanked by the IR/DR sequences required for binding and mobilization by SB100X. In this example, both the transposase and template expression cassettes additionally comprise the hAAT promoter (Table 2) for hepatocyte-specific expression and a miRNA recognition sequence complementary to the seed sequence of miR-142 (Table 3) for downregulating expression in hematopoetic cells.
Gene Writer™ polypeptide component: rAAV2/8.hAAT.SB100X
Mutated Gene Writer™ polypeptide: rAAV2/8.hAAT.dSB100X
Gene Writer™ template component: rAAV2/8.hAAT.OTC
Reporter Gene Writer™ template component: rAAV2/8.hAAT.GFP

Either one to two day-old or eight to 12-week-old female Otc-deficient Spf^{ash} mice (C57BL/6/C3H-F1 background) are treated with the Gene Writer™s packaged in AAV8, a hepatotropic AAV capsid. The following vectors are administered by i.p. injection:
Active Gene Writing™ of therapeutic: (1) and (3)
Active Gene Writing™ of reporter: (1) and (4)
No integration machinery therapeutic control: (2) and (3)
No integration machinery reporter control: (2) and (4)

After 5 weeks, DNA and RNA are subsequently extracted from liver lysates by standard methods. OTC expression is subsequently assayed by performing RT-qPCR on isolated RNA samples using sequence-specific primers. To confirm integration of construct and analyze genomic locations, unidirectional sequencing is performed on genomic DNA samples by using specific primers annealing to the inserted gene to read outward into the surrounding genomic sequence on a MiSeq.

### Example 3: Formulation of Lipid Nanoparticles Encapsulating Firefly Luciferase mRNA

In this example, a reporter mRNA encoding firefly luciferase was formulated into lipid nanoparticles comprising different ionizable lipids. Lipid nanoparticle (LNP) components (ionizable lipid, helper lipid, sterol, PEG) were dissolved in 100% ethanol with the lipid component. These were then prepared at molar ratios of 50:10:38.5:1.5 using ionizable lipid LIPIDV004 or LIPIDV005 (Table 32), DSPC, cholesterol, and DMG-PEG 2000, respectively. Firefly Luciferase mRNA-LNPs containing the ionizable lipid LIPIDV003 (Table 32) were prepared at a molar ratio of 45:9:44:2 using LIPIDV003, DSPC, cholesterol, and DMG-PEG 2000, respectively. Firefly luciferase mRNA used in these formulations was produced by in vitro transcription and encoded the Firefly Luciferase protein, further comprising a 5′ cap, 5′ and 3′ UTRs, and a polyA tail. The mRNA was synthesized under standard conditions for T7 RNA polymerase in vitro transcription with co-transcriptional capping, but with the nucleotide triphosphate UTP 100% substituted with N1-methyl-pseudouridine triphosphate in the reaction. Purified mRNA was dissolved in 25 mM sodium citrate, pH 4 to a concentration of 0.1 mg/mL.

Firefly Luciferase mRNA was formulated into LNPs with a lipid amine to RNA phosphate (N:P) molar ratio of 6. The LNPs were formed by microfluidic mixing of the lipid and RNA solutions using a Precision Nanosystems NanoAssemblr™ Benchtop Instrument, using the manufacturer's recommended settings. A 3:1 ratio of aqueous to organic solvent was maintained during mixing using differential flow rates. After mixing, the LNPs were collected and dialyzed in 15 mM Tris, 5% sucrose buffer at 4° C. overnight. The Firefly Luciferase mRNA-LNP formulation was concentrated by centrifugation with Amicon 10 kDa centrifugal filters (Millipore). The resulting mixture was then filtered using a 0.2 μm sterile filter. The final LNP was stored at −80° C. until further use.

TABLE 32
Ionizable Lipids used in Example 3

| LIPID | | Molecular | |
|---|---|---|---|
| ID | Chemical Name | Weight | Structure |
| LIPIDV 003 | (9Z,12Z)-3- ((4,4-bis (octyloxy) butano yl)oxy)- 2-((((3- (diethylamino) propoxy) carbonyl)oxy) methyl)propyl octadeca-9, 12- dienoate | 852.29 | 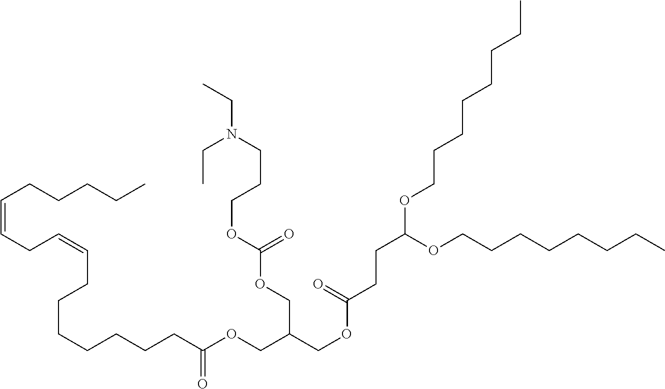 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV 004 | Heptadecan- 9-y1 8-((2- hydroxyethyl) (8-(nonyloxy)- 8-oxooctyl) amino)oct anoate | 710.18 | 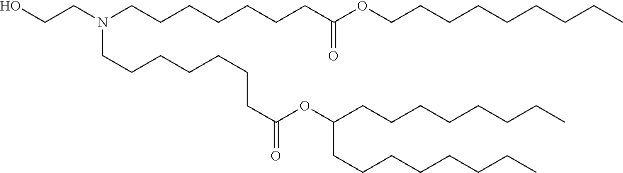 [SMILES: CCCCCCCCCOC(=O)CCCCCCCN(CCO)CCCCCCCC(=O)OC(CCCCCCCC)CCCCCCCC] |
| LIPIDV 005 | | 919.56 | 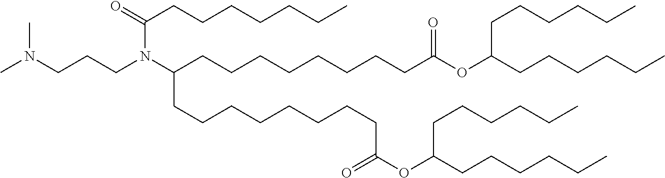 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

Prepared LNPs were analyzed for size, uniformity, and % RNA encapsulation. The size and uniformity measurements were performed by dynamic light scattering using a Malvern Zetasizer DLS instrument (Malvern Panalytical). LNPs were diluted in PBS prior to being measured by DLS to determine the average particle size (nanometers, nm) and polydispersity index (pdi). The particle sizes of the Firefly Luciferase mRNA-LNPs are shown in Table 33.

TABLE 33
LNP particle size and uniformity

| LNP ID | Ionizable Lipid | Particle Size (nm) | pdi |
|---|---|---|---|
| LNPV019-002 | LIPIDV005 | 77 | 0.04 |
| LNPV006-006 | LIPIDV004 | 71 | 0.08 |
| LNPV011-003 | LIPIDV003 | 87 | 0.08 |

The percent encapsulation of luciferase mRNA was measured by the fluorescence-based RNA quantification assay Ribogreen (ThermoFisher Scientific). LNP samples were diluted in 1×TE buffer and mixed with the Ribogreen reagent per manufacturer's recommendations and measured on a i3 SpectraMax spectrophotomer (Molecular Devices) using 644 nm excitation and 673 nm emission wavelengths. To determine the percent encapsulation, LNPs were measured using the Ribogreen assay with intact LNPs and disrupted LNPs, where the particles were incubated with 1×TE buffer containing 0.2% (w/w) Triton-X100 to disrupt particles to allow encapsulated RNA to interact with the Ribogreen reagent. The samples were again measured on the i3 SpectraMax spectrophotometer to determine the total amount of RNA present. Total RNA was subtracted from the amount of RNA detected when the LNPs were intact to determine the fraction encapsulated. Values were multiplied by 100 to determine the percent encapsulation. The Firefly Luciferase mRNA-LNPs that were measured by Ribogreen and the percent RNA encapsulation is reported in Table 34.

TABLE 34
RNA encapsulation after LNP formulation

| LNP ID | Ionizable Lipid | % mRNA encapsulation |
|---|---|---|
| LNPV019-002 | LIPIDV005 | 98 |
| LNPV006-006 | LIPIDV004 | 92 |
| LNPV011-003 | LIPIDV003 | 97 |

### Example 4: In Vitro Activity Testing of mRNA-LNPs in Primary Hepatocytes

In this example, LNPs comprising the luciferase reporter mRNA were used to deliver the RNA cargo into cells in culture. Primary mouse or primary human hepatocytes were thawed and plated in collagen-coated 96-well tissue culture plates at a density of 30,000 or 50,000 cells per well, respectively. The cells were plated in 1× William's Media E with no phenol red and incubated at 37° C. with 5% CO₂. After 4 hours, the medium was replaced with maintenance medium (1× William's Media E with no phenol containing Hepatocyte Maintenance Supplement Pack (ThermoFisher Scientific)) and cells were grown overnight at 37° C. with 5% CO₂. Firefly Luciferase mRNA-LNPs were thawed at 4° C. and gently mixed. The LNPs were diluted to the appropriate concentration in maintenance media containing 7.5% fetal bovine serum. The LNPs were incubated at 37° C. for 5 minutes prior to being added to the plated primary hepatocytes. To assess delivery of RNA cargo to cells, LNPs were incubated with primary hepatocytes for 24 hours and cells were then harvested and lysed for a Luciferase activity assay. Briefly, medium was aspirated from each well followed by a wash with 1×PBS. The PBS was aspirated from each well and 200 μL passive lysis buffer (PLB) (Promega) was added back to each well and then placed on a plate shaker for 10 minutes. The lysed cells in PLB were frozen and stored at −80° C. until luciferase activity assay was performed.

To perform the luciferase activity assay, cellular lysates in passive lysis buffer were thawed, transferred to a round bottom 96-well microtiter plate and spun down at 15,000 g at 4° C. for 3 min to remove cellular debris. The concentration of protein was measured for each sample using the Pierce™ BCA Protein Assay Kit (ThermoFisher Scientific) according to the manufacturer's instructions. Protein concentrations were used to normalize for cell numbers and determine appropriate dilutions of lysates for the luciferase assay. The luciferase activity assay was performed in white-walled 96-well microtiter plates using the luciferase assay reagent (Promega) according to manufacturer's instructions and luminescence was measured using an i3X SpectraMax plate reader (Molecular Devices). The results of the dose-response of Firefly luciferase activity mediated by the Firefly mRNA-LNPs are shown in FIG. 6A and indicate successful LNP-mediated delivery of RNA into primary cells in culture. As shown in FIG. 6A, LNPs formulated as according to Example 3 were analyzed for delivery of cargo to primary human (FIG. 6A) and mouse (FIG. 6B) hepatocytes, as according to Example 4. The luciferase assay revealed dose-responsive luciferase activity from cell lysates, indicating successful delivery of RNA to the cells and expression of Firefly luciferase from the mRNA cargo.

### Example 5: LNP-Mediated Delivery of RNA to the Mouse Liver

To measure the effectiveness of LNP-mediated delivery of firefly luciferase containing particles to the liver, LNPs were formulated and characterized as described in Example 3 and tested in vitro prior (Example 4) to administration to mice. C57BL/6 male mice (Charles River Labs) at approximately 8 weeks of age were dosed with LNPs via intravenous (i.v.) route at 1 mg/kg. Vehicle control animals were dosed i.v. with 300 μL phosphate buffered saline. Mice were injected via intraperitoneal route with dexamethasone at 5 mg/kg 30 minutes prior to injection of LNPs. Tissues were collected at necropsy at or 6, 24, 48 hours after LNP administration with a group size of 5 mice per time point. Liver and other tissue samples were collected, snap-frozen in liquid nitrogen, and stored at −80° C. until analysis.

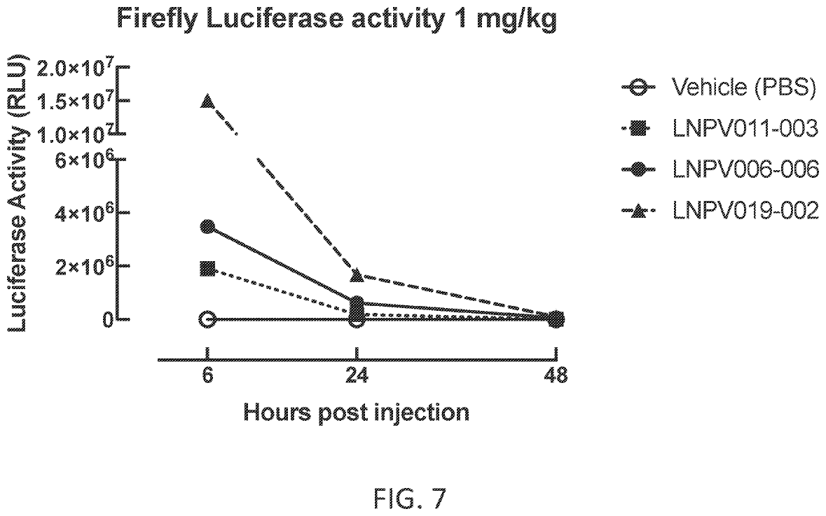

Frozen liver samples were pulverized on dry ice and transferred to homogenization tubes containing lysing matrix D beads (MP Biomedical). Ice-cold 1× luciferase cell culture lysis reagent (CCLR) (Promega) was added to each tube and the samples were homogenized in a Fast Prep-24 5G Homogenizer (MP Biomedical) at 6 m/s for 40 seconds. The samples were transferred to a clean microcentrifuge tube and clarified by centrifugation. Prior to luciferase activity assay, the protein concentration of liver homogenates was determined for each sample using the Pierce™ BCA Protein Assay Kit (ThermoFisher Scientific) according to the manufacturer's instructions. Luciferase activity was measured with 200 μg (total protein) of liver homogenate using the luciferase assay reagent (Promega) according to manufacturer's instructions using an i3X SpectraMax plate reader (Molecular Devices). Liver samples revealed successful delivery of mRNA by all lipid formulations, with reporter activity following the ranking LIPIDV005>LIPIDV004>LIPIDV003 (FIG. 7). As shown in FIG. 7, Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours. Post-administration. This assay validated the use of these ionizable lipids and their respective formulations for RNA systems for delivery to the liver.

### Example 6: Selection of Lipid Reagents with Reduced Aldehyde Content

In this example, lipids are selected for downstream use in lipid nanoparticle formulations containing Gene Writing component nucleic acid(s), and lipids are selected based at least in part on having an absence or low level of contaminating aldehydes. Reactive aldehyde groups in lipid reagents may cause chemical modifications to component nucleic acid(s), e.g., RNA, e.g., template RNA, during LNP formulation. Thus, in some embodiments, the aldehyde content of lipid reagents is minimized.

Liquid chromatography (LC) coupled with tandem mass spectrometry (MS/MS) can be used to separate, characterize, and quantify the aldehyde content of reagents, e.g., as described in Zurek et al. The Analyst 124(9):1291-1295 (1999), incorporated herein by reference. Here, each lipid reagent is subjected to LC-MS/MS analysis. The LC/MS-MS method first separates the lipid and one or more impurities with a C8 HPLC column and follows with the detection and structural determination of these molecules with the mass spectrometer. If an aldehyde is present in a lipid reagent, it is quantified using a staple-isotope labeled (SIL) standard that is structurally identical to the aldehyde, but is heavier due to C13 and N15 labeling. An appropriate amount of the SIL standard is spiked into the lipid reagent. The mixture is then subjected to LC-MS/MS analysis. The amount of contaminating aldehyde is determined by multiplying the amount of SIL standard and the peak ratio (unknown/SIL). Any identified aldehyde(s) in the lipid reagents is quantified as described. In some embodiments, lipid raw materials selected for LNP formulation are not found to contain any contaminating aldehyde content above a chosen level. In some embodiments, one or more, and optionally all, lipid reagents used for formulation comprise less than 3% total aldehyde content. In some embodiments, one or more, and optionally all, lipid reagents used for formulation comprise less than 0.3% of any single aldehyde species. In some embodiments, one or more, and optionally all, lipid reagents used in formulation comprise less than 0.3% of any single aldehyde species and less than 3% total aldehyde content.

### Example 7: Quantification of RNA Modification Caused by Aldehydes During Formulation

In this example, the RNA molecules are analyzed post-formulation to determine the extent of any modifications that may have happened during the formulation process, e.g., to detect chemical modifications caused by aldehyde contamination of the lipid reagents (see, e.g., Example 6).

RNA modifications can be detected by analysis of ribonucleosides, e.g., as according to the methods of Su et al. Nature Protocols 9:828-841 (2014), incorporated herein by reference in its entirety. In this process, RNA is digested to a mix of nucleosides, and then subjected to LC-MS/MS analysis. RNA post-formulation is contained in LNPs and must first be separated from lipids by coprecipitating with GlycoBlue in 80% isopropanol. After centrifugation, the pellets containing RNA are carefully transferred to a new Eppendorf tube, to which a cocktail of enzymes (benzonase, Phosphodiesterase type 1, phosphatase) is added to digest the RNA into nucleosides. The Eppendorf tube is placed on a preheated Thermomixer at 37° C. for 1 hour. The resulting nucleosides mix is directly analyzed by a LC-MS/MS method that first separates nucleosides and modified nucleosides with a C18 column and then detects them with mass spectrometry.

If aldehyde(s) in lipid reagents have caused chemical modification, data analysis will associate the modified nucleoside(s) with the aldehyde(s). A modified nucleoside can be quantified using a SIL standard which is structurally identical to the native nucleoside except heavier due to C13 and N15 labeling. An appropriate amount of the SIL standard is spiked into the nucleoside digest, which is then subjected to LC-MS/MS analysis. The amount of the modified nucleoside is obtained by multiplying the amount of SIL standard and the peak ratio (unknown/SIL). LC-MS/MS is capable of quantifying all the targeted molecules simultaneously.

In some embodiments, the use of lipid reagents with higher contaminating aldehyde content results in higher levels of RNA modification as compared to the use of higher purity lipid reagents as materials during the lipid nanoparticle formulation process. Thus, in preferred embodiments, higher purity lipid reagents are used that result in RNA modification below an acceptable level.

### Example 8: Formulation of Lipid Nanoparticles Encapsulating SB100X mRNA

The lipid nanoparticle (LNP) components (ionizable lipid, helper lipid, sterol, PEG) were dissolved in 100% ethanol with the lipid component. The lipid components used to make the SB100X mRNA-LNPs were prepared at molar ratios of 50:10:38.5:1.5 using ionizable LIPIDV005 (Table 35), DSPC, cholesterol, DMG-PEG 2000, respectively. The mRNA that was used in the formulations encodes the Sleeping Beauty 100X (SB100X) transposase protein and the transcript was made by in vitro transcription where it contained a 5′ cap, 5′ and 3′ UTRs, and a polyA tail. The mRNA was synthesized under standard conditions for T7 RNA polymerase in vitro transcription where co-transcriptional capping is performed except that the nucleotide triphosphate UTP was 100% substituted with N1-methyl-pseudouridine triphosphate in the reaction. The purified mRNA was dissolved in 25 mM sodium citrate, pH 4 resulting in a concentration of RNA at 0.1 mg/mL.

The SB100X mRNA was formulated into LNPs with a lipid amine to RNA phosphate (N:P) molar ratio of 6. The LNPs were formed by microfluidic mixing of the lipid and RNA solutions using a Precision Nanosystems NanoAssemblr™ Benchtop Instrument, using the manufacturer's recommended settings. A 3:1 ratio of aqueous to organic solvent was maintained during mixing using differential flow rates. After mixing, the LNPs were collected and dialyzed in 15 mM Tris, 5% sucrose buffer at 4° C. overnight. The SB100X mRNA-LNP formulation was concentrated by centrifugation with Amicon 10 kDa centrifugal filters (Millipore). The resulting mixture was then filtered using a 0.2 μm sterile filter. The final LNP was stored at −80° C. until further use.

TABLE 35
Ionizable Lipid used to make SB 100X mRNA-LNPs

| | | Molecular | |
|---|---|---|---|
| LIPID ID | Chemical Name | Weight | Structure |
| LIPIDV 005 | di(tridecan-7-yl) 10-(N-(3- (dimethylamino) propyl) octanamido) nonadecanedioate | 919.56 | 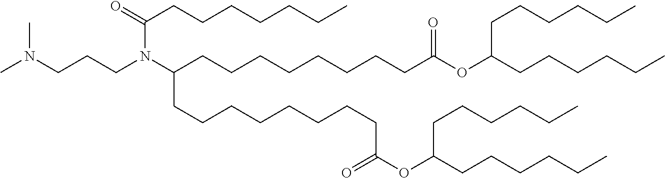 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

### Example 9: Analytics of LNPs

The prepared LNPs were analyzed for their size, uniformity, and % RNA encapsulation. The size and uniformity measurements were performed by dynamic light scattering using a Malvern Zetasizer DLS instrument (Malvern Panalytical). LNPs were diluted in PBS prior to being measured by DLS to determine the average particle size (nanometers, nm) and polydispersity index (pdi). The particle sizes of the SB100X mRNA-LNPs are shown in Table 36.

TABLE 36
LNP particle size and uniformity

| LNP ID | Ionizable Lipid | Particle Size (nm) | pdi |
|---|---|---|---|
| SB100X mRNA LNP | LIPIDV005 | 78 | 0.04 |
| (LNPV022-001) | | | |

The percent encapsulation of the mRNA was measured by the fluorescence-based RNA quantification assay Ribogreen (ThermoFisher Scientific). LNP samples were diluted in 1×TE buffer and mixed with the Ribogreen reagent per manufacturer's recommendations and measured on a i3 SpectraMax spectrophotomer (Molecular Devices) using 644 nm excitation and 673 nm emission wavelengths. To determine the percent encapsulation, the LNPs were measured using the Ribogreen assay with the LNPs intact and then the LNPs were incubated with 1×TE buffer containing 0.2% (w/w) Triton-X100 to disrupt LNP to allow all the RNA to interact with the Ribogreen reagent. The samples were measured again on the i3 SpectraMax spectrophotometer to determine the total amount of RNA present. The total RNA amount was subtracted from the amount of RNA detected when the LNPs were intact to determine the fraction encapsulated. Values were multiplied by 100 to determine the percent encapsulation. The SB100X mRNA-LNPs that were measured by Ribogreen and the percent RNA encapsulation is reported in Table 37.

The concentration of the final concentration of the SB100X mRNA-LNP was determined by performing the Ribogreen assay above alongside a standard curve generated with non-formulate SB100X mRNA. Total concentration of the LNP is determined by the total RNA adjusted for percent encapsulated.

TABLE 37
RNA encapsulation after LNP formulation

| LNP ID | Ionizable Lipid | % mRNA encapsulation |
|---|---|---|
| LNPV022-001 | LIPIDV005 | 98 |

### Example 10: In Vitro Integration of mKate2 Mediated by SB100X mRNA LNP in Human Culture Hepatocytes

HuH-7 cells were plated in 48-well tissue culture plates at a density of 60,000 cells per well. The cells were plated in 1×DMEM+10% FBS and incubated at 37° C. with 5% CO2. Cells were either untreated, treated with the AAVDJ-mKate2 SB100X transposon alone (AAV-DJ comprising an mKate2 cassette flanked by IR/ITR/TIR sequences recognized by SB100X transposase), or SB100X mRNA-LNP (transposase mRNA formulated in an LNP)+AAVDJ-mKate2 SB100X transposon. For wells treated with the mKate2 Sleeping Beauty 100X transposon (alone or with LNP), the AAV was diluted in Opti-MEM and added to wells at a final concentration of 1×10⁴ vg per cell or 6×10⁸ vg per well. SB100X mRNA-LNPs were thawed at 4° C. and gently mixed. The LNPs were diluted to the appropriate concentration in Opti-MEM containing 7.5% FBS. The LNPs were incubated at 37° C. for 5 minutes prior to being added to the HuH-7 cells. After transfection and/or transduction, cells were monitored by flow cytometry for mKate2 expression. Briefly cells were dissociated from wells with TrypLE and re-suspended in DMEM+10% FBS with 1/3 of the cell suspension replated in a 48-well and the other 2/3 was measured on a flow cytometer for mKate2 fluorescence. Cells were cultured and measured over the course of 32 days.

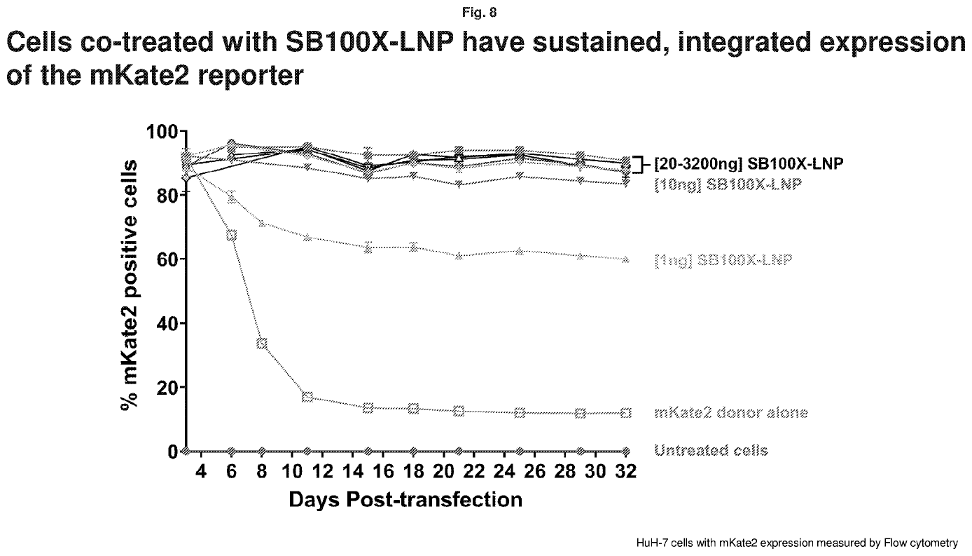

FIG. 8 shows the mKate2 expression over time after transfection and/or transduction of the SB100X mRNA LNP and AAVDJ-mKate2 SB100X transposon. AAVDJ-mKate2 SB100X transposon alone shows a decrease in mKate2 expression over time, indicating episomal AAV loss following multiple cell divisions. The cells that were co-treated with SB100X mRNA LNP and AAVDJ-mKate2 SB100X transposon show sustained fluorescence over time. The sustained expression represents integration into the genome that is not lost with cell division.

### Example 11: In Vitro Integration of mKate2 Mediated by SB100X mRNA LNP in Primary Human Hepatocytes

Primary human hepatocytes were thawed and plated in collagen-coated 96-well tissue culture plates at a density of 55,000 cells per well. The cells were plated in 1×William's Media E with no phenol red and incubated at 37° C. with 5% CO₂. The medium was changed 4 hours after plating to maintenance medium (1× William's Media E with no phenol containing Hepatocyte Maintenance Supplement Pack (ThermoFisher Scientific)) and cells were grown overnight at 37° C. with 5% CO2. Medium was changed from maintenance medium to Cellartis Power Primary HEP Medium (Takara Bio) prior to transfection and/or transduction.

Cells were either untreated, treated with the AAVDJ-mKate2 SB100X transposon alone, or SB100X mRNA-LNP+AAVDJ-mKate2 SB100X transposon. For wells treated with the mKate2 Sleeping Beauty 100X transposon (alone or with LNP), the AAV was diluted in Cellartis Power Primary HEP Medium and added to wells at a final concentration of 5×10⁵ vg per cell or 2.75×10¹⁰ vg per well. SB100X mRNA-LNPs were thawed at 4° C. and gently mixed. The LNPs were diluted to the appropriate concentration in Cellartis Power Primary HEP Medium containing 7.5% FBS. The LNPs were incubated at 37° C. for 5 minutes prior to being added to the plated primary hepatocytes. The LNPs were incubated with primary hepatocytes over the course of 12 days with fluorescence microscopy, brightfield microscopy, and total fluorescence measurements taken periodically. The total fluorescence was measured on a Synergy Neo2 plate reader (Biotek). Briefly, Cellartis Power Primary HEP Medium was aspirated and replaced with phenol-free maintenance medium. Fluorescence endpoint measurements were recorded using the following parameters: excitation: 588/20, emission: 633/20, Gain: 100 and Optics: top. Fluorescence values were calculated as the mean fold change difference between the treated with the AAVDJ-mKate2 SB transposon alone, or SB100X mRNA-LNP+AAVDJ-mKate2 SB transposon over untreated cells.

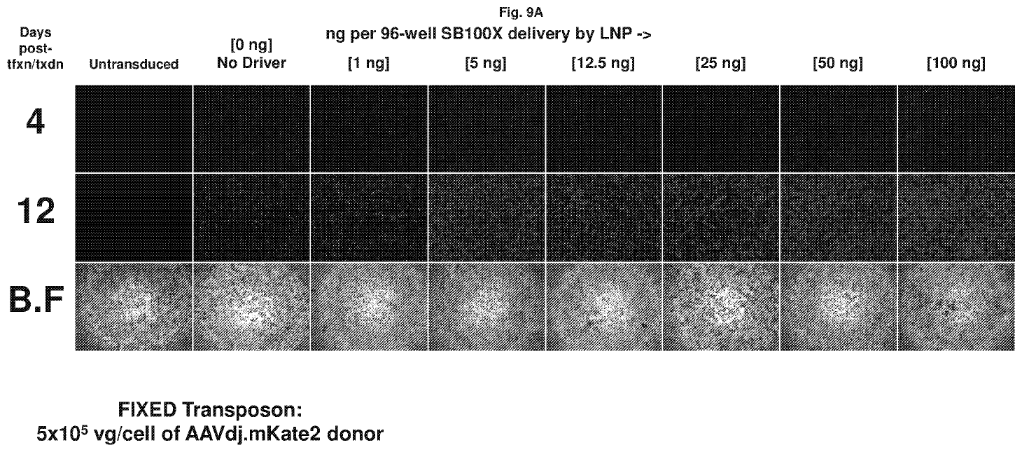

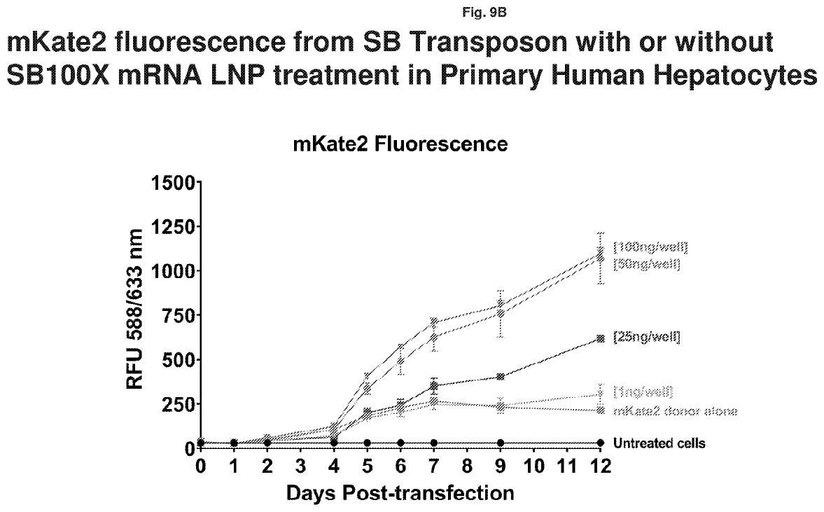

FIG. 9A shows fluorescence images of primary hepatocytes taken either 4 or 7 days after transfection and/or transduction. Brightfield images were taken on day 12. Primary hepatocytes do not divide and there is no expectation of a loss of mKate2 fluorescence expression over time after AAV expression (data not shown). Total fluorescence of episomal expressed mKate2 transposon alone (images at 0 ng SB100X) was weaker when compared to wells that had greater than 1 ng of SB100X mRNA LNP added to them (FIG. 9B). There is no amplification of the AAV in these non-dividing cells thus the integration of mKate2 mediated by SB100X leads to greater mKate2 fluorescence when compared to the fluorescence from the AAV episome only.

### Example 12: Sleeping Beauty 100X Mediated Integration in Neonatal Mice Mediated by LNP/AAV Delivery

Sleeping Beauty 100X mediated integration of the mKate2 gene was evaluated in a neonatal mouse model to distinguish the expression from genome integrated expression versus AAV episomal expression of mKate2 over time. CD-1 mice at age post-natal day 1 or 2 were injected IV through the facial temporal vein with either AAV(s) for delivery of mKate2 template alone, LNP for delivery of mRNA encoding SB100X alone, or the LNP mixed with the AAV. LNP plus AAV or two AAVs were mixed just prior to dosing. Mice were dosed at a final volume of 50 μL where the amount of LNP was dosed based on the average body weight and the AAV was dosed at a vector genome per mouse pup. Injections were performed by cryo-anesthetizing mice on ice, IV injection, and then warming prior to returning mice to dam. After injection, mice were euthanized at various time points over the course of 6 weeks to measure mKate2 expression.

Frozen liver samples were transferred to homogenization tubes containing lysing matrix D beads (MP Biomedical). Ice-cold 1× luciferase cell culture lysis reagent (CCLR) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Prior to the measurement of mKate2 fluorescence from the liver homogenates, the protein concentration was determined for each sample using the Pierce™ BCA Protein Assay Kit (ThermoFisher Scientific) according to the manufacturer's instructions. To measure mKate expression, 200 μg of total protein from the liver homogenates was added to a black, flat-bottom 96-well microtiter plate and the fluorescence was measured at an excitation of 588 nm with detection at 633 nm emission on the Biotek Neo2 plate reader. The concentration of mKate2 protein was determined by using a standard curve of recombinant mKate2 or Red fluorescent protein (identical values for relative fluorescence per μg of purified protein, data not shown).

Genomic DNA was isolated from liver lysate using the gDNA Blood and Tissue extraction kit (Qiagen), and quantified using Quant-IT fluorescence (Thermo) compared to a DNA standard curve. DNA integrity was confirmed using gDNA TapeStation (Agilent Technologies). AAV copy numbers were quantified by ddPCR using primers and probe targeting the WPRE element.

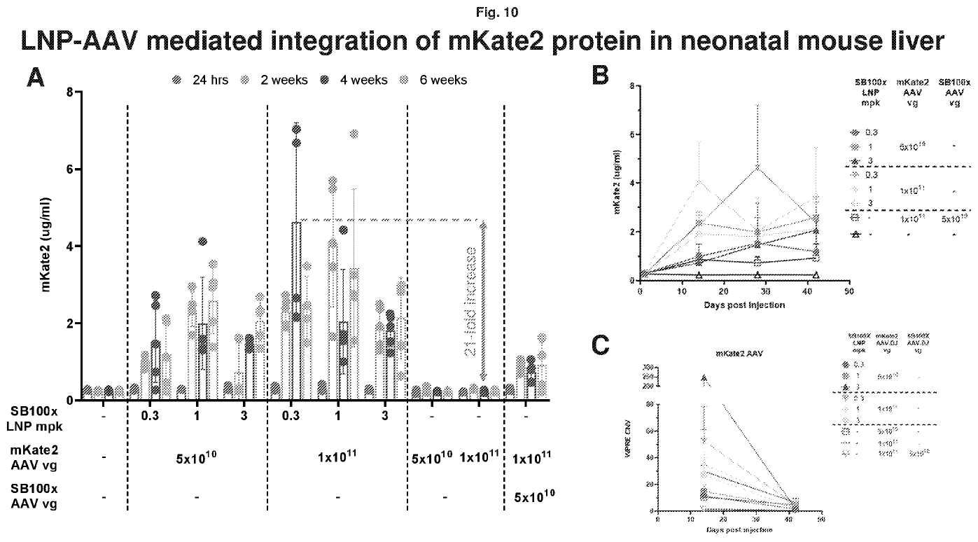

FIG. 10A shows the comparison of mKate2 fluorescence over time after administration of SB100X transposase mRNA-LNP and a Sleeping Beauty 100X transposon containing the mKate2 gene. When SB100X was expressed via an mRNA delivered by LNP it increased expression of mKate2 protein approximately 20 times higher than AAV transposon alone. Expression was sustained over the course of 6 weeks in a dose-dependent fashion where optimal expression of SB100X at 1 mg per kg mediated highest levels of mKate2 expression mediated by the integration activity of the transposase.

FIG. 10B shows the increased mKate2 fluorescence in treated mice over 6-weeks post dosing with transposon and SB100X transposase compared to AAV-transposon alone. Animals which received the SB100X transposase with the mKate2 transposon produced up to 20-fold more mKate2 fluorescence.

FIG. 10C shows AAV copy numbers in mouse livers following AAV transduction with mKate2 transposon. Copies per genome were quantified from purified gDNA using primers and probes against the WPRE element and normalized to RPP30. All animals demonstrated a significant decrease in AAV copies from week 2 to week 6. At week 6, AAV copies were equally low in all mice, suggesting the persistent mKate2 fluorescence is due to genome integration.

### Example 13: Sleeping Beauty 100X Mediated Integration in Adult Mice Mediated by LNP/AAV Delivery

C57BL/6 male mice (Jackson Labs) at approximately 8 weeks of age were dosed with SB100X mRNA LNP alone, AAV for delivering mKate2 Sleeping Beauty 100X transposon alone, or a mixture of the LNP and the AAV via intravenous tail vein at various concentrations and a fixed concentration of AAV, 1×10¹² vg per mouse. Vehicle control animals were dosed with phosphate buffered saline containing 0.01% w/v Pluronic F-68. The mice were sacrificed by carbon dioxide euthanasia at 5 days after administration. Tissues were collected at necropsy including liver which was collected and snap-frozen in liquid nitrogen. Tissue samples were stored at −80° C.

Frozen liver samples were transferred to homogenization tubes containing lysing matrix D beads (MP Biomedical). Ice-cold 1× luciferase cell culture lysis reagent (CCLR) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Prior to the measurement of mKate2 fluorescence from the liver homogenates, the protein concentration was determined for each sample using the Pierce™ BCA Protein Assay Kit (ThermoFisher Scientific) according to the manufacturer's instructions. To measure mKate expression, 200 μg of total protein from the liver homogenates was added to a black, flat-bottom 96-well microtiter plate and the fluorescence was measured at an excitation of 588 nm with detection at 633 nm emission on the Biotek Neo2 plate reader. The concentration of mKate2 protein was determined by using a standard curve of recombinant mKate2 or Red Fluorescent Protein (identical values for relative fluorescence per μg of purified protein, data not shown).

FIG. 11 shows the comparison of mKate2 fluorescence after dosing mice (n=3) with different concentrations of SB100X transposase mRNA-LNP and a fixed concentration of AAV-Sleeping Beauty 100X transposon containing the mKate2 gene (1×10¹² vg per mouse). When SB100X was expressed via an mRNA delivered by LNP it increased expression of mKate2 protein approximately 85 times higher than AAV transposon alone. Sleeping Beauty 100X mediated integration of mKate2 and 85-fold increased fluorescence plateaus at a dose of 2 mg/kg and higher concentrations (3 mpk) did not show increased levels of fluorescence.

### Example 14: Sleeping Beauty 100X Mediated Integration in Adult Mice Mediated by LNP/AAV Delivery

An experiment was conducted to test that SB100X mediated integration of a template reporter gene will result in higher levels of expression compared to the reporter gene being expressed from the AAV episome alone. Additionally, this experiment evaluates the in vivo efficacy of SB100X mRNA delivered via lipid nanoparticle in combination with an AAV delivered transposon.

The Gene Writer™ polypeptide component comprises an expression cassette for the Sleeping Beauty transposase derivative SB100X (Table Z2) and the template component comprises an expression cassette for a reporter gene, mKate2, flanked by the IR/DR sequences required for binding and mobilization by SB100X.

Gene Writer™ polypeptide component: SB100X mRNA encapsulated in a lipid nanoparticle. The SB100X mRNA contains a 5′UTR, Kozak sequence, coding sequence for the SB100X polypeptide, 3′ UTR, and a polyA tail.

Reporter Gene Writer™ template component: AAV-DJ-T2-Ef1a-mKate2-WPRE that is a recombinant adeno-associated serotype DJ virus with AAV2 ITRs that flank the SB100X template sequence. The SB100X template sequence has T2 inverted repeats that flank the mKate2 reporter gene that has an Elongation factor 1-alpha (Ef1a) promoter that precedes the coding sequence for the fluorescent protein mKate2 that is then followed by the Woodchuck hepatitis virus Post-transcriptional Regulatory Element (WPRE) then a Human Growth Hormone poly-adenylation signal (hGH polyA).

C57BL/6 male mice (Taconic Biosciences) at approximately 8 weeks of age were dosed with SB100X mRNA LNP alone, AAV for delivering mKate2 transposon alone, or a mixture of the LNP and the AAV via intravenous tail vein at various concentrations and a fixed concentration of AAV, 1×10 12 vg per mouse. The AAV transposon/template was dosed alone to control for episomal expression alone. The SB100X mRNA LNP alone was dosed to control for SB100X expression. Vehicle control animals were dosed with phosphate buffered saline containing 0.001% w/v Pluronic F-68. The mice were sacrificed by carbon dioxide euthanasia at days after administration. Tissues were collected at necropsy including liver which was collected where half the liver was fixed in 10% neutral buffered formalin and half the liver was snap-frozen in liquid nitrogen. Fixed tissue was transferred to 70% ethanol after 24 hours and stored at 4° C. Snap-frozen tissue samples were stored at −80° C.

Frozen liver samples were transferred to homogenization tubes containing lysing matrix D beads (MP Biomedical). Ice-cold 1× cell culture lysis reagent (CCLR) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Prior to the measurement of mKate2 fluorescence from the liver homogenates, the protein concentration was determined for each sample using the Pierce™ BCA Protein Assay Kit (ThermoFisher Scientific) according to the manufacturer's instructions. To measure mKate2 expression, 62.5 μg of total protein from the liver homogenates was added to a black, flat-bottom 96-well microtiter plate and the fluorescence was measured at an excitation of 588 nm with detection at 633 nm emission on the Biotek Neo2 plate reader. The concentration of mKate2 protein was determined by using a standard curve of recombinant mKate2 or Red Fluorescent Protein (identical values for relative fluorescence per μg of purified protein, data not shown).

Genomic and nuclear episomal DNA was isolated from liver tissue using the DNeasy Blood and Tissue kit (Qiagen) and quantified using Quant-iT™ dsDNA detection kit (Thermo Fisher). AAV copy numbers were determined by ddPCR using primer/probes which amplify the WPRE sequence within the AAV transgene and normalized to RPP30 ribonuclease.

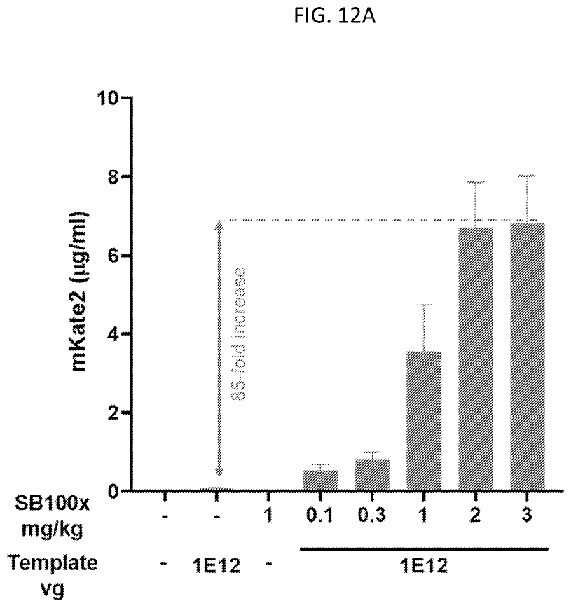

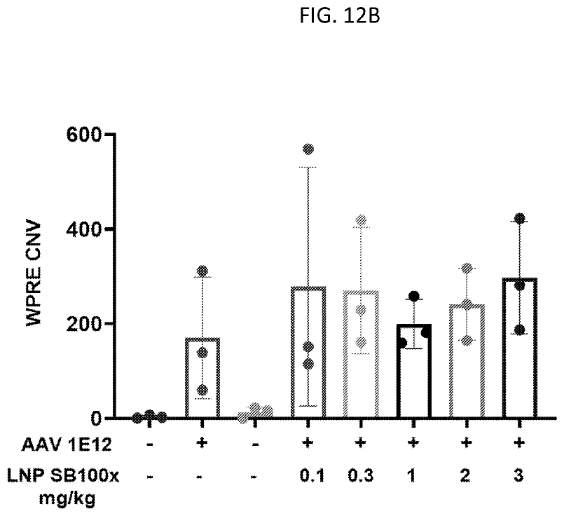

Results: As shown in FIG. 12A, mKate2 fluorescence increases after dosing mice (n=3) with increasing concentrations of LNP SB100X transposase (dose amount 0.1, 0.3, 1, 2, or 3 mg/kg) and a fixed concentration of AAV transposon containing the mKate2 cDNA (1×1012 vg per mouse). When SB100X was expressed via an mRNA delivered by LNP it increased expression of mKate2 protein approximately 85 times higher than AAV transposon alone. Sleeping Beauty 100X mediated integration of mKate2 and 85-fold increased fluorescence plateaus at a dose of 2 mg/kg and higher concentrations (3 mpk) did not show increased levels of fluorescence. As shown in FIG. 12B, AAV copy numbers are consistent across all groups that received the viral vector. Addition of SB100X LNP did not affect AAV transduction of mouse livers.

### Example 15: Tissue Targeted Delivery of Sleeping Beauty 100X Mediated Integration of rhCG Reporter in Mice Mediated by LNP/AAV Delivery

An experiment was conducted to test the SB100X Gene Writer™ system to integrate a secreted reporter gene (rhCG) and compare the levels of expression to either the template/transposon alone or expression from an AAV alone. As shown in the results below, SB100X-mediated integration results in higher levels of expression compared to episomal expression.

The Gene Writer™ polypeptide component: SB100X mRNA encapsulated in a lipid nanoparticle. The SB100X mRNA contains a 5′UTR, Kozak sequence, coding sequence for the SB100X polypeptide, 3′ UTR, and a polyA tail.

Reporter Gene Writer™ template component: AAV8-T2-SerpENH-TTRmin-rhCG-WPRE-bGH pA that is a recombinant adeno-associated serotype 8 virus with AAV2 ITRs that flank the SB100X template sequence. The SB100X template sequence has T2 inverted repeats that flank the Rhesus Macaque Chorionic Gonadotropin (rhCG) reporter gene that has a Serpin A1 enhancer and Transthyretin minimal promoter combination for liver specific expression that precedes the coding sequence for the secreted protein rhCG that is then followed by the Woodchuck hepatitis virus Post-transcriptional Regulatory Element (WPRE) then a Bovine Growth Hormone poly-adenylation signal (bGH polyA).

C57BL/6 male mice (Taconic Biosciences) at approximately 8 weeks of age were dosed with AAV for delivering rhCG transposon alone, AAV for delivering the rhCG transgene alone, or a mixture of the LNP and the AAV transposon via intravenous tail vein at various concentrations and a fixed concentration of AAV, 1×10¹² vg per mouse. The controls for rhCG expression mediated by the AAV episome were either the AAV transposon or an AAV that expressed rhCG without having the Sleeping Beauty inverted repeats, I.e. transgene. Vehicle control animals were dosed with phosphate buffered saline containing 0.001% w/v Pluronic F-68. Serum was collected 1 day prior to dosing, 24 hours, 7 days, and 14 days after dosing. The mice were sacrificed by carbon dioxide euthanasia at 14 days after administration. Tissues were collected at necropsy including liver which was collected where half the liver was fixed in 10% neutral buffered formalin and half the liver was snap-frozen in liquid nitrogen. Fixed tissue was transferred to 70% ethanol after 24 hours and stored at 4° C. Snap-frozen tissue samples were stored at −80° C.

Frozen liver samples were transferred to homogenization tubes containing lysing matrix D beads (MP Biomedical). Ice-cold 1× passive lysis buffer (PLB) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Liver mRNA transcripts were isolated from frozen tissue using the SV Total RNA Isolation System (Promega). Concentrations were determined using Quant-iT™ RNA assay kit (Thermo Fisher). Complimentary DNA was produced using the High Capacity cDNA Reverse Transcription Kit (Applied Biosystems). FAM probe/primers against the rhCG sequence were used for qPCR analysis on the CFX384 Touch Thermocycler (Bio-Rad) and reported as Cq values.

Genomic and nuclear episomal DNA was isolated from liver tissue using the DNeasy Blood and Tissue kit (Qiagen) and quantified using Quant-iT™ dsDNA detection kit (Thermo Fisher). AAV copy numbers were determined by ddPCR using primer/probes which amplify the WPRE sequence within the AAV transgene and normalized to RPP30 ribonuclease.

Results:

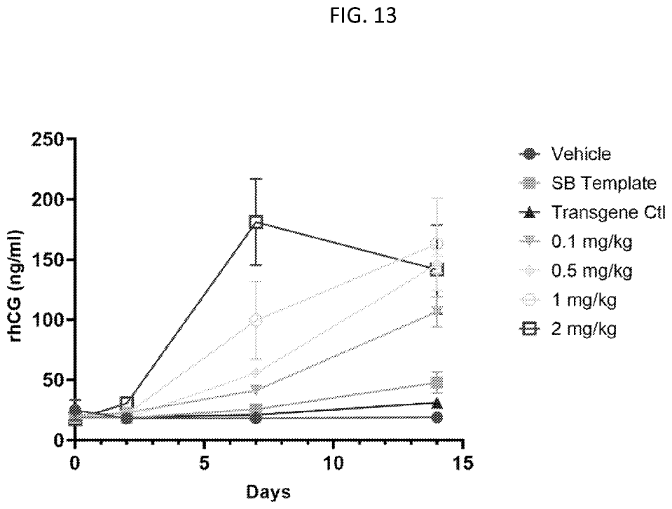

FIG. 13 depicts rhCG serum concentration over two weeks measured by radioimmunoassay. Peak rhCG levels were observed at weeks 1 and 2 post administration of 2 and 1 mg/kg, respectively. Reduced levels of transposase resulted in decreased rhCG production Peak rhCG concentrations were 4-5 fold greater than template or transgene AAV alone.

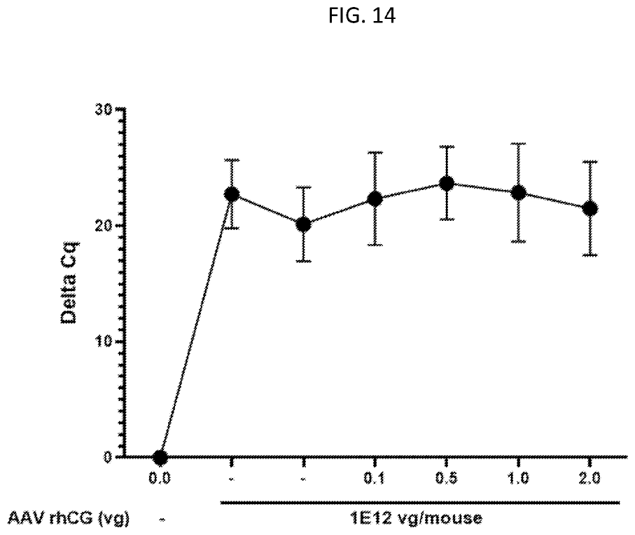

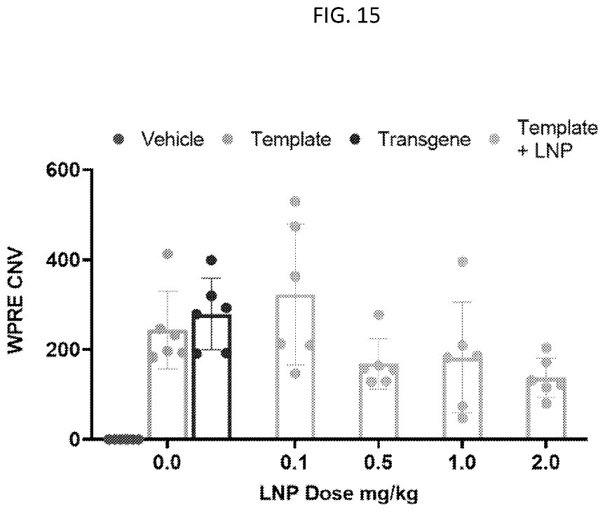

FIG. 14 shows qRT PCR analysis of rhCG transcripts in AAV treated mouse livers. Groups treated with template AAV or transgene AAV display increased delta Cq values 20-23 on average after normalization to beta-tubulin. FIG. 15 depicts AAV copy numbers in transduced mouse livers as determined by ddPCR. Copy numbers are equivalent across AAV treated groups (n=6) indicating that differences in rhCG levels are driven by transposase concentrations.

### Example 16: Tissue Targeted Delivery of eGFP in Adult Mice by AAV

An experiment was conducted to compare AAV8 transgene vectors under two separate promoters for reporter gene expression in adult mice.

C57BL/6 male mice (Taconic Biosciences) at approximately 8 weeks of age were dosed with AAV8 containing the eGFP cDNA under the SerpTTR minimal or the ApoE-hAAT promoter via intravenous tail vein at three concentrations of 5×10¹¹, 1×10¹², or 2.5×10¹² vg per mouse. Vehicle control animals were dosed with phosphate buffered saline containing 0.001% w/v Pluronic F-68. The mice were sacrificed by carbon dioxide euthanasia at 5 days after administration. Tissues were collected at necropsy including liver which was collected and snap-frozen in liquid nitrogen. Tissue samples were stored at −80° C.

Frozen liver samples were transferred to homogenization tubes containing lysing matrix D beads (MP Biomedical). Ice-cold 1× Passive Lysis Buffer (PLB) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Prior to the measurement of eGFP antigen concentration from the liver homogenates, the total protein concentration was determined for each sample using the Pierce™ BCA Protein Assay Kit (ThermoFisher Scientific) according to the manufacturer's instructions. The concentration of eGFP protein was determined by ELISA using manufacturer's instructions (Abcam).

Results:

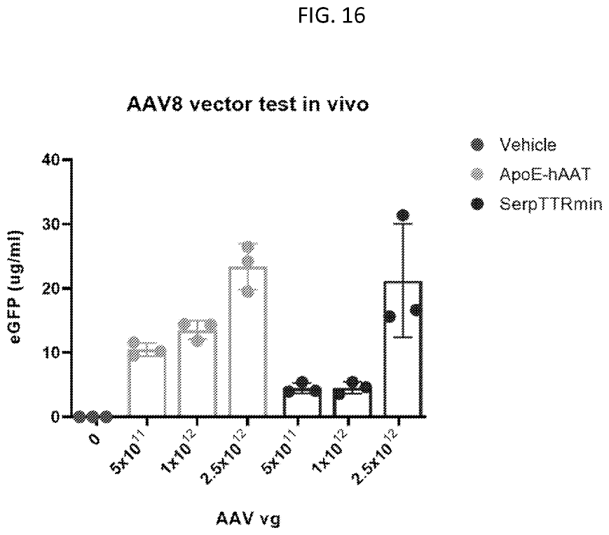

FIG. 16 demonstrates the ApoE-hAAT and SerpTTRmin promoters increasing eGFP production with increasing dose of AAV. While the ApoE-hAAT promoter produces increased eGFP at lower vector doses relative to SerpTTRmin, at 2.5 E12 vg/mouse, the two promoters display equivalent maximum eGFP. Thus, the choice of promoter can have lead to different dose-dependent effects.

### Example 17: Tissue Targeted Delivery of eGFP in Non-Human Primates Macaca fascicularis by AAV

An experiment was conducted to evaluate AAV8 transgene vectors under two separate promoters for reporter gene expression in non-human primates with and without neutralizing inhibitors to AAV.

Reporter template component: (a) rAAV8/NP59.SerpTTRmin.eGFP or (b) rAAV8/NP59.hAAT.eGFP is a recombinant adeno-associated serotype 8 virus with AAV2 ITRs that flank the eGFP reporter sequence. In configuration (a), the eGFP reporter gene has a Serpin A1 enhancer and Transthyretin minimal promoter combination for liver specific expression that precedes it and is then followed by the Woodchuck hepatitis virus Post-transcriptional Regulatory Element (WPRE) then a Bovine Growth Hormone poly-adenylation signal (bGH polyA). In configuration (b), the eGFP reporter has an ApoE enhancer-human alpha anti-trypsin enhancer-promoter sequence that precedes a kozak sequence that is just before the coding sequence for the eGFP cDNA that is then followed by the Woodchuck hepatitis virus Post-transcriptional Regulatory Element (WPRE) then a bovine Growth Hormone poly-adenylation signal (bGH polyA).

Male and female Macaca fascicularis monkeys were dosed with AAV for delivering eGFP reporter gene via intravenous injection at various concentrations. As a negative control, animals were dosed with phosphate buffered saline containing 0.001% w/v Pluronic F-68 (Vehicle control). Prior to AAV treatment, animals were treated with methylprednisolone (40 mg/animal administered intramuscularly [IM]) twice, on Days 8 and Day 1 prior to dosing. In a first phase of the experiment, Macaca fascicularis monkeys without inhibitors (n=2) and one (1) monkey with neutralizing inhibitor titer of 5 were each injected with 5×10¹² vg/kg AAV8 vectors with each configuration (a) and (b) described above. In a second phase of the experiment, two Macaca fascicularis monkeys without neutralizing inhibitors were injected with 1×10¹³ or 5×10¹³ vg/kg of SerpTTRmin AAV vector (configuration (a)) only. In a third phase of the experiment, Macaca fascicularis monkeys with neutralizing inhibitor titers of 10 or 20 were injected with 3.95×10¹³ vg/kg. of the SerpTTRmin construct.

The non-human primates were sacrificed by carbon dioxide euthanasia after administration. Liver collection was performed by sectioning the liver into eight (8) segments followed by bi-section of segments with one (1) bisection to be fixed in 10% neutral buffered formalin for 24 hours followed by being placed in 70% ethanol and one (1) bisection snap frozen in liquid nitrogen Frozen tissue samples were stored at −80° C.

Frozen liver samples were transferred to homogenization tubes containing lysing matrix D beads (MP Biomedical). Ice-cold 1× passive lysis buffer (PLB) (Promega) containing HALT protease and phosphatase inhibitors (ThermoFisher) was added to each tube and the samples were homogenized in a Fast Prep-24 5G Homogenizer (MP Biomedical) at 6 m/s for 40 seconds. The samples were transferred to a clean microcentrifuge tube or deep-well plate and clarified by centrifugation.

Prior to the measurement of eGFP protein from the liver homogenates, the total protein concentration was determined for each sample using the Pierce™ BCA Protein Assay Kit (ThermoFisher Scientific) according to the manufacturer's instructions. To measure eGFP concentration, an ELISA was performed according to manufacturer's instructions (Abcam).

Genomic and nuclear episomal DNA was isolated from liver tissue using the DNeasy Blood and Tissue kit (Qiagen) and quantified using Quant-iT™ dsDNA detection kit (Thermo Fisher). AAV copy numbers were determined by ddPCR using primer/probes which amplify the WPRE sequence within the AAV transgene and normalized to RPP30 ribonuclease.

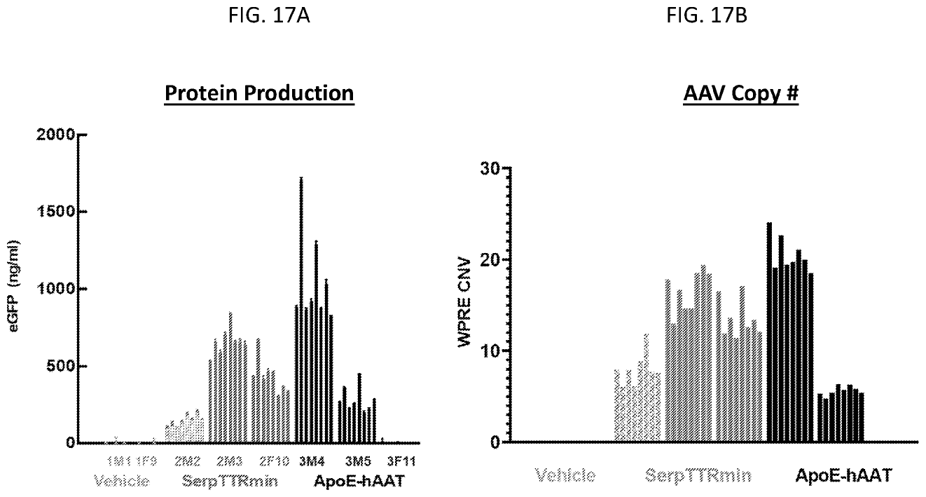

Results: FIG. 17A demonstrates that vector constructs delivered reporter gene to tissue throughout the target organ: eGFP was observed in all liver sections of animals treated with AAV, (2M2, 2M3, 2F10, 3M4, and 3M5) as determined by eGFP ELISA. Each vertical bar represents one of the eight liver sections separated during necropsy. Maximum eGFP signal was observed with the ApoE-hAAT promoter in animal 3M4, however, variability of expression was reduced with SerpTTRmin promoter in animals 2M3 and 2F10. Animals 2M2 and 3F11 each possessed neutralizing inhibitor titers of 5 prior to AAV administration. The Serp TTRmin promoter was selected in follow-up studies due to its ability to produce eGFP in animal 2M2 whereas the ApoE-hAAT construct failed to produce eGFP in animal 3F11 with equivalent inhibitor titer. eGFP concentrations were approximately 5× lower than in mice as shown in Example 16.

FIG. 17B demonstrates that AAV copy number correlated with eGFP signal in each animal and variability was less with the SerpTTRmin construct. No copies were observed in 3F11 however AAV copies were detected in 2M2.

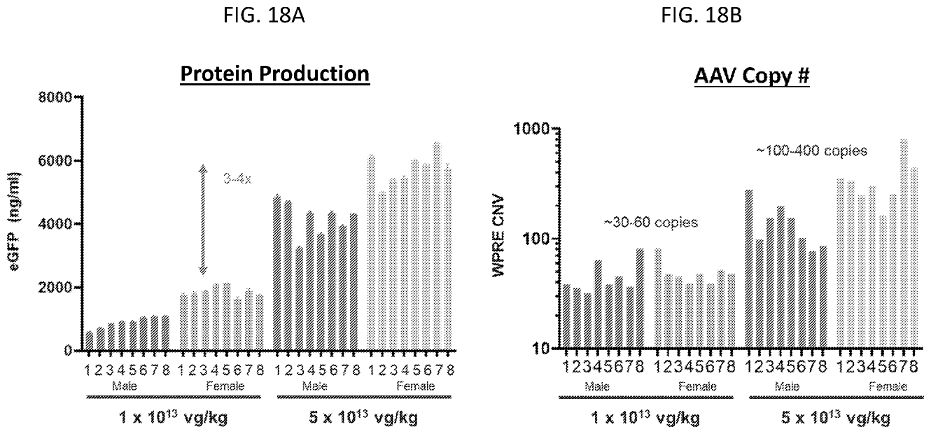

FIGS. 18A-18B show that dose escalation by 5× increased eGFP signal 3-4 fold, along with AAV copy numbers. Equal distribution across the liver was again observed.

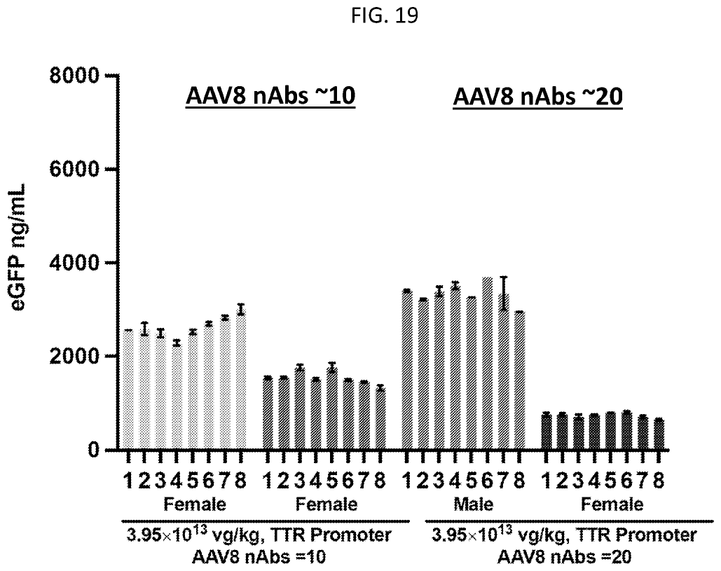

FIG. 19 shows that animals with either 10 or 20 nAbs titers had reduced eGFP levels by a factor of 2-6 fold compared to animals without nAbs. Nevertheless, eGFP was consistently observed in all four (4) animals.

TABLE Z2
Sequences

| | | | SEQ ID |
|---|---|---|---|
| Name | Type | Sequences | NO: |
| SB100X | RNA | Cap: 7mG- | 1650 |
| mRNA | | 5 ′UTR: AAGGAAAUAAGAGAGAAAAGAAGAGUAAGAAGAAAUAUAAGAGCCACC (SEQ ID | |
| UTR | | NO: 1614) | |
| | | SB100X_CDS: | |
| | | AUGGGCAAGUCCAAGGAGAUCUCUCAGGACCUGAGAAAGAGGAUCGUGGAUCUGCACAAGAGCG | |
| | | GAAGCUCCCUGGGAGCAAUCUCCAAGCGCCUGGCAGUGCCUCGGUCUAGCGUGCAGACCAUCGU | |
| | | GCGCAAGUACAAGCACCACGGCACCACACAGCCUUCUUAUCGGAGCGGCCGGAGAAGGGUGCUG | |
| | | AGCCCAAGGGACGAGCGGACACUGGUGCGCAAGGUGCAGAUCAACCCCCGGACCACAGCCAAGG | |
| | | AUCUGGUGAAGAUGCUGGAGGAGACCGGCACAAAGGUGUCCAUCUCUACCGUGAAGAGAGUGCU | |
| | | GUACAGGCACAACCUGAAGGGCCACUCCGCCAGAAAGAAGCCUCUGCUGCAGAAUAGGCACAAG | |
| | | AAGGCAAGGCUGAGGUUCGCAACCGCACACGGCGACAAGGAUCGCACAUUUUGGCGGAACGUGC | |
| | | UGUGGUCUGACGAGACCAAGAUCGAGCUGUUCGGCCACAAUGAUCACAGAUACGUGUGGAGGAA | |
| | | GAAGGGCGAGGCCUGCAAGCCCAAGAAUACCAUCCCUACAGUGAAGCACGGAGGAGGAUCCAUC | |
| | | AUGCUGUGGGGAUGUUUUGCAGCAGGAGGAACAGGCGCCCUGCACAAGAUCGACGGCAUCAUGG | |
| | | AUGCCGUGCAGUAUGUGGACAUCCUGAAGCAGCACCUGAAGACCUCUGUGAGAAAGCUGAAGCU | |
| | | GGGCAGGAAGUGGGUGUUCCAGCACGACAACGAUCCAAAGCACACAAGCAAGGUGGUGGCCAAG | |
| | | UGGCUGAAGGACAAUAAGGUGAAGGUGCUGGAGUGGCCCAGCCAGUCCCCUGAUCUGAACCCAA | |
| | | UCGAGAAUCUGUGGGCCGAGCUGAAGAAGAGAGUGAGGGCCCGGAGACCCACCAACCUGACACA | |
| | | GCUGCACCAGCUGUGCCAGGAGGAGUGGGCCAAGAUCCACCCAAAUUACUGUGGCAAGCUGGUG | |
| | | GAGGGCUAUCCCAAGAGGCUGACCCAGGUGAAGCAGUUUAAGGGCAACGCCACAAAGUAU | |
| | | (SEQ ID NO: 1615) | |
| | | 3 ′UTR: | |
| | | UGAUUAAUUAAGCUGCCUUCUGCGGGGCUUGCCUUCUGGCCAUGCCCUUCUUCUCUCCCUUGCA | |
| | | CCUGUACCUCUUGGUCUUUGAAUAAAGCCUGAGUAGGAAGUCUAG (SEQ ID NO: 1616) | |
| | | PolyA_tail: AAAAAAAAAAAAAAAAAAAAAAAAAAAAAAAAAAAAAAAAAAAAAAAAAAAAA | |
| | | AAAAAAAAAAAAAAAAAAAAAAA (SEQ ID NO: 1617) | |
| T2-Ef1a- | DNA | AAV2 | 1537 |
| mKate2- | | ITR: CCTGCAGGCAGCTGCGCGCTCGCTCGCTCACTGAGGCCGCCCGGGCGTCGGGCGACCTT | |
| WPRE | | TGGTCGCCCGGCCTCAGTGAGCGAGCGAGCGCGCAGAGAGGGAGTGGCCAACTCCATCACTAG | |
| | | GGGTTCCT (SEQ ID NO: 1618) | |
| | | GCGGCCGCACGCGTCTAGTTATTAATAGTAATCAATTACGGGGTCATTAGTTCATAGCCCATA | |
| | | TATGGAGTTCCGATCCCTATACAGTTGAAGTCGGAAGTTTACATACACTTA (SEQ ID NO: | |
| | | 1619) | |
| | | 5′ SB pT2 IR: | |
| | | AGTTGGAGTCATTAAAACTCGTTTTTCAACTACTCCACAAATTTCTTGTTAACAAACAATAGT | |
| | | TTTGGCAAGTCAGTTAGGACATCTACTTTGTGCATGACACAAGTCATTTTTCCAACAATTGTT | |
| | | TACAGACAGATTATTTCACTTATAATTCACTGTATCACAATTC (SEQ ID NO: 1620) | |
| | | CAGTGGGTCAGAAGTTTACATACACTAAGTTGACTGTGCCTTTAAACAGCTTGGAAAATTCCA | |
| | | GAAAATGATGTCATGGCTTTAGAAGCTAACATGTGCGACGTAGCTTGGGTAGGTGAGCGATTA | |
| | | ACCGTCCCTTTAGGTACCACTAGT (SEQ ID NO: 1621) | |
| | | Ef1a promoter: | |
| | | GGCTCCGGTGCCCGTCAGTGGGCAGAGCGCACATCGCCCACAGTCCCCGAGAAGTTGGGGGGA | |
| | | GGGGTCGGCAATTGAACCGGTGCCTAGAGAAGGTGGCGCGGGGTAAACTGGGAAAGTGATGTC | |
| | | GTGTACTGGCTCCGCCTTTTTCCCGAGGGTGGGGGAGAACCGTATATAAGTGCAGTAGTCGCC | |
| | | GTGAACGTTCTTTTTCGCAACGGGTTTGCCGCCAGAACACAGGTAAGTGCCGTGTGTGGTTCC | |
| | | CGCGGGCCTGGCCTCTTTACGGGTTATGGCCCTTGCGTGCCTTGAATTACTTCCACCTGGCTG | |
| | | CAGTACGTGATTCTTGATCCCGAGCTTCGGGTTGGAAGTGGGTGGGAGAGTTCGAGGCCTTGC | |
| | | GCTTAAGGAGCCCCTTCGCCTCGTGCTTGAGTTGAGGCCTGGCCTGGGCGCTGGGGCCGCCGC | |
| | | GTGCGAATCTGGTGGCACCTTCGCGCCTGTCTCGCTGCTTTCGATAAGTCTCTAGCCATTTAA | |
| | | AATTTTTGATGACCTGCTGCGACGCTTTTTTTCTGGCAAGATAGTCTTGTAAATGCGGGCCAA | |
| | | GATCTGCACACTGGTATTTCGGTTTTTGGGGCCGCGGGCGGCGACGGGGCCCGTGCGTCCCAG | |
| | | CGCACATGTTCGGCGAGGCGGGGCCTGCGAGCGCGGCCACCGAGAATCGGACGGGGGTAGTCT | |
| | | CAAGCTGGCCGGCCTGCTCTGGTGCCTGGTCTCGCGCCGCCGTGTATCGCCCCGCCCTGGGCG | |
| | | GCAAGGCTGGCCCGGTCGGCACCAGTTGCGTGAGCGGAAAGATGGCCGCTTCCCGGCCCTGCT | |
| | | GCAGGGAGCTCAAAATGGAGGACGCGGCGCTCGGGAGAGCGGGCGGGTGAGTCACCCACACAA | |
| | | AGGAAAAGGGCCTTTCCGTCCTCAGCCGTCGCTTCATGTGACTCCACGGAGTACCGGGCGCCG | |
| | | TCCAGGCACCTCGATTAGTTCTCGAGCTTTTGGAGTACGTCGTCTTTAGGTTGGGGGGAGGGG | |
| | | TTTTATGCGATGGAGTTTCCCCACACTGAGTGGGTGGAGACTGAAGTTAGGCCAGCTTGGCAC | |
| | | TTGATGTAATTCTCCTTGGAATTTGCCCTTTTTGAGTTTGGATCTTGGTTCATTCTCAAGCCT | |
| | | CAGACAGTGGTTCAAAGTTTTTTTCTTCCATTTCAGGTGTCGTGA (SEQ ID NO: 1622) | |
| | | TAATACGACTCAGCTAGCGTTTAAACTTAAGCTTGAGCTCGGATCCCCAGTGTGGTGGAATTC | |
| | | (SEQ ID NO: 1623) | |
| | | Kozak: GCCACC | |
| | | mKate2 CDS: | |
| | | ATGGTTTCCGAGCTGATCAAAGAAAACATGCACATGAAGCTGTACATGGAAGGCACCGTGAAC | |
| | | AACCACCACTTCAAGTGCACCAGCGAAGGCGAGGGCAAGCCTTATGAGGGCACCCAGACCATG | |
| | | AGAATCAAGGCCGTTGAAGGCGGCCCTCTGCCTTTCGCCTTTGATATCCTGGCCACCAGCTTT | |
| | | ATGTACGGCAGCAAGACCTTCATCAATCACACCCAGGGCATCCCCGATTTCTTCAAGCAGAGC | |
| | | TTCCCCGAGGGCTTCACCTGGGAGAGAGTGACCACATACGAGGATGGCGGCGTGCTGACAGCC | |
| | | ACACAGGATACAAGTCTGCAGGACGGCTGCCTGATCTACAACGTGAAGATCCGGGGCGTGAAC | |
| | | TTCCCCAGCAATGGCCCCGTGATGCAGAAGAAAACCCTCGGCTGGGAAGCCAGCACCGAGACA | |
| | | CTGTATCCTGCCGATGGTGGCCTGGAAGGCAGAGCTGATATGGCCCTGAAACTCGTTGGCGGC | |
| | | GGACACCTGATCTGCAATCTGAAAACCACCTACCGGTCCAAGAAGCCCGCCAAGAACCTGAAG | |
| | | ATGCCCGGCGTGTACTACGTGGACAGACGGCTGGAACGGATCAAAGAGGCCGACAAAGAAACC | |
| | | TACGTGGAACAGCACGAGGTGGCCGTGGCCAGATACTGTGATCTGCCTTCTAAGCTGGGCCAC | |
| | | AGA (SEQ ID NO: 1624) | |
| | | Stop codon: TGA | |
| | | TAATCTAGAGTCGACCTGCAGAAGCTTGATATCACCGGTCGAT (SEQ ID NO: 1625) | |
| | | WPRE: AATCAACCTCTGGATTACAAAATTTGTGAAAGATTGACTGGTATTCTTAACTATGTTG | |
| | | CTCCTTTTACGCTATGTGGATACGCTGCTTTAATGCCTTTGTATCATGCTATTGCTTCCCGTA | |
| | | TGGCTTTCATTTTCTCCTCCTTGTATAAATCCTGGTTGCTGTCTCTTTATGAGGAGTTGTGGC | |
| | | CCGTTGTCAGGCAACGTGGCGTGGTGTGCACTGTGTTTGCTGACGCAACCCCCACTGGTTGGG | |
| | | GCATTGCCACCACCTGTCAGCTCCTTTCCGGGACTTTCGCTTTCCCCCTCCCTATTGCCACGG | |
| | | CGGAACTCATCGCCGCCTGCCTTGCCCGCTGCTGGACAGGGGCTCGGCTGTTGGGCACTGACA | |
| | | ATTCCGTGGTGTTGTCGGGGAAATCATCGTCCTTTCCTTGGCTGCTCGCCTGTGTTGCCACCT | |
| | | GGATTCTGCGCGGGACGTCCTTCTGCTACGTCCCTTCGGCCCTCAATCCAGCGGACCTTCCTT | |
| | | CCCGCGGCCTGCTGCCGGCTCTGCGGCCTCTTCCGCGTCTTCGCCTTCGCCCTCAGACGAGTC | |
| | | GGATCTCCCTTTGGGCCGCCTCCCCGC (SEQ ID NO: 1626) | |
| | | ATAGCGCTGCTCGAGAGATCTAC (SEQ ID NO: 1627) | |
| | | BGH polyA signal: | |
| | | GGGTGGCATCCCTGTGACCCCTCCCCAGTGCCTCTCCTGGCCCTGGAAGTTGCCACTCCAGTG | |
| | | CCCACCAGCCTTGTCCTAATAAAATTAAGTTGCATCATTTTGTCTGACTAGGTGTCCTTCTAT | |
| | | AATATTATGGGGTGGAGGGGGGTGGTATGGAGCAAGGGGCAAGTTGGGAAGACAACCTGTAGG | |
| | | GCCTGCGGGGTCTATTGGGAACCAAGCTGGAGTGCAGTGGCACAATCTTGGCTCACTGCAATC | |
| | | TCCGCCTCCTGGGTTCAAGCGATTCTCCTGCCTCAGCCTCCCGAGTTGTTGGGATTCCAGGCA | |
| | | TGCATGACCAGGCTCAGCTAATTTTTGTTTTTTTGGTAGAGACGGGGTTTCACCATATTGGCC | |
| | | AGGCTGGTCTCCAACTCCTAATCTCAGGTGATCTACCCACCTTGGCCTCCCAAATTGCTGGGA | |
| | | TTACAGGCGTGAACCACTGCTCCCTTCCCTGTCCTT (SEQ ID NO: 1628) | |
| | | TCCGGAGCGGCCGCGTTTAATTGAGTTGTCATATGTTAATAACGGTATGTGGAAGGCTACTCG | |
| | | AAATGTTTGACCCAAGTTAAACAATTTAAAGGCAATGCTACCAAATACTAATTGAGTGTATGT | |
| | | AAACTTCTGACCCACTG (SEQ ID NO: 1629) | |
| | | SB pT2 3 IR: | |
| | | GGAATGTGATGAAAGAAATAAAAGCTGAAATGAATCATTCTCTCTACTATTATTCTGATATTT | |
| | | CACATTCTTAAAATAAAGTGGTGATCCTAACTGACCTAAGACAGGGAATTTTTACTAGGATTA | |
| | | AATGTCAGGAATTGTGAAAAAGTGAGTTTAAATGTATTTGGCT (SEQ ID NO: 1630) | |
| | | AAGGTGTATGTAAACTTCCGACTTCAACTGTATAGGGATCCTCTAGCTACTGATTTTGTAGGT | |
| | | AACCACGTGCGGACCGAGCGGCCGC (SEQ ID NO: 1631) | |
| | | AAV2 ITR: | |
| | | AGGAACCCCTAGTGATGGAGTTGGCCACTCCCTCTCTGCGCGCTCGCTCGCTCACTGAGGCCG | |
| | | GGCGACCAAAGGTCGCCCGACGCCCGGGCTTTGCCCGGGCGGCCTCAGTGAGCGAGCGAGCGC | |
| | | GCAGCTGCCTGCAGG (SEQ ID NO: 1632) | |
| SB100X | Protein | MGKSKEISQDLRKRIVDLHKSGSSLGAISKRLAVPRSSVQTIVRKYKHHGTTQPSYRSGRRRV | 1530 |
| polypeptide | | LSPRDERTLVRKVQINPRTTAKDLVKMLEETGTKVSISTVKRVLYRHNLKGHSARKKPLLQNR | |
| | | HKKARLRFATAHGDKDRTFWRNVLWSDETKIELFGHNDHRYVWRKKGEACKPKNTIPTVKHGG | |
| | | GSIMLWGCFAAGGTGALHKIDGIMDAVOYVDILKQHLKTSVRKLKLGRKWVFQHDNDPKHTSK | |
| | | VVAKWLKDNKVKVLEWPSQSPDLNPIENLWAELKKRVRARRPTNLTQLHQLCQEEWAKIHPNY | |
| | | CGKLVEGYPKRLTQVKOFKGNATKY | |
| AAV8-T2- | DNA | AAV2 ITR: | 1538 |
| SerpENH- | | CCTGCAGGCAGCTGCGCGCTCGCTCGCTCACTGAGGCCGCCCGGGCGTCGGGCGACCTTTGGT | |
| TTRmin- | | CGCCCGGCCTCAGTGAGCGAGCGAGCGCGCAGAGAGGGAGTGGCCAACTCCATCACTAGGGGT | |
| rhCG- | | TCCT (SEQ ID NO: 1618) | |
| WPRE- | | GCGGCCATTCGGTACAATTCACGCGTGAGACGTACAAAAAAGAGCAAGAAGCTAAAAAAGATT | |
| bGH pA | | TAAAAATTATTTTTAGCGCAGTTAATGGAACAGGAACTAAATTTACCCCAAAAATATTACGTG | |
| | | AATCAGGATATAACGTTATTGAGGTTGAAGAGCATGCATTTGAAGATGAAACATTTAAAAATG | |
| | | TTGTAAATCCAAATCCAGAATTTGATCCTGCATGAAAAATACCGCTTGAATATGGTATTAAAC | |
| | | ATGATGCAGATATTATTATTATGAATGACCCAGATGCTGACAGATTTGGAATGGCAATAAAAC | |
| | | ATGATGGTCATTTTGTAAGATTAGATGGAAATCAAACAGGACCAATTTTAATTGATTGAAAAT | |
| | | TATCAAATCTAAAACGCTTAAATAGCATTCCAAAAAATCCGGCTCTATATTCAAGTTTTGTAA | |
| | | CAAGTGATTTGGGTGATAGAATCGCTCATGAAAAATATGGAGTTAATATTGTAAAAACTTTAA | |
| | | CTGGATTTAAATGAATGGGTAGAGAAATTGCTAAAGAAGAAGATAACGGATTAAATTTTGTTT | |
| | | TTGCTTATGAAGAAAGTTATGGATATGTAATTGATGACTCAGCTAGAGATAAAGATGGAATAC | |
| | | AAGCTTCTATATTAATAGCAGAGGCTGCTTGATTTTATAAAAAACAAAATAAAACATTAGTAG | |
| | | ACTATTTAGAAGATTTATTTAAAGAAATGGGTGCATATTACACTTTCACTTTAAACTTGAATT | |
| | | TTAAACCAGAAGAAAAGAAATTAAAAATTGAACCATTAATGAAATCATTGAGAGCAACACCCT | |
| | | TAACTCAAATTGCTGGACTTAAAGTTGTTAATGTTGAAGACTACATCGATGGAATGTATAATA | |
| | | TGCCAGGACAAGACTTACTAAAATTTTATTTAGAAGATAAGTCATGATTTGCTGTTCGCCCAA | |
| | | GTGGAACTGAACCTAAACTAAAAATTTATTTTATAGGTGTTGGTGAATCTGTTCAAAACGCTA | |
| | | AAGTTAAAGTAGACGAAATTATTAAAGAATTAAAATTAAAAATGAATATATAGGAGAAAAAAT | |
| | | GAAACTAAACAAATATATAGATCACACATTATTAAAACAAGATGCTACGAAAGCTGAAATTAA | |
| | | ACAATTATGTGATGAAGCAATTGAATTTGATTTTGCAACAGTTTGTGTTAATTCATATTGAAC | |
| | | AAGCTATTGTAAAGAATTATTAAAAGGCACAAATGTAGGAATAACAAATGTTGTAGGTTTTCC | |
| | | TCTAGGTGCATGCACAACAGCTACAAAAGCATTCGAAGTTTCTGAAGCAATTAAAGATGGTGC | |
| | | AACAGAAATTGATATGGTATTAAATATTGGTGCATTAAAAGACAAAAATTATGAATTAGTTTT | |
| | | AGAAGACATGAAAGCTGTAAAAAAAGCAGCTGGATCACATGTTGTTAAATGTATTATGGAAAA | |
| | | TTGTTTATTAACAAAAGAAGAAATCATGAAAGCTTGTGAAATAGCTGTTGAAGCTGGATTAGA | |
| | | ATTTGTTAAAACATCAACAGGATTTTCAAAATCAGGTGCAACATTTGAAGATGTTAAACTAAT | |
| | | GATCCCTATACAGTTGAAGTCGGAAGTTTACATACACTTA (SEQ ID NO: 1633) | |
| | | 5′ SB pT2 IR: | |
| | | AGTTGGAGTCATTAAAACTCGTTTTTCAACTACTCCACAAATTTCTTGTTAACAAACAATAGT | |
| | | TTTGGCAAGTCAGTTAGGACATCTACTTTGTGCATGACACAAGTCATTTTTCCAACAATTGTT | |
| | | TACAGACAGATTATTTCACTTATAATTCACTGTATCACAATTC (SEQ ID NO: 1620) | |
| | | CAGTGGGTCAGAAGTTTACATACACTAAGTTGACTGTGCCTTTAAACAGCTTGGAAAATTCCA | |
| | | GAAAATGATGTCATGGCTTTAGAAGCTAACATGTGCGACGTAGCTTGGGTAGGTGAGCGATTA | |
| | | ACCGTCCCTTTA (SEQ ID NO: 1634) | |
| | | Serpin Enhancer-TTR minimal promoter: | |
| | | GGGGGAGGCTGCTGGTGAATATTAACCAAGGTCACCCCAGTTATCGGAGGAGCAAACAGGGGC | |
| | | TAAGTCCACACGCGTGGTACCGTCTGTCTGCACATTTCGTAGAGCGAGTGTTCCGATACTCTA | |
| | | ATCTCCCTAGGCAAGGTTCATATTTGTGTAGGTTACTTATTCTCCTTTTGTTGACTAAGTCAA | |
| | | TAATCAGAATCAGCAGGTTTGGAGTCAGCTTGGCAGGGATCAGCAGCCTGGGTTGGAAGGAGG | |
| | | GGGTATAAAAGCCCCTTCACCAGGAGAAGCCGTCACACAGATCCACAAGCTCCTG (SEQ ID | |
| | | NO: 1635) | |
| | | Kozak: GCCGCCACC | |
| | | rhCG CDS: | |
| | | ATGGAGATGCTCCAGGGGCTGCTGCTGTGGCTGCTGCTGAGCATGGGGGGGGCACGGGCATCC | |
| | | AGGGAGCCGCTGCGGCCACTGTGCCGCCCCATCAATGCCACCCTGGCTGCCGAGAAGGAGGCC | |
| | | TGCCCCGTGTGCATCACCGTCAACACCACCATCTGTGCCGGCTACTGCCCCACCATGATGCGG | |
| | | GTGCTGCAGGCGGTCCTGCCGCCAGTGCCCCAGGTGGTGCGCAACTACCGCGAGGTGCGCTTC | |
| | | GAGTCCATCCGGCTCCCTGGCTGCCCGCCTGGCGTGGACCCCGTGGTCTCCGTTCCCGTGGCT | |
| | | CTCAGCTGTCGTTGTGCACTCTGCCGCCGCAGCACCTCTGACTGTGGGGGTCCCAAGGACCAC | |
| | | CCTTTGACCTGTGATGACCCCCACCTCCAGGCCTCCTCTTCCTCAAAGGACCCTCCCCCCAGC | |
| | | CCTCCAAGTCCATCCGGACTCCTGGAGCCAGCAGACAACCCGTTCCTCCCGCAA (SEQ ID | |
| | | NO: 1636) | |
| | | Stop codon: TAA | |
| | | WPRE: | |
| | | AATCAACCTCTGGATTACAAAATTTGTGAAAGATTGACTGGTATTCTTAACTATGTTGCTCCT | |
| | | TTTACGCTATGTGGATACGCTGCTTTAATGCCTTTGTATCATGCTATTGCTTCCCGTATGGCT | |
| | | TTCATTTTCTCCTCCTTGTATAAATCCTGGTTGCTGTCTCTTTATGAGGAGTTGTGGCCCGTT | |
| | | GTCAGGCAACGTGGCGTGGTGTGCACTGTGTTTGCTGACGCAACCCCCACTGGTTGGGGCATT | |
| | | GCCACCACCTGTCAGCTCCTTTCCGGGACTTTCGCTTTCCCCCTCCCTATTGCCACGGCGGAA | |
| | | CTCATCGCCGCCTGCCTTGCCCGCTGCTGGACAGGGGCTCGGCTGTTGGGCACTGACAATTCC | |
| | | GTGGTGTTGTCGGGGAAATCATCGTCCTTTCCTTGGCTGCTCGCCTGTGTTGCCACCTGGATT | |
| | | CTGCGCGGGACGTCCTTCTGCTACGTCCCTTCGGCCCTCAATCCAGCGGACCTTCCTTCCCGC | |
| | | GGCCTGCTGCCGGCTCTGCGGCCTCTTCCGCGTCTTCGCCTTCGCCCTCAGACGAGTCGGATC | |
| | | TCCCTTTGGGCCGCCTCCCCGC (SEQ ID NO: 1626) | |
| | | ATCGATACCGTCGACTCGCTGATCAGCCTCGA (SEQ ID NO: 1637) | |
| | | BGH polyA signal: | |
| | | CTGTGCCTTCTAGTTGCCAGCCATCTGTTGTTTGCCCCTCCCCCGTGCCTTCCTTGACCCTGG | |
| | | AAGGTGCCACTCCCACTGTCCTTTCCTAATAAAATGAGGAAATTGCATCGCATTGTCTGAGTA | |
| | | GGTGTCATTCTATTCTGGGGGGTGGGGTGGGGCAGGACAGCAAGGGGGAGGATTGGGAAGACA | |
| | | ATAGCAGGCATGCTGGGGATGCGGTGGGCTCTATGG (SEQ ID NO: 1638) | |
| | | GTTTAATTGAGTTGTCATATGTTAATAACGGTATGTGGAAGGCTACTCGAAATGTTTGACCCA | |
| | | AGTTAAACAATTTAAAGGCAATGCTACCAAATACTAATTGAGTGTATGTAAACTTCTGACCCA | |
| | | CTG (SEQ ID NO: 1639) | |
| | | SB pT2 3 IR: | |
| | | GGAATGTGATGAAAGAAATAAAAGCTGAAATGAATCATTCTCTCTACTATTATTCTGATATTT | |
| | | CACATTCTTAAAATAAAGTGGTGATCCTAACTGACCTAAGACAGGGAATTTTTACTAGGATTA | |
| | | AATGTCAGGAATTGTGAAAAAGTGAGTTTAAATGTATTTGGCT (SEQ ID NO: 1630) | |
| | | AAGGTGTATGTAAACTTCCGACTTCAACTGTATATCTAGATCCGGAGAGCTCCTCGAGGCGGC | |
| | | CGC (SEQ ID NO: 1640) | |
| | | AAV2 ITR: | |
| | | AGGAACCCCTAGTGATGGAGTTGGCCACTCCCTCTCTGCGCGCTCGCTCGCTCACTGAGGCCG | |
| | | GGCGACCAAAGGTCGCCCGACGCCCGGGCTTTGCCCGGGCGGCCTCAGTGAGCGAGCGAGCGC | |
| | | GCAGCTGCCTGCAGG (SEQ ID NO: 1632) | |
| AAV8-No | DNA | AAV2 ITR: | 1539 |
| SB IRs- | | CCTGCAGGCAGCTGCGCGCTCGCTCGCTCACTGAGGCCGCCCGGGCGTCGGGCGACCTTTGGT | |
| SerpENH- | | CGCCCGGCCTCAGTGAGCGAGCGAGCGCGCAGAGAGGGAGTGGCCAACTCCATCACTAGGGGT | |
| TTRmin- | | TCCT (SEQ ID NO: 1618) | |
| rhCG- | | GCGGCCATTCGGTACAATTCACGCGTGAGACGTACAAAAAAGAGCAAGAAGCTAAAAAAGATT | |
| WPRE- | | TAAAAATTATTTTTAGCGCAGTTAATGGAACAGGAACTAAATTTACCCCAAAAATATTACGTG | |
| bGH pA | | AATCAGGATATAACGTTATTGAGGTTGAAGAGCATGCATTTGAAGATGAAACATTTAAAAATG | |
| | | TTGTAAATCCAAATCCAGAATTTGATCCTGCATGAAAAATACCGCTTGAATATGGTATTAAAC | |
| | | ATGATGCAGATATTATTATTATGAATGACCCAGATGCTGACAGATTTGGAATGGCAATAAAAC | |
| | | ATGATGGTCATTTTGTAAGATTAGATGGAAATCAAACAGGACCAATTTTAATTGATTGAAAAT | |
| | | TATCAAATCTAAAACGCTTAAATAGCATTCCAAAAAATCCGGCTCTATATTCAAGTTTTGTAA | |
| | | CAAGTGATTTGGGTGATAGAATCGCTCATGAAAAATATGGAGTTAATATTGTAAAAACTTTAA | |
| | | CTGGATTTAAATGAATGGGTAGAGAAATTGCTAAAGAAGAAGATAACGGATTAAATTTTGTTT | |
| | | TTGCTTATGAAGAAAGTTATGGATATGTAATTGATGACTCAGCTAGAGATAAAGATGGAATAC | |
| | | AAGCTTCTATATTAATAGCAGAGGCTGCTTGATTTTATAAAAAACAAAATAAAACATTAGTAG | |
| | | ACTATTTAGAAGATTTATTTAAAGAAATGGGTGCATATTACACTTTCACTTTAAACTTGAATT | |
| | | TTAAACCAGAAGAAAAGAAATTAAAAATTGAACCATTAATGAAATCATTGAGAGCAACACCCT | |
| | | TAACTCAAATTGCTGGACTTAAAGTTGTTAATGTTGAAGACTACATCGATGGAATGTATAATA | |
| | | TGCCAGGACAAGACTTACTAAAATTTTATTTAGAAGATAAGTCATGATTTGCTGTTCGCCCAA | |
| | | GTGGAACTGAACCTAAACTAAAAATTTATTTTATAGGTGTTGGTGAATCTGTTCAAAACGCTA | |
| | | AAGTTAAAGTAGACGAAATTATTAAAGAATTAAAATTAAAAATGAATATATAGGAGAAAAAAT | |
| | | GAAACTAAACAAATATATAGATCACACATTATTAAAACAAGATGCTACGAAAGCTGAAATTAA | |
| | | ACAATTATGTGATGAAGCAATTGAATTTGATTTTGCAACAGTTTGTGTTAATTCATATTGAAC | |
| | | AAGCTATTGTAAAGAATTATTAAAAGGCACAAATGTAGGAATAACAAATGTTGTAGGTTTTCC | |
| | | TCTAGGTGCATGCACAACAGCTACAAAAGCATTCGAAGTTTCTGAAGCAATTAAAGATGGTGC | |
| | | AACAGAAATTGATATGGTATTAAATATTGGTGCATTAAAAGACAAAAATTATGAATTAGTTTT | |
| | | AGAAGACATGAAAGCTGTAAAAAAAGCAGCTGGATCACATGTTGTTAAATGTATTATGGAAAA | |
| | | TTGTTTATTAACAAAAGAAGAAATCATGAAAGCTTGTGAAATAGCTGTTGAAGCTGGATTAGA | |
| | | ATTTGTTAAAACATCAACAGGATTTTCAAAATCAGGTGCAACATTTGAAGATGTTAAACTAAT | |
| | | GATCCTTGACTGTGCCTTTAAACAGCTTGGAAAATTCCAGAAAATGATGTCATGGCTTTAGAA | |
| | | GCTAACATGTGCGACGTAGCTTGGGTAGGTGAGCGATTAACCGTCCCTTTA (SEQ ID NO: | |
| | | 1641) | |
| | | Serpin Enhancer-TTR minimal promoter: | |
| | | GGGGGAGGCTGCTGGTGAATATTAACCAAGGTCACCCCAGTTATCGGAGGAGCAAACAGGGGC | |
| | | TAAGTCCACACGCGTGGTACCGTCTGTCTGCACATTTCGTAGAGCGAGTGTTCCGATACTCTA | |
| | | ATCTCCCTAGGCAAGGTTCATATTTGTGTAGGTTACTTATTCTCCTTTTGTTGACTAAGTCAA | |
| | | TAATCAGAATCAGCAGGTTTGGAGTCAGCTTGGCAGGGATCAGCAGCCTGGGTTGGAAGGAGG | |
| | | GGGTATAAAAGCCCCTTCACCAGGAGAAGCCGTCACACAGATCCACAAGCTCCTG (SEQ ID | |
| | | NO: 1635) | |
| | | Kozak: GCCGCCACC | |
| | | rhCG CDS: | |
| | | ATGGAGATGCTCCAGGGGCTGCTGCTGTGGCTGCTGCTGAGCATGGGGGGGGCACGGGCATCC | |
| | | AGGGAGCCGCTGCGGCCACTGTGCCGCCCCATCAATGCCACCCTGGCTGCCGAGAAGGAGGCC | |
| | | TGCCCCGTGTGCATCACCGTCAACACCACCATCTGTGCCGGCTACTGCCCCACCATGATGCGG | |
| | | GTGCTGCAGGCGGTCCTGCCGCCAGTGCCCCAGGTGGTGCGCAACTACCGCGAGGTGCGCTTC | |
| | | GAGTCCATCCGGCTCCCTGGCTGCCCGCCTGGCGTGGACCCCGTGGTCTCCGTTCCCGTGGCT | |
| | | CTCAGCTGTCGTTGTGCACTCTGCCGCCGCAGCACCTCTGACTGTGGGGGTCCCAAGGACCAC | |
| | | CCTTTGACCTGTGATGACCCCCACCTCCAGGCCTCCTCTTCCTCAAAGGACCCTCCCCCCAGC | |
| | | CCTCCAAGTCCATCCGGACTCCTGGAGCCAGCAGACAACCCGTTCCTCCCGCAA (SEQ ID | |
| | | NO: 1636) | |
| | | Stop codon: TAA | |
| | | WPRE: | |
| | | AATCAACCTCTGGATTACAAAATTTGTGAAAGATTGACTGGTATTCTTAACTATGTTGCTCCT | |
| | | TTTACGCTATGTGGATACGCTGCTTTAATGCCTTTGTATCATGCTATTGCTTCCCGTATGGCT | |
| | | TTCATTTTCTCCTCCTTGTATAAATCCTGGTTGCTGTCTCTTTATGAGGAGTTGTGGCCCGTT | |
| | | GTCAGGCAACGTGGCGTGGTGTGCACTGTGTTTGCTGACGCAACCCCCACTGGTTGGGGCATT | |
| | | GCCACCACCTGTCAGCTCCTTTCCGGGACTTTCGCTTTCCCCCTCCCTATTGCCACGGCGGAA | |
| | | CTCATCGCCGCCTGCCTTGCCCGCTGCTGGACAGGGGCTCGGCTGTTGGGCACTGACAATTCC | |
| | | GTGGTGTTGTCGGGGAAATCATCGTCCTTTCCTTGGCTGCTCGCCTGTGTTGCCACCTGGATT | |
| | | CTGCGCGGGACGTCCTTCTGCTACGTCCCTTCGGCCCTCAATCCAGCGGACCTTCCTTCCCGC | |
| | | GGCCTGCTGCCGGCTCTGCGGCCTCTTCCGCGTCTTCGCCTTCGCCCTCAGACGAGTCGGATC | |
| | | TCCCTTTGGGCCGCCTCCCCGC (SEQ ID NO: 1626) | |
| | | BGH polyA signal: | |
| | | ATCGATACCGTCGACTCGCTGATCAGCCTCGACTGTGCCTTCTAGTTGCCAGCCATCTGTTGT | |
| | | TTGCCCCTCCCCCGTGCCTTCCTTGACCCTGGAAGGTGCCACTCCCACTGTCCTTTCCTAATA | |
| | | AAATGAGGAAATTGCATCGCATTGTCTGAGTAGGTGTCATTCTATTCTGGGGGGTGGGGTGGG | |
| | | GCAGGACAGCAAGGGGGAGGATTGGGAAGACAATAGCAGGCATGCTGGGGATGCGGTGGGCTC | |
| | | TATGG (SEQ ID NO: 1642) | |
| | | GTTTAATTGAGTTGTCATATGTTAATAACGGTATGTGGAAGGCTACTCGAAATGTTTGACCCA | |
| | | AGTTAAACAATTTAAAGGCAATGCTACCAAATACTAATCTAGATCCGGAGAGCTCCTCGAGGC | |
| | | GGCCGC (SEQ ID NO: 1643) | |
| | | AAV2 ITR: | |
| | | AGGAACCCCTAGTGATGGAGTTGGCCACTCCCTCTCTGCGCGCTCGCTCGCTCACTGAGGCCG | |
| | | GGCGACCAAAGGTCGCCCGACGCCCGGGCTTTGCCCGGGCGGCCTCAGTGAGCGAGCGAGCGC | |
| | | GCAGCTGCCTGCAGG (SEQ ID NO: 1632) | |
| AAV8- | DNA | AAV2 ITR: | 1540 |
| 2xApoE- | | CCTGCAGGCAGCTGCGCGCTCGCTCGCTCACTGAGGCCGCCCGGGCGTCGGGCGACCTTTGGT | |
| HCR1 | | CGCCCGGCCTCAGTGAGCGAGCGAGCGCGCAGAGAGGGAGTGGCCAACTCCATCACTAGGGGT | |
| hAAT- | | TCCT (SEQ ID NO: 1618) | |
| EGFP- | | GCGGCCATTCGGTACAATTCACGCGTCTAAGTTAATTAACTGCAG (SEQ ID NO: 1644) | |
| WPRE | | 2xApoE-HCR1 Enhancer-hAAT Promoter: | |
| bGH pA | | GCTCAGAGGCACACAGGAGTTTCTGGGCTCACCCTGCCCCCTTCCAACCCCTCAGTTCCCATC | |
| | | CTCCAGCAGCTGTTTGTGTGCTGCCTCTGAAGTCCACACTGAACAAACTTCAGCCTACTCATG | |
| | | TCCCTAAAATGGGCAAACATTGCAAGCAGCAAACAGCAAACACACAGCCCTCCCTGCCTGCTG | |
| | | ACCTTGGAGCTGGGGCAGAGGTCAGAGACCTCTCTGGGCCCATGCCACCTCCAACATCCACTC | |
| | | GACCCCTTGGAATTTCGGTGGAGAGGAGCAGAGGTTGTCCTGGCGTGGTTTAGGTAGTGTGAG | |
| | | AGGGTCCGGCGATTAACTGCAGGCTCAGAGGCACACAGGAGTTTCTGGGCTCACCCTGCCCCC | |
| | | TTCCAACCCCTCAGTTCCCATCCTCCAGCAGCTGTTTGTGTGCTGCCTCTGAAGTCCACACTG | |
| | | AACAAACTTCAGCCTACTCATGTCCCTAAAATGGGCAAACATTGCAAGCAGCAAACAGCAAAC | |
| | | ACACAGCCCTCCCTGCCTGCTGACCTTGGAGCTGGGGCAGAGGTCAGAGACCTCTCTGGGCCC | |
| | | ATGCCACCTCCAACATCCACTCGACCCCTTGGAATTTCGGTGGAGAGGAGCAGAGGTTGTCCT | |
| | | GGCGTGGTTTAGGTAGTGTGAGAGGGTCCGGCGATTAAGATCTTGCTACCAGTGGAACAGCCA | |
| | | CTAAGGATTCTGCAGTGAGAGCAGAGGGCCAGCTAAGTGGTACTCTCCCAGAGACTGTCTGAC | |
| | | TCACGCCACCCCCTCCACCTTGGACACAGGACGCTGTGGTTTCTGAGCCAGGTACAATGACTC | |
| | | CTTTCGGTAAGTGCAGTGGAAGCTGTACACTGCCCAGGCAAAGCGTCCGGGCAGCGTAGGCGG | |
| | | GCGACTCAGATCCCAGCCAGTGGACTTAGCCCCTGTTTGCTCCTCCGATAACTGGGGTGACCT | |
| | | TGGTTAATATTCACCAGCAGCCTCCCCCGTTGCCCCTCTGGATCCACTGCTTAAATACGGACG | |
| | | AGGACAGGGCCCTGTCTCCTCAGCTTCAGGCACCACCACTGACCTGGGACAGTGAAT (SEQ | |
| | | ID NO: 1645) | |
| | | GCGGCCGCTCTAGAACTAGTGGATCCCCCGGGCTGCAGGAATTCACTAGTGATTTC (SEQ | |
| | | ID NO: 1646) | |
| | | Kozak: GCCGCCACC | |
| | | eGFP CDS: | |
| | | ATGGTGAGCAAGGGCGAGGAGCTGTTCACCGGGGTGGTGCCCATCCTGGTCGAGCTGGACGGC | |
| | | GACGTAAACGGCCACAAGTTCAGCGTGTCCGGCGAGGGCGAGGGCGATGCCACCTACGGCAAG | |
| | | CTGACCCTGAAGTTCATCTGCACCACCGGCAAGCTGCCCGTGCCCTGGCCCACCCTCGTGACC | |
| | | ACCCTGACCTACGGCGTGCAGTGCTTCAGCCGCTACCCCGACCACATGAAGCAGCACGACTTC | |
| | | TTCAAGTCCGCCATGCCCGAAGGCTACGTCCAGGAGCGCACCATCTTCTTCAAGGACGACGGC | |
| | | AACTACAAGACCCGCGCCGAGGTGAAGTTCGAGGGCGACACCCTGGTGAACCGCATCGAGCTG | |
| | | AAGGGCATCGACTTCAAGGAGGACGGCAACATCCTGGGGCACAAGCTGGAGTACAACTACAAC | |
| | | AGCCACAACGTCTATATCATGGCCGACAAGCAGAAGAACGGCATCAAGGTGAACTTCAAGATC | |
| | | CGCCACAACATCGAGGACGGCAGCGTGCAGCTCGCCGACCACTACCAGCAGAACACCCCCATC | |
| | | GGCGACGGCCCCGTGCTGCTGCCCGACAACCACTACCTGAGCACCCAGTCCGCCCTGAGCAAA | |
| | | GACCCCAACGAGAAGCGCGATCACATGGTCCTGCTGGAGTTCGTGACCGCCGCCGGGATCACT | |
| | | CTCGGCATGGACGAGCTGTACAAG (SEQ ID NO: 1647) | |
| | | Stop codon: TAA | |
| | | GATATCAAGCTTATCGAT (SEQ ID NO: 1648) | |
| | | WPRE: | |
| | | AATCAACCTCTGGATTACAAAATTTGTGAAAGATTGACTGGTATTCTTAACTATGTTGCTCCT | |
| | | TTTACGCTATGTGGATACGCTGCTTTAATGCCTTTGTATCATGCTATTGCTTCCCGTATGGCT | |
| | | TTCATTTTCTCCTCCTTGTATAAATCCTGGTTGCTGTCTCTTTATGAGGAGTTGTGGCCCGTT | |
| | | GTCAGGCAACGTGGCGTGGTGTGCACTGTGTTTGCTGACGCAACCCCCACTGGTTGGGGCATT | |
| | | GCCACCACCTGTCAGCTCCTTTCCGGGACTTTCGCTTTCCCCCTCCCTATTGCCACGGCGGAA | |
| | | CTCATCGCCGCCTGCCTTGCCCGCTGCTGGACAGGGGCTCGGCTGTTGGGCACTGACAATTCC | |
| | | GTGGTGTTGTCGGGGAAATCATCGTCCTTTCCTTGGCTGCTCGCCTGTGTTGCCACCTGGATT | |
| | | CTGCGCGGGACGTCCTTCTGCTACGTCCCTTCGGCCCTCAATCCAGCGGACCTTCCTTCCCGC | |
| | | GGCCTGCTGCCGGCTCTGCGGCCTCTTCCGCGTCTTCGCCTTCGCCCTCAGACGAGTCGGATC | |
| | | TCCCTTTGGGCCGCCTCCCCGC (SEQ ID NO: 1626) | |
| | | BGH polyA signal: | |
| | | ATCGATACCGTCGACTCGCTGATCAGCCTCGACTGTGCCTTCTAGTTGCCAGCCATCTGTTGT | |
| | | TTGCCCCTCCCCCGTGCCTTCCTTGACCCTGGAAGGTGCCACTCCCACTGTCCTTTCCTAATA | |
| | | AAATGAGGAAATTGCATCGCATTGTCTGAGTAGGTGTCATTCTATTCTGGGGGGTGGGGTGGG | |
| | | GCAGGACAGCAAGGGGGAGGATTGGGAAGACAATAGCAGGCATGCTGGGGATGCGGTGGGCTC | |
| | | TATGG (SEQ ID NO: 1642) | |
| | | CTTCTGAGGCGGAAAGAACCAATTGCATTCATTTTATGTTTCAGGTTCAGGGGGAGATGTGGG | |
| | | AGGTTTTTTAAAGCGGCCGC (SEQ ID NO: 1649) | |
| | | AAV2 ITR: | |
| | | AGGAACCCCTAGTGATGGAGTTGGCCACTCCCTCTCTGCGCGCTCGCTCGCTCACTGAGGCCG | |
| | | GGCGACCAAAGGTCGCCCGACGCCCGGGCTTTGCCCGGGCGGCCTCAGTGAGCGAGCGAGCGC | |
| | | GCAGCTGCCTGCAGG (SEQ ID NO: 1632) | |
| AAV8- | DNA | AAV2 | 1541 |
| SerpENH | | ITR: CCTGCAGGCAGCTGCGCGCTCGCTCGCTCACTGAGGCCGCCCGGGCGTCGGGCGACCTT | |
| TTRmin- | | TGGTCGCCCGGCCTCAGTGAGCGAGCGAGCGCGCAGAGAGGGAGTGGCCAACTCCATCACTAG | |
| | | GGGTTCCT (SEQ ID NO: 1618) | |
| EGFP- | | GCGGCCATTCGGTACAATTCACGCGTGAGACGTACAAAAAAGAGCAAGAAGCTAAAAAAGATT | |
| WPRE | | TAAAAATTATTTTTAGCGCAGTTAATGGAACAGGAACTAAATTTACCCCAAAAATATTACGTG | |
| bGH pA | | AATCAGGATATAACGTTATTGAGGTTGAAGAGCATGCATTTGAAGATGAAACATTTAAAAATG | |
| | | TTGTAAATCCAAATCCAGAATTTGATCCTGCATGAAAAATACCGCTTGAATATGGTATTAAAC | |
| | | ATGATGCAGATATTATTATTATGAATGACCCAGATGCTGACAGATTTGGAATGGCAATAAAAC | |
| | | ATGATGGTCATTTTGTAAGATTAGATGGAAATCAAACAGGACCAATTTTAATTGATTGAAAAT | |
| | | TATCAAATCTAAAACGCTTAAATAGCATTCCAAAAAATCCGGCTCTATATTCAAGTTTTGTAA | |
| | | CAAGTGATTTGGGTGATAGAATCGCTCATGAAAAATATGGAGTTAATATTGTAAAAACTTTAA | |
| | | CTGGATTTAAATGAATGGGTAGAGAAATTGCTAAAGAAGAAGATAACGGATTAAATTTTGTTT | |
| | | TTGCTTATGAAGAAAGTTATGGATATGTAATTGATGACTCAGCTAGAGATAAAGATGGAATAC | |
| | | AAGCTTCTATATTAATAGCAGAGGCTGCTTGATTTTATAAAAAACAAAATAAAACATTAGTAG | |
| | | ACTATTTAGAAGATTTATTTAAAGAAATGGGTGCATATTACACTTTCACTTTAAACTTGAATT | |
| | | TTAAACCAGAAGAAAAGAAATTAAAAATTGAACCATTAATGAAATCATTGAGAGCAACACCCT | |
| | | TAACTCAAATTGCTGGACTTAAAGTTGTTAATGTTGAAGACTACATCGATGGAATGTATAATA | |
| | | TGCCAGGACAAGACTTACTAAAATTTTATTTAGAAGATAAGTCATGATTTGCTGTTCGCCCAA | |
| | | GTGGAACTGAACCTAAACTAAAAATTTATTTTATAGGTGTTGGTGAATCTGTTCAAAACGCTA | |
| | | AAGTTAAAGTAGACGAAATTATTAAAGAATTAAAATTAAAAATGAATATATAGGAGAAAAAAT | |
| | | GAAACTAAACAAATATATAGATCACACATTATTAAAACAAGATGCTACGAAAGCTGAAATTAA | |
| | | ACAATTATGTGATGAAGCAATTGAATTTGATTTTGCAACAGTTTGTGTTAATTCATATTGAAC | |
| | | AAGCTATTGTAAAGAATTATTAAAAGGCACAAATGTAGGAATAACAAATGTTGTAGGTTTTCC | |
| | | TCTAGGTGCATGCACAACAGCTACAAAAGCATTCGAAGTTTCTGAAGCAATTAAAGATGGTGC | |
| | | AACAGAAATTGATATGGTATTAAATATTGGTGCATTAAAAGACAAAAATTATGAATTAGTTTT | |
| | | AGAAGACATGAAAGCTGTAAAAAAAGCAGCTGGATCACATGTTGTTAAATGTATTATGGAAAA | |
| | | TTGTTTATTAACAAAAGAAGAAATCATGAAAGCTTGTGAAATAGCTGTTGAAGCTGGATTAGA | |
| | | ATTTGTTAAAACATCAACAGGATTTTCAAAATCAGGTGCAACATTTGAAGATGTTAAACTAAT | |
| | | GATCCTTGACTGTGCCTTTAAACAGCTTGGAAAATTCCAGAAAATGATGTCATGGCTTTAGAA | |
| | | GCTAACATGTGCGACGTAGCTTGGGTAGGTGAGCGATTAACCGTCCCTTTA (SEQ ID NO: | |
| | | 1641) | |
| | | Serpin Enhancer-TTR minimal promoter: | |
| | | GGGGGAGGCTGCTGGTGAATATTAACCAAGGTCACCCCAGTTATCGGAGGAGCAAACAGGGGC | |
| | | TAAGTCCACACGCGTGGTACCGTCTGTCTGCACATTTCGTAGAGCGAGTGTTCCGATACTCTA | |
| | | ATCTCCCTAGGCAAGGTTCATATTTGTGTAGGTTACTTATTCTCCTTTTGTTGACTAAGTCAA | |
| | | TAATCAGAATCAGCAGGTTTGGAGTCAGCTTGGCAGGGATCAGCAGCCTGGGTTGGAAGGAGG | |
| | | GGGTATAAAAGCCCCTTCACCAGGAGAAGCCGTCACACAGATCCACAAGCTCCTG (SEQ ID | |
| | | NO: 1635) | |
| | | Kozak: GCCGCCACC | |
| | | eGFP CDS: | |
| | | ATGGTGAGCAAGGGCGAGGAGCTGTTCACCGGGGTGGTGCCCATCCTGGTCGAGCTGGACGGC | |
| | | GACGTAAACGGCCACAAGTTCAGCGTGTCCGGCGAGGGCGAGGGCGATGCCACCTACGGCAAG | |
| | | CTGACCCTGAAGTTCATCTGCACCACCGGCAAGCTGCCCGTGCCCTGGCCCACCCTCGTGACC | |
| | | ACCCTGACCTACGGCGTGCAGTGCTTCAGCCGCTACCCCGACCACATGAAGCAGCACGACTTC | |
| | | TTCAAGTCCGCCATGCCCGAAGGCTACGTCCAGGAGCGCACCATCTTCTTCAAGGACGACGGC | |
| | | AACTACAAGACCCGCGCCGAGGTGAAGTTCGAGGGCGACACCCTGGTGAACCGCATCGAGCTG | |
| | | AAGGGCATCGACTTCAAGGAGGACGGCAACATCCTGGGGCACAAGCTGGAGTACAACTACAAC | |
| | | AGCCACAACGTCTATATCATGGCCGACAAGCAGAAGAACGGCATCAAGGTGAACTTCAAGATC | |
| | | CGCCACAACATCGAGGACGGCAGCGTGCAGCTCGCCGACCACTACCAGCAGAACACCCCCATC | |
| | | GGCGACGGCCCCGTGCTGCTGCCCGACAACCACTACCTGAGCACCCAGTCCGCCCTGAGCAAA | |
| | | GACCCCAACGAGAAGCGCGATCACATGGTCCTGCTGGAGTTCGTGAC CGCCGGGATCACT | |
| | | CTCGGCATGGACGAGCTGTACAAG (SEQ ID NO: 1647) | |
| | | Stop Codon: TAA | |
| | | GATATCAAGCTTATCGAT (SEQ ID NO: 1648) | |
| | | WPRE: | |
| | | AATCAACCTCTGGATTACAAAATTTGTGAAAGATTGACTGGTATTCTTAACTATGTTGCTCCT | |
| | | TTTACGCTATGTGGATACGCTGCTTTAATGCCTTTGTATCATGCTATTGCTTCCCGTATGGCT | |
| | | TTCATTTTCTCCTCCTTGTATAAATCCTGGTTGCTGTCTCTTTATGAGGAGTTGTGGCCCGTT | |
| | | GTCAGGCAACGTGGCGTGGTGTGCACTGTGTTTGCTGACGCAACCCCCACTGGTTGGGGCATT | |
| | | GCCACCACCTGTCAGCTCCTTTCCGGGACTTTCGCTTTCCCCCTCCCTATTGCCACGGCGGAA | |
| | | CTCATCGCCGCCTGCCTTGCCCGCTGCTGGACAGGGGCTCGGCTGTTGGGCACTGACAATTCC | |
| | | GTGGTGTTGTCGGGGAAATCATCGTCCTTTCCTTGGCTGCTCGCCTGTGTTGCCACCTGGATT | |
| | | CTGCGCGGGACGTCCTTCTGCTACGTCCCTTCGGCCCTCAATCCAGCGGACCTTCCTTCCCGC | |
| | | GGCCTGCTGCCGGCTCTGCGGCCTCTTCCGCGTCTTCGCCTTCGCCCTCAGACGAGTCGGATC | |
| | | TCCCTTTGGGCCGCCTCCCCGC (SEQ ID NO: 1626) | |
| | | BGH polyA signal: | |
| | | ATCGATACCGTCGACTCGCTGATCAGCCTCGACTGTGCCTTCTAGTTGCCAGCCATCTGTTGT | |
| | | TTGCCCCTCCCCCGTGCCTTCCTTGACCCTGGAAGGTGCCACTCCCACTGTCCTTTCCTAATA | |
| | | AAATGAGGAAATTGCATCGCATTGTCTGAGTAGGTGTCATTCTATTCTGGGGGGTGGGGTGGG | |
| | | GCAGGACAGCAAGGGGGAGGATTGGGAAGACAATAGCAGGCATGCTGGGGATGCGGTGGGCTC | |
| | | TATGG (SEQ ID NO: 1642) | |
| | | GTTTAATTGAGTTGTCATATGTTAATAACGGTATGTGGAAGGCTACTCGAAATGTTTGACCCA | |
| | | AGTTAAACAATTTAAAGGCAATGCTACCAAATACTAATCTAGATCCGGAGAGCTCCTCGAGGC | |
| | | GGCCGC (SEQ ID NO: 1643) | |
| | | AAV2 ITR: | |
| | | AGGAACCCCTAGTGATGGAGTTGGCCACTCCCTCTCTGCGCGCTCGCTCGCTCACTGAGGCCG | |
| | | GGCGACCAAAGGTCGCCCGACGCCCGGGCTTTGCCCGGGCGGCCTCAGTGAGCGAGCGAGCGC | |
| | | GCAGCTGCCTGCAGG (SEQ ID NO: 1632) | |

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

What is claimed is:

1. A system for modifying DNA in a target tissue comprising:
a) a transposase protein or a nucleic acid encoding the same;
b) a template nucleic acid comprising i) a sequence specifically bound by the transposase, and ii) a heterologous object sequence;
c) one or more first tissue-specific expression-control sequences specific to the target tissue, optionally wherein the one or more first tissue-specific expression-control sequences specific to the target tissues comprise a sequence selected from Table 2 or Table 3, wherein the one or more first tissue-specific expression-control sequences specific to the target tissue are in operative association with (a), (b), or (a) and (b), wherein, when associated with (a), (a) comprises a nucleic acid encoding the transposase.

2. A system for modifying DNA in a target tissue comprising:
a) a transposase protein or a nucleic acid encoding the same;
b) a template nucleic acid comprising i) a sequence specifically bound by the transposase ii) a heterologous object sequence, optionally wherein the heterologous object sequence comprises a sequence selected from Table 4, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB; and optionally
c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the transposase.

3. The system of any one of the preceding claims wherein the nucleic acid in (b) comprises RNA.

4. The system of any one of claims 1-3 wherein the nucleic acid in (b) comprises DNA.

5. The system of any one of the preceding claims, wherein the nucleic acid in (b):
i. is single-stranded or comprises a single-stranded segment, e.g., is single-stranded DNA or comprises a single-stranded segment and one or more double stranded segments;
ii. has inverted terminal repeats; or
iii. both (i) and (ii).

6. The system of any one of the preceding claims, wherein the nucleic acid in (b) is double-stranded or comprises a double-stranded segment.

7. The system of any one of the preceding claims, wherein (a) comprises a nucleic acid encoding the transposase.

8. The system of claim 7, wherein the nucleic acid in (a) comprises RNA.

9. The system of any one of claim 7 or 8, wherein the nucleic acid in (a) comprises DNA.

10. The system of any one of claims 7-9, wherein the nucleic acid in (a):
i. is single-stranded or comprises a single-stranded segment, e.g., is single-stranded DNA or comprises a single-stranded segment and one or more double stranded segments;
ii. has inverted terminal repeats; or
iii. both (i) and (ii).

11. The system of any one of claims 7-10, wherein the nucleic acid in (a) is double-stranded or comprises a double-stranded segment.

12. The system of any one of the preceding claims, wherein the nucleic acid in (a), (b), or (a) and (b) is linear.

13. The system of any one of the preceding claims, wherein the nucleic acid in (a), (b), or (a) and (b) is circular, e.g., a plasmid or minicircle.

14. The system of any one of the preceding claims, wherein the heterologous object sequence is in operative association with a first promoter.

15. The system of any one of the preceding claims, wherein the one or more first tissue-specific expression-control sequences comprises a tissue specific promoter.

16. The system of claim 15, wherein the tissue-specific promoter comprises a first promoter in operative association with:
i. the heterologous object sequence,
ii. a nucleic acid encoding the transposase, or
iii. (i) and (ii).

17. The system of any one of the preceding claims, wherein the one or more first tissue-specific expression-control sequences comprises a tissue-specific microRNA recognition sequence in operative association with:
i. the heterologous object sequence,
ii. a nucleic acid encoding the transposase, or
iii. (i) and (ii).

18. The system of any one of the preceding claims, comprising a tissue-specific promoter, the system further comprising one or more tissue-specific microRNA recognition sequences, wherein:
i. the tissue specific promoter is in operative association with:
I. the heterologous object sequence,
II. a nucleic acid encoding the transposase, or
III. (i) and (ii);
ii. The one or more tissue-specific microRNA recognition sequences are in operative association with:
I. the heterologous object sequence,
II. a nucleic acid encoding the transposase, or
III. (i) and (ii).

19. The system of any one of the preceding claims, comprising a nucleic acid encoding the transposase protein, wherein the nucleic acid comprises a promoter in operative association with the nucleic acid encoding the transposase protein.

20. The system of claim 19, wherein the nucleic acid encoding the transposase protein comprises one or more second tissue-specific expression-control sequences specific to the target tissue in operative association with the transposase coding sequence.

21. The system of claim 20, wherein the one or more second tissue-specific expression-control sequences comprises a tissue specific promoter.

22. The system of claim 21, wherein the tissue-specific promoter is the promoter in operative association with the nucleic acid encoding the transposase protein.

23. The system of any one of claims 19-22, wherein the one or more second tissue-specific expression-control sequences comprises a tissue-specific microRNA recognition sequence.

24. The system of any one of claims 19-23, wherein the promoter in operative association with the nucleic acid encoding the transposase protein is a tissue-specific promoter, the system further comprising one or more tissue-specific microRNA recognition sequences.

25. The system of any one of the preceding claims, wherein the one or more first tissue-specific expression-control sequences and, if present, one or more second tissue-specific expression-control sequences comprise a tissue-specific promoter selected from a promoter described in Table 2.

26. The system of any one of the preceding claims, wherein the one or more first tissue-specific expression-control sequences and, if present, one or more second tissue-specific expression-control sequences comprises a tissue-specific microRNA recognition sequence described in Table 3.

27. The system of any one of the preceding claims, further comprising a first recombinant adeno-associated virus (rAAV) capsid protein; wherein at least one of (a) or (b) is associated with the first rAAV capsid protein, wherein the at least one of (a) or (b) is flanked by AAV inverted terminal repeats (ITRs).

28. The system of claim 27, wherein (a) and (b) are associated with the first rAAV capsid protein, e.g., wherein (a) and (b) are on a single nucleic acid.

29. The system any one of claims 27-28, further comprising a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.

30. The system of any one of the preceding claims, wherein (a) and (b), respectively are associated with:
a) a first rAAV capsid protein and a second rAAV capsid protein
b) a nanoparticle and a first rAAV capsid protein
c) a first rAAV capsid protein
d) a first adenovirus capsid protein
e) a first nanoparticle and a second nanoparticle
f) a first nanoparticle.

31. The system of any one of the preceding claims, wherein the target tissue is selected from liver, lung, kidney, skin, stem cell, hematopoietic stem cell, blood cell, immune cell, T cell, NK cell; such as mammalian: liver, lung, kidney, skin, stem cell, hematopoietic stem cell, blood cell, immune cell, T cell, NK cell; such as human: liver, lung, kidney, skin, stem cell, hematopoietic stem cell, blood cell, immune cell, T cell, NK cell.

32. The system of any one of the preceding claims, wherein the heterologous object sequence encodes a polypeptide of at least 25, 50, 100, 150, 200, 250, 300, 400, 500, 600, 700, 800, 900, 1000 residues, or more.

33. The system of any one of the preceding claims, wherein the heterologous object sequence encodes an enzyme (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, a storage protein, and immune receptor, a synthetic protein (e.g. a chimeric antigen receptor), an antibody, or combinations thereof.

34. The system of any one of the preceding claims, wherein the heterologous object sequence comprises a sequence selected from:
i. a tissue specific promoter or enhancer;
ii. a non-coding RNA, such as regulatory RNA, a microRNA, an siRNA, an antisense RNA;
iii. a polyadenylation sequence;
iv. a splice signal;
v. a sequence encoding a polypeptide of greater than 250, 300, 400, 500, or 1,000 amino acids, and optionally up to 7,500 amino acids;
vi. a sequence encoding a fragment of a mammalian gene but does not encode the full mammalian gene, e.g., encodes one or more exons but does not encode a full-length protein;
vii. a sequence encoding one or more introns;
viii. a sequence encoding a polypeptide other than a GFP, e.g., is other than a fluorescent protein or is other than a reporter protein;
ix. is other than a sequence encoding ornithine transcarbamylase, arginosuccinate synthase, ABCB4;
x. is other than a sequence encoding factor ix;
xi. is other than CFTR;
xii. or a combination of the foregoing.

35. The system of any one of the preceding claims further comprising a pharmaceutically acceptable carrier or diluent.

36. A method of making the system of any one of claims 27-34, comprising transforming an AAV packaging cell line with a nucleic acid encoding (a), (b), or (a) and (b) and collecting the first rAAV capsid protein, second rAAV, or first and second rAAV capsid protein and associated nucleic acid(s).

37. An AAV packaging cell line comprising a nucleic acid encoding (a), (b), or (a) and (b) of the system of any one of the preceding claims.

38. A method of modifying a target DNA strand in a cell, tissue or subject, comprising administering the system of any preceding claim to the cell, tissue or subject, wherein the system inserts the heterologous object sequence into the target DNA strand, thereby modifying the target DNA strand.

39. The method of claim 38, wherein the heterologous object sequence is expressed in the cell, tissue, or subject.

40. The method of claim 38 or 39, wherein the cell, tissue or subject is a mammalian (e.g., human) cell, tissue or subject.

41. The method of any one of the preceding claims, wherein the cell is a hepatocyte.

42. The method of any one of the preceding claims, wherein the cell is lung epithelium.

43. The method of any one of the preceding claims, wherein the cell is an ionocyte.

44. The method of any one of the preceding claims, wherein the cell is a primary cell.

45. The method of any one of the preceding claims, where in the cell is not immortalized.

46. A method of treating a mammalian tissue comprising administering the system of any one of claims 1-35 to the mammal, thereby treating the tissue, wherein the tissue is deficient in the heterologous object sequence.

47. The method of any one of the preceding claims, wherein the transposase nucleic acid is present transiently.

48. The method of any one of the preceding claims, wherein the heterologous object sequence is expressed permanently.

49. An isolated nucleic acid a template nucleic acid comprising i) a sequence specifically bound by a transposase ii) a heterologous object sequence, the heterologous object sequence comprising one or more first tissue-specific expression-control sequences specific to a target tissue, optionally wherein the one or more first tissue-specific expression-control sequences specific to the target tissues comprise a sequence selected from Table 2 or Table 3, wherein the one or more first tissue-specific expression-control sequences specific to the target tissue are in operative association with the heterologous object sequence.

50. An isolated nucleic acid a template nucleic acid comprising i) a sequence specifically bound by a transposase ii) a heterologous object sequence, the heterologous object sequence comprising a sequence selected from Table 4, or all or a fragment of any of the following genes: SERPINA1, CFTR, DNAI1, DNAH5, ARMC4, CCDC39, CCDC40, CCDC65, CCDC103, CCDC114, CFAP298, DNAAF1, DNAAF2, DNAAF3, DNAAF4, DNAAF5, DNAH8, DNAH11, DNAI2, DNAL1, DRC1, HYDIN, LRRC6, NME8, OFD1, RPGR, RSPH1, RSPH4A, RSPH9, SPAG1, ZMYND10, or SFTPB, the heterologous object sequence further comprising one or more first tissue-specific expression-control sequences specific to a target tissue, optionally wherein the one or more first tissue-specific expression-control sequences specific to the target tissues comprise a sequence selected from Table 2 or Table 3.

51. A method of modifying a target DNA strand in a cell, tissue, or subject, the method comprising providing a system comprising:
a) an mRNA encoding a DNA transposase, wherein the mRNA is formulated as a lipid nanoparticle (LNP); and
b) a template nucleic acid comprising i) a sequence that specifically binds the transposase, and ii) a heterologous object sequence, wherein the template nucleic acid is associated with an AAV capsid protein; and
administering the system to the cell, tissue, or subject, wherein the system inserts the heterologous object sequence into the target DNA strand, thereby modifying the target DNA strand.

52. A method of modifying a target DNA strand in a cell, tissue, or subject, the method comprising providing a system comprising:
a) an mRNA encoding a DNA transposase, wherein the mRNA is formulated as a lipid nanoparticle (LNP); and
b) a template nucleic acid comprising i) a sequence that specifically binds the transposase, and ii) a heterologous object sequence, wherein the template nucleic acid is associated with a viral capsid protein, e.g., an AAV capsid protein, e.g., a recombinant adeno-associated virus (rAAV) capsid protein; and
administering the system to the cell, tissue, or subject, wherein the system inserts the heterologous object sequence into the target DNA strand, thereby modifying the target DNA strand.

53. A system comprising:
a) an mRNA encoding a DNA transposase, wherein the mRNA is formulated as a lipid nanoparticle (LNP); and
b) a template nucleic acid comprising i) a sequence that specifically binds the transposase, and ii) a heterologous object sequence, wherein the template nucleic acid is associated with a viral capsid protein, e.g., an AAV capsid protein, e.g., a recombinant adeno-associated virus (rAAV) capsid protein
wherein the system optionally further comprises a pharmaceutically acceptable carrier or diluent.
